(19) United States
(12) Patent Application Publication
(10) Pub. No.: US 2026/0115326 A1
(43) Pub. Date: Apr. 30, 2026

(54) LIPID NANOPARTICLES WITH RNA FOR COSMETIC TREATMENT

(71) Applicant: Lipovectra Inc., San Antonio, TX (US)

(72) Inventors: Jason Bourgeois, San Antonio, TX (US); Frank Ho Pak LAU, River Ridge, LA (US)

(73) Assignee: Lipovectra Inc., San Antonio, TX (US)

(21) Appl. No.: 19/431,261

(22) Filed: Dec. 23, 2025

Related U.S. Application Data
(63) Continuation-in-part of application No. PCT/US25/31590, filed on May 30, 2025.
(60) Provisional application No. 63/653,319, filed on May 30, 2024.
(60) Provisional application No. 63/771,940, filed on Mar. 14, 2025.
(60) Provisional application No. 63/771,940, filed on Mar. 14, 2025.
(60) Provisional application No. 63/868,661, filed on Aug. 22, 2025.

(51) Int. Cl. A61K 48/00; A61K 38/17

(52) U.S. Cl. CPC A61K 48/0058; A61K 38/17; A61K 48/0041

(57) ABSTRACT

A method for directing a change in cell state of white adipose tissue (WAT) which involve treating WAT cells with mRNA encapsulated inside lipid nanoparticles (LNPs). The mRNA encodes UCP1 or other proteins that may transition WAT to brown adipose tissue (BAT). The LNP may contain (C14-4:NCL:Cholesterol:PEG/Lipid Conjugate) at the molar ratio of (35:16:46.5:2.5), (50:10:38.5:1.5) or (56.5:10:31.8:1.7).

### SEQUENCE LISTING

The instant application contains a Sequence Listing which has been submitted electronically in XML file format and is hereby incorporated by reference in its entirety. Said XML copy, created on Dec. 23, 2025, is named 12447_012652-US3_SL.xml and is 241,154 bytes in size.

### BACKGROUND

Traditionally, adipose tissue is categorized into two main types with distinct functions: white adipose tissue (WAT) and brown adipose tissue (BAT). WAT, composed primarily of large, unilocular adipocytes, serves as the body's principal site for storing energy in the form of triglycerides. Conversely, BAT is specialized for energy expenditure through non-shivering thermogenesis, a process driven by the unique mitochondrial protein, Uncoupling Protein 1 (UCP1). Dysfunction within these adipose depots, particularly the excessive expansion and inflammation of WAT characteristic of overweight/obesity, is intimately linked to the development of metabolic disorders and diseases, including impaired glucose tolerance, metabolic syndrome, type 2 diabetes, cardiovascular disease, non-alcoholic fatty liver disease, and at least thirteen different types of cancer. Therefore, understanding and manipulating adipose tissue function represents a significant frontier in treating human disease.

A paradigm shift in adipose tissue biology occurred with the confirmation of metabolically active BAT in adult humans. That paradigm shift was further expanded by the discovery of inducible “browning” or “beigeing” of WAT. Beige adipose tissue (BeAT) is defined by the emergence or development of cells, in WAT, that express UCP1. The “beigeing” process can be activated by specific stimuli such as chronic cold exposure or treatment with 8-adrenergic agonists. Like classical brown adipocytes, beige cells possess multilocular lipid droplets, abundant mitochondria, and express UCP1, enabling them to consume caloric fuel via heat production. This process increases the body's thermogenic capacity.

UCP1, also known historically as thermogenin, is an inner mitochondrial membrane protein, primarily found in brown and beige adipocytes. UCP1 provides an alternative pathway for proton re-entry into the mitochondrial matrix, effectively bypassing ATP synthase. This regulated “proton leak” dissipates the energy stored in the proton motive force (PMF) directly as heat, rather than converting it into chemical energy in the form of ATP. This conversion of the PMF to heat is the core mechanism of non-shivering thermogenesis mediated by brown and beige fat.

There is an unmet need in the art for an agent to induce UCP1 expression in WAT and other tissues, thereby bypassing natural upstream controls. This strategy offers the potential advantage of inducing thermogenesis independent of external stimuli like cold, while also circumventing the body's inherent negative controls on upregulating heat production. An ideal agent would thus increase local energy expenditure by generating heat and thereby reduce the size of adipose tissue deposits.

### SUMMARY

To address the above-mentioned unmet needs, the present technology presents methods, compositions and systems for directing a change in the cell state of human WAT to BeAT, agents to achieve that change and systems/protocols for taking advantage of the thermogenic and calorie-expending properties of BeAT in order to achieve local reductions of adipose tissue and systemic change in the properties of adipose tissue.

The present disclosure provides an RNA molecule comprising (consisting essentially of, or consisting of) a nucleotide sequence about 80% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleotide sequence set forth in any of SEQ ID NOs:1 to 130, including any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, and SEQ ID NO:130.

Also encompassed by the present disclosure is a lipid nanoparticle comprising (i) an RNA molecule encoding human uncoupling protein 1 (UCP1); and (ii) an ionizable cationic lipid (ICL), a non-cationic lipid (NCL), a sterol, and a polyethylene glycol (PEG)-modified lipid, at a molar ratio of ICL (20-80):NCL (2-40):sterol (10-70):PEG-modified lipid (0.2-10); at a molar ratio of ICL (25-75):NCL (4-35):sterol (15-65):PEG-modified lipid (0.5-8); at a molar ratio of ICL (30-70):NCL (6-30):sterol (20-60):PEG-modified lipid (0.5-6); at a molar ratio of ICL (35-65):NCL (8-25):sterol (25-55):PEG-modified lipid (1-4); at a molar ratio of ICL (35-60):NCL (10-16):sterol (30-50):PEG-modified lipid (1.5-2.5); or at a molar ratio of ICL (40-50):NCL (9-10):sterol (35-45):PEG-modified lipid (1.5-2.5).

The RNA molecule may comprise (consist essentially of, or consist of) a nucleotide sequence about 80% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleotide sequence set forth in any of SEQ ID NOs:1 to 130.

In certain embodiments, one or more uridine nucleotides of the RNA molecule are substituted with pseudouridines. In certain embodiments, all uridine nucleotides of the RNA molecule are substituted with pseudouridines. In certain embodiments, at least or about 10%, at least or about 20%, at least or about 30%, at least or about 40%, at least or about 50%, at least or about 60%, at least or about 70%, at least or about 80%, at least or about 90%, at least or about 95%, at least or about 96%, at least or about 97%, at least or about 98%, at least or about 99%, or about 100%, uridine nucleotides of the RNA molecule are substituted with pseudouridines.

In certain embodiments, one or more uridine nucleotides of the RNA molecule are substituted with N1-Methylpseudouridines. In certain embodiments, all uridine nucleotides of the RNA molecule are substituted with N1-Methylpseudouridines. In certain embodiments, at least or about 10%, at least or about 20%, at least or about 30%, at least or about 40%, at least or about 50%, at least or about 60%, at least or about 70%, at least or about 80%, at least or about 90%, at least or about 95%, at least or about 96%, at least or about 97%, at least or about 98%, at least or about 99%, or about 100%, uridine nucleotides of the RNA molecule are substituted with N1-Methylpseudouridines.

The RNA molecule may be a messenger RNA (mRNA).

Ionizable cationic lipids (ICLs) include, but are not limited to, C14-4, SM-102, ALC-0315, DLin-MC3-DMA, LP-01, 4A3-SC8, cKK-E12, 306ᵢ₁₀, C3-K2-E14, Lipid A9, OF-02, TCL053, DLin-DMA, C12-200, DLin-KC2-DMA, and mixtures thereof.

Non-cationic lipids (NCLs) include, but are not limited to, distearoylphosphatidylcholine (DSPC), dioleoylphosphandylcholine (DOPC), dipalmitoylphosphatidylcholine (DPPC), dioleoylphosphatidylglycerol (DOPG), dipalmitoylphosphatidlylglycerol (DPPG), dioleoylphosphatidylethanolamine (DOPE), palmitoyloleoylphosphatidylcholine (POPC), palmitoyloleol-phosphatidylethanolamine (POPE). dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanoiamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, and mixtures thereof.

Sterols include, but are not limited to, cholesterol, fecosterol, sitosterol, ergosterol, campesterol, stigmasterol, brassicasterol, tomatidine, tomatine, ursolic acid, alpha-tocopherol, and mixtures thereof.

PEG-modified lipids include, but are not limited to, a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

Non-limiting examples of PEG-lipids include 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

The present disclosure provides a pharmaceutical composition comprising: a synthetic mRNA comprising (consisting essentially of, or consisting of) the nucleotide sequence set forth in SEQ ID NO: 6, SEQ ID NO: 3, or SEQ ID NO: 4, wherein the synthetic mRNA is packaged in a lipid nanoparticle (LNP).

In certain embodiments, the lipid nanoparticle comprises (C14-4:NCL:cholesterol PEG-modified lipid) at a molar ratio of (35:16:46.5:2.5), (50:10:38.5:1.5) or (56.5:10:31.8:1.7).

In certain embodiments, the lipid nanoparticle comprises (SM-102:NCL:Cholesterol:PEG-modified lipid) at a molar ratio of (50:10:38.5:1.5), (48.3:9.1:40.5:2.1) or (46.6:10.4:41.2:1.8).

The present disclosure further provides a pharmaceutical composition comprising the RNA molecule. The present disclosure also provides a pharmaceutical composition comprising the lipid nanoparticle.

The present disclosure provides a method of treating a condition in a subject. The method comprises administering the present RNA molecule, lipid nanoparticle, or pharmaceutical composition to the subject.

The condition may be overweight/obesity. The condition may be a metabolic disorder. The conditions include, but are not limited to, impaired glucose tolerance, metabolic syndrome, diabetes (e.g., type 2 diabetes), cardiovascular diseases, non-alcoholic fatty liver disease, lipedema, cancer (such as breast cancer, colon cancer, melanoma, and prostate cancer), hypertension (e.g., treatment-resistant hypertension), and heart failure (e.g., heart failure with preserved ejection fraction).

The present disclosure provides a method of treating a subject for targeted body fat reduction in an area of the subject. The method comprises administering the present RNA molecule, lipid nanoparticle, or pharmaceutical composition to the area of the subject.

The present disclosure provides methods and compositions for increasing the quantities and/or the ratios of brown/beige adipocytes in WAT. In various aspects, the cellular manipulations described herein are guided and/or mediated by messenger RNA (mRNA) that may be in vitro transcribed (“IVT” or “synthetic”). Proteins encoded by the mRNA can provide cellular instructions for transitioning a cell from one state to a different cellular state. In one embodiment, lipid nanoparticle (LNP)-packaged synthetic mRNA is introduced into cells that reside in WAT (WAT-cells). Examples of WAT-cells include, but are not limited to, adipocytes, mesenchymal stem cells, stromal cells, fibroblasts, vascular cells, perivascular cells, nerve cells, and immune cells.

An aspect of the present disclosure relates to a method for generating UCP1-positive WAT-cells. This converts WAT (a tissue comprised of many WAT-cells) into beige adipose tissue (BeAT) or brown adipose tissue (BAT). BeAT may include beige adipocytes or brown adipocytes. The method may comprise, contacting WAT-cells with mRNA encapsulated in an LNP, wherein the mRNA encodes UCP1, or other proteins/enzymes (e.g. Adipose Triglyceride Lipase and Hormone-Sensitive Lipase to mobilize fatty acids from triglycerides; Creatine kinase and phosphocreatine phosphatase to promote futile creatine cycling) that may induce UCP1 protein production in WAT-cells.

An aspect of the present disclosure relates to a pharmaceutical composition comprising: an mRNA encoding UCP1, said mRNA encapsulated in an LNP. The pharmaceutical composition may be introduced into WAT-cells.

An aspect of the present disclosure relates to a composition of a treatment for directing a change in state of any WAT-cells comprising: contacting said WAT-cells with a payload of mRNA, protein, plasmid DNA or oligonucleic acid encapsulated in MITO-Porter (a liposome-based carrier for delivering a payload to the mitochondria via membrane fusion) for targeting the payload to the mitochondria. The payload may effectuate a state change transition of the WAT-cells to UCP1-positive.

In one embodiment, the disclosure may be a pharmaceutical composition comprising an mRNA comprising a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in SEQ ID NO: 130. The mRNA may be packaged in a lipid nanoparticle (LNP).

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NO: 1 to SEQ ID NO: 130.

In another embodiment, the LNP is of an LNP component molar ratio substantially similar to that of LNP ID I (Table 1).

In another embodiment, the LNP is of an LNP component molar ratio substantially similar to that of LNP ID D (Table 1).

In another embodiment, the mRNA comprises one or more modified uridine nucleotides.

In another embodiment, the modified uridine nucleotide is pseudouridine.

In another embodiment, the modified uridine nucleotide is N1-Methylpseudouridine.

In a second embodiment, the disclosure provides a method of preparing a pharmaceutical composition, the method comprising preparing a synthetic mRNA comprising a nucleotide sequence set forth in SEQ ID NO: 2, SEQ ID NO: 8, or SEQ ID NO: 18; and packaging said synthetic mRNA in a lipid nanoparticle (LNP).

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NO: 2, SEQ ID NO: 8, or SEQ ID NO: 18.

In another embodiment, the LNP is of an LNP component molar ratio substantially similar to that of LNP ID I (Table 1).

In another embodiment, the LNP is of an LNP component molar ratio substantially similar to that of LNP ID D (Table 1).

In another embodiment, the mRNA comprises one or more modified uridine nucleotides.

In another embodiment, the modified uridine nucleotide is pseudouridine.

In another embodiment, the modified uridine nucleotide is N1-Methylpseudouridine.

In a third embodiment, the disclosure provides a method of treating a subject for targeted body fat reduction in an area of the subject. The method may comprise delivering to the area a pharmaceutical composition comprising an mRNA; wherein the mRNA comprises a nucleotide sequence at least or about 70% identical to the nucleotide sequence set forth in SEQ ID NO: 3, SEQ ID NO: 9, or SEQ ID NO: 17.

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NO: 3, SEQ ID NO: 9, or SEQ ID NO: 17.

The method may further comprise identifying an area of the subject for targeted fat reduction.

In another embodiment, the area comprises the abdomen, the outer arms, flanks, upper back, lumbar region, calves, submental fat, dorsal cervical fat, axillary fat, lateral chest wall, outer thighs, inner thighs, or combinations thereof.

In another embodiment, the delivery is carried out in a single bolus injection.

In another embodiment, the delivery is carried out in a series of injections in the area.

In another embodiment, the delivery is carried out transdermally by a penetration enhancer.

In another embodiment, the delivery is carried out transdermally by a vesicular carrier.

In another embodiment, the delivery is carried out transdermally by a microneedle injection.

In another embodiment, the delivery is carried out transdermally by iontophoresis, sonophoresis or jet injection.

In one embodiment, the present disclosure provides a pharmaceutical composition comprising UCP1 protein (NCBI RefSeq: NP_001735.1) encapsulated in MITO-Porter. WO/2006/095837. U.S. Ser. No. 11/814,640. Yamada et al., Dual function MITO-Porter, a nano carrier integrating both efficient cytoplasmic delivery and mitochondrial macromolecule delivery, Mol Ther. 2011, 19(8):1449-56. Cojocaru et al., Mitochondrial Dysfunction, Oxidative Stress, and Therapeutic Strategies in Diabetes, Obesity, and Cardiovascular Disease, Antioxidants (Basel), 2023; 12(3):658. Norota et al., Lipid nanoparticle delivery of the CRISPR/Cas9 system directly into the mitochondria of cells carrying m.7778G>T mutation in MtDNA (mt-Atp8), Scientific Reports, volume 15, Article number: 18717 (2025). Yasuzaki et al., Mitochondrial matrix delivery using MITO-Porter, a liposome-based carrier that specifies fusion with mitochondrial membranes, Biochem. Biophys. Res. Commun. 2010; 397(2):181-6. Yamada et al., MITO-Porter: A liposome-based carrier system for delivery of macromolecules into mitochondria via membrane fusion, Biochim. Biophys. Acta. 2008; 1778(2):423-32. Yamada et al., Mitochondrial drug delivery systems for macromolecule and their therapeutic application to mitochondrial diseases, Adv. Drug Deliv. Rev. 2008; 60(13-14):1439-62.

In another embodiment, the UCP1 protein may be optimized to increase its active life by including factors which inhibit the effects of mitochondrial proteases LONP1 and ClpXP.

In another embodiment, the UCP1 protein may be optimized to increase its binding affinity to the inner mitochondrial matrix by optimizing the mitochondrial targeting signal (MTS) of the UCP1 protein, introduction of destabilizing elements, and/or the introduction of chaperone binding sites.

### BRIEF DESCRIPTION OF THE DRAWINGS

FIG. 1 is a microscope image of human adipose tissue transfected with the mRNA of SEQ ID NO: 130 encoding UCP1 encapsulated in LNPs.

FIG. 2 is a first and a last frame of a 6-day time lapse showing a negative control study of untreated human adipose tissue in culture.

FIG. 3 is a first and a last frame of a 6-day time lapse showing a full dose study (Example 3) of human white adipose tissue transfected with an mRNA construct expressing UCP1. The mRNA caused shrinkage of said white adipose tissue.

FIG. 4 is a first and a last frame of a 6-day time lapse showing a half dose study (Example 4) of human adipose tissue transfected with an mRNA construct expressing UCP1. The mRNA caused shrinkage of said white adipose tissue.

FIG. 5 is a Western Blot result showing relative protein expression levels for varying doses of an mRNA construct of SEQ ID NO: 130 expressing UCP1 in human adipose tissue.

FIG. 6 is a Western Blot result showing relative protein expression levels for varying doses of an mRNA construct of SEQ ID NO: 130 expressing UCP1 encapsulated in LNP I in human adipose tissue.

FIG. 7 is a series of Western Blot results showing protein expression by cells derived from human white adipose tissue transfected with mRNA constructs of SEQ ID NO: 2 through SEQ ID NO: 21.

FIG. 8, FIG. 9, FIG. 10, FIG. 11, FIG. 12, FIG. 13, and FIG. 14 show shrinkage of the transfected human WAT in one day intervals from the start to the end point at t+6 days after treatment with a 1 μg dose of mRNA of SEQ ID NO: 130. FIG. 8: t=0; FIG. 9: t+1 day; FIG. 10: t+2 days; FIG. 11: t+3 days; FIG. 12: t+4 days; FIG. 13: t+5 days; FIG. 14: t+6 days.

FIG. 15, FIG. 16, FIG. 17, FIG. 18, FIG. 19, FIG. 20, and FIG. 21 show shrinkage of the transfected human WAT in one day intervals from the start to the end point at t+6 days after treatment with a 5 μg dose of mRNA of SEQ ID NO: 130. FIG. 15: t=0; FIG. 16: t+1 day; FIG. 17: t+2 days; FIG. 18: t+3 days; FIG. 19: t+4 days; FIG. 20: t+5 days; FIG. 21: t+6 days.

FIG. 22 is a Western Blot result showing dose responsiveness of UCP1 expression 24 hours after transfection of cells derived from human white adipose tissue with 5 μg and 10 μg doses of SEQ ID NO: 130 encapsulated in LNP D.

FIG. 23 is a Western Blot result showing UCP1 expression 24 hours after transfection of cells derived from human white adipose tissue with 1 μg of SEQ ID NO: 130 encapsulated in LNP I.

FIG. 24 is a Western Blot Result showing UCP1 expression 24 hours after transfection of cells derived from human white adipose tissue with 1 μg of mRNA of SEQ ID NOs: 2 through 21 encapsulated with MessengerMAX™ transfection agent.

FIG. 25 is a Western Blot Result showing UCP1 expression 24 hours after transfection of cells derived from human white adipose tissue with 1 μg of mRNA of SEQ ID NOs: 130 and 2 through 16, encapsulated in LNP I. SEQ ID NOs: 130, 1, 6, and 13 were run in triplicate.

FIG. 26 is a Western Blot Result showing UCP1 expression at 24, 48, 72 and 96 hours after transfection of cells derived from human white adipose tissue with 1p g of mRNA of SEQ ID NOs: 3, 4, 6, 11, 13, 14, 16, and 130.

FIG. 27 is a series of time lapse images of cells derived from human white adipose tissue beginning at 24 hours after transfection with 10 μg of mRNA of SEQ ID NO: 2 encapsulated in LNP ID I (C14-4). Top row, from left to right: Day 1, Day 2, Day 3. Bottom row, from left to right: Day 4, Day 5, Day 6.

FIG. 28 is a series of time lapse images of cells derived from human white adipose tissue beginning at 24 hours after transfection with 10 μg of mRNA of SEQ ID NO: 3 encapsulated in LNP ID I (C14-4). Top row, from left to right: Day 1, Day 2, Day 3. Bottom row, from left to right: Day 4, Day 5, Day 6.

FIG. 29 is a series of time lapse images of cells derived from human white adipose tissue beginning at 24 hours after transfection with 10 μg of mRNA of SEQ ID NO: 4 encapsulated in LNP C14-4. Top row, from left to right: Day 1, Day 2, Day 3. Bottom row, from left to right: Day 4, Day 5, Day 6.

FIG. 30 is a series of time lapse images of cells derived from human white adipose tissue beginning at 24 hours after transfection with 2 μg of mRNA of SEQ ID NO: 6 encapsulated in LNP C14-4. Top row, from left to right: Day 1, Day 2, Day 3. Bottom row, from left to right: Day 4, Day 5, Day 6.

FIG. 31 is a series of time lapse images of cells derived from human white adipose tissue beginning at 24 hours after transfection with 10 μg of mRNA of SEQ ID NO: 6 encapsulated in LNP C14-4. Top row, from left to right: Day 1, Day 2, Day 3. Bottom row, from left to right: Day 4, Day 5, Day 6.

FIG. 32 is a series of time lapse images of cells derived from human white adipose tissue beginning at 24 hours after transfection with 10 μg of mRNA of SEQ ID NO: 14 encapsulated in LNP C14-4. Top row, from left to right: Day 1, Day 2, Day 3. Bottom row, from left to right: Day 4, Day 5, Day 6.

FIG. 33 is a series of time lapse images of cells derived from human white adipose tissue beginning at 24 hours after transfection with 2 μg of mRNA of SEQ ID NO: 16 encapsulated in LNP C14-4. Top row, from left to right: Day 1, Day 2, Day 3. Bottom row, from left to right: Day 4, Day 5, Day 6.

### DETAILED DESCRIPTION

Detailed descriptions of one or more embodiments are provided herein. It is to be understood, however, that the present disclosure may be embodied in various forms. Therefore, specific details disclosed herein are not to be interpreted as limiting, but rather as a basis for the claims and as a representative basis for teaching one skilled in the art to employ the present invention in any appropriate manner.

UCP1-positive cells, including brown and/or beige adipocytes (BAs), are able to uncouple the proton motive force from ATP generation in order to produce thermal energy. This mechanism allows mammals, through non-shivering thermogenesis, to regulate body temperature in cold environments. Because of its metabolically active nature, UCP1-positive WAT-cells represent a promising pathway for non-surgical intervention for diseases whose pathophysiology is driven by excess fat. Examples of such diseases include obesity, diabetes, lipedema, cancer (such as breast cancer, colon cancer, melanoma, and prostate cancer), hypertension (e.g., treatment-resistant hypertension), heart failure (e.g., heart failure with preserved ejection fraction), and other indications.

Provided herein are compositions, methods, formulations and devices to: 1) increase UCP1 levels in WAT-cells, thereby enhancing the metabolic activity of the treated tissue, and 2) activate the thermogenic activity of the BAs in the treated tissue so as to consume fatty acids through thermogenesis thereby reducing the volume of adipose tissue in the treated tissue.

Referring to the Sequence Listing incorporated by reference herein, SEQ ID NO:130 is an optimized version of the mRNA encoding for human UCP1, including modifications to the 5′ and 3′ UTRs such as inserting a ribosome binding site, a Kozak sequence and a double stop codon. SEQ ID NOs: 2-21 were created as further optimizations of SEQ ID NO: 130 using GenScript Biotech Corporation GenSmart tool with several modifications including modifications selected from deleting the 5′ UTR, incorporating Translation Initiator of 5′ UTR (TISU), the genomic human UCP1 5′ UTR, the consensus genomic human fatty acid binding protein 4 (FABP4) 5′ UTR, the consensus genomic human UCP1 3′ UTR, the Cominarty 3′ UTR, and the consensus genomic human beta-globin (HBB) 3′ UTR in tandem tail-to-head sequence. For example, SEQ ID NO: 2 is 928 nucleotides long which corresponds to nucleotides 54 through 981 of SEQ ID NO: 130. As another example, SEQ ID NO: 3 is 932 nucleotides long which corresponds to nucleotides 50 through 981 of SEQ ID NO: 130. As another example, SEQ ID NO: 8 is 1397 nucleotides long whose nucleotides 1 through 929 correspond to nucleotides 50 through 978 of SEQ ID NO: 130. As another example, SEQ ID NO: 17 is 1142 nucleotides long whose nucleotides 1 through 928 correspond to nucleotides 54 through 981 of SEQ ID NO: 130.

SEQ ID NOs: 22 through 129 are optimizations of SEQ ID NOs: 130 and 2 through 21 using various UTR sequences. The optimizations of the UTR regions of SEQ ID NOs: 22 through 129 are designed to prevent the formation of secondary and tertiary RNA structures that can inhibit translation. The 5′ UTR sequences were selected to produce high protein expression level in human WAT.

SEQ ID NOs: 22 through 24: codon optimized human UCP1 mRNA using custom 5′ and 3′ UTRs. The 5′ UTR: Modified genomic human FABP4 5′ UTR with a Kozak sequence inserted. The 3′ UTR: double stop codon.

SEQ ID NOs: 25 through 27: codon optimized human UCP1 mRNA using custom 5′ and 3′ UTRs. The 5′ UTR: Modified genomic human LIPE 5′ UTR with a Kozak sequence inserted. The 3′ UTR: double stop codon.

SEQ ID NOs: 28 through 30: codon optimized human UCP1 mRNA using custom 5′ and 3′ UTRs. The 5′ UTR: Modified genomic human PLIN1 5′ UTR with a Kozak sequence inserted. The 3′ UTR: double stop codon.

SEQ ID NOs: 31 through 33: codon optimized human UCP1 mRNA using custom 5′ and 3′ UTRs. The 5′ UTR: Modified genomic human COL1A 5′ UTR with a Kozak sequence inserted. The 3′ UTR mRNA: double stop codon.

SEQ ID NOs: 34 through 36: codon optimized human UCP1 mRNA using custom 5′ and 3′ UTRs. The 5′ UTR: Modified genomic human LEP 5′ UTR with a Kozak sequence inserted. The 3′ UTR for these CDS: double stop codon.

SEQ ID NOs: 37 through 39: codon optimized human UCP1 mRNA using custom 5′ and 3′ UTRs. The 5′ UTR: Modified genomic human ADIPOQ 5′ UTR with a Kozak sequence inserted. The 3′ UTR: double stop codon.

SEQ ID NOs: 40 through 44: codon optimized human UCP1 mRNA using custom 5′ and 3′ UTRs. The 5′ UTR: Consensus genomic human FABP4 5′ UTR. The 3′ UTR: double stop codon.

SEQ ID NOs: 45 through 49: codon optimized human UCP1 mRNA using custom 5′ and 3′ UTRs. The 5′ UTR: Consensus genomic human Lipase E, Hormone Sensitive Type (LIPE) 5′ UTR. The 3′ UTR: double stop codon.

SEQ ID NOs: 50 through 54: codon optimized human UCP1 mRNA using custom 5′ and 3′ UTRs. The 5′ UTR: Consensus genomic human perilipin 1 (PLIN1) 5′ UTR. The 3′ UTR: double stop codon.

SEQ ID NOs: 55 through 59: codon optimized human UCP1 mRNA using custom 5′ and 3′ UTRs. The 5′ UTR: Consensus genomic human collagen 1A (COL1A) 5′ UTR. The 3′ UTR: double stop codon.

SEQ ID NOs: 60 through 64: codon optimized human UCP1 mRNA using custom 5′ and 3′ UTRs. The 5′ UTR: Consensus genomic human leptin (LEP) 5′ UTR. The 3′ UTR: double stop codon.

SEQ ID NOs: 65 through 69: codon optimized human UCP1 mRNA using custom 5′ and 3′ UTRs. The 5′ UTR: Consensus genomic human adiponectin (ADIPOQ) 5′ UTR. The 3′ UTR: double stop codon.

SEQ ID NOs: 70 through 74: codon optimized human UCP1 mRNA using custom 5′ and 3′ UTRs. The 5′ UTR: Modified genomic human FABP4 5′ UTR with a Kozak sequence inserted. The 3′ UTR: double stop codon.

SEQ ID NOs: 75 through 79: codon optimized human UCP1 mRNA using custom 5′ and 3′ UTRs. The 5′ UTR: Modified genomic human LIPE 5′ UTR with a Kozak sequence inserted. The 3′ UTR: double stop codon.

SEQ ID NOs: 80 through 84: codon optimized human UCP1 mRNA using custom 5′ and 3′ UTRs. The 5′ UTR: Modified genomic human PLIN1 5′ UTR with a Kozak sequence inserted. The 3′ UTR: double stop codon.

SEQ ID NOs: 85 through 89: codon optimized human UCP1 mRNA using custom 5′ and 3′ UTRs. The 5′ UTR: Modified genomic human COL1A 5′ UTR with a Kozak sequence inserted. The 3′ UTR: double stop codon.

SEQ ID NOs: 90 through 94: codon optimized human UCP1 mRNA using custom 5′ and 3′ UTRs. The 5′ UTR: Modified genomic human LEP 5′ UTR with a Kozak sequence inserted. The 3′ UTR: double stop codon.

SEQ ID NOs: 95 through 99: codon optimized human UCP1 mRNA using custom 5′ and 3′ UTRs. The 5′ UTR: Modified genomic human ADIPOQ 5′ UTR with a Kozak sequence inserted. The 3′ UTR: double stop codon.

SEQ ID NOs: 100 through 104: codon optimized human UCP1 mRNA using custom 5′ and 3′ UTRs. The 5′ UTR: Consensus genomic human FABP4 5′ UTR. The 3′ UTR: double stop codon.

SEQ ID NOs: 105 through 109: codon optimized human UCP1 mRNA using custom 5′ and 3′ UTRs. The 5′ UTR: Consensus genomic human LIPE 5′ UTR. The 3′ UTR: double stop codon.

SEQ ID NOs: 110 through 114: codon optimized human UCP1 mRNA using custom 5′ and 3′ UTRs. The 5′ UTR: Consensus genomic human PLIN1 5′ UTR. The 3′ UTR: double stop codon.

SEQ ID NOs: 115 through 119: codon optimized human UCP1 mRNA using custom 5′ and 3′ UTRs. The 5′ UTR: Consensus genomic human COL1A 5′ UTR. The 3′ UTR: double stop codon.

SEQ ID NOs: 120 through 124: codon optimized human UCP1 mRNA using custom 5′ and 3′ UTRs. The 5′ UTR: Consensus genomic human LEP 5′ UTR. The 3′ UTR: double stop codon.

SEQ ID NOs: 125 through 129: codon optimized human UCP1 mRNA using custom 5′ and 3′ UTRs. The 5′ UTR: Consensus genomic human ADIPOQ 5′ UTR. The 3′ UTR: double stop codon.

The genomic coding sequence of UCP1 for Homo sapiens is known. UCP homologs from non-human animals, such as lower primates, horses, cows, pigs, sheep, goats, and chicken can also be used with the lipid nanoparticles.

The synthetic mRNA may further comprise siRNA, circular RNA, self-replicating mRNA, or mRNA designed for gene editing, including but not limited to, CRISPR-Cas9 systems. The LNP vesicle may be tailored specifically to enhance transfection of the target adipocytes and WAT. Tailoring of the LNP may include adjusting the molar ratios of ionizable lipids, helper phospholipids, polyethylene glycol (PEG)-lipids, and sterols, as well as immunogenicity-reducing molecules including but not limited to steroids and steroid pro-drugs.

The “lipid nanoparticle” comprises one or more lipids (e.g., cationic lipids, non-cationic lipids, and/or PEG-modified lipids) which are formulated to deliver one or more mRNA to one or more target cells. Suitable lipids include, for example, phosphatidyl (e.g., phosphatidylglycerol, phosphatidylcholine, phosphatidylserine, phosphatidylethanolamine, sphingolipids, cerebrosides, and gangliosides). Various polymers may also be incorporated into the lipid nanoparticles, including, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

The lipid nanoparticles may range in size from about 20 nm to about 200 nm, about 50 nm to about 200 nm, about 60 nm to about 150 nm, about 50 nm to about 150 nm, about 80 nm to about 150 nm, about 60 nm to about 80 nm, about 80 nm to about 100 nm, about 100 nm to about 120 nm, or about 120 nm to about 150 nm.

The lipid nanoparticles can incorporate an ionizable cationic lipid (ICL) to encapsulate the mRNA and/or enhance the delivery of mRNA into the target cell. A “cationic lipid” refers to any lipid species that carry a net positive charge at a selected pH, such as physiological pH. Ionizable cationic lipids may contain one, two, three, or more fatty acid or fatty alkyl chains and a pH-titratable amino head group (e.g., an alkylamino or dialkylamino head group). An ionizable cationic lipid is typically protonated (i.e., positively charged) at a pH below the pKa of the amino head group and is substantially not charged at a pH above the pKa.

Ionizable cationic lipids include, but are not limited to, DLin-DMA, DLin-D-DMA, DLin-MC3-DMA, DLin-KC2-DMA, DODMA and amino alcohol lipids. The amino alcohol cationic lipid can be the lipids described in and/or made by the methods described in U.S. Patent Publication No. US20130150625. As a non-limiting example, the cationic lipid can be 2-amino-3-[(9Z,12Z)-octadeca-9,12-dien-1-yloxy]-2-{[(9Z,2Z)-octadeca-9,12-dien-1-yloxy]methyl}propan-1-ol; 2-amino-3-[(9Z)-octadec-9-en-1-yloxy]-2-{[(9Z)-octadec-9-en-1-yloxy]methyl}propan-1-ol; 2-amino-3-[(9Z,12Z)-octadeca-9,12-dien-1-yloxy]-2-[(octyloxy)methyl]propan-1-ol; and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol; or any pharmaceutically acceptable salt or stereoisomer thereof.

Non-limiting examples of ionizable lipids include 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(30)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), DLinDMA, (2S)-2-({8-[(30)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), and mixtures thereof. In addition to these, an ionizable cationic lipid may also be a lipid including a cyclic amine group.

U.S. Pat. No. 9,738,593 is incorporated by reference in its entirety.

Non-cationic lipids (NCL) may also be used in the lipid nanoparticles. Non-cationic lipids include, but are not limited to, distearoylphosphatidylcholine (DSPC), dioleoylphosphandylcholine (DOPC), dipalmitoylphosphatidylcholine (DPPC), dioleoylphosphatidylglycerol (DOPG), dipalmitoylphosphatidlylglycerol (DPPG), dioleoylphosphatidylethanolamine (DOPE), palmitoyloleoylphosphatidylcholine (POPC), palmitoyloleol-phosphatidylethanolamine (POPE). dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanoiamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

Such non-cationic lipids may be used alone, or may be used in combination with other excipients, for example, cationic lipids. When used in combination with a cationic lipid, the non-cationic lipid may comprise a molar percentage of 5% to about 90%, or about 10% to about 70% of the total lipid present in the lipid nanoparticle.

Polyethylene glycol (PEG)-modified lipids, such as PEG-modified phospholipids, PEG/lipid conjugates and derivatized lipids such as DMG-PEG(2000), derivatized ceramides (PEG-CER), including N-Octanoyl-Sphingosine-1-[Succinyl(Methoxy Polyethylene Glycol)-2000](C8 PEG-2000 ceramide), or ALC-0159 can be incorporated into the lipid nanoparticle. The PEG-modified lipids include, but are not limited to, a polyethylene glycol chain of up to 5 kDa covalently attached to a lipid with alkyl chain(s) of C₆-C₂₀ length. The PEG-modified lipids and derivatized lipids can be present at varying molar percentages, e.g., from about 0% to about 20%, about 0.5% to about 20%, about 1% to about 15%, about 4% to about 10%, or about 2% of the total lipids present in the lipid nanoparticle.

The terms “PEG-modified lipid” and “PEG-lipid” are used interchangeably and can refer to polyethylene glycol (PEG)-modified lipids. Non-limiting examples of PEG-lipids include PEG-modified phosphatidylethanolamine, PEG-modified phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG-lipid may be PEG-c-DOMG, PEG-DMG, PEG-DLPE, PEG-DMPE, PEG-DPPC, PEG-S-DSG, PEG-c-DMA, or a PEG-DSPE lipid.

PEG-modified lipids include, but are not limited to, a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the PEG-lipids include, but are not limited to, 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

Non-limiting examples of PEG-modified lipids include, PEG-c-DOMG (R-3-[(o-methoxy-poly(ethyleneglycol)2000)carbamoyl)]-1,2-dimyristyloxypropyl-3-amine) (also referred to herein as PEG-DOMG), PEG-DSG (1,2-Distearoyl-sn-glycerol, methoxypolyethylene glycol), PEG-DMG (1,2-Dimyristoyl-sn-glycerol), PEG-DPG (1,2-Dipalmitoyl-sn-glycerol, methoxypolyethylene glycol), and mixtures thereof.

In one embodiment, the lipid nanoparticles described herein may comprise a PEG lipid which is a non-diffusible PEG. Non-limiting examples of non-diffusible PEGs include PEG-DSG and PEG-DSPE.

In some embodiments, the lipid moiety of the PEG-lipids includes those having lengths of from about C14 to about C22, or from about C14 to about C16. In some embodiments, a PEG moiety, for example an mPEG-NH2, has a size of about 1000, 2000, 5000, 10,000, 15,000 or 20,000 daltons. In one embodiment, the PEG-lipid is PEG2k-DMG.

U.S. Pat. Nos. 10,406,113; 8,158,601 and 10,821,175 are incorporated herein by reference in their entirety.

In some embodiments, the ratio of PEG in the lipid nanoparticle (LNP) is increased or decreased and/or the carbon chain length of the PEG lipid is modified from C14 to C18 to alter the pharmacokinetics and/or biodistribution of the LNP. As a non-limiting example, LNPs contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-modified lipid.

In certain embodiments, to package mRNA into an LNP, a lipid mixture of four components (ICL, NCL, Cholesterol, PEG/Lipid Conjugate) is dissolved in ethanol and is rapidly mixed with an mRNA solution in an acidic aqueous buffer. As ethanol is diluted by the aqueous buffer, the change in solvent polarity drives the self-assembly of the lipids and encapsulated mRNA into a core-shell nanostructure. Subsequently, the acidic buffer is exchanged with PBS to achieve a neutral pH, neutralizing the ionizable lipid, and stabilizing the final LNP formulation. This process yields nanoparticles that protect the mRNA payload and facilitates its delivery into target cells.

To reduce immunogenicity and inflammation, active and pro-drug forms of anti-inflammatory steroids can be incorporated into the LNPs. For example, the ester prodrugs of rofleponide and/or budesonide can be incorporated into an LNP using amino lipids such as Dlin-MC3-DMA or L608.

LNPs may include the following lipids shown in Table 1.

TABLE 1

| | | LNP Component Molar Ratios | | | |
|---|---|---|---|---|---|
| LNP | | | | | PEG/Lipid |
| ID | ICL Molecule | ICL | NCL | Cholesterol | Conjugate |
| A | ALC-0315 | 50 | 10 | 38.5 | 1.5 |
| | | 46.3 | 9.4 | 42.7 | 1.6 |
| | | 47.2 | 9.2 | 42.2 | 1.4 |
| B | DLin-MC3-DMA | 50 | 10 | 38.5 | 1.5 |
| | | 51.2 | 10 | 37.4 | 1.4 |
| | | 49.4 | 11.2 | 37.8 | 1.6 |
| C | LP-01 | 45 | 9 | 44 | 2 |
| | | 43.5 | 10 | 44.3 | 2.2 |
| | | 46.3 | 9.4 | 42.3 | 2 |
| D | SM-102 | 50 | 10 | 38.5 | 1.5 |
| | | 48.3 | 9.1 | 40.5 | 2.1 |
| | | 46.6 | 10.4 | 41.2 | 1.8 |
| E | 4A3-SC8 | 38.5 | 30 | 30 | 1.5 |
| | | 35 | 16 | 46.5 | 2.5 |
| | | 45 | 11 | 42.5 | 1.5 |
| F | cKK-E12 | 35 | 16 | 46.5 | 2.5 |
| | | 45 | 11 | 42.5 | 1.5 |
| | | 50 | 10 | 38.5 | 1.5 |
| G | 306Oi10 | 35 | 16 | 46.5 | 2.5 |
| | | 50 | 10 | 38.5 | 1.5 |
| | | 40 | 32.5 | 25 | 2.5 |
| H | C3-K2-E14 | 50 | 10 | 38.5 | 1.5 |
| | | 56.5 | 10 | 31.8 | 1.7 |
| | | 35 | 16 | 46.5 | 2.5 |
| I | C14-4 | 35 | 16 | 46.5 | 2.5 |
| | | 50 | 10 | 38.5 | 1.5 |
| | | 56.5 | 10 | 31.8 | 1.7 |
| J | Lipid A9 | 50 | 10 | 38.5 | 1.5 |
| | | 35 | 16 | 46.5 | 2.5 |
| | | 40 | 32.5 | 25 | 2.5 |
| K | OF-02 | 40.3 | 14.9 | 43.7 | 1.1 |
| | | 56.5 | 10 | 31.8 | 1.7 |
| | | 52.7 | 17.5 | 28.7 | 1.1 |
| L | TCL053 | 60 | 10.6 | 27.3 | 2.1 |
| | | 50 | 10 | 38.5 | 1.5 |
| | | 35 | 16 | 46.5 | 2.5 |

The above list is intended to be exemplary and LNPs of other component molar ratios as described herein may be appropriate for the specific application contemplated herein.

For example, the composition of the LNP (e.g. ICL, Cholesterol, DSPC, and DMG-PEG2000) can be combined to yield different molar ratios. In one embodiment, the molar ratio of ICL to Cholesterol to DSPC to PEG/LIPID CONJUGATE are (3 to 80):(3 to 80):(1 to 50):(0.2 to 10). In another embodiment, the molar ratio of ICL to Cholesterol to DSPC to PEG/LIPID CONJUGATE are (7 to 80):(7 to 80):(3 to 50):(1.0 to 10). In another embodiment, the molar ratio of ICL to Cholesterol to DSPC to PEG/LIPID CONJUGATE are (10 to 80):(10 to 80):(4 to 50):(1.5 to 10). In another embodiment, the molar ratio of ICL to Cholesterol to DSPC to PEG/LIPID CONJUGATE are (13 to 80):(13 to 80):(5 to 50):(2.0 to 10). In another embodiment, the molar ratio of ICL to Cholesterol to DSPC to PEG/LIPID CONJUGATE are (3 to 75):(3 to 75):(1 to 45):(0.2 to 7). In another embodiment, the molar ratio of ICL to Cholesterol to DSPC to PEG/LIPID CONJUGATE are (7 to 75):(7 to 75):(3 to 45):(1.0 to 7). In another embodiment, the molar ratio of ICL to Cholesterol to DSPC to PEG/LIPID CONJUGATE are (10 to 75):(10 to 75):(4 to 45):(1.5 to 7). In another embodiment, the molar ratio of ICL to Cholesterol to DSPC to PEG/LIPID CONJUGATE are (13 to 75):(13 to 75):(5 to 45):(2.0 to 7). In another embodiment, the molar ratio of ICL to Cholesterol to DSPC to PEG/LIPID CONJUGATE are (3 to 70):(3 to 70):(1 to 40):(0.2 to 5). In another embodiment, the molar ratio of ICL to Cholesterol to DSPC to PEG/LIPID CONJUGATE are (7 to 70):(7 to 70):(3 to 40):(1.0 to 5). In another embodiment, the molar ratio of ICL to Cholesterol to DSPC to PEG/LIPID CONJUGATE are (10 to 70):(10 to 70):(4 to 40):(1.5 to 5). In another embodiment, the molar ratio of ICL to Cholesterol to DSPC to PEG/LIPID CONJUGATE are (13 to 70):(13 to 70):(5 to 40):(2.0 to 5).

In a second embodiment, the molar ratio of ICL to Cholesterol to DSPC to PEG/LIPID CONJUGATE are (3 to 80):(3 to 70):(1 to 50):(0.2 to 10). In another embodiment, the molar ratio of ICL to Cholesterol to DSPC to PEG/LIPID CONJUGATE are (7 to 80):(7 to 70):(3 to 50):(1.0 to 10). In another embodiment, the molar ratio of ICL to Cholesterol to DSPC to PEG/LIPID CONJUGATE are (10 to 80):(10 to 70):(4 to 50):(1.5 to 10). In another embodiment, the molar ratio of ICL to Cholesterol to DSPC to PEG/LIPID CONJUGATE are (13 to 80):(13 to 70):(5 to 50):(2.0 to 10). In another embodiment, the molar ratio of ICL to Cholesterol to DSPC to PEG/LIPID CONJUGATE are (3 to 75):(3 to 80):(1 to 45):(0.2 to 7). In another embodiment, the molar ratio of ICL to Cholesterol to DSPC to PEG/LIPID CONJUGATE are (7 to 75):(7 to 80):(3 to 45):(1.0 to 7). In another embodiment, the molar ratio of ICL to Cholesterol to DSPC to PEG/LIPID CONJUGATE are (10 to 75):(10 to 80):(4 to 45):(1.5 to 7). In another embodiment, the molar ratio of ICL to Cholesterol to DSPC to PEG/LIPID CONJUGATE are (13 to 75):(13 to 80):(5 to 45):(2.0 to 7). In another embodiment, the molar ratio of ICL to Cholesterol to DSPC to PEG/LIPID CONJUGATE are (3 to 70):(3 to 75):(1 to 40):(0.2 to 5). In another embodiment, the molar ratio of ICL to Cholesterol to DSPC to PEG/LIPID CONJUGATE are (7 to 70):(7 to 75):(3 to 40):(1.0 to 5). In another embodiment, the molar ratio of ICL to Cholesterol to DSPC to PEG/LIPID CONJUGATE are (10 to 70):(10 to 75):(4 to 40):(1.5 to 5). In another embodiment, the molar ratio of ICL to Cholesterol to DSPC to PEG/LIPID CONJUGATE are (13 to 70):(13 to 75):(5 to 40):(2.0 to 5).

In a third embodiment, the molar ratio of ICL to Cholesterol to DSPC to PEG/LIPID CONJUGATE are (3 to 80):(3 to 80):(1 to 40):(0.2 to 10). In another embodiment, the molar ratio of ICL to Cholesterol to DSPC to PEG/LIPID CONJUGATE are (7 to 80):(7 to 80):(3 to 40):(1.0 to 10). In another embodiment, the molar ratio of ICL to Cholesterol to DSPC to PEG/LIPID CONJUGATE are (10 to 80):(10 to 80):(4 to 40):(1.5 to 10). In another embodiment, the molar ratio of ICL to Cholesterol to DSPC to PEG/LIPID CONJUGATE are (13 to 80):(13 to 80):(5 to 40):(2.0 to 10). In another embodiment, the molar ratio of ICL to Cholesterol to DSPC to PEG/LIPID CONJUGATE are (3 to 75):(3 to 75):(1 to 50):(0.2 to 7). In another embodiment, the molar ratio of ICL to Cholesterol to DSPC to PEG/LIPID CONJUGATE are (7 to 75):(7 to 75):(3 to 50):(1.0 to 7). In another embodiment, the molar ratio of ICL to Cholesterol to DSPC to PEG/LIPID CONJUGATE are (10 to 75):(10 to 75):(4 to 50):(1.5 to 7). In another embodiment, the molar ratio of ICL to Cholesterol to DSPC to PEG/LIPID CONJUGATE are (13 to 75):(13 to 75):(5 to 50):(2.0 to 7). In another embodiment, the molar ratio of ICL to Cholesterol to DSPC to PEG/LIPID CONJUGATE are (3 to 70):(3 to 70):(1 to 45):(0.2 to 5). In another embodiment, the molar ratio of ICL to Cholesterol to DSPC to PEG/LIPID CONJUGATE are (7 to 70):(7 to 70):(3 to 45):(1.0 to 5). In another embodiment, the molar ratio of ICL to Cholesterol to DSPC to PEG/LIPID CONJUGATE are (10 to 70):(10 to 70):(4 to 45):(1.5 to 5). In another embodiment, the molar ratio of ICL to Cholesterol to DSPC to PEG/LIPID CONJUGATE are (13 to 70):(13 to 70):(5 to 45):(2.0 to 5).

In a fourth embodiment, the molar ratio of ICL to Cholesterol to DSPC to PEG/LIPID CONJUGATE are (3 to 80):(3 to 80):(1 to 50):(0.2 to 5). In another embodiment, the molar ratio of ICL to Cholesterol to DSPC to PEG/LIPID CONJUGATE are (7 to 80):(7 to 80):(3 to 50):(1.0 to 5). In another embodiment, the molar ratio of ICL to Cholesterol to DSPC to PEG/LIPID CONJUGATE are (10 to 80):(10 to 80):(4 to 50):(1.5 to 5). In another embodiment, the molar ratio of ICL to Cholesterol to DSPC to PEG/LIPID CONJUGATE are (13 to 80):(13 to 80):(5 to 50):(2.0 to 5). In another embodiment, the molar ratio of ICL to Cholesterol to DSPC to PEG/LIPID CONJUGATE are (3 to 75):(3 to 75):(1 to 45):(0.2 to 10). In another embodiment, the molar ratio of ICL to Cholesterol to DSPC to PEG/LIPID CONJUGATE are (7 to 75):(7 to 75):(3 to 45):(1.0 to 10). In another embodiment, the molar ratio of ICL to Cholesterol to DSPC to PEG/LIPID CONJUGATE are (10 to 75):(10 to 75):(4 to 45):(1.5 to 10). In another embodiment, the molar ratio of ICL to Cholesterol to DSPC to PEG/LIPID CONJUGATE are (13 to 75):(13 to 75):(5 to 45):(2.0 to 10). In another embodiment, the molar ratio of ICL to Cholesterol to DSPC to PEG/LIPID CONJUGATE are (3 to 70):(3 to 70):(1 to 40):(0.2 to 7). In another embodiment, the molar ratio of ICL to Cholesterol to DSPC to PEG/LIPID CONJUGATE are (7 to 70):(7 to 70):(3 to 40):(1.0 to 7). In another embodiment, the molar ratio of ICL to Cholesterol to DSPC to PEG/LIPID CONJUGATE are (10 to 70):(10 to 70):(4 to 40):(1.5 to 7). In another embodiment, the molar ratio of ICL to Cholesterol to DSPC to PEG/LIPID CONJUGATE are (13 to 70):(13 to 70):(5 to 40):(2.0 to 7).

### LNP ID A:

### ICL: ALC-0315

Formal Name: 2-hexyl-decanoic acid, 1,1′-[[(4-hydroxybutyl)imino]di-6,1-hexanediyl]ester Alternative Names: ((4-Hydroxybutyl)azanediyl)bis(hexane-6,1-diyl) bis(2-hexyldecanoate)
CAS Number: 2036272-55-4
Molecular Formula: C₄₈H₉₅NO₅

### LNP ID B:

### ICL: DLin-MC3-DMA

Formal Name: 4-(dimethylamino)-butanoic acid, (10Z,13Z)-1-(9Z,12Z)-9,12-octadecadien-1-yl-10,13-nonadecadien-1-yl ester
Alternative Names: (6Z,9Z,28Z,31Z)-heptatriaconta-6,9,28,31-tetraen-19-yl 4-(dimethylamino)butanoate
CAS Number: 1224606-06-7
Molecular Formula: C₄₃H₇₉NO₂

### LNP ID C:

### ICL: LP-01

Formal Name: 9Z,12Z-octadecadienoic acid, 3-[4,4-bis(octyloxy)-1-oxobutoxy]-2-[[[[3-(diethylamino)propoxy]carbonyl]oxy]methyl]propyl ester
Alternative Names: CIN-16645, 9Z,12Z-octadecadienoic acid, 3-[4,4-bis(octyloxy)-1-oxobutoxy]-2-[[[[3-(diethylamino)propoxy]carbonyl]oxy]methyl]propyl ester
   CAS Number: 1799316-64-5
   Molecular Formula: C₅₀H₉₃NO₉

### LNP ID D:

### ICL: SM-102

Formal Name: 8-[(2-hydroxyethyl)[6-oxo-6-(undecyloxy)hexyl]amino]-octanoic acid, 1-octylnonyl ester
Alternative Names: Lipid H, Heptadecan-9-yl 8-((2-hydroxyethyl)(6-oxo-6-(undecyloxy)hexyl)amino)-octanoate
CAS Number: 2089251-47-6
Molecular Formula: C₄₄H₈₇NO₅

### LNP ID E:

### ICL: 4A3-SC8

CAS Number: 1857340-78-3
Molecular Formula: C₇₅H₁₃₉N₃O₁₆S₄

### LNP ID F

ICL: cKK-E12
Formal Name: 3,6-bis[4-[bis(2-hydroxydodecyl)amino]butyl]-2,5-piperazinedione
Alternative Names: 3,6-bis(4-(bis(2-hydroxydodecyl)amino)butyl)piperazine-2,5-dione
CAS Number: 1432494-65-9
Molecular Formula: C₆₀H₁₂₀N₄O₆

### LNP ID G:

### ICL: 306ᵢ₁₀

Formal Name: tetrakis(8-methylnonyl) 3,3′,3″,3′″-(((methylazanediyl)bis(propane-3,1-diyl))bis(azanetriyl))tetrapropionate
CAS Number: 2322290-93-5
Molecular Formula: C₅₉H₁₁₅N₃O₈

### LNP ID H:

### ICL: C3-K2-E14

Formal Name: 3,3′-(propylazanediyl)bis(N-(2-(bis(2-hydroxytetradecyl)amino)ethyl)propanamide)
CAS Number: 2933215-86-0
Molecular Formula: C₆₉H₁₄₁N₅O₆

### LNP ID I:

### ICL: C14-4

Formal Name: 1,1′-[[2-[2-[4-[2-[[2-[2-[bis(2-hydroxytetradecyl)amino]ethoxy]ethyl](2-hydroxytetradecyl)amino]ethyl]-1-piperazinyl]ethoxy]ethyl]imino]bis-2-tetradecanol
CAS Number: 2639634-80-1
Molecular Formula: C₈₄H₁₇₃N₅O₇

### LNP ID J:

### ICL: Lipid A9

Formal Name: bis(2-butyloctyl) 10-(N-(3-(dimethylamino)propyl)nonanamido)nonadecanedioate
Alternative Names: 1,19-Bis(2-butyloctyl) 10-[[3-(dimethylamino)propyl](1-oxononyl)amino]nonadecanedioate
CAS Number: 2036272-50-9
Molecular Formula: C₅₇H₁₁₂N₂O₅

### LNP ID K:

### ICL: OF-02

Formal Name: 3,6-bis[4-[bis[(9Z,12Z)-2-hydroxy-9,12-octadecadien-1-yl]amino]butyl]-2,5-piperazinedione
Alternative Names: 3,6-bis(4-(bis((9Z,12Z)-2-hydroxyoctadeca-9,12-dien-1-yl)amino)butyl)piperazine-2,5-dione
CAS Number: 1883431-67-1
Molecular Formula: C₈₄H₁₅₂N₄O₆

### LNP ID L:

### ICL: TCL053

Formal Name: 2-(((4-(dimethylamino)butanoyl)oxy)methyl)-2-((((Z)-tetradec-9-enoyl)oxy)methyl)propane-1,3-diyl (9Z,9′Z)-bis(tetradec-9-enoate)
CAS Number: 2361162-70-9
Molecular Formula: C₅₃H₉₅NO₈

In another embodiment, the 5′ UTR region may comprise a ribosome binding site, a Kozak sequence, a modified genomic human LIPE 5′ UTR, a modified genomic human LEP 5′ UTR, a modified genomic human COL1A 5′ UTR, a modified genomic human ADIPOQ 5′ UTR, a modified genomic human PLIN1 5′ UTR, a translation initiator of short 5′ UTR (TISU), a consensus genomic human LIPE 5′ UTR, a consensus genomic human UCP1 5′ UTR, a consensus genomic human PLIN1 5′ UTR, a consensus genomic human COL1A 5′ UTR, a consensus genomic human LEP 5′ UTR, a consensus genomic human ADIPOQ 5′ UTR, a consensus genomic human FABP4 5′ UTR, or combinations thereof.

In another embodiment, the 3′ UTR region may comprise a double stop codon, a consensus genomic human UCP1 3′ UTR, a Cominarity 3′ UTR, or combinations thereof. The Cominarity 3′ UTR is a combination and optimization of segments from the human mitochondrial 12S rRNA (mtRNR1) and the human AES/TLE5 gene. The AES segment of 136 nt with two C→Ψ mutations was added after two trinucleotides following the second stop codon. The mtRNR1 segment of 139 nt was added immediately after.

In another embodiment, the mRNA of the present disclosure comprises (consists essentially of, or consists of) a nucleotide sequence about 80% to about 100%, about 85% to about 100%, about 90% to about 100%, or about 95% to about 100%, identical to the amino acid sequence set forth in SEQ ID NO:130.

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth SEQ ID NO: 130.

In another embodiment, the mRNA of the present disclosure comprises (consists essentially of, or consists of) a nucleotide sequence about 80% to about 100%, about 85% to about 100%, about 90% to about 100%, or about 95% to about 100%, identical to the amino acid sequence set forth in any of SEQ ID NO:2 through SEQ ID NO: 21.

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NO:2 through SEQ ID NO:21.

In an alternative embodiment, the mRNA of the present disclosure comprises (consists essentially of, or consists of) a nucleotide sequence about 80% to about 100%, about 85% to about 100%, about 90% to about 100%, or about 95% to about 100%, identical to the amino acid sequence set forth in any of SEQ ID NO:22 through SEQ ID NO:129.

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NO:22 through SEQ ID NO: 129.

In another embodiment, the sequence of the mRNA may be selected from one of SEQ ID NO: 1 to SEQ ID NO: 130, encoding for UCP1 by the ribosomes of a target cell packaged in a lipid nanoparticle (LNP) for transfection of WAs or ASCs with the packaged mRNA.

In certain embodiments, the mRNA comprises (consists essentially of, or consists of) a nucleotide sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the nucleotide sequence set forth in any of SEQ ID NO:1 through SEQ ID NO:130.

In another embodiment, cells transfected with a pharmaceutical composition comprising the present mRNA will express UCP1 and undergo a process of lipolysis through non-ATP generating respiration, generating heat and reducing the overall FFA content of the transfected and nearby cells.

In another embodiment, a physician may administer the pharmaceutical composition comprising the present mRNA packaged in an LNP to a patient seeking to accomplish spot fat reduction.

In another embodiment, a physician administering the present mRNA packaged in an LNP to a patient seeking to accomplish spot fat reduction may administer the pharmaceutical composition in a bolus injection in the targeted area.

In another embodiment, a physician administering the present mRNA packaged in an LNP to a patient seeking to accomplish spot fat reduction may administer the pharmaceutical composition in a series of injections in the targeted area.

In another embodiment, a physician administering the present mRNA packaged in an LNP to a patient seeking to accomplish spot fat reduction may administer the pharmaceutical composition transdermally. The transdermal administration may be accomplished through the use of penetration enhancers (e.g. DMSO, oleic acid, lauric acid, sodium lauryl sulfate, sodium ducosate, sodium oleate, cetyltrimethylammonium bromide, benzalkonium chloride, polysorbates, sorbitan esters, polyoxyethylene alkyl ethers, Laurocarpam, propylene glycol monolaurate, lecithin, terpenes), vesicular carriers (e.g. liposomes, ethosomes, niosomes) microneedle injection, iontophoresis, sonophoresis, jet injection or other suitable means.

Microneedle injectors utilize microscopic needles, typically ranging from 50 to 900 micrometers in length, to bypass the skin's outermost layer, the stratum corneum, and deliver therapeutic agents directly into the underlying epidermal or dermal layers. The microneedles can be disposed in an array on a patch wherein several hundred or several thousand microneedles are disposed.

Iontophoresis utilizes a low-level electrical current to facilitate the transport of ions across biological membranes, most notably the skin. The medication is disposed on a patch with an electrode that conducts electricity to drive the ions across the membrane.

Sonophoresis, also known as phonophoresis, is a non-invasive technique that utilizes ultrasound waves to temporarily increase the permeability of the skin. This enhanced permeability allows for the improved delivery of therapeutic agents, such as medications and cosmetics, across the skin barrier and into deeper tissues.

Jet injection employs a high-velocity stream of liquid that penetrates the skin to deliver the substance into the underlying tissues. A jet injector creates a very fine, high-pressure jet of liquid that is powerful enough to pierce the skin and reach the desired tissue depth—typically the intradermal, subcutaneous, or intramuscular layer. This is achieved by rapidly forcing a pre-measured dose of medication through a tiny orifice in the injector's nozzle.

In another embodiment, a physician administering the present mRNA packaged in an LNP to a patient seeking to accomplish spot fat reduction may administer the pharmaceutical composition several times over the course of periodic patient encounters.

In another embodiment, a physician administering the present mRNA packaged in an LNP to a patient seeking to accomplish spot fat reduction may administer the pharmaceutical composition in a manner prescribed by an algorithm, LLM or machine learning implementation to accomplish the desired spot fat reduction.

In another embodiment, an mRNA, comprising the nucleotide sequence set forth in one of SEQ ID NO: 2 through SEQ ID NO: 21 disclosed in the Sequence Listing incorporated by reference herein, encoding for UCP1 is packaged in a lipid nanoparticle (LNP). The packaged synthetic mRNA may be for transfection of WAs or ASCs.

In another embodiment, cells transfected with a pharmaceutical composition comprising mRNA of any of SEQ ID NO: 2 through SEQ ID NO: 21 will express UCP1 and undergo a process of lipolysis through non-ATP generating respiration, generating heat and reducing the overall FFA content of the transfected cells.

In another embodiment, a physician may administer the pharmaceutical composition comprising the mRNA of any of SEQ ID NOs: 2 through 21 packaged in an LNP to a patient seeking to accomplish spot fat reduction.

In another embodiment, the mRNA comprising the nucleotide sequence set forth in any of SEQ ID NOs: 2 through 21 packaged in an LNP is administered to a patient seeking to accomplish spot fat reduction. The packaged mRNA may be administered in a bolus injection in the targeted area.

In another embodiment, the mRNA comprising the nucleotide sequence set forth in any of SEQ ID NOs: 2 through 21 packaged in an LNP is administered to a patient seeking to accomplish spot fat reduction in a series of injections in the targeted area.

In another embodiment, the mRNA comprising the nucleotide sequence set forth in any of SEQ ID NOs: 2 through 21 packaged in an LNP is administered transdermally to a patient seeking to accomplish spot fat reduction. The transdermal administration may be accomplished through the use of penetration enhancers (e.g. DMSO, oleic acid, lauric acid, sodium lauryl sulfate, sodium ducosate, sodium oleate, cetyltrimethylammonium bromide, benzalkonium chloride, polysorbates, sorbitan esters, polyoxyethylene alkyl ethers, Laurocarpam, propylene glycol monolaurate, lecithin, terpenes), vesicular carriers (e.g. liposomes, ethosomes, niosomes) microneedle injection, iontophoresis, sonophoresis, jet injection or other suitable means.

In another embodiment, the mRNA comprising the nucleotide sequence set forth in any of SEQ ID NOs: 2 through 21 packaged in an LNP is administered to a patient seeking to accomplish spot fat reduction several times over the course of periodic patient encounters.

In another embodiment, a physician administering the mRNA comprising the nucleotide sequence set forth in any of SEQ ID NOs: 2 through 21 packaged in an LNP to a patient seeking to accomplish spot fat reduction may administer the pharmaceutical composition in a manner prescribed by an algorithm, LLM or machine learning implementation to accomplish the desired spot fat reduction.

In alternative embodiment, a synthetic mRNA, comprising the nucleotide sequence set forth in any of SEQ ID NOs: 22 through 129 disclosed in the Sequence Listing incorporated by reference herein, encoding for UCP1 is packaged in a lipid nanoparticle (LNP).

In an alternative embodiment, cells transfected with a pharmaceutical composition comprising mRNA comprising the nucleotide sequence set forth in any of SEQ ID NO: 22 through SEQ ID NO: 129 will express UCP1 and undergo a process of lipolysis through non-ATP generating respiration, generating heat and reducing the overall FFA content of the transfected cells.

In an alternative embodiment, a physician may administer the pharmaceutical composition comprising the mRNA of SEQ ID NOs: 22 through 129 packaged in an LNP to a patient seeking to accomplish spot fat reduction.

In an alternative embodiment, a physician administering the mRNA comprising the nucleotide sequence set forth in any of SEQ ID NOs: 22 through 129 packaged in an LNP to a patient seeking to accomplish spot fat reduction may administer the pharmaceutical composition in a bolus injection in the targeted area.

In an alternative embodiment, a physician administering the mRNA comprising the nucleotide sequence set forth in any of SEQ ID NOs: 22 through 129 packaged in an LNP to a patient seeking to accomplish spot fat reduction may administer the pharmaceutical composition in a series of injections in the targeted area.

In an alternative embodiment, a physician administering the mRNA comprising the nucleotide sequence set forth in any of SEQ ID NOs: 22 through 129 packaged in an LNP to a patient seeking to accomplish spot fat reduction may administer the pharmaceutical composition transdermally. The transdermal administration may be accomplished through the use of penetration enhancers (e.g. DMSO, oleic acid, lauric acid, sodium lauryl sulfate, sodium ducosate, sodium oleate, cetyltrimethylammonium bromide, benzalkonium chloride, polysorbates, sorbitan esters, polyoxyethylene alkyl ethers, Laurocarpam, propylene glycol monolaurate, lecithin, terpenes), vesicular carriers (e.g. liposomes, ethosomes, niosomes) microneedle injection, iontophoresis, sonophoresis, jet injection or other suitable means.

In an alternative embodiment, a physician administering the mRNA comprising the nucleotide sequence set forth in any of SEQ ID NOs: 22 through 129 packaged in an LNP to a patient seeking to accomplish spot fat reduction may administer the pharmaceutical composition several times over the course of periodic patient encounters.

In an alternative embodiment, a physician administering the mRNA comprising the nucleotide sequence set forth in any of SEQ ID NOs: 22 through 129 packaged in an LNP to a patient seeking to accomplish spot fat reduction may administer the pharmaceutical composition in a manner prescribed by an algorithm, LLM or machine learning implementation to accomplish the desired spot fat reduction.

In another embodiment, the synthetic mRNA may in addition to or alternatively encode for the B3 adrenoreceptor, PR/SET domain 16 (PRDM16), Peroxisome proliferator-activated receptor gamma coactivator-1 alpha (PGC-1alpha), CCAAT/enhancer binding protein beta (C/EPB-Beta), Krüppel-like Factor 11 (KLF11), and/or nuclear receptor interacting protein 1 (NRIP1). In certain embodiments, the present pharmaceutical composition may comprise a first mRNA encoding UPC1, and one or more mRNAs encoding the B3 adrenoreceptor, PR/SET domain 16 (PRDM16), Peroxisome proliferator-activated receptor gamma coactivator-1 alpha (PGC-1alpha), CCAAT/enhancer binding protein beta (C/EPB-Beta), Krüppel-like Factor 11 (KLF11), nuclear receptor interacting protein 1 (NRIP1), or combinations thereof.

In another embodiment, the synthetic mRNA may be modified with a 5′ cap, a 5′ untranslated region, polymorphisms in the target protein coding sequence, nucleic acid substitutions to reduce immunogenicity and/or enhance mRNA longevity within the cell, and/or a 3′ untranslated region including one or more poly-A tails.

The mRNA can be chemically or biologically modified. Modifications to an mRNA include the depletion of a base (e.g., by deletion or by the substitution of one nucleotide for another) or modification of a base, chemically by covalent addition pendant groups which are not naturally found in such mRNA molecules. For example, the number of C and/or U residues in an mRNA sequence may be reduced. In another embodiment, the number of C and/or U residues is reduced by substitution of one codon encoding a particular amino acid for another codon encoding the same or a related amino acid. The mRNA nucleic acids can also incorporate pseudouridines. Substitutions and modifications to the mRNA of the present invention may be performed by methods readily known to one of ordinary skill in the art.

Modifications to the mRNA can also be made to the 3′ and 5′ ends of an mRNA molecule. These modifications include a poly A tail or a longer poly A tail, the alteration of the 3′ UTR or the 5′ UTR or complexing the mRNA with a protein or complementary nucleic acid molecule.

Exemplary mRNA sequences are provided in the Sequence Listing incorporated by reference herein.

The present mRNA or pharmaceutical composition may be delivered via local delivery directly into WAT. Dosages will range from 0.001 mg/kg to 1.0 mg/kg. The frequency of dosing into a given anatomic area may be no more than once per week, per five days, per three days, or per day. Total duration of treatment will be determined by the patient and their healthcare provider, accounting for factors including but not limited to desired treatment endpoint, tolerability of the drug's delivery, tolerability of the drug's side effects, and patient's lifestyle preferences (i.e. how often they want to get the treatment).

In another embodiment, the synthetic mRNA encoding for UCP1 packaged in an LNP is formulated for injectable delivery to a target area. This may include a dilute formulation that may be injected into a large area of subcutaneous fat using a blunt canula or other means fit for such purpose. This injectable delivery may alternatively include direct injection to a target site of adipose tissue. This formulation may include a carrier solution that protects the LNP vesicles during transport and injection and may further comprise a buffering solution fit for that purpose.

In another embodiment, the synthetic mRNA encoding for UCP1 packaged in an LNP is formulated for transdermal delivery to a target area. This formulation may include a transdermal carrier such as a nanostructured lipid carrier molecule suspended in a cream or gel to be rubbed on the skin over the target area. Transdermal delivery may be further effectuated by iontophoresis, a microneedle patch or a topical formulation to be applied after disruption of the skin barrier via methods including but not limited to microneedling, laser treatment, and chemical peels.

In another embodiment, the synthetic mRNA encoding for UCP1 packaged in an LNP is formulated for systemic delivery to multiple sites within a patient's body. This formulation may include a carrier solution that protects the LNP vesicles during transport and injection/intravenous delivery and may further comprise a buffering solution fit for that purpose.

In a second embodiment, a patient treated with a synthetic mRNA encoding for UCP1 packaged in an LNP employs an apparatus to cool the pharmaceutically generated BeAT in order to activate the thermogenic process of BeAT. This apparatus may comprise: ice packs, a cold-water tub or a system that circulates chilled water through a pad or patch positioned on the surface of the skin over a target area. This apparatus may comprise an internal cooling mechanism for precisely regulating the temperature of the cooling apparatus or of the pad or patch positioned on the surface of the skin over a target area.

In another embodiment, the cooling apparatus may be used as part of a protocol prescribed by a treating physician for activating the thermogenic properties of the BeAT for predetermined time periods in order to achieve the specific goals of the patient with regards to reduction of the appearance of adipose tissue in the target area.

In another embodiment, the BeAT may be activated by B3 agonists (e.g. mirabegron), caffeine and like molecules, ibuprofen and like molecules, 4-heptylbenzoic acid; bromododecanoic acid; dodecanoic acid; nonadecanoic acid; oleic acid; retinoic acid; tetradecylthioacetic acid; TTNPB; TUG-891.

In another embodiment, the protocol prescribed by the treating physician may include a time frame for repeat treatment of the target area with the synthetic mRNA encoding for UCP1 packaged in an LNP. The time frame may reflect the rate of turnover of UCP1 in the target cells.

In another embodiment, a state change transition of target cells from white adipocytes to beige or brown adipocytes may include contacting cells from WAT with a payload of mRNA, protein, plasmid DNA or oligonucleic acid encapsulated in MITO-Porter (a liposome-based carrier for delivering a payload to the mitochondria via membrane fusion) for targeting the payload to the mitochondria.

In another embodiment, the invention may be a pharmaceutical composition comprising UCP1 protein (NCBI RefSeq: NP_001735.1) encapsulated in MITO-Porter.

In another embodiment, the UCP1 protein may be optimized to increase its active life by including factors which inhibit the effects of mitochondrial proteases LONP1 and ClpXP.

In certain embodiments, factors which may inhibit the effects of mitochondrial proteases LONP1 and ClpXP may include but are not limited to: modifying protease cleavage sites, glycosylation, incorporation of non-natural amino acids, and/or enhancing protein structural stability.

In certain embodiments, modifying a protease cleavage site may include substitution of one or more amino acids such as lysine or arginine with threonine or glutamine to prevent protease activity at a preferred cleavage site.

In certain embodiments, modifying a protease cleavage site may include the introduction of proline next to the preferred cleavage site of the target protease.

In certain embodiments, glycosylation may include adding glycans to the protein to prevent the protease from accessing the cleavage site.

In certain embodiments, the glycans may be N-linked, and for example, attached to asparagine.

In certain embodiments, the glycans may be O-linked, and for example, attached to serine or threonine.

In certain embodiments, the incorporation of non-natural amino acids may include the inclusion of D-amino acids at the cleavage site to prevent protease recognition of the cleavage site.

In certain embodiments, the incorporation of non-natural amino acids may include the inclusion of Beta-amino acids at the cleavage site to reduce the affinity of the protease for the cleavage site.

In certain embodiments, enhancing protein structural stability may include the introduction of disulfide bonds between two or more amino acids in the protein or the introduction of salt bridges in the protein's core.

In another embodiment, the UCP1 protein may be optimized to increase its binding affinity to the inner mitochondrial matrix by optimizing the mitochondrial targeting signal (MTS) of the UCP1 protein, introduction of destabilizing elements, and/or the introduction of chaperone binding sites.

In certain embodiments, enhancing the MTS may include increasing the positive charge of the protein to improve recognition of the protein by TOM20 and TOM22.

In certain embodiments, increasing the positive charge of the protein may include substituting neutral or negatively charged amino acids with positively charged amino acids such as arginine or lysine.

In certain embodiments, enhancing the MTS may include optimizing the amphipathicity of the protein.

In certain embodiments, optimizing the amphipathicity of the protein may include improving the balance of amino acids in the amphipathic alpha helix that forms the MTS such that the positively charged side of the helix includes as many positively charged amino acids as possible and the hydrophobic side of the helix includes as many hydrophobic amino acids as possible.

In certain embodiments, enhancing the MTS may include substitution of the extant MTS with a highly efficient MTS such as that of human cytochrome c oxidase subunit 8 (COX8) or that of superoxide dismutase 2 (SOD2).

In certain embodiments, enhancing the MTS may include optimizing the length of the MTS.

In certain embodiments, the introduction of folding destabilizing elements may increase the proportion of unfolded protein available for import to the mitochondria.

In certain embodiments, the introduction of chaperone binding sites may include engineering binding sites for mitochondrial chaperone molecules such as Hsp70 to increase the efficiency of its import to the mitochondria.

The present invention may be better understood by reference to the following non-limiting examples, which are presented in order to more fully illustrate the preferred embodiments of the invention. They should in no way be construed to limit the broad scope of the invention.

### EXAMPLES

### Example 1

Synthetic mRNA expressing mCherry fluorescent protein was synthesized via in vitro transcription from a linearized DNA plasmid. Specifically, high-purity plasmid was linearized with a restriction enzyme at a site downstream of the IVT mRNA sequence that was to be transcribed. Following linearization, the resulting linear template DNA was purified with phenol/chloroform extraction using DNAse-free reagents. The final, air-dried DNA pellet was resuspended in nuclease-free water at a concentration of 0.5-1 μg/l.

To synthesize the IVT mRNA, a reaction mix comprised of sufficient nuclease-free water to produce a total reaction volume of 20 μL, 2 μL of 10×T7 CleanCap Reagent AG Reaction Buffer (New England Biolabs), 2 μL of 60 mM ATP (final concentration 6 mM), 2 μL of 50 mM CTP (final concentration 5 mM), 2 μL of 50 mM N1-Methyl-Pseudouridine-5′-Triphosphate (final concentration 5 mM), 2 μL of 50 mM GTP (final concentration 5 mM), 2 μL of 40 mM AG cap analog (final concentration 4 mM), 1 μg of linearized template DNA, 1 μL of 0.1M dithiothreitol (final concentration 5 mM), and 2 μL of T7 RNA Polymerase Mix (New England Biolabs) was prepared. The reaction was allowed to proceed for 2-16 hours, with reaction time adjusted based on reaction yield. Following the IVT reaction, DNAse I was added to the mix to degrade the template DNA, thereby purifying the IVT mRNA transcript. The IVT mRNA was further purified with gel electrophoresis or the Monarch RNA Cleanup Kit (New England Biolabs).

A poly(A) tail was added to the transcript by preparing a reaction mix comprised of sufficient nuclease-free water to produce a total reaction volume of 20 μL, 1-10 ug of purified IVT mRNA, 2 μL of 10× E. coli Poly(A) Polymerase Reaction Buffer (New England Biolabs), 2 μL of 10 mM ATP, and 1 μL of E. coli Poly(A) Polymerase (final amount 5 units, New England Biolabs). The reaction was incubated for 30 minutes and then stopped by EDTA addition. The final IVT mRNA product was purified with phenol-chloroform extraction, column-based cleanup, or the Monarch RNA Cleanup Kit (New England Biolabs). QA/QC was performed via Sanger sequencing and UV Light Absorbance.

The synthetic mRNA was encapsulated into lipid nanoparticles, yielding a reporter gene test article. Specifically, 0.3 mL of an ethanolic solution of the lipid components (see Table 1) in a syringe pump and 0.9 mL of a solution of the mRNA in RNase-free citrate buffer (100 mM, pH 3) in a syringe pump were mixed simultaneously at flow rates of 3 mL/min for the lipids and 9 mL/min for the mRNA (total flow rate of 12 mL/min) using a microfluidic chip. Following micro fluidic mixing, the resulting mCherry mRNA LNPs were dialyzed overnight against 500 sample volume of PBS (pH 7.4). Agrawal et al., Standardizing a Protocol for Streamlined Synthesis and Characterization of Lipid Nanoparticles to Enable Preclinical Research and Education, bioRxiv, 2025, doi.org/10.1101/2025.07.31.667476

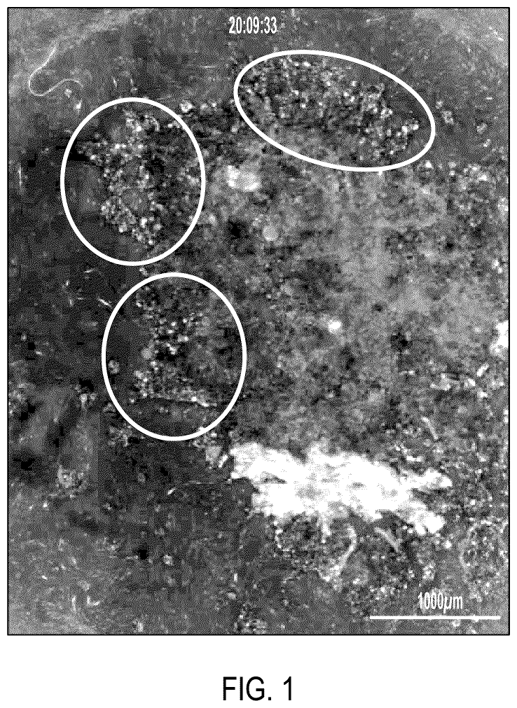

Subcutaneous fat was donated by a patient undergoing elective surgery under an IRB-approved protocol. The fat was stabilized for long-term ex vivo culture: 50 mg of fat was sandwiched between two sheets of adipose-derived stromal cells in a culture medium. The culture medium was comprised of DMEM, 10% fetal bovine serum, and antibiotic and antifungal prophylactics. The ex vivo fat was treated with the mCherry reporter gene test article by adding the mCherry mRNA-LNP to the culture medium in doses ranging from 0.1 to 10 mg of mRNA per kg of fat. Fluorescence and brightfield microscopy images were captured every 4 to 6 hours using an automated system for a total of 48 hours. New red fluorescence in previously non-fluorescent cells in FIG. 1 indicated successful transfection of ex vivo human fat. The white circles in FIG. 1 indicate cells expressing mCherry.

### Example 2

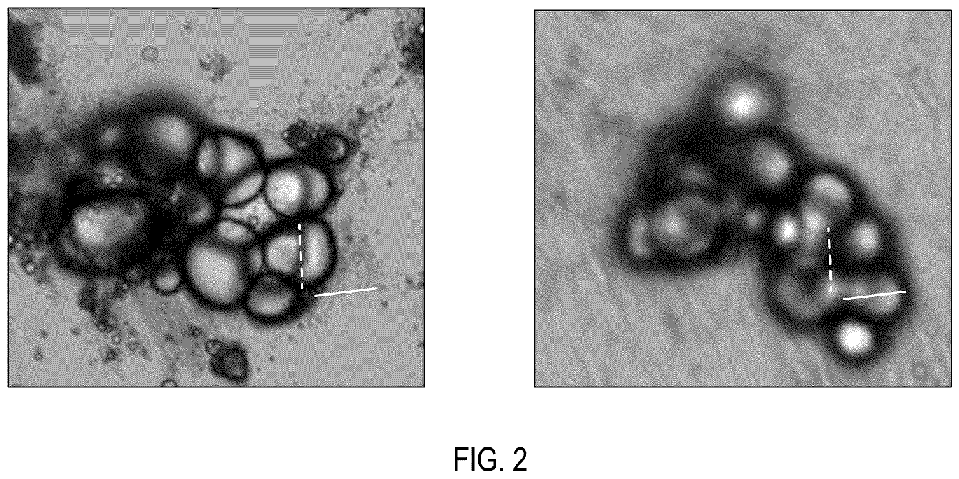

Subcutaneous fat was donated by a patient undergoing elective surgery under an IRB-approved protocol. The fat was stabilized for long-term ex vivo culture. This study served as a negative control where the ex vivo cultured fat was not treated. The culture medium was comprised of DMEM, 10% fetal bovine serum, and antibiotic and antifungal prophylactics. Brightfield microscopy images were captured every 4 to 6 hours using an automated system for a total of 6 days (144 hours). No significant change in the adipocytes' diameter was observed. In FIG. 2, the dashed line marked the diameter of one representative mature adipocyte within the adipose tissue cluster at the start of the experiment (hour 0). The solid line marked the diameter of the same adipocyte at the end of the experiment (hour 144).

### Example 3

Synthetic mRNA of SEQ ID NO: 130 expressing UCP1 was synthesized via in vitro transcription from a linearized DNA plasmid. The synthetic mRNA was encapsulated as described above into lipid nanoparticles of composition LNP D of component molar ratio (SM-102:DSPC:Cholesterol:PEG/Lipid Conjugate) of 50:10:38.5:1.5, yielding a drug candidate test article.

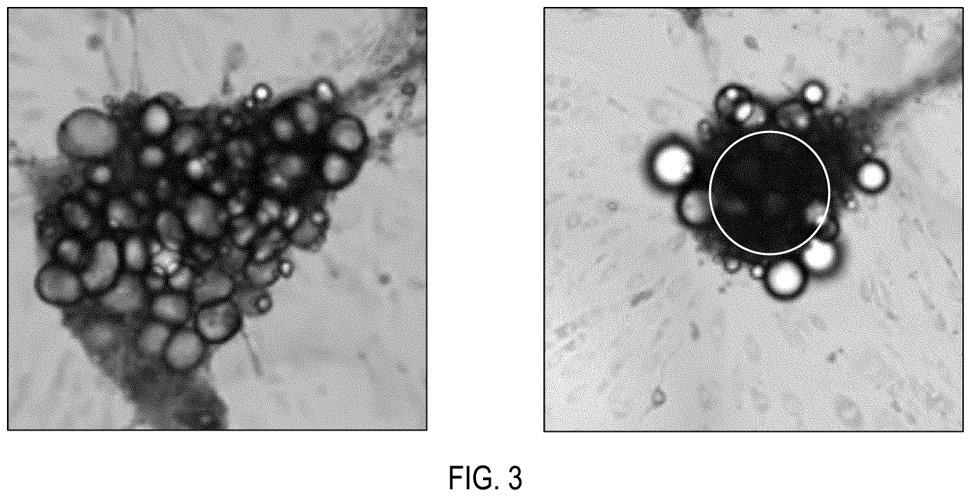

Subcutaneous fat was donated by a patient undergoing elective surgery under an IRB-approved protocol. The same donated fat was used in this study as in the negative control study (Example 2). The fat was stabilized for long-term ex vivo culture. The culture media was comprised of DMEM, 10% fetal bovine serum, and antibiotic and antifungal prophylactics. The ex vivo fat was exposed to the drug candidate test article at full dose (5 μg drug candidate per 100 mg of ex vivo fat) by adding 5 μg of mRNA to the culture media. Brightfield microscopy images were captured every 4 to 6 hours using an automated system for a total of 6 days (144 hours). FIG. 3 shows that the diameters of treated fat cells shrank by up to 65% as observed in visual microscopy, with a majority of the unilocular adipocytes shrinking to the point where they could no longer be visually identified as mature, unilocular adipocytes. The white circle in the right panel of FIG. 3 indicates cells that had shrunk to a point that they are no longer individually visually identifiable at the original level of magnification and depth of field.

### Example 4

Synthetic mRNA of SEQ ID NO: 130 expressing UCP1 was synthesized via in vitro transcription from a linearized DNA plasmid. The synthetic mRNA was encapsulated into lipid nanoparticles as described above in Example 3, yielding a drug candidate test article.

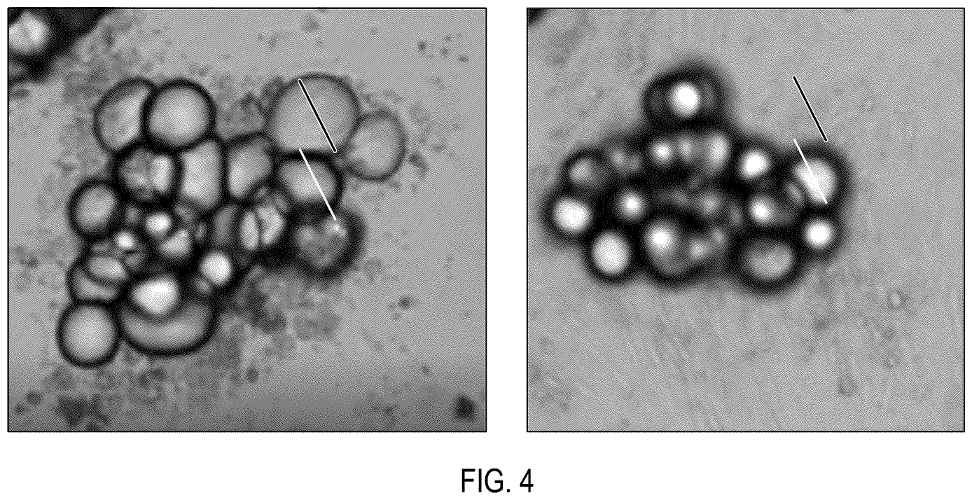

Subcutaneous fat was donated by a patient undergoing elective surgery under an IRB-approved protocol. The same donated fat was used in this study as in the negative control study (Example 2) and the full-dose study (Example 3). The fat was stabilized for long-term ex vivo culture. The culture media was comprised of DMEM, 10% fetal bovine serum, and antibiotic and antifungal prophylactics. The ex vivo fat was treated with the drug candidate test article at half dose (2.5 μg drug candidate per 100 mg of ex vivo fat) by adding 2.5 ug of mRNA to the culture media. Brightfield microscopy images were captured every 4 to 6 hours using an automated system for a total of 6 days (144 hours). FIG. 4 shows that every treated fat cluster shrank significantly, but less so than with the full-dose. The lines in FIG. 4 denote the diameter of one representative mature adipocyte within the adipose tissue cluster at the start of the experiment (hour 0) and at the end of the experiment (hour 144).

### Example 5

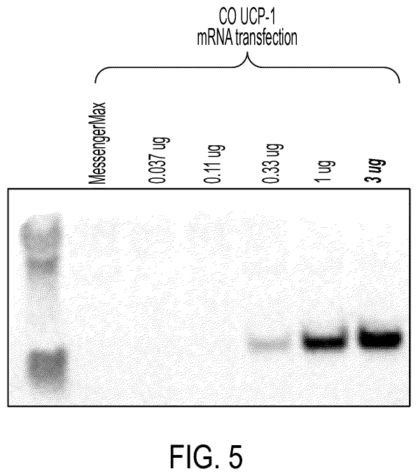

Synthetic mRNA of SEQ ID NO: 130 expressing UCP1 was synthesized via in vitro transcription from a linearized DNA plasmid. The synthetic mRNA was encapsulated using Lipofectamine™ MessengerMAX™ Transfection Reagent. Varying doses of the synthetic mRNA (0.037 μg, 0.11 μg, 0.33 μg, 1 μg and 3 μg) were used to treat human adipose-derived stem cells (ASCs) for 48 hours. Treatment was performed by adding drug test articles to the ASC culture media (DMEM, 10% fetal bovine serum, and antibiotic and antifungal prophylactics.) After 48 hours, the ASCs were lysed, and total protein was isolated. The protein isolated were assayed via Western Blot to provide a semi-quantitative assessment of the amount of UCP1 protein (FIG. 5).

### Example 6

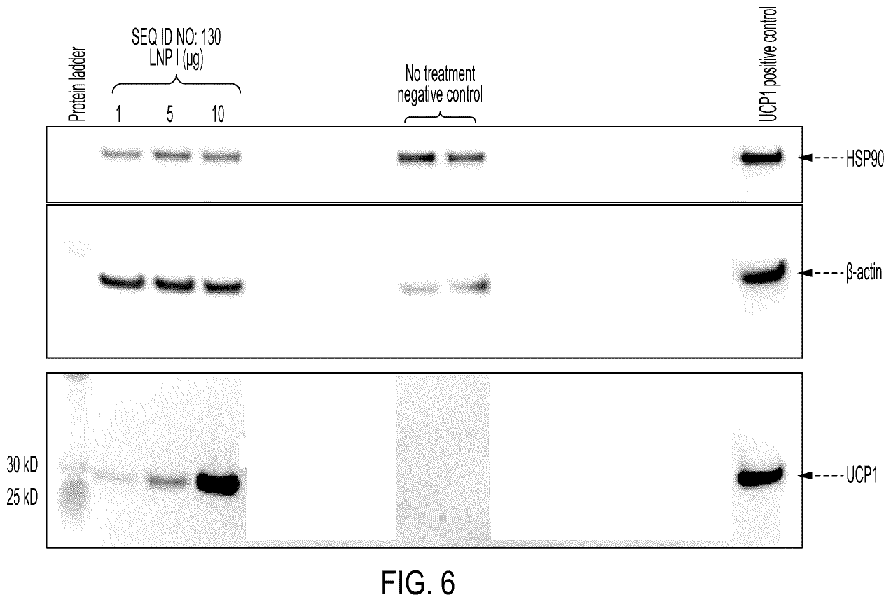

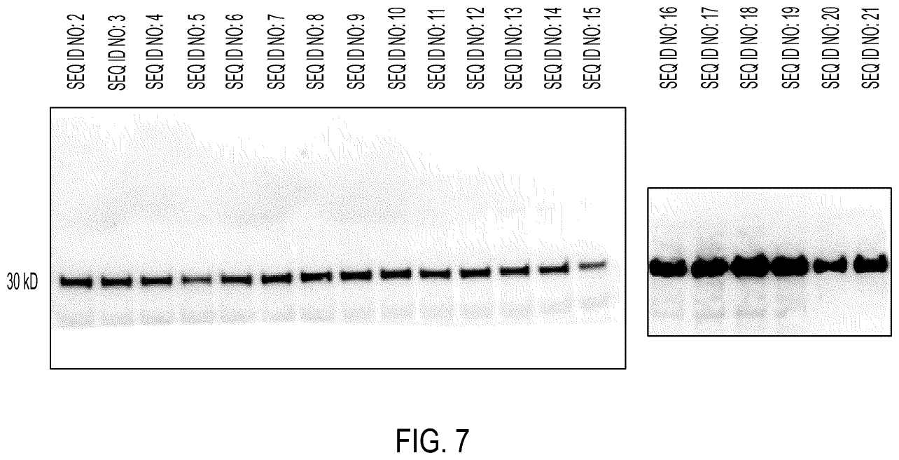

The sequence of mRNA of SEQ ID NO: 130 was created using A plasmid Editor (ApE). The coding sequence (CDS) was optimized using the GenScript Biotech Corporation GenSmart tool. The mRNA was manufactured by GeneFab Inc. using N1-methylpseudouridine as a substitute for uridine and packaged in LNP I as described above. The packaged mRNA was used to treat cultured, ex vivo human white adipose tissue that was prepared as described above. Treatment was performed by adding our drug test articles to the ex vivo fat culture media (DMEM, 10% fetal bovine serum, and antibiotic and antifungal prophylactics.) After 24 hours, culture media was aspirated from the dish; cells were washed in chilled phosphate buffered saline (PBS) and then PBS was aspirated completely. Lysis buffer was added to the culture dish and supplemented with protease and phosphatase inhibitors. Cells and cell lysate were transferred to a pre-chilled microcentrifuge tube. The lysate was incubated on ice for 15-30 mins and then centrifuged at high speed for 20 minutes to pellet cell debris. Supernatant was loaded into a new microcentrifuge tube and protein quantified. Sodium Dodecyl Sulfate-Polyacrylamide Gel Electrophoresis (SDS-PAGE), blotting, immunodetection and imaging were performed. FIG. 6 presents the resulting images confirming the presence of UCP1 in the transfected WAT, as well as expected levels of housekeeping genes heat shock protein 90 (HSP90) and beta-actin (β-actin).

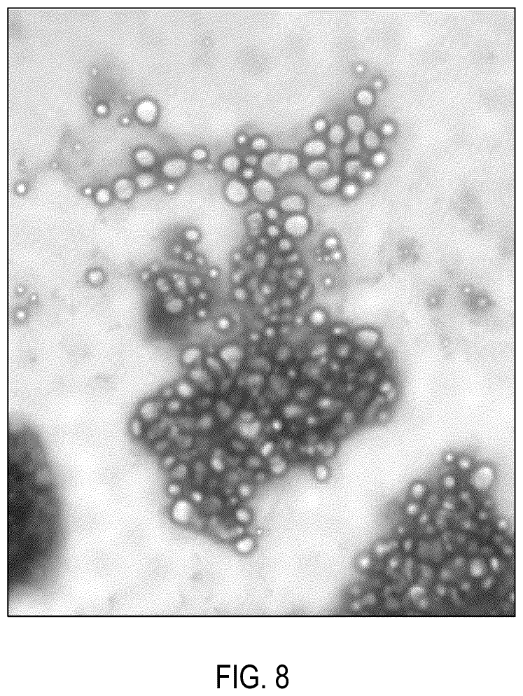

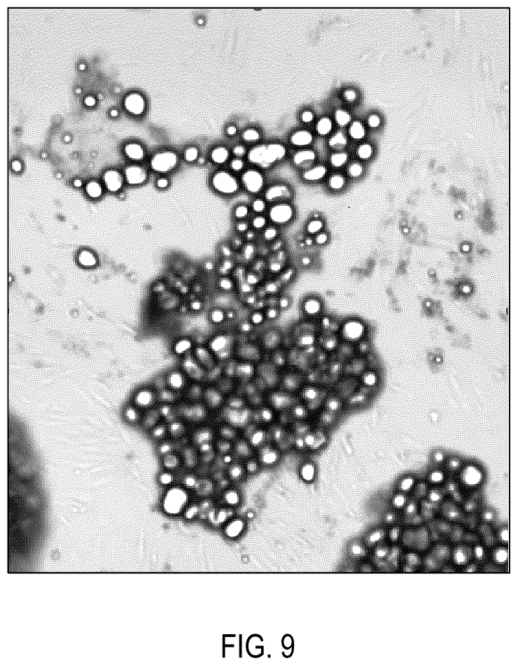

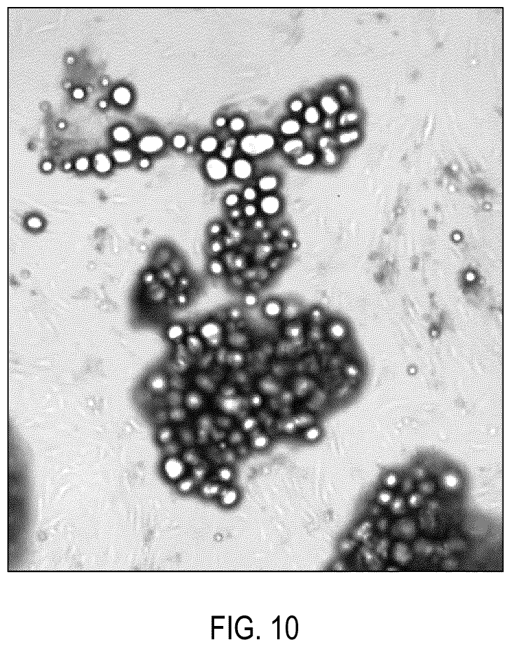

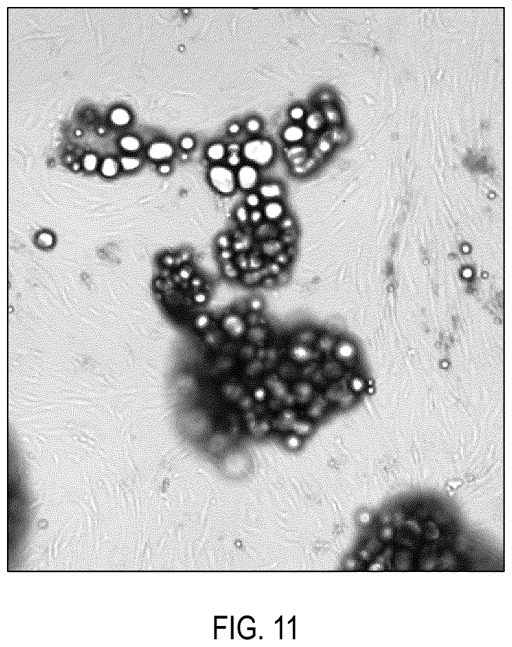

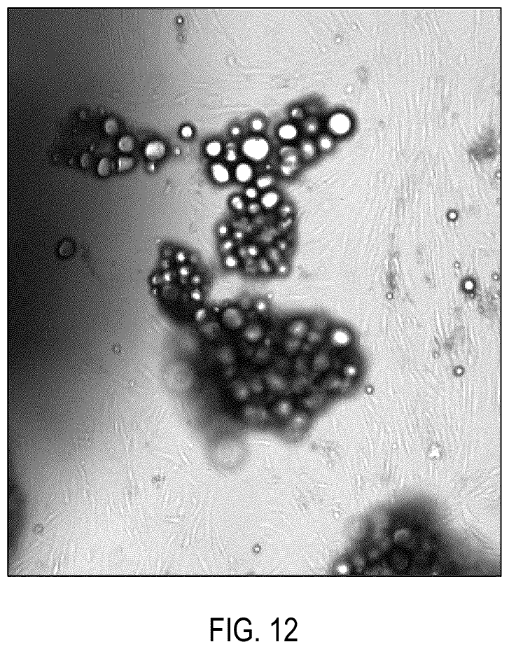

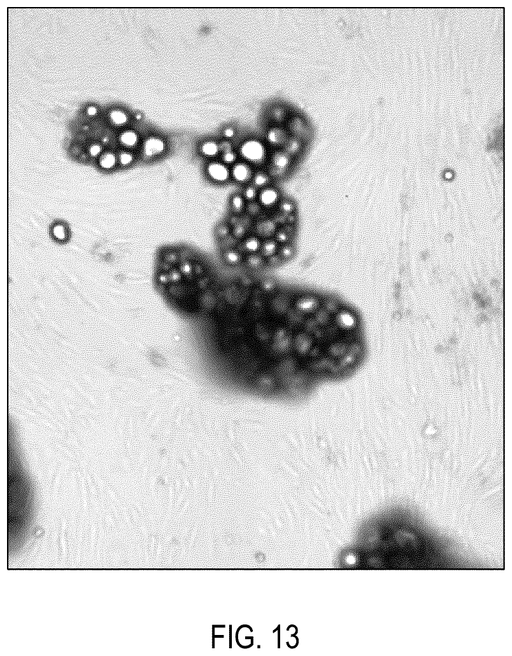

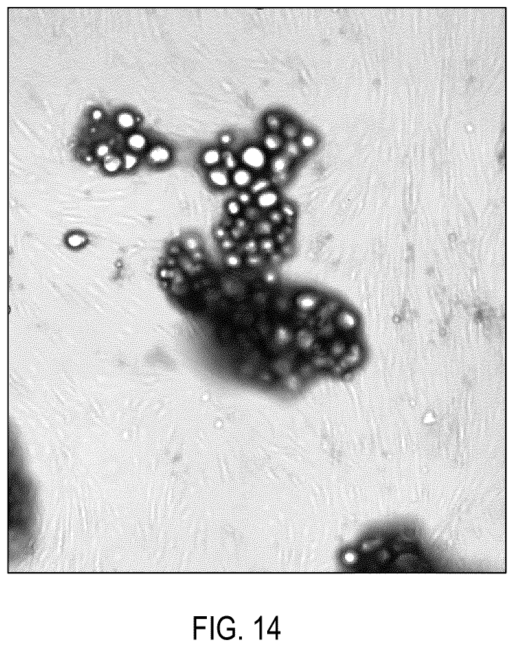

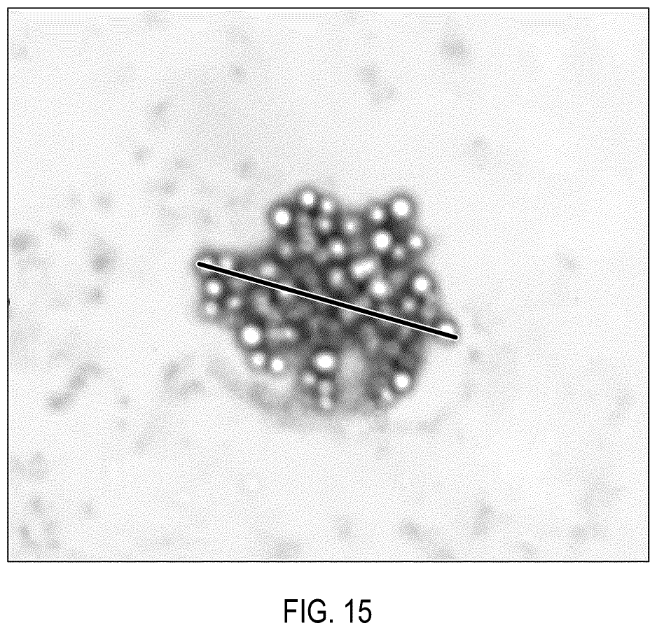

FIG. 6 demonstrates the presence of UCP1 24 hours after 1 μg, 5 μg and 10 μg doses for each 100 mg of ex vivo human WAT and the dose response curve at 1 μg, 5 μg and 10 μg doses. Untreated WAT did not express UCP1 protein. FIGS. 8 through 14 were generated using automated brightfield microscopy. These images show shrinkage of the transfected WAT in one day intervals from the start to the end point at t+6 days after treatment with a 1 μg dose of mRNA of SEQ ID NO: 130. FIGS. 15 through 21 show shrinkage of the transfected WAT in one day intervals from the start to the end point at t+6 days after treatment with a 5 μg dose of mRNA of SEQ ID NO: 130. The black line in FIG. 15 drawn diagonally across the cluster of cells is replicated in FIGS. 16 through 21. The length and position of the line as well as the magnification of the cell cluster depicted are held constant throughout FIGS. 15 through 21. The black line serves as a reference point to demonstrate the degree of shrinkage of the cell cluster over the period of treatment. In FIG. 15, the line spans the entire cluster. In FIG. 21, the line is roughly twice the diameter of the cluster.

### Example 7

The sequence of mRNA of SEQ ID NO: 2 was created using A plasmid Editor (ApE). The coding sequence (CDS) was optimized using the GenScript Biotech Corporation GenSmart tool. The mRNA was manufactured by GeneFab Inc. using N1-methylpseudouridine as a substitute for uridine and packaged in LNP D as described above. 5 μg or 10 μg of the packaged mRNA was introduced to cultured, ex vivo human white adipose tissue that was prepared as described above. Treatment was performed by adding our drug test articles to the ex vivo fat culture medium (DMEM, 10% fetal bovine serum, and antibiotic and antifungal prophylactics.) After 24 hours, the culture medium was aspirated from the dish; cells were washed in chilled phosphate buffered saline (PBS) and PBS was aspirated completely after wash. A lysis buffer supplemented with protease and phosphatase inhibitors was added to the cell culture. The cells and cell lysate were transferred to a pre-chilled microcentrifuge tube. The lysate was incubated on ice for 15-30 mins and then centrifuged for 20 minutes to pellet cell debris. Supernatant was loaded into a new microcentrifuge tube and protein quantified. Western blot was performed to confirm the expression of UCP1 in the transfected WAT, as well as expected levels of housekeeping gene HSP90.

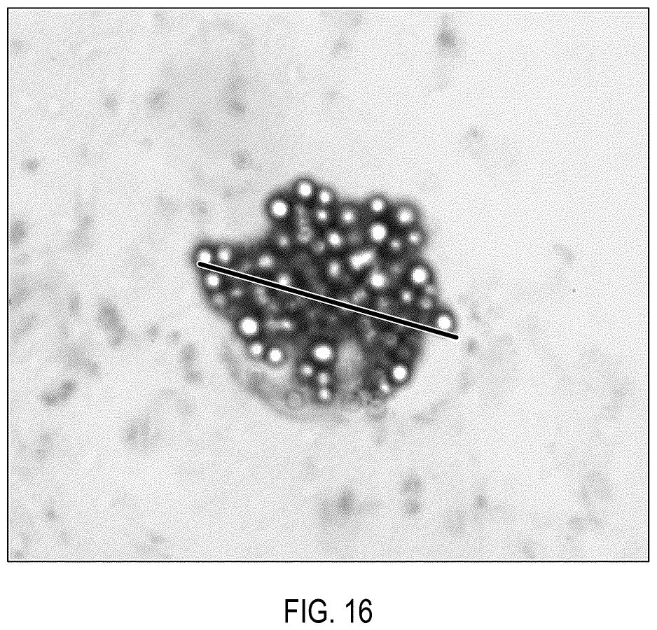

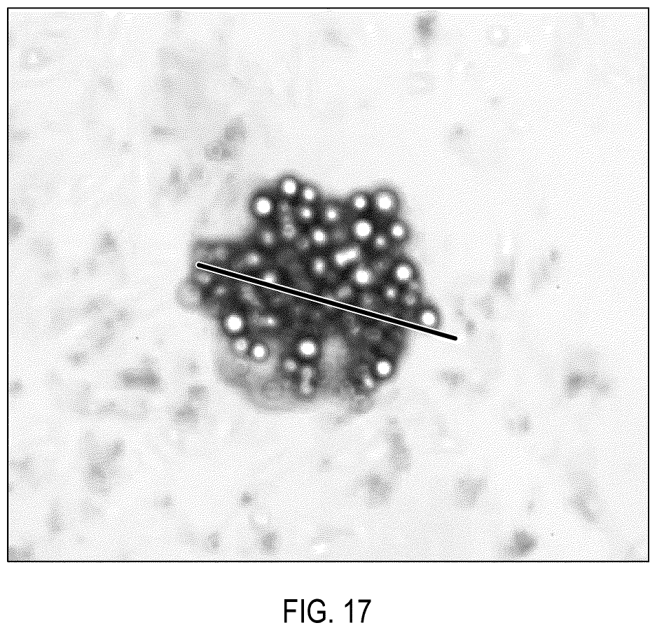

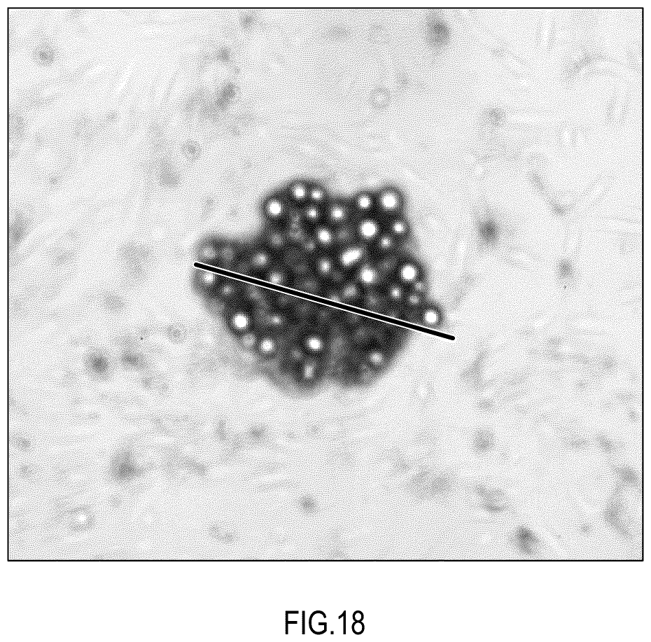

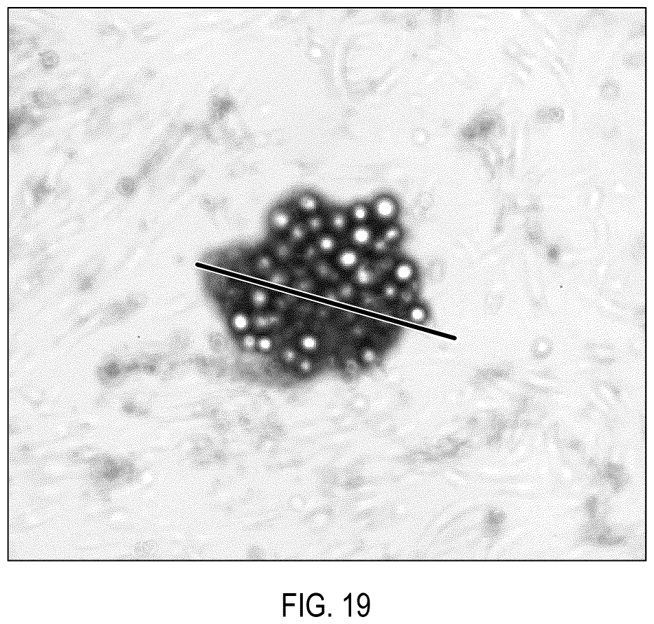

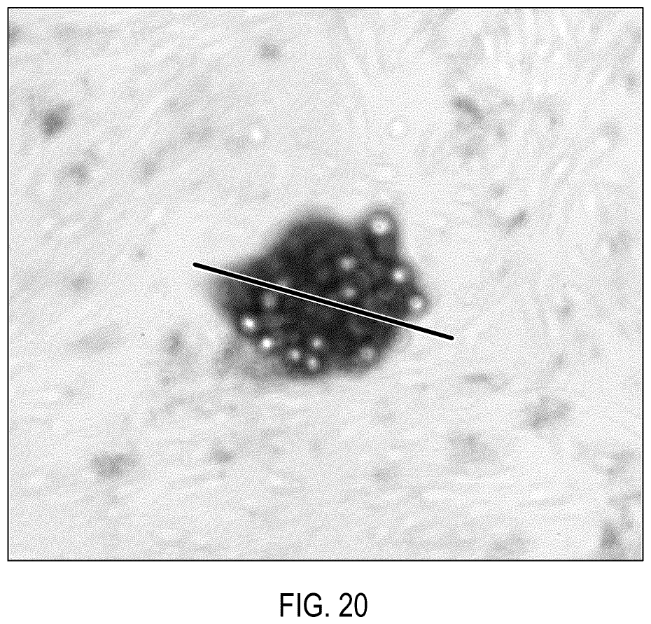

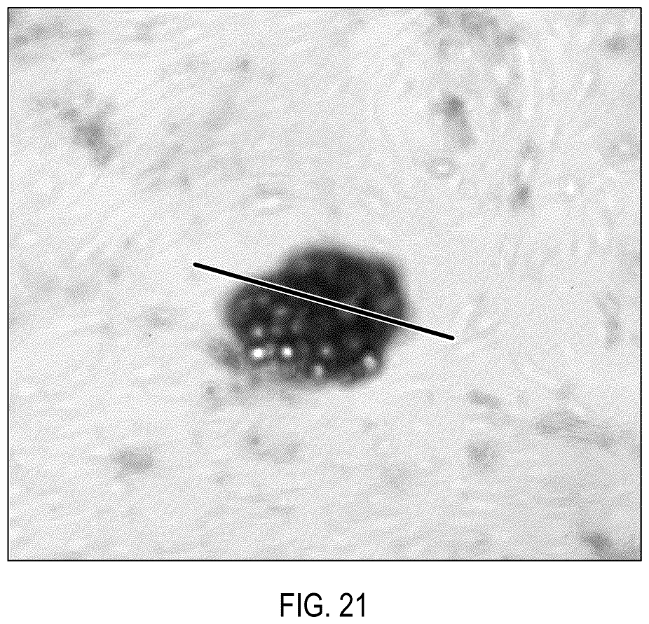

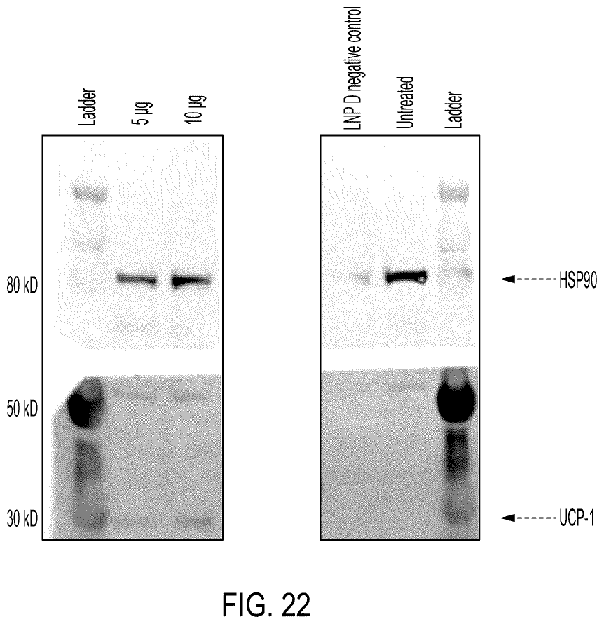

FIG. 22 demonstrates expression of UCP1 after 24 hours of incubation. Negative control test articles (LNP D encapsulating synthetic mRNA encoding mCherry in lieu of UCP1) and untreated WAT did not express UCP1 protein.

### Example 8

The sequence of mRNA of SEQ ID NO: 130 was created using A plasmid Editor (ApE). The coding sequence (CDS) was optimized using the GenScript Biotech Corporation GenSmart tool. The mRNA was manufactured by GeneFab Inc. using N1-methylpseudouridine as a substitute for uridine and packaged in LNP I as described above. One microgram of the packaged mRNA was added to a confluent culture of human adipose derived stem cells (ASCs) in the culture medium (DMEM, 10% fetal bovine serum, and antibiotic and antifungal prophylactics.) After 24 hours, the culture medium was aspirated from the dish; cells were washed in chilled phosphate buffered saline (PBS) and PBS was aspirated completely after wash. A lysis buffer supplemented with protease and phosphatase inhibitors was added to the cell culture. The cells and cell lysate were transferred to a pre-chilled microcentrifuge tube. The lysate was incubated on ice for 15-30 mins and then centrifuged for 20 minutes to pellet cell debris. Supernatant was loaded into a new microcentrifuge tube and protein quantified. Western blot was performed to confirm the expression of UCP1 in the transfected WAT, as well as expected levels of housekeeping genes heat shock protein 90 (HSP90) and beta-actin (0-actin).

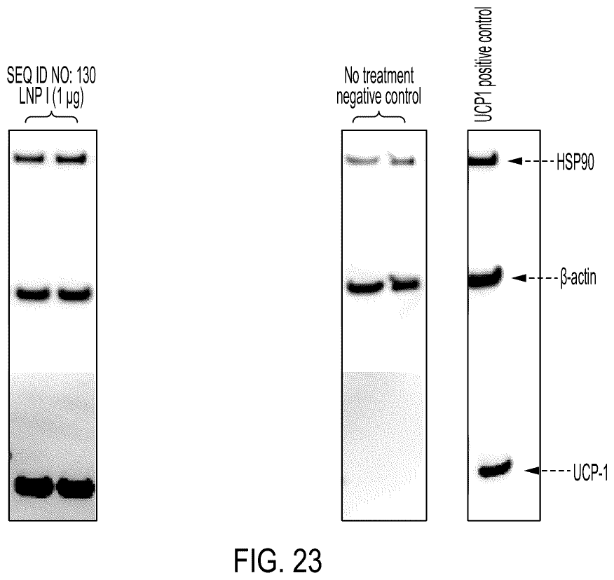

FIG. 23 demonstrates the expression level of UCP1 24 hours after adding 1 μg of mRNA.

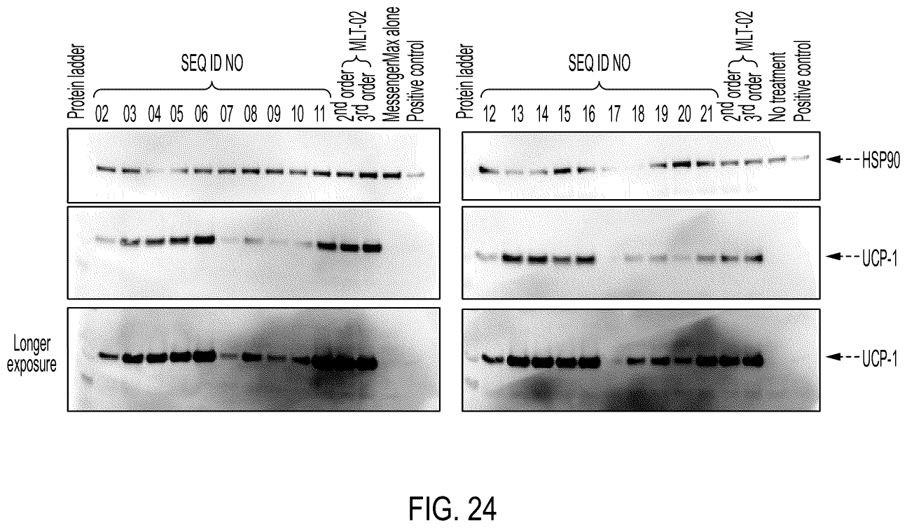

FIG. 24 demonstrates the expression level of UCP1 24 hours after adding 1 μg doses of mRNA of SEQ ID NO: 2 through 21 encapsulated in MessengerMax™ transfection agent to 100 mg of ex vivo human WAT. As shown in the controls untreated WAT did not express UCP1 protein.

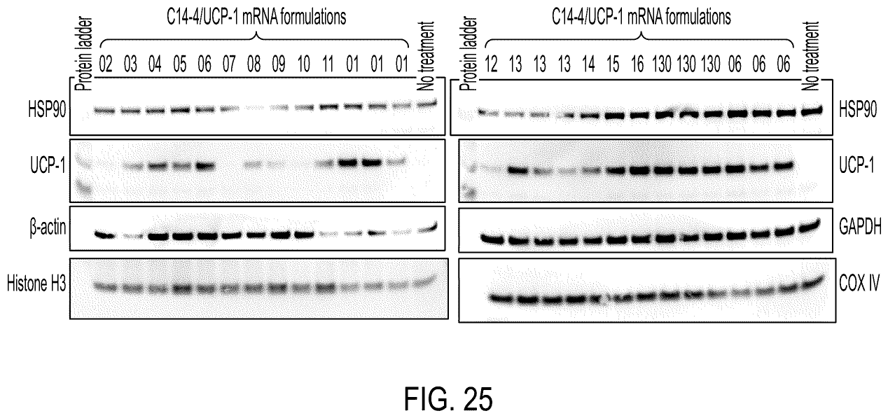

FIG. 25 demonstrates the expression level of UCP1 24 hours after adding 1 μg doses of mRNA of SEQ ID NO: 2 through 16 as well as SEQ ID NO: 130 and 1 which, along with SEQ ID NO: 6 were run in triplicate, encapsulated in LNP I in 100 mg of ex vivo human WAT.

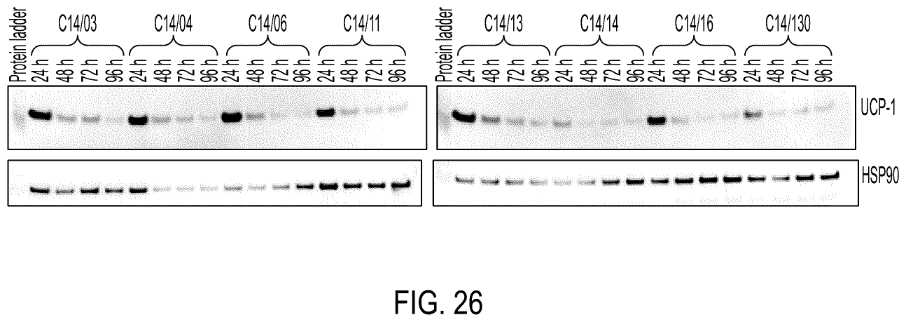

FIG. 26 demonstrates the presence of UCP1 at time points 24, 48, 72 and 96 hours post transfection of 100 mg of ex vivo human WAT with 1 μg mRNA of SEQ ID NO: 3, 4, 6, 11, 13, 14, 16 and 130 encapsulated in LNP I.

FIG. 27 was generated using automated brightfield microscopy. These images, from left to right and top to bottom, show shrinkage of the transfected WAT as observed in visual microscopy in one day intervals from the start to the end point at t+6 days after treatment as described above with a 10 μg dose of mRNA of SEQ ID NO: 130 encapsulated in LNP I of component ratio (C14-4:DSPC:Cholesterol:PEG/Lipid Conjugate) of 35:16:46.5:2.5. The white line in the upper left panel of FIG. 27 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly twice the diameter of the cluster.

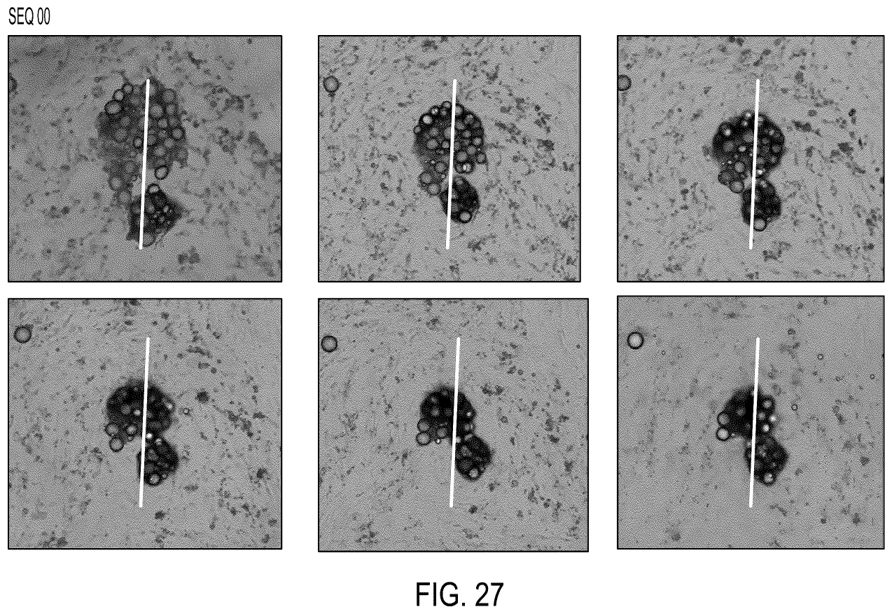

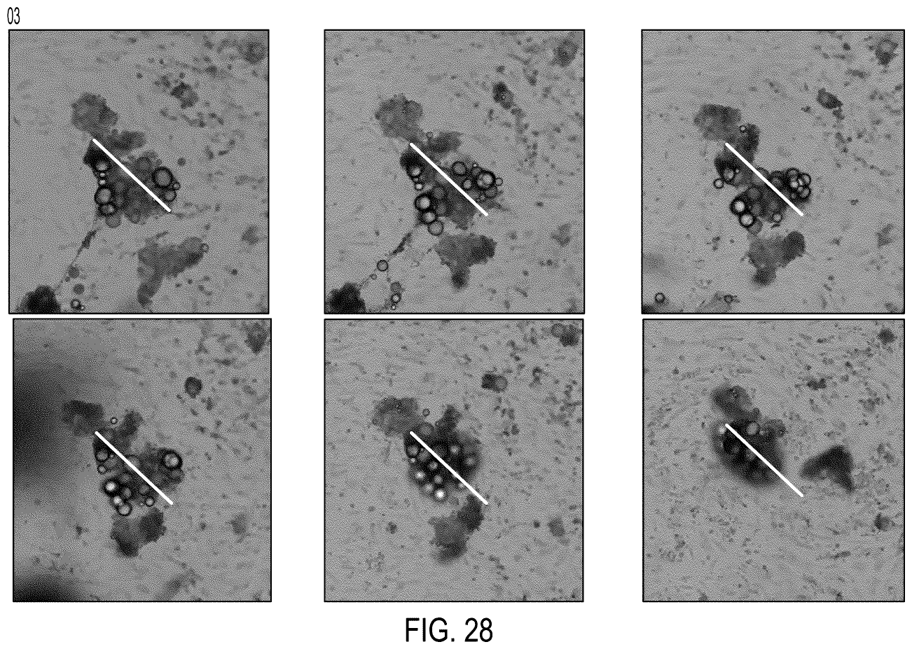

FIG. 28 was generated using automated brightfield microscopy. These images, read from left to right and top to bottom, show shrinkage of the transfected WAT in one day intervals from the start to the end point at t+6 days after treatment as described above with a 10 μg dose of mRNA of SEQ ID NO: 3 encapsulated in LNP I of component ratio (C14-4:DSPC:Cholesterol:PEG/Lipid Conjugate) of 35:16:46.5:2.5. The white line in the upper left panel of FIG. 28 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly one and a half times the diameter of the cluster.

FIG. 29 was generated using automated brightfield microscopy. These images, read from left to right and top to bottom, show shrinkage of the transfected WAT in one day intervals from the start to the end point at t+6 days after treatment as described above with a 10 μg dose of mRNA of SEQ ID NO: 4 encapsulated in LNP I of component ratio (C14-4:DSPC:Cholesterol:PEG/Lipid Conjugate) of 35:16:46.5:2.5. The white line in the upper left panel of FIG. 29 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly one and a half times the diameter of the cluster.

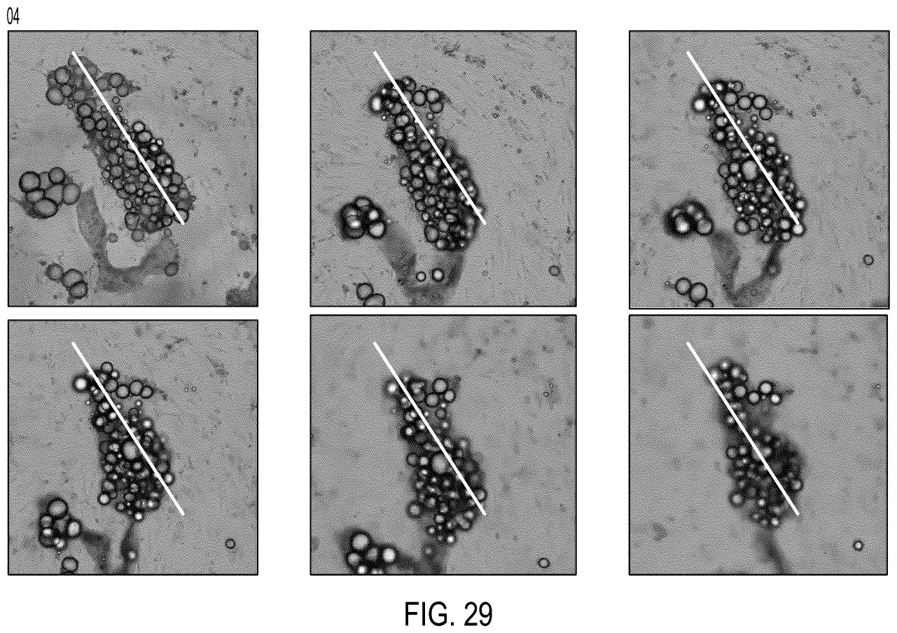

FIG. 30 was generated using automated brightfield microscopy. These images, read from left to right and top to bottom, show shrinkage of the transfected WAT in one day intervals from the start to the end point at t+6 days after treatment as described above with a 2 μg dose of mRNA of SEQ ID NO: 6 encapsulated in LNP I of component ratio (C14-4:DSPC:Cholesterol:PEG/Lipid Conjugate) of 35:16:46.5:2.5. The white line in the upper left panel of FIG. 30 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly one and a half times the diameter of the cluster.

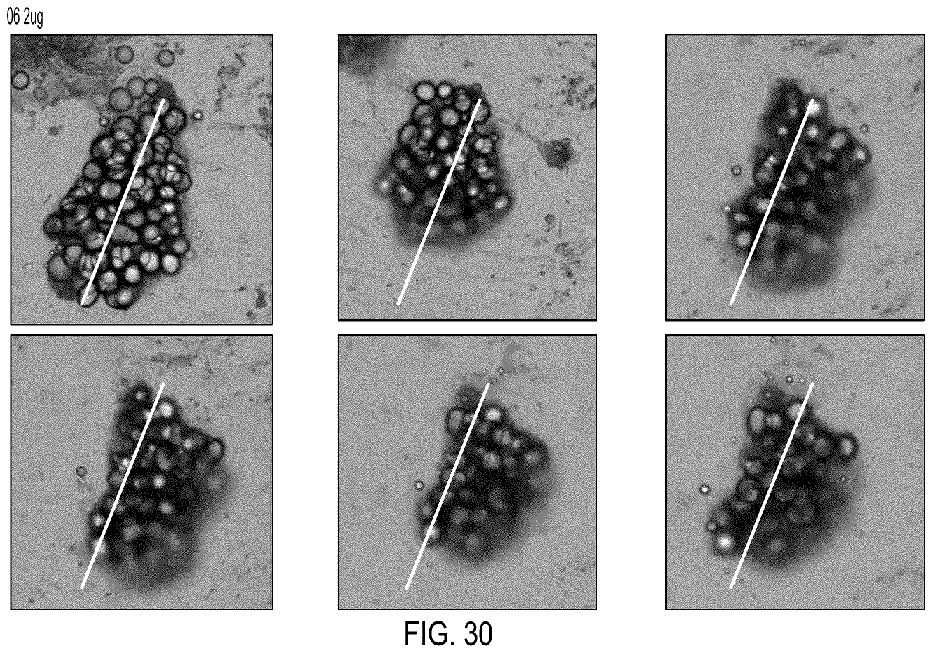

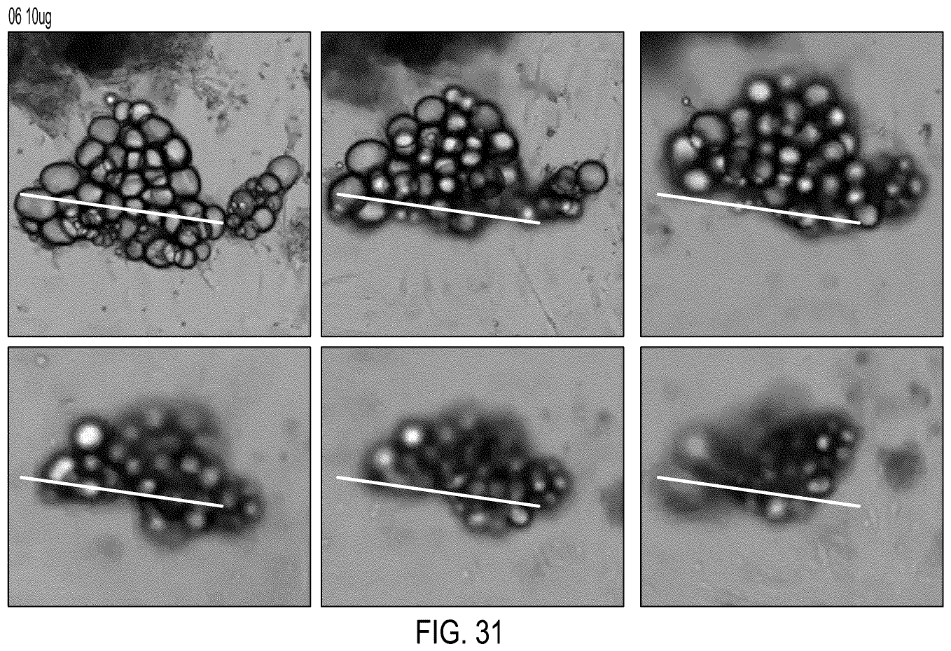

FIG. 31 was generated using automated brightfield microscopy. These images, read from left to right and top to bottom, show shrinkage of the transfected WAT in one day intervals from the start to the end point at t+6 days after treatment as described above with a 10 μg dose of mRNA of SEQ ID NO: 6 encapsulated in LNP I of component ratio (C14-4:DSPC:Cholesterol:PEG/Lipid Conjugate) of 35:16:46.5:2.5. The white line in the upper left panel of FIG. 31 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly one and a half times the diameter of the cluster.

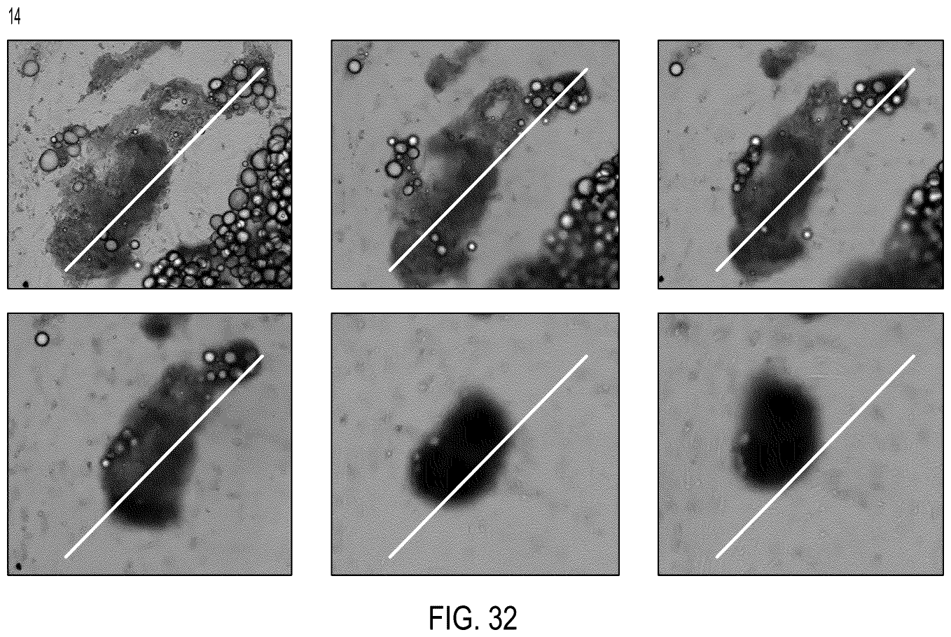

FIG. 32 was generated using automated brightfield microscopy. These images, read from left to right and top to bottom, show shrinkage of the transfected WAT in one day intervals from the start to the end point at t+6 days after treatment as described above with a 10 μg dose of mRNA of SEQ ID NO: 14 encapsulated in LNP I of component ratio (C14-4:DSPC:Cholesterol:PEG/Lipid Conjugate) of 35:16:46.5:2.5. The white line in the upper left panel of FIG. 32 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly twice the diameter of the cluster.

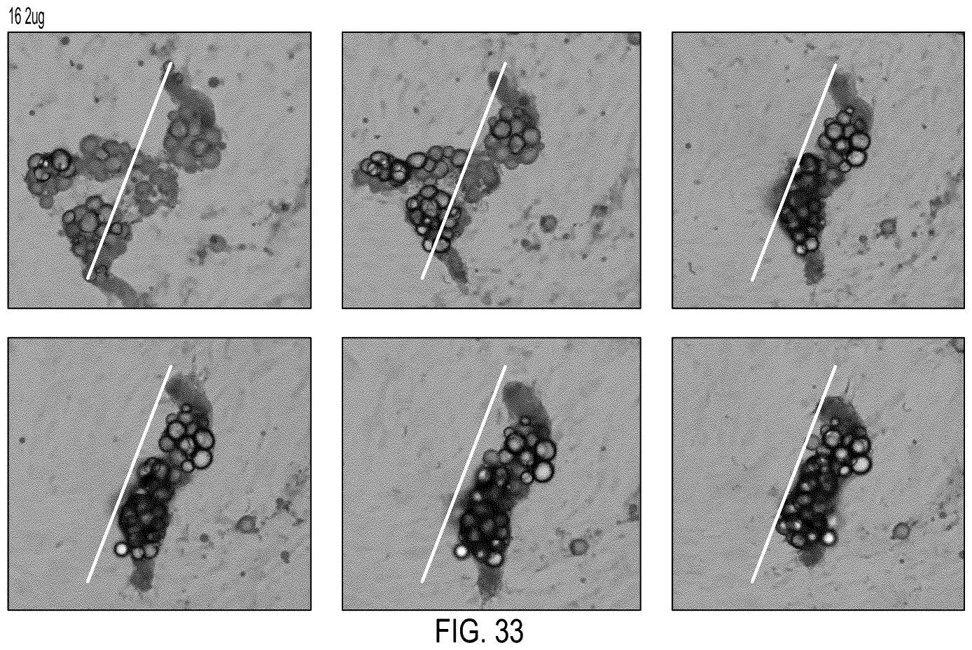

FIG. 33 was generated using automated brightfield microscopy. These images, read from left to right and top to bottom, show shrinkage of the transfected WAT in one day intervals from the start to the end point at t+6 days after treatment as described above with a 2 μg dose of mRNA of SEQ ID NO: 16 encapsulated in LNP I of component ratio (C14-4:DSPC:Cholesterol:PEG/Lipid Conjugate) of 35:16:46.5:2.5. The white line in the upper left panel of FIG. 31 drawn diagonally across the cluster of cells is replicated in each of the succeeding images. The length and position of the line as well as the magnification of the cell cluster depicted are held constant throughout the images. The white line serves as a reference point to demonstrate the degree of shrinkage of the cell cluster over the period of treatment. In the first image, the line spans the entire cluster. In the last image, the line is roughly one and a half times the diameter of the cluster.

### Example 9

Synthetic mRNA of SEQ ID NO: 130 expressing UCP1 was synthesized via in vitro transcription from a linearized DNA plasmid. The synthetic mRNA was encapsulated into lipid nanoparticles of LNP I. Subcutaneous fat was donated by a patient undergoing elective surgery under an IRB-approved protocol. The fat was stabilized for long-term ex vivo culture. The ex vivo fat was treated with the packaged mRNA at a dose of 10 μg per 100 mg of ex vivo fat. Brightfield microscopy images were captured every 4 to 6 hours using an automated system for a total of 6 days (144 hours). In FIG. 27, every treated fat cluster shrank significantly with a majority of the unilocular adipocytes shrinking to the point where they could no longer be visually identified as mature, unilocular adipocytes.

### Example 10

Synthetic mRNA of SEQ ID NO: 3 expressing UCP1 was synthesized via in vitro transcription from a linearized DNA plasmid. The synthetic mRNA was encapsulated into lipid nanoparticles of LNP I, yielding a drug candidate test article. Subcutaneous fat was donated by a patient undergoing elective surgery under an IRB-approved protocol. The fat was stabilized for long-term ex vivo culture. The ex vivo fat was treated with the drug candidate test article at a dose of 10 μg drug candidate per 100 mg of ex vivo fat. Brightfield microscopy images were captured every 4 to 6 hours using an automated system for a total of 6 days (144 hours). In FIG. 28, every treated fat cluster shrank significantly with a majority of the unilocular adipocytes shrinking to the point where they could no longer be visually identified as mature, unilocular adipocytes.

### Example 11

Synthetic mRNA of SEQ ID NO: 4 expressing UCP1 was synthesized via in vitro transcription from a linearized DNA plasmid. The synthetic mRNA was encapsulated into lipid nanoparticles of LNP I. Subcutaneous fat was donated by a patient undergoing elective surgery under an IRB-approved protocol. The fat was stabilized for long-term ex vivo culture. The ex vivo fat was treated with the packaged mRNA at a dose of 10 μg per 100 mg of ex vivo fat. Brightfield microscopy images were captured every 4 to 6 hours using an automated system for a total of 6 days (144 hours). In FIG. 29, every treated fat cluster shrank significantly with a majority of the unilocular adipocytes shrinking to the point where they could no longer be visually identified as mature, unilocular adipocytes.

### Example 12

Synthetic mRNA of SEQ ID NO: 6 expressing UCP1 was synthesized via in vitro transcription from a linearized DNA plasmid. The synthetic mRNA was encapsulated into lipid nanoparticles of LNP I, yielding a drug candidate test article. Subcutaneous fat was donated by a patient undergoing elective surgery under an IRB-approved protocol. The fat was stabilized for long-term ex vivo culture. The ex vivo fat was treated with the packaged mRNA at a dose of 2 μg per 100 mg of ex vivo fat. Brightfield microscopy images were captured every 4 to 6 hours using an automated system for a total of 6 days (144 hours). In FIG. 30, every treated fat cluster shrank significantly with a majority of the unilocular adipocytes shrinking to the point where they could no longer be visually identified as mature, unilocular adipocytes.

### Example 13

Synthetic mRNA of SEQ ID NO: 6 expressing UCP1 was synthesized via in vitro transcription from a linearized DNA plasmid. The synthetic mRNA was encapsulated into lipid nanoparticles of LNP I. Subcutaneous fat was donated by a patient undergoing elective surgery under an IRB-approved protocol. The fat was stabilized for long-term ex vivo culture. The ex vivo fat was treated with the packaged mRNA at a dose of 10 μg per 100 mg of ex vivo fat. Brightfield microscopy images were captured every 4 to 6 hours using an automated system for a total of 6 days (144 hours). In FIG. 31, every treated fat cluster shrank significantly with a majority of the unilocular adipocytes shrinking to the point where they could no longer be visually identified as mature, unilocular adipocytes.

### Example 14

Synthetic mRNA of SEQ ID NO: 14 expressing UCP1 was synthesized via in vitro transcription from a linearized DNA plasmid. The synthetic mRNA was encapsulated into lipid nanoparticles of LNP I. Subcutaneous fat was donated by a patient undergoing elective surgery under an IRB-approved protocol. The fat was stabilized for long-term ex vivo culture. The ex vivo fat was treated with the packaged mRNA at a dose of 10 μg per 100 mg of ex vivo fat. Brightfield microscopy images were captured every 4 to 6 hours using an automated system for a total of 6 days (144 hours). In FIG. 32, every treated fat cluster shrank significantly with a majority of the unilocular adipocytes shrinking to the point where they could no longer be visually identified as mature, unilocular adipocytes.

### Example 15

Synthetic mRNA of SEQ ID NO: 16 expressing UCP1 was synthesized via in vitro transcription from a linearized DNA plasmid. The synthetic mRNA was encapsulated into lipid nanoparticles of LNP I. Subcutaneous fat was donated by a patient undergoing elective surgery under an IRB-approved protocol. The fat was stabilized for long-term ex vivo culture. The ex vivo fat was treated with the packaged mRNA at a dose of 2 μg per 100 mg of ex vivo fat. Brightfield microscopy images were captured every 4 to 6 hours using an automated system for a total of 6 days (144 hours). In FIG. 33, every treated fat cluster shrank significantly with a majority of the unilocular adipocytes shrinking to the point where they could no longer be visually identified as mature, unilocular adipocytes.

SEQUENCES
Sequence total quantity: 130 (Sequences are shown 5′ to 3′)

| SEQ ID | | |
|---|---|---|
| NO | | RNA Sequence |
| 130 | mol_type = | GAGACCCAAGCUGGCUAGCGGAAUUCGUCGACUGGAUCCGGUAC |
| | RNA | CGAGGAGAUCUGCCGCCGCGAUCGCCAUGGGCGGGCUGACAGCC |
| | origin = | AGCGACGUGCACCCAACCCUGGGAGUGCAGCUGUUUUCCGCCGG |
| | synthetic | CAUUGCUGCCUGUCUGGCUGAUGUGAUCACUUUCCCACUGGACA |
| | construct | CAGCUAAAGUGCGGCUGCAGGUGCAGGGCGAGUGCCCAACAAGC |
| | | UCUGUGAUCAGGUACAAGGGCGUGCUGGGGACCAUCACCGCCGU |
| | | GGUGAAGACCGAGGGCAGAAUGAAACUGUAUAGUGGUCUUCCCG |
| | | CCGGCCUGCAGAGACAGAUUUCUAGCGCCUCACUGCGCAUUGGC |
| | | CUGUAUGAUACAGUGCAGGAGUUUCUGACUGCCGGCAAGGAAAC |
| | | UGCUCCAUCCCUGGGCAGCAAAAUCCUGGCCGGACUCACAACUG |
| | | GCGGCGUGGCCGUCUUCAUCGGGCAGCCCACUGAGGUGGUGAAG |
| | | GUGCGCCUGCAGGCCCAGAGCCACUUGCAUGGGAUCAAACCCAG |
| | | AUACACAGGAACCUACAACGCUUACAGAAUCAUCGCCACCACCG |
| | | AGGGCCUGACUGGGCUGUGGAAGGGCACUACCCCAAACCUGAUG |
| | | CGGAGUGUGAUCAUUAAUUGUACUGAGCUGGUGACCUAUGAUC |
| | | UGAUGAAGGAGGCCUUUGUGAAGAACAACAUCCUGGCCGACGAC |
| | | GUGCCUUGUCACCUGGUGAGCGCCCUGAUCGCCGGCUUCUGCGC |
| | | CACAGCCAUGAGCUCCCCCGUGGACGUGGUGAAGACAAGAUUCA |
| | | UCAAUAGCCCACCUGGCCAGUAUAAAUCCGUGCCUAAUUGCGCC |
| | | AUGAAGGUGUUCACCAAUGAGGGGCCCACCGCCUUUUUCAAGGG |
| | | GCUGGUGCCCUCCUUCCUGAGGCUGGGCAGUUGGAACGUGAUCA |
| | | UGUUCGUGUGUUUCGAACAGCUGAAAAGAGAGCUGUCCAAGUCU |
| | | AGACAGACCAUGGACUGCGCCACAUGAUGACCCGGGAGGGCCCG |
| | | CGGCCGCUCGAGUCUAGAGGGCCCGUUUAAACGUCUGAGUGGGC |
| | | GGCGAAUUCAAAAAAAAAAAAAAAAAAAAAAAAAAAAAAAAAA |
| | | AAAAAAAAAAAAAAAAAAAAAAAAAAAAAAAAAAAA |
| 1 | mol_type = | GGAGAAUUCGUCGACUGGAUCCGGUACCGAGGAGAUCUGCCGCC |
| | RNA | GCGAUCGCCAUGGGCGGGCUGACAGCCAGCGACGUGCACCCAAC |
| | origin = | CCUGGGAGUGCAGCUGUUUUCCGCCGGCAUUGCUGCCUGUCUGG |
| | synthetic | CUGAUGUGAUCACUUUCCCACUGGACACAGCUAAAGUGCGGCUG |
| | construct | CAGGUGCAGGGCGAGUGCCCAACAAGCUCUGUGAUCAGGUACAA |
| | | GGGCGUGCUGGGGACCAUCACCGCCGUGGUGAAGACCGAGGGCA |
| | | GAAUGAAACUGUAUAGUGGUCUUCCCGCCGGCCUGCAGAGACAG |
| | | AUUUCUAGCGCCUCACUGCGCAUUGGCCUGUAUGAUACAGUGCA |
| | | GGAGUUUCUGACUGCCGGCAAGGAAACUGCUCCAUCCCUGGGCA |
| | | GCAAAAUCCUGGCCGGACUCACAACUGGCGGCGUGGCCGUCUUC |
| | | AUCGGGCAGCCCACUGAGGUGGUGAAGGUGCGCCUGCAGGCCCA |
| | | GAGCCACUUGCAUGGGAUCAAACCCAGAUACACAGGAACCUACA |
| | | ACGCUUACAGAAUCAUCGCCACCACCGAGGGCCUGACUGGGCUG |
| | | UGGAAGGGCACUACCCCAAACCUGAUGCGGAGUGUGAUCAUUAA |
| | | UUGUACUGAGCUGGUGACCUAUGAUCUGAUGAAGGAGGCCUUU |
| | | GUGAAGAACAACAUCCUGGCCGACGACGUGCCUUGUCACCUGGU |
| | | GAGCGCCCUGAUCGCCGGCUUCUGCGCCACAGCCAUGAGCUCCC |
| | | CCGUGGACGUGGUGAAGACAAGAUUCAUCAAUAGCCCACCUGGC |
| | | CAGUAUAAAUCCGUGCCUAAUUGCGCCAUGAAGGUGUUCACCAA |
| | | UGAGGGGCCCACCGCCUUUUUCAAGGGGCUGGUGCCCUCCUUCC |
| | | UGAGGCUGGGCAGUUGGAACGUGAUCAUGUUCGUGUGUUUCGA |
| | | ACAGCUGAAAAGAGAGCUGUCCAAGUCUAGACAGACCAUGGACU |
| | | GCGCCACAUGAUGACCCGGGGAUCCAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAGAAGAGCUCUAGAGU |
| | | CGACCUGCAGGCAUGCAAGCUUCUCGAGAUGUGAGCAAAAGGCC |
| | | AGCAAAAGGCCAGGAACCGUAAAAAGGCCGCGUUGCUGGCGUUU |
| | | UUCCAUAGGCUCCGCCCCCCUGACGAGCAUCACAAAAAUCGACG |
| | | CUCAAGUCAGAGGUGGCGAAACCCGACAGGACUAUAAAGAUACC |
| | | AGGCGUUUCCCCCUGGAAGCUCCCUCGUGCGCUCUCCUGUUCCG |
| | | ACCCUGCCGCUUACCGGAUACCUGUCCGCCUUUCUCCCUUCGGG |
| | | AAGCGUGGCGCUUUCUCAUAGCUCACGCUGUAGGUAUCUCAGUU |
| | | CGGUGUAGGUCGUUCGCUCCAAGCUGGGCUGUGUGCACGAACCC |
| | | CCCGUUCAGCCCGACCGCUGCGCCUUAUCCGGUAACUAUCGUCU |
| | | UGAGUCCAACCCGGUAAGACACGACUUAUCGCCACUGGCAGCAG |
| | | CCACUGGUAACAGGAUUAGCAGAGCGAGGUAUGUAGGCGGUGCU |
| | | ACAGAGUUCUUGAAGUGGUGGCCUAACUACGGCUACACUAGAAG |
| | | AACAGUAUUUGGUAUCUGCGCUCUGCUGAAGCCAGUUACCUUCG |
| | | GAAAAAGAGUUGGUAGCUCUUGAUCCGGCAAACAAACCACCGCU |
| | | GGUAGCGGUGGUUUUUUUGUUUGCAAGCAGCAGAUUACGCGCA |
| | | GAAAAAAAGGAUCUCAAGAAGAUCCUUUGAUCUUUUCUACGGG |
| | | GUCUGACGCUCAGUGGAACGAAAACUCACGUUAAGGGAUUUUGG |
| | | UCAUGAGCCUAGGCGCCGUCCCGUCAAGUCAGCGUAAUGCUCUG |
| | | CCAGUGUUACAACCAAUUAACCAAUUCUGAUUAGAAAAACUCAU |
| | | CGAGCAUCAAAUGAAACUGCAAUUUAUUCAUAUCAGGAUUAUCA |
| | | AUACCAUAUUUUUGAAAAAGCCGUUUCUGUAAUGAAGGAGAAA |
| | | ACUCACCGAGGCAGUUCCAUAGGAUGGCAAGAUCCUGGUAUCGG |
| | | UCUGCGAUUCCGACUCGUCCAACAUCAAUACAACCUAUUAAUUU |
| | | CCCCUCGUCAAAAAUAAGGUUAUCAAGUGAGAAAUCACCAUGAG |
| | | UGACGACUGAAUCCGGUGAGAAUGGCAAAAGCUUAUGCAUUUCU |
| | | UUCCAGACUUGUUCAACAGGCCAGCCAUUACGCUCGUCAUCAAA |
| | | AUCACUCGCAUCAACCAAACCGUUAUUCAUUCGUGAUUGCGCCU |
| | | GAGCGAGACGAAAUACGCGAUCGCUGUUAAAAGGACAAUUACAA |
| | | ACAGGAAUCGAAUGCAACCGGCGCAGGAACACUGCCAGCGCAUC |
| | | AACAAUAUUUUCACCUGAAUCAGGAUAUUCUUCUAAUACCUGGA |
| | | AUGCUGUUUUCCCGGGGAUCGCAGUGGUGAGUAACCAUGCAUCA |
| | | UCAGGAGUACGGAUAAAAUGCUUGAUGGUCGGAAGAGGCAUAA |
| | | AUUCCGUCAGCCAGUUUAGUCUGACCAUCUCAUCUGUAACAUCA |
| | | UUGGCAACGCUACCUUUGCCAUGUUUCAGAAACAACUCUGGCGC |
| | | AUCGGGCUUCCCAUAAAGUCGAUAGAUUGUCGCACCUGAUUGCC |
| | | CGACAUUAUCGCGAGCCCAUUUAUACCCAUAUAAAUCAGCAUCC |
| | | AUGUUGGAAUUUAAUCGCGGCCUUGAGCAAGACGUUUCCCGUUG |
| | | AAUAUGGCUCAUAACACCCCUUGUAUUACUGUUUAUGUAAGCAG |
| | | ACAGUUUUAUUGUUCAUGAUGAUAUAUUUUUAUCUUGUGCAAU |
| | | GUAACAUCAGAGAUUUUGAGACACAACGUG |
| 2 | mol_type = | AUGGGCGGGCUGACAGCCAGCGACGUGCACCCAACCCUGGGAGU |
| | RNA | GCAGCUGUUUUCCGCCGGCAUUGCUGCCUGUCUGGCUGAUGUGA |
| | origin = | UCACUUUCCCACUGGACACAGCUAAAGUGCGGCUGCAGGUGCAG |
| | synthetic | GGCGAGUGCCCAACAAGCUCUGUGAUCAGGUACAAGGGCGUGCU |
| | construct | GGGGACCAUCACCGCCGUGGUGAAGACCGAGGGCAGAAUGAAAC |
| | | UGUAUAGUGGUCUUCCCGCCGGCCUGCAGAGACAGAUUUCUAGC |
| | | GCCUCACUGCGCAUUGGCCUGUAUGAUACAGUGCAGGAGUUUCU |
| | | GACUGCCGGCAAGGAAACUGCUCCAUCCCUGGGCAGCAAAAUCC |
| | | UGGCCGGACUCACAACUGGCGGCGUGGCCGUCUUCAUCGGGCAG |
| | | CCCACUGAGGUGGUGAAGGUGCGCCUGCAGGCCCAGAGCCACUU |
| | | GCAUGGGAUCAAACCCAGAUACACAGGAACCUACAACGCUUACA |
| | | GAAUCAUCGCCACCACCGAGGGCCUGACUGGGCUGUGGAAGGGC |
| | | ACUACCCCAAACCUGAUGCGGAGUGUGAUCAUUAAUUGUACUGA |
| | | GCUGGUGACCUAUGAUCUGAUGAAGGAGGCCUUUGUGAAGAAC |
| | | AACAUCCUGGCCGACGACGUGCCUUGUCACCUGGUGAGCGCCCU |
| | | GAUCGCCGGCUUCUGCGCCACAGCCAUGAGCUCCCCCGUGGACG |
| | | UGGUGAAGACAAGAUUCAUCAAUAGCCCACCUGGCCAGUAUAAA |
| | | UCCGUGCCUAAUUGCGCCAUGAAGGUGUUCACCAAUGAGGGGCC |
| | | CACCGCCUUUUUCAAGGGGCUGGUGCCCUCCUUCCUGAGGCUGG |
| | | GCAGUUGGAACGUGAUCAUGUUCGUGUGUUUCGAACAGCUGAA |
| | | AAGAGAGCUGUCCAAGUCUAGACAGACCAUGGACUGCGCCACAU |
| | | GAUGAC |
| 3 | mol_type = | CGCCAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGAC |
| 4 | mol_type = | CAAGAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGAC |
| 5 | mol_type = | AGAGGGUCCUGCUGGCGCGAGGGUGGGUAGGAGGGGACGCGGG |
| | RNA | GACUCGGCCCCCAACACCGCGCUCCGUCUGCAGCCGCCGCCUCUG |
| | origin = | CACCGCCGCUGCCCGGCGGUCGGUUCAAAAAACAGAAAUCGGGU |
| | synthetic | UUGCUGCCCGGCGGACAGGCGUGAAGAGCAAGGGAAAGGAACUU |
| | construct | CCUCCACCUUCGGGGCUGGAGCCCUUUUCCUCUGCAUCUCCAGU |
| | | CUCUGAGUGAAGAUGGGGGGCUGACAGCCAGCGACGUGCACCC |
| | | AACCCUGGGAGUGCAGCUGUUUUCCGCCGGCAUUGCUGCCUGUC |
| | | UGGCUGAUGUGAUCACUUUCCCACUGGACACAGCUAAAGUGCGG |
| | | CUGCAGGUGCAGGGCGAGUGCCCAACAAGCUCUGUGAUCAGGUA |
| | | CAAGGGCGUGCUGGGGACCAUCACCGCCGUGGUGAAGACCGAGG |
| | | GCAGAAUGAAACUGUAUAGUGGUCUUCCCGCCGGCCUGCAGAGA |
| | | CAGAUUUCUAGCGCCUCACUGCGCAUUGGCCUGUAUGAUACAGU |
| | | GCAGGAGUUUCUGACUGCCGGCAAGGAAACUGCUCCAUCCCUGG |
| | | GCAGCAAAAUCCUGGCCGGACUCACAACUGGCGGCGUGGCCGUC |
| | | UUCAUCGGGCAGCCCACUGAGGUGGUGAAGGUGCGCCUGCAGGC |
| | | CCAGAGCCACUUGCAUGGGAUCAAACCCAGAUACACAGGAACCU |
| | | ACAACGCUUACAGAAUCAUCGCCACCACCGAGGGCCUGACUGGG |
| | | CUGUGGAAGGGCACUACCCCAAACCUGAUGCGGAGUGUGAUCAU |
| | | UAAUUGUACUGAGCUGGUGACCUAUGAUCUGAUGAAGGAGGCC |
| | | UUUGUGAAGAACAACAUCCUGGCCGACGACGUGCCUUGUCACCU |
| | | GGUGAGCGCCCUGAUCGCCGGCUUCUGCGCCACAGCCAUGAGCU |
| | | CCCCCGUGGACGUGGUGAAGACAAGAUUCAUCAAUAGCCCACCU |
| | | GGCCAGUAUAAAUCCGUGCCUAAUUGCGCCAUGAAGGUGUUCAC |
| | | CAAUGAGGGGCCCACCGCCUUUUUCAAGGGGCUGGUGCCCUCCU |
| | | UCCUGAGGCUGGGCAGUUGGAACGUGAUCAUGUUCGUGUGUUUC |
| | | GAACAGCUGAAAAGAGAGCUGUCCAAGUCUAGACAGACCAUGGA |
| | | CUGCGCCACAUGAUGAC |
| 6 | mol_type = | ACAGCACCCUCCUGAAAACUGCAGCUUCCUUCUCACCUUGAAGA |
| | RNA | AUAAUCCUAGAAAACUCACAAAAUGGGCGGGCUGACAGCCAGCG |
| | origin = | ACGUGCACCCAACCCUGGGAGUGCAGCUGUUUUCCGCCGGCAUU |
| | synthetic | GCUGCCUGUCUGGCUGAUGUGAUCACUUUCCCACUGGACACAGC |
| | construct | UAAAGUGCGGCUGCAGGUGCAGGGCGAGUGCCCAACAAGCUCUG |
| | | UGAUCAGGUACAAGGGCGUGCUGGGGACCAUCACCGCCGUGGUG |
| | | AAGACCGAGGGCAGAAUGAAACUGUAUAGUGGUCUUCCCGCCGG |
| | | CCUGCAGAGACAGAUUUCUAGCGCCUCACUGCGCAUUGGCCUGU |
| | | AUGAUACAGUGCAGGAGUUUCUGACUGCCGGCAAGGAAACUGCU |
| | | CCAUCCCUGGGCAGCAAAAUCCUGGCCGGACUCACAACUGGCGG |
| | | CGUGGCCGUCUUCAUCGGGCAGCCCACUGAGGUGGUGAAGGUGC |
| | | GCCUGCAGGCCCAGAGCCACUUGCAUGGGAUCAAACCCAGAUAC |
| | | ACAGGAACCUACAACGCUUACAGAAUCAUCGCCACCACCGAGGG |
| | | CCUGACUGGGCUGUGGAAGGGCACUACCCCAAACCUGAUGCGGA |
| | | GUGUGAUCAUUAAUUGUACUGAGCUGGUGACCUAUGAUCUGAU |
| | | GAAGGAGGCCUUUGUGAAGAACAACAUCCUGGCCGACGACGUGC |
| | | CUUGUCACCUGGUGAGCGCCCUGAUCGCCGGCUUCUGCGCCACA |
| | | GCCAUGAGCUCCCCCGUGGACGUGGUGAAGACAAGAUUCAUCAA |
| | | UAGCCCACCUGGCCAGUAUAAAUCCGUGCCUAAUUGCGCCAUGA |
| | | AGGUGUUCACCAAUGAGGGGCCCACCGCCUUUUUCAAGGGGCUG |
| | | GUGCCCUCCUUCCUGAGGCUGGGCAGUUGGAACGUGAUCAUGUU |
| | | CGUGUGUUUCGAACAGCUGAAAAGAGAGCUGUCCAAGUCUAGAC |
| | | AGACCAUGGACUGCGCCACAUGAUGAC |
| 7 | mol_type = | AUGGGCGGGCUGACAGCCAGCGACGUGCACCCAACCCUGGGAGU |
| | RNA | GCAGCUGUUUUCCGCCGGCAUUGCUGCCUGUCUGGCUGAUGUGA |
| | origin = | UCACUUUCCCACUGGACACAGCUAAAGUGCGGCUGCAGGUGCAG |
| | synthetic | GGCGAGUGCCCAACAAGCUCUGUGAUCAGGUACAAGGGCGUGCU |
| | construct | GGGGACCAUCACCGCCGUGGUGAAGACCGAGGGCAGAAUGAAAC |
| | | UGUAUAGUGGUCUUCCCGCCGGCCUGCAGAGACAGAUUUCUAGC |
| | | GCCUCACUGCGCAUUGGCCUGUAUGAUACAGUGCAGGAGUUUCU |
| | | GACUGCCGGCAAGGAAACUGCUCCAUCCCUGGGCAGCAAAAUCC |
| | | UGGCCGGACUCACAACUGGCGGCGUGGCCGUCUUCAUCGGGCAG |
| | | CCCACUGAGGUGGUGAAGGUGCGCCUGCAGGCCCAGAGCCACUU |
| | | GCAUGGGAUCAAACCCAGAUACACAGGAACCUACAACGCUUACA |
| | | GAAUCAUCGCCACCACCGAGGGCCUGACUGGGCUGUGGAAGGGC |
| | | ACUACCCCAAACCUGAUGCGGAGUGUGAUCAUUAAUUGUACUGA |
| | | GCUGGUGACCUAUGAUCUGAUGAAGGAGGCCUUUGUGAAGAAC |
| | | AACAUCCUGGCCGACGACGUGCCUUGUCACCUGGUGAGCGCCCU |
| | | GAUCGCCGGCUUCUGCGCCACAGCCAUGAGCUCCCCCGUGGACG |
| | | UGGUGAAGACAAGAUUCAUCAAUAGCCCACCUGGCCAGUAUAAA |
| | | UCCGUGCCUAAUUGCGCCAUGAAGGUGUUCACCAAUGAGGGGCC |
| | | CACCGCCUUUUUCAAGGGGCUGGUGCCCUCCUUCCUGAGGCUGG |
| | | GCAGUUGGAACGUGAUCAUGUUCGUGUGUUUCGAACAGCUGAA |
| | | AAGAGAGCUGUCCAAGUCUAGACAGACCAUGGACUGCGCCACAU |
| | | GAUGACUCAGCUUCAAGAAAAUGAUGUAACAUACCAGUGGGAA |
| | | UCUUGCUGACUGGAUCAUAAAAACAAACAAAACUUAUUCACUUA |
| | | UUUUAACCUAAAAAGAUAAAGGAAUUUUGGCAGAGAAUUUUGG |
| | | ACUUUUUUAUAUAAAAAAGAGGAAAAUUAAUGCCUAUUUCAUA |
| | | UAACUUUUUUUUUUUCUCAGUGUCUUAAGAAGGGGAAAGCAAA |
| | | ACAUUCAGCAUAUACCCUGGCAAAUGUAAUGCAGAUAAGCUACU |
| | | GCAUUUGACCAUUUCUGGAGUGCAAUUGUGUGAAUGAAUGUGA |
| | | AGAACUUUAACAUGUUUUAAUUACAAUUCCAACUGGUGGAAAA |
| | | GAAACUGAGUGAAAUGCAGUUUAUAUUUAUAAAUACUUAAAAA |
| | | UGAAGUUAUUAAAAAUAUUAGUUUUUAUUAACCACAGUUGUCA |
| | | GUUAAUAUAUUCAAUAAAGUAUUGCUAAUACCUUUUAAA |
| 8 | mol_type = | CGCCAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGACUCAGCUUCAAGAAAAUGAUGUAACAUACCAGUGGG |
| | | AAUCUUGCUGACUGGAUCAUAAAAACAAACAAAACUUAUUCACU |
| | | UAUUUUAACCUAAAAAGAUAAAGGAAUUUUGGCAGAGAAUUUU |
| | | GGACUUUUUUAUAUAAAAAAGAGGAAAAUUAAUGCCUAUUUCA |
| | | UAUAACUUUUUUUUUUUCUCAGUGUCUUAAGAAGGGGAAAGCA |
| | | AAACAUUCAGCAUAUACCCUGGCAAAUGUAAUGCAGAUAAGCUA |
| | | CUGCAUUUGACCAUUUCUGGAGUGCAAUUGUGUGAAUGAAUGU |
| | | GAAGAACUUUAACAUGUUUUAAUUACAAUUCCAACUGGUGGAA |
| | | AAGAAACUGAGUGAAAUGCAGUUUAUAUUUAUAAAUACUUAAA |
| | | AAUGAAGUUAUUAAAAAUAUUAGUUUUUAUUAACCACAGUUGU |
| | | CAGUUAAUAUAUUCAAUAAAGUAUUGCUAAUACCUUUUAAA |
| 9 | mol_type = | CAAGAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGACUCAGCUUCAAGAAAAUGAUGUAACAUACCAGUGGG |
| | | AAUCUUGCUGACUGGAUCAUAAAAACAAACAAAACUUAUUCACU |
| | | UAUUUUAACCUAAAAAGAUAAAGGAAUUUUGGCAGAGAAUUUU |
| | | GGACUUUUUUAUAUAAAAAAGAGGAAAAUUAAUGCCUAUUUCA |
| | | UAUAACUUUUUUUUUUUCUCAGUGUCUUAAGAAGGGGAAAGCA |
| | | AAACAUUCAGCAUAUACCCUGGCAAAUGUAAUGCAGAUAAGCUA |
| | | CUGCAUUUGACCAUUUCUGGAGUGCAAUUGUGUGAAUGAAUGU |
| | | GAAGAACUUUAACAUGUUUUAAUUACAAUUCCAACUGGUGGAA |
| | | AAGAAACUGAGUGAAAUGCAGUUUAUAUUUAUAAAUACUUAAA |
| | | AAUGAAGUUAUUAAAAAUAUUAGUUUUUAUUAACCACAGUUGU |
| | | CAGUUAAUAUAUUCAAUAAAGUAUUGCUAAUACCUUUUAAA |
| 10 | mol_type = | AGAGGGUCCUGCUGGCGCGAGGGUGGGUAGGAGGGGACGCGGG |
| | RNA | GACUCGGCCCCCAACACCGCGCUCCGUCUGCAGCCGCCGCCUCUG |
| | origin = | CACCGCCGCUGCCCGGCGGUCGGUUCAAAAAACAGAAAUCGGGU |
| | synthetic | UUGCUGCCCGGCGGACAGGCGUGAAGAGCAAGGGAAAGGAACUU |
| | construct | CCUCCACCUUCGGGGCUGGAGCCCUUUUCCUCUGCAUCUCCAGU |
| | | CUCUGAGUGAAGAUGGGCGGGCUGACAGCCAGCGACGUGCACCC |
| | | AACCCUGGGAGUGCAGCUGUUUUCCGCCGGCAUUGCUGCCUGUC |
| | | UGGCUGAUGUGAUCACUUUCCCACUGGACACAGCUAAAGUGCGG |
| | | CUGCAGGUGCAGGGCGAGUGCCCAACAAGCUCUGUGAUCAGGUA |
| | | CAAGGGCGUGCUGGGGACCAUCACCGCCGUGGUGAAGACCGAGG |
| | | GCAGAAUGAAACUGUAUAGUGGUCUUCCCGCCGGCCUGCAGAGA |
| | | CAGAUUUCUAGCGCCUCACUGCGCAUUGGCCUGUAUGAUACAGU |
| | | GCAGGAGUUUCUGACUGCCGGCAAGGAAACUGCUCCAUCCCUGG |
| | | GCAGCAAAAUCCUGGCCGGACUCACAACUGGCGGCGUGGCCGUC |
| | | UUCAUCGGGCAGCCCACUGAGGUGGUGAAGGUGCGCCUGCAGGC |
| | | CCAGAGCCACUUGCAUGGGAUCAAACCCAGAUACACAGGAACCU |
| | | ACAACGCUUACAGAAUCAUCGCCACCACCGAGGGCCUGACUGGG |
| | | CUGUGGAAGGGCACUACCCCAAACCUGAUGCGGAGUGUGAUCAU |
| | | UAAUUGUACUGAGCUGGUGACCUAUGAUCUGAUGAAGGAGGCC |
| | | UUUGUGAAGAACAACAUCCUGGCCGACGACGUGCCUUGUCACCU |
| | | GGUGAGCGCCCUGAUCGCCGGCUUCUGCGCCACAGCCAUGAGCU |
| | | CCCCCGUGGACGUGGUGAAGACAAGAUUCAUCAAUAGCCCACCU |
| | | GGCCAGUAUAAAUCCGUGCCUAAUUGCGCCAUGAAGGUGUUCAC |
| | | CAAUGAGGGGCCCACCGCCUUUUUCAAGGGGCUGGUGCCCUCCU |
| | | UCCUGAGGCUGGGCAGUUGGAACGUGAUCAUGUUCGUGUGUUUC |
| | | GAACAGCUGAAAAGAGAGCUGUCCAAGUCUAGACAGACCAUGGA |
| | | CUGCGCCACAUGAUGACUCAGCUUCAAGAAAAUGAUGUAACAUA |
| | | CCAGUGGGAAUCUUGCUGACUGGAUCAUAAAAACAAACAAAACU |
| | | UAUUCACUUAUUUUAACCUAAAAAGAUAAAGGAAUUUUGGCAG |
| | | AGAAUUUUGGACUUUUUUAUAUAAAAAAGAGGAAAAUUAAUGC |
| | | CUAUUUCAUAUAACUUUUUUUUUUUCUCAGUGUCUUAAGAAGG |
| | | GGAAAGCAAAACAUUCAGCAUAUACCCUGGCAAAUGUAAUGCAG |
| | | AUAAGCUACUGCAUUUGACCAUUUCUGGAGUGCAAUUGUGUGA |
| | | AUGAAUGUGAAGAACUUUAACAUGUUUUAAUUACAAUUCCAAC |
| | | UGGUGGAAAAGAAACUGAGUGAAAUGCAGUUUAUAUUUAUAAA |
| | | UACUUAAAAAUGAAGUUAUUAAAAAUAUUAGUUUUUAUUAACC |
| | | ACAGUUGUCAGUUAAUAUAUUCAAUAAAGUAUUGCUAAUACCU |
| | | UUUAAA |
| 11 | mol_type = | ACAGCACCCUCCUGAAAACUGCAGCUUCCUUCUCACCUUGAAGA |
| | RNA | AUAAUCCUAGAAAACUCACAAAAUGGGCGGGCUGACAGCCAGCG |
| | origin = | ACGUGCACCCAACCCUGGGAGUGCAGCUGUUUUCCGCCGGCAUU |
| | synthetic | GCUGCCUGUCUGGCUGAUGUGAUCACUUUCCCACUGGACACAGC |
| | construct | UAAAGUGCGGCUGCAGGUGCAGGGCGAGUGCCCAACAAGCUCUG |
| | | UGAUCAGGUACAAGGGCGUGCUGGGGACCAUCACCGCCGUGGUG |
| | | AAGACCGAGGGCAGAAUGAAACUGUAUAGUGGUCUUCCCGCCGG |
| | | CCUGCAGAGACAGAUUUCUAGCGCCUCACUGCGCAUUGGCCUGU |
| | | AUGAUACAGUGCAGGAGUUUCUGACUGCCGGCAAGGAAACUGCU |
| | | CCAUCCCUGGGCAGCAAAAUCCUGGCCGGACUCACAACUGGCGG |
| | | CGUGGCCGUCUUCAUCGGGCAGCCCACUGAGGUGGUGAAGGUGC |
| | | GCCUGCAGGCCCAGAGCCACUUGCAUGGGAUCAAACCCAGAUAC |
| | | ACAGGAACCUACAACGCUUACAGAAUCAUCGCCACCACCGAGGG |
| | | CCUGACUGGGCUGUGGAAGGGCACUACCCCAAACCUGAUGCGGA |
| | | GUGUGAUCAUUAAUUGUACUGAGCUGGUGACCUAUGAUCUGAU |
| | | GAAGGAGGCCUUUGUGAAGAACAACAUCCUGGCCGACGACGUGC |
| | | CUUGUCACCUGGUGAGCGCCCUGAUCGCCGGCUUCUGCGCCACA |
| | | GCCAUGAGCUCCCCCGUGGACGUGGUGAAGACAAGAUUCAUCAA |
| | | UAGCCCACCUGGCCAGUAUAAAUCCGUGCCUAAUUGCGCCAUGA |
| | | AGGUGUUCACCAAUGAGGGGCCCACCGCCUUUUUCAAGGGGCUG |
| | | GUGCCCUCCUUCCUGAGGCUGGGCAGUUGGAACGUGAUCAUGUU |
| | | CGUGUGUUUCGAACAGCUGAAAAGAGAGCUGUCCAAGUCUAGAC |
| | | AGACCAUGGACUGCGCCACAUGAUGACUCAGCUUCAAGAAAAUG |
| | | AUGUAACAUACCAGUGGGAAUCUUGCUGACUGGAUCAUAAAAAC |
| | | AAACAAAACUUAUUCACUUAUUUUAACCUAAAAAGAUAAAGGA |
| | | AUUUUGGCAGAGAAUUUUGGACUUUUUUAUAUAAAAAAGAGGA |
| | | AAAUUAAUGCCUAUUUCAUAUAACUUUUUUUUUUUCUCAGUGU |
| | | CUUAAGAAGGGGAAAGCAAAACAUUCAGCAUAUACCCUGGCAAA |
| | | UGUAAUGCAGAUAAGCUACUGCAUUUGACCAUUUCUGGAGUGCA |
| | | AUUGUGUGAAUGAAUGUGAAGAACUUUAACAUGUUUUAAUUAC |
| | | AAUUCCAACUGGUGGAAAAGAAACUGAGUGAAAUGCAGUUUAU |
| | | AUUUAUAAAUACUUAAAAAUGAAGUUAUUAAAAAUAUUAGUUU |
| | | UUAUUAACCACAGUUGUCAGUUAAUAUAUUCAAUAAAGUAUUG |
| | | CUAAUACCUUUUAAA |
| 12 | mol_type = | AUGGGCGGGCUGACAGCCAGCGACGUGCACCCAACCCUGGGAGU |
| | RNA | GCAGCUGUUUUCCGCCGGCAUUGCUGCCUGUCUGGCUGAUGUGA |
| | origin = | UCACUUUCCCACUGGACACAGCUAAAGUGCGGCUGCAGGUGCAG |
| | synthetic | GGCGAGUGCCCAACAAGCUCUGUGAUCAGGUACAAGGGCGUGCU |
| | construct | GGGGACCAUCACCGCCGUGGUGAAGACCGAGGGCAGAAUGAAAC |
| | | UGUAUAGUGGUCUUCCCGCCGGCCUGCAGAGACAGAUUUCUAGC |
| | | GCCUCACUGCGCAUUGGCCUGUAUGAUACAGUGCAGGAGUUUCU |
| | | GACUGCCGGCAAGGAAACUGCUCCAUCCCUGGGCAGCAAAAUCC |
| | | UGGCCGGACUCACAACUGGCGGCGUGGCCGUCUUCAUCGGGCAG |
| | | CCCACUGAGGUGGUGAAGGUGCGCCUGCAGGCCCAGAGCCACUU |
| | | GCAUGGGAUCAAACCCAGAUACACAGGAACCUACAACGCUUACA |
| | | GAAUCAUCGCCACCACCGAGGGCCUGACUGGGCUGUGGAAGGGC |
| | | ACUACCCCAAACCUGAUGCGGAGUGUGAUCAUUAAUUGUACUGA |
| | | GCUGGUGACCUAUGAUCUGAUGAAGGAGGCCUUUGUGAAGAAC |
| | | AACAUCCUGGCCGACGACGUGCCUUGUCACCUGGUGAGCGCCCU |
| | | GAUCGCCGGCUUCUGCGCCACAGCCAUGAGCUCCCCCGUGGACG |
| | | UGGUGAAGACAAGAUUCAUCAAUAGCCCACCUGGCCAGUAUAAA |
| | | UCCGUGCCUAAUUGCGCCAUGAAGGUGUUCACCAAUGAGGGGCC |
| | | CACCGCCUUUUUCAAGGGGCUGGUGCCCUCCUUCCUGAGGCUGG |
| | | GCAGUUGGAACGUGAUCAUGUUCGUGUGUUUCGAACAGCUGAA |
| | | AAGAGAGCUGUCCAAGUCUAGACAGACCAUGGACUGCGCCACAU |
| | | GAUGACCUCGAGCUGGUACUGCAUGCACGCAAUGCUAGCUGCCC |
| | | CUUUCCCGUCCUGGGUACCCCGAGUCUCCCCCGACCUCGGGUCCC |
| | | AGGUAUGCUCCCACCUCCACCUGCCCCACUCACCACCUCUGCUAG |
| | | UUCCAGACACCUCCCAAGCACGCAGCAAUGCAGCUCAAAACGCU |
| | | UAGCCUAGCCACACCCCCACGGGAAACAGCAGUGAUUAACCUUU |
| | | AGCAAUAAACGAAAGUUUAACUAAGCUAUACUAACCCCAGGGUU |
| | | GGUCAAUUUCGUGCCAGCCACACCCUGGAGCUAGC |
| 13 | mol_type = | CGCCAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGACCUCGAGCUGGUACUGCAUGCACGCAAUGCUAGCUG |
| | | CCCCUUUCCCGUCCUGGGUACCCCGAGUCUCCCCCGACCUCGGGU |
| | | CCCAGGUAUGCUCCCACCUCCACCUGCCCCACUCACCACCUCUGC |
| | | UAGUUCCAGACACCUCCCAAGCACGCAGCAAUGCAGCUCAAAAC |
| | | GCUUAGCCUAGCCACACCCCCACGGGAAACAGCAGUGAUUAACC |
| | | UUUAGCAAUAAACGAAAGUUUAACUAAGCUAUACUAACCCCAGG |
| | | GUUGGUCAAUUUCGUGCCAGCCACACCCUGGAGCUAGC |
| 14 | mol_type = | CAAGAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGACCUCGAGCUGGUACUGCAUGCACGCAAUGCUAGCUG |
| | | CCCCUUUCCCGUCCUGGGUACCCCGAGUCUCCCCCGACCUCGGGU |
| | | CCCAGGUAUGCUCCCACCUCCACCUGCCCCACUCACCACCUCUGC |
| | | UAGUUCCAGACACCUCCCAAGCACGCAGCAAUGCAGCUCAAAAC |
| | | GCUUAGCCUAGCCACACCCCCACGGGAAACAGCAGUGAUUAACC |
| | | UUUAGCAAUAAACGAAAGUUUAACUAAGCUAUACUAACCCCAGG |
| | | GUUGGUCAAUUUCGUGCCAGCCACACCCUGGAGCUAGC |
| 15 | mol_type = | AGAGGGUCCUGCUGGCGCGAGGGUGGGUAGGAGGGGACGCGGG |
| | RNA | GACUCGGCCCCCAACACCGCGCUCCGUCUGCAGCCGCCGCCUCUG |
| | origin = | CACCGCCGCUGCCCGGCGGUCGGUUCAAAAAACAGAAAUCGGGU |
| | synthetic | UUGCUGCCCGGCGGACAGGCGUGAAGAGCAAGGGAAAGGAACUU |
| | construct | CCUCCACCUUCGGGGCUGGAGCCCUUUUCCUCUGCAUCUCCAGU |
| | | CUCUGAGUGAAGAUGGGCGGGCUGACAGCCAGCGACGUGCACCC |
| | | AACCCUGGGAGUGCAGCUGUUUUCCGCCGGCAUUGCUGCCUGUC |
| | | UGGCUGAUGUGAUCACUUUCCCACUGGACACAGCUAAAGUGCGG |
| | | CUGCAGGUGCAGGGCGAGUGCCCAACAAGCUCUGUGAUCAGGUA |
| | | CAAGGGCGUGCUGGGGACCAUCACCGCCGUGGUGAAGACCGAGG |
| | | GCAGAAUGAAACUGUAUAGUGGUCUUCCCGCCGGCCUGCAGAGA |
| | | CAGAUUUCUAGCGCCUCACUGCGCAUUGGCCUGUAUGAUACAGU |
| | | GCAGGAGUUUCUGACUGCCGGCAAGGAAACUGCUCCAUCCCUGG |
| | | GCAGCAAAAUCCUGGCCGGACUCACAACUGGCGGCGUGGCCGUC |
| | | UUCAUCGGGCAGCCCACUGAGGUGGUGAAGGUGCGCCUGCAGGC |
| | | CCAGAGCCACUUGCAUGGGAUCAAACCCAGAUACACAGGAACCU |
| | | ACAACGCUUACAGAAUCAUCGCCACCACCGAGGGCCUGACUGGG |
| | | CUGUGGAAGGGCACUACCCCAAACCUGAUGCGGAGUGUGAUCAU |
| | | UAAUUGUACUGAGCUGGUGACCUAUGAUCUGAUGAAGGAGGCC |
| | | UUUGUGAAGAACAACAUCCUGGCCGACGACGUGCCUUGUCACCU |
| | | GGUGAGCGCCCUGAUCGCCGGCUUCUGCGCCACAGCCAUGAGCU |
| | | CCCCCGUGGACGUGGUGAAGACAAGAUUCAUCAAUAGCCCACCU |
| | | GGCCAGUAUAAAUCCGUGCCUAAUUGCGCCAUGAAGGUGUUCAC |
| | | CAAUGAGGGGCCCACCGCCUUUUUCAAGGGGCUGGUGCCCUCCU |
| | | UCCUGAGGCUGGGCAGUUGGAACGUGAUCAUGUUCGUGUGUUUC |
| | | GAACAGCUGAAAAGAGAGCUGUCCAAGUCUAGACAGACCAUGGA |
| | | CUGCGCCACAUGAUGACCUCGAGCUGGUACUGCAUGCACGCAAU |
| | | GCUAGCUGCCCCUUUCCCGUCCUGGGUACCCCGAGUCUCCCCCGA |
| | | CCUCGGGUCCCAGGUAUGCUCCCACCUCCACCUGCCCCACUCACC |
| | | ACCUCUGCUAGUUCCAGACACCUCCCAAGCACGCAGCAAUGCAG |
| | | CUCAAAACGCUUAGCCUAGCCACACCCCCACGGGAAACAGCAGU |
| | | GAUUAACCUUUAGCAAUAAACGAAAGUUUAACUAAGCUAUACU |
| | | AACCCCAGGGUUGGUCAAUUUCGUGCCAGCCACACCCUGGAGCU |
| | | AGC |
| 16 | mol_type = | ACAGCACCCUCCUGAAAACUGCAGCUUCCUUCUCACCUUGAAGA |
| | RNA | AUAAUCCUAGAAAACUCACAAAAUGGGCGGGCUGACAGCCAGCG |
| | origin = | ACGUGCACCCAACCCUGGGAGUGCAGCUGUUUUCCGCCGGCAUU |
| | synthetic | GCUGCCUGUCUGGCUGAUGUGAUCACUUUCCCACUGGACACAGC |
| | construct | UAAAGUGCGGCUGCAGGUGCAGGGCGAGUGCCCAACAAGCUCUG |
| | | UGAUCAGGUACAAGGGCGUGCUGGGGACCAUCACCGCCGUGGUG |
| | | AAGACCGAGGGCAGAAUGAAACUGUAUAGUGGUCUUCCCGCCGG |
| | | CCUGCAGAGACAGAUUUCUAGCGCCUCACUGCGCAUUGGCCUGU |
| | | AUGAUACAGUGCAGGAGUUUCUGACUGCCGGCAAGGAAACUGCU |
| | | CCAUCCCUGGGCAGCAAAAUCCUGGCCGGACUCACAACUGGCGG |
| | | CGUGGCCGUCUUCAUCGGGCAGCCCACUGAGGUGGUGAAGGUGC |
| | | GCCUGCAGGCCCAGAGCCACUUGCAUGGGAUCAAACCCAGAUAC |
| | | ACAGGAACCUACAACGCUUACAGAAUCAUCGCCACCACCGAGGG |
| | | CCUGACUGGGCUGUGGAAGGGCACUACCCCAAACCUGAUGCGGA |
| | | GUGUGAUCAUUAAUUGUACUGAGCUGGUGACCUAUGAUCUGAU |
| | | GAAGGAGGCCUUUGUGAAGAACAACAUCCUGGCCGACGACGUGC |
| | | CUUGUCACCUGGUGAGCGCCCUGAUCGCCGGCUUCUGCGCCACA |
| | | GCCAUGAGCUCCCCCGUGGACGUGGUGAAGACAAGAUUCAUCAA |
| | | UAGCCCACCUGGCCAGUAUAAAUCCGUGCCUAAUUGCGCCAUGA |
| | | AGGUGUUCACCAAUGAGGGGCCCACCGCCUUUUUCAAGGGGCUG |
| | | GUGCCCUCCUUCCUGAGGCUGGGCAGUUGGAACGUGAUCAUGUU |
| | | CGUGUGUUUCGAACAGCUGAAAAGAGAGCUGUCCAAGUCUAGAC |
| | | AGACCAUGGACUGCGCCACAUGAUGACCUCGAGCUGGUACUGCA |
| | | UGCACGCAAUGCUAGCUGCCCCUUUCCCGUCCUGGGUACCCCGA |
| | | GUCUCCCCCGACCUCGGGUCCCAGGUAUGCUCCCACCUCCACCUG |
| | | CCCCACUCACCACCUCUGCUAGUUCCAGACACCUCCCAAGCACGC |
| | | AGCAAUGCAGCUCAAAACGCUUAGCCUAGCCACACCCCCACGGG |
| | | AAACAGCAGUGAUUAACCUUUAGCAAUAAACGAAAGUUUAACU |
| | | AAGCUAUACUAACCCCAGGGUUGGUCAAUUUCGUGCCAGCCACA |
| | | CCCUGGAGCUAGC |
| 17 | mol_type = | AUGGGCGGGCUGACAGCCAGCGACGUGCACCCAACCCUGGGAGU |
| | RNA | GCAGCUGUUUUCCGCCGGCAUUGCUGCCUGUCUGGCUGAUGUGA |
| | origin = | UCACUUUCCCACUGGACACAGCUAAAGUGCGGCUGCAGGUGCAG |
| | synthetic | GGCGAGUGCCCAACAAGCUCUGUGAUCAGGUACAAGGGCGUGCU |
| | construct | GGGGACCAUCACCGCCGUGGUGAAGACCGAGGGCAGAAUGAAAC |
| | | UGUAUAGUGGUCUUCCCGCCGGCCUGCAGAGACAGAUUUCUAGC |
| | | GCCUCACUGCGCAUUGGCCUGUAUGAUACAGUGCAGGAGUUUCU |
| | | GACUGCCGGCAAGGAAACUGCUCCAUCCCUGGGCAGCAAAAUCC |
| | | UGGCCGGACUCACAACUGGCGGCGUGGCCGUCUUCAUCGGGCAG |
| | | CCCACUGAGGUGGUGAAGGUGCGCCUGCAGGCCCAGAGCCACUU |
| | | GCAUGGGAUCAAACCCAGAUACACAGGAACCUACAACGCUUACA |
| | | GAAUCAUCGCCACCACCGAGGGCCUGACUGGGCUGUGGAAGGGC |
| | | ACUACCCCAAACCUGAUGCGGAGUGUGAUCAUUAAUUGUACUGA |
| | | GCUGGUGACCUAUGAUCUGAUGAAGGAGGCCUUUGUGAAGAAC |
| | | AACAUCCUGGCCGACGACGUGCCUUGUCACCUGGUGAGCGCCCU |
| | | GAUCGCCGGCUUCUGCGCCACAGCCAUGAGCUCCCCCGUGGACG |
| | | UGGUGAAGACAAGAUUCAUCAAUAGCCCACCUGGCCAGUAUAAA |
| | | UCCGUGCCUAAUUGCGCCAUGAAGGUGUUCACCAAUGAGGGGCC |
| | | CACCGCCUUUUUCAAGGGGCUGGUGCCCUCCUUCCUGAGGCUGG |
| | | GCAGUUGGAACGUGAUCAUGUUCGUGUGUUUCGAACAGCUGAA |
| | | AAGAGAGCUGUCCAAGUCUAGACAGACCAUGGACUGCGCCACAU |
| | | GAUGACGCUCGCUUUCUUGCUGUCCAAUUUCUAUUAAAGGUUCC |
| | | UUUGUUCCCUAAGUCCAACUACUAAACUGGGGGAUAUUAUGAAG |
| | | GGCCUUGAGCAUCUGGAUUCUGCCUGCUCGCUUUCUUGCUGUCC |
| | | AAUUUCUAUUAAAGGUUCCUUUGUUCCCUAAGUCCAACUACUAA |
| | | ACUGGGGGAUAUUAUGAAGGGCCUUGAGCAUCUGGAUUCUGCCU |
| 18 | mol_type = | CGCCAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGACGCUCGCUUUCUUGCUGUCCAAUUUCUAUUAAAGGU |
| | | UCCUUUGUUCCCUAAGUCCAACUACUAAACUGGGGGAUAUUAUG |
| | | AAGGGCCUUGAGCAUCUGGAUUCUGCCUGCUCGCUUUCUUGCUG |
| | | UCCAAUUUCUAUUAAAGGUUCCUUUGUUCCCUAAGUCCAACUAC |
| | | UAAACUGGGGGAUAUUAUGAAGGGCCUUGAGCAUCUGGAUUCU |
| | | GCCU |
| 19 | mol_type = | CAAGAUGGGCGGGCUGACAGCCAGCGACGUGCACCCAACCCUGG |
| | RNA | GAGUGCAGCUGUUUUCCGCCGGCAUUGCUGCCUGUCUGGCUGAU |
| | origin = | GUGAUCACUUUCCCACUGGACACAGCUAAAGUGCGGCUGCAGGU |
| | synthetic | GCAGGGCGAGUGCCCAACAAGCUCUGUGAUCAGGUACAAGGGCG |
| | construct | UGCUGGGGACCAUCACCGCCGUGGUGAAGACCGAGGGCAGAAUG |
| | | AAACUGUAUAGUGGUCUUCCCGCCGGCCUGCAGAGACAGAUUUC |
| | | UAGCGCCUCACUGCGCAUUGGCCUGUAUGAUACAGUGCAGGAGU |
| | | UUCUGACUGCCGGCAAGGAAACUGCUCCAUCCCUGGGCAGCAAA |
| | | AUCCUGGCCGGACUCACAACUGGCGGCGUGGCCGUCUUCAUCGG |
| | | GCAGCCCACUGAGGUGGUGAAGGUGCGCCUGCAGGCCCAGAGCC |
| | | ACUUGCAUGGGAUCAAACCCAGAUACACAGGAACCUACAACGCU |
| | | UACAGAAUCAUCGCCACCACCGAGGGCCUGACUGGGCUGUGGAA |
| | | GGGCACUACCCCAAACCUGAUGCGGAGUGUGAUCAUUAAUUGUA |
| | | CUGAGCUGGUGACCUAUGAUCUGAUGAAGGAGGCCUUUGUGAA |
| | | GAACAACAUCCUGGCCGACGACGUGCCUUGUCACCUGGUGAGCG |
| | | CCCUGAUCGCCGGCUUCUGCGCCACAGCCAUGAGCUCCCCCGUG |
| | | GACGUGGUGAAGACAAGAUUCAUCAAUAGCCCACCUGGCCAGUA |
| | | UAAAUCCGUGCCUAAUUGCGCCAUGAAGGUGUUCACCAAUGAGG |
| | | GGCCCACCGCCUUUUUCAAGGGGCUGGUGCCCUCCUUCCUGAGG |
| | | CUGGGCAGUUGGAACGUGAUCAUGUUCGUGUGUUUCGAACAGCU |
| | | GAAAAGAGAGCUGUCCAAGUCUAGACAGACCAUGGACUGCGCCA |
| | | CAUGAUGACGCUCGCUUUCUUGCUGUCCAAUUUCUAUUAAAGGU |
| | | UCCUUUGUUCCCUAAGUCCAACUACUAAACUGGGGGAUAUUAUG |
| | | AAGGGCCUUGAGCAUCUGGAUUCUGCCUGCUCGCUUUCUUGCUG |
| | | UCCAAUUUCUAUUAAAGGUUCCUUUGUUCCCUAAGUCCAACUAC |
| | | UAAACUGGGGGAUAUUAUGAAGGGCCUUGAGCAUCUGGAUUCU |
| | | GCCU |
| 20 | mol_type = | AGAGGGUCCUGCUGGCGCGAGGGUGGGUAGGAGGGGACGCGGG |
| | RNA | GACUCGGCCCCCAACACCGCGCUCCGUCUGCAGCCGCCGCCUCUG |
| | origin = | CACCGCCGCUGCCCGGCGGUCGGUUCAAAAAACAGAAAUCGGGU |
| | synthetic | UUGCUGCCCGGCGGACAGGCGUGAAGAGCAAGGGAAAGGAACUU |
| | construct | CCUCCACCUUCGGGGCUGGAGCCCUUUUCCUCUGCAUCUCCAGU |
| | | CUCUGAGUGAAGAUGGGCGGGCUGACAGCCAGCGACGUGCACCC |
| | | AACCCUGGGAGUGCAGCUGUUUUCCGCCGGCAUUGCUGCCUGUC |
| | | UGGCUGAUGUGAUCACUUUCCCACUGGACACAGCUAAAGUGCGG |
| | | CUGCAGGUGCAGGGCGAGUGCCCAACAAGCUCUGUGAUCAGGUA |
| | | CAAGGGCGUGCUGGGGACCAUCACCGCCGUGGUGAAGACCGAGG |
| | | GCAGAAUGAAACUGUAUAGUGGUCUUCCCGCCGGCCUGCAGAGA |
| | | CAGAUUUCUAGCGCCUCACUGCGCAUUGGCCUGUAUGAUACAGU |
| | | GCAGGAGUUUCUGACUGCCGGCAAGGAAACUGCUCCAUCCCUGG |
| | | GCAGCAAAAUCCUGGCCGGACUCACAACUGGCGGCGUGGCCGUC |
| | | UUCAUCGGGCAGCCCACUGAGGUGGUGAAGGUGCGCCUGCAGGC |
| | | CCAGAGCCACUUGCAUGGGAUCAAACCCAGAUACACAGGAACCU |
| | | ACAACGCUUACAGAAUCAUCGCCACCACCGAGGGCCUGACUGGG |
| | | CUGUGGAAGGGCACUACCCCAAACCUGAUGCGGAGUGUGAUCAU |
| | | UAAUUGUACUGAGCUGGUGACCUAUGAUCUGAUGAAGGAGGCC |
| | | UUUGUGAAGAACAACAUCCUGGCCGACGACGUGCCUUGUCACCU |
| | | GGUGAGCGCCCUGAUCGCCGGCUUCUGCGCCACAGCCAUGAGCU |
| | | CCCCCGUGGACGUGGUGAAGACAAGAUUCAUCAAUAGCCCACCU |
| | | GGCCAGUAUAAAUCCGUGCCUAAUUGCGCCAUGAAGGUGUUCAC |
| | | CAAUGAGGGGCCCACCGCCUUUUUCAAGGGGCUGGUGCCCUCCU |
| | | UCCUGAGGCUGGGCAGUUGGAACGUGAUCAUGUUCGUGUGUUUC |
| | | GAACAGCUGAAAAGAGAGCUGUCCAAGUCUAGACAGACCAUGGA |
| | | CUGCGCCACAUGAUGACGCUCGCUUUCUUGCUGUCCAAUUUCUA |
| | | UUAAAGGUUCCUUUGUUCCCUAAGUCCAACUACUAAACUGGGGG |
| | | AUAUUAUGAAGGGCCUUGAGCAUCUGGAUUCUGCCUGCUCGCUU |
| | | UCUUGCUGUCCAAUUUCUAUUAAAGGUUCCUUUGUUCCCUAAGU |
| | | CCAACUACUAAACUGGGGGAUAUUAUGAAGGGCCUUGAGCAUCU |
| | | GGAUUCUGCC |
| 21 | mol_type = | ACAGCACCCUCCUGAAAACUGCAGCUUCCUUCUCACCUUGAAGA |
| | RNA | AUAAUCCUAGAAAACUCACAAAAUGGGCGGGCUGACAGCCAGCG |
| | origin = | ACGUGCACCCAACCCUGGGAGUGCAGCUGUUUUCCGCCGGCAUU |
| | synthetic | GCUGCCUGUCUGGCUGAUGUGAUCACUUUCCCACUGGACACAGC |
| | construct | UAAAGUGCGGCUGCAGGUGCAGGGCGAGUGCCCAACAAGCUCUG |
| | | UGAUCAGGUACAAGGGCGUGCUGGGGACCAUCACCGCCGUGGUG |
| | | AAGACCGAGGGCAGAAUGAAACUGUAUAGUGGUCUUCCCGCCGG |
| | | CCUGCAGAGACAGAUUUCUAGCGCCUCACUGCGCAUUGGCCUGU |
| | | AUGAUACAGUGCAGGAGUUUCUGACUGCCGGCAAGGAAACUGCU |
| | | CCAUCCCUGGGCAGCAAAAUCCUGGCCGGACUCACAACUGGCGG |
| | | CGUGGCCGUCUUCAUCGGGCAGCCCACUGAGGUGGUGAAGGUGC |
| | | GCCUGCAGGCCCAGAGCCACUUGCAUGGGAUCAAACCCAGAUAC |
| | | ACAGGAACCUACAACGCUUACAGAAUCAUCGCCACCACCGAGGG |
| | | CCUGACUGGGCUGUGGAAGGGCACUACCCCAAACCUGAUGCGGA |
| | | GUGUGAUCAUUAAUUGUACUGAGCUGGUGACCUAUGAUCUGAU |
| | | GAAGGAGGCCUUUGUGAAGAACAACAUCCUGGCCGACGACGUGC |
| | | CUUGUCACCUGGUGAGCGCCCUGAUCGCCGGCUUCUGCGCCACA |
| | | GCCAUGAGCUCCCCCGUGGACGUGGUGAAGACAAGAUUCAUCAA |
| | | UAGCCCACCUGGCCAGUAUAAAUCCGUGCCUAAUUGCGCCAUGA |
| | | AGGUGUUCACCAAUGAGGGGCCCACCGCCUUUUUCAAGGGGCUG |
| | | GUGCCCUCCUUCCUGAGGCUGGGCAGUUGGAACGUGAUCAUGUU |
| | | CGUGUGUUUCGAACAGCUGAAAAGAGAGCUGUCCAAGUCUAGAC |
| | | AGACCAUGGACUGCGCCACAUGAUGACGCUCGCUUUCUUGCUGU |
| | | CCAAUUUCUAUUAAAGGUUCCUUUGUUCCCUAAGUCCAACUACU |
| | | AAACUGGGGGAUAUUAUGAAGGGCCUUGAGCAUCUGGAUUCUG |
| | | CCUGCUCGCUUUCUUGCUGUCCAAUUUCUAUUAAAGGUUCCUUU |
| | | GUUCCCUAAGUCCAACUACUAAACUGGGGGAUAUUAUGAAGGGC |
| | | CUUGAGCAUCUGGAUUCUGCCU |
| 22 | mol_type = | AUGGGCGGCCUGACUGCCAGCGACGUGCACCCCACCCUCGGCGU |
| | RNA | GCAGCUGUUUAGCGCCGGAAUCGCCGCCUGUCUGGCUGAUGUGA |
| | origin = | UUACCUUCCCUCUGGACACCGCCAAGGUGCGGCUGCAGGUGCAA |
| | synthetic | GGCGAGUGCCCUACCAGCAGCGUGAUUAGAUACAAGGGAGUUCU |
| | construct | GGGCACCAUCACAGCUGUGGUGAAGACAGAAGGCAGAAUGAAAC |
| | | UGUACAGCGGCCUGCCAGCCGGCCUGCAGAGACAGAUCUCUAGC |
| | | GCCAGCCUGAGGAUCGGCCUGUAUGAUACCGUGCAGGAGUUCCU |
| | | GACAGCCGGAAAGGAAACCGCUCCUAGCCUGGGCUCUAAGAUCC |
| | | UGGCUGGUCUGACCACAGGCGGAGUGGCCGUGUUCAUCGGCCAG |
| | | CCUACAGAGGUGGUCAAAGUCCGGCUGCAAGCCCAGUCUCACCU |
| | | GCAUGGCAUCAAGCCCAGAUACACCGGCACCUACAACGCCUACA |
| | | GAAUCAUCGCCACCACCGAGGGCUUGACAGGCCUGUGGAAGGGC |
| | | ACAACACCUAAUCUGAUGCGGAGCGUGAUCAUCAACUGCACCGA |
| | | ACUGGUGACCUACGACCUGAUGAAAGAGGCCUUCGUGAAAAACA |
| | | ACAUCCUGGCUGAUGACGUGCCCUGCCACCUGGUGUCCGCUCUG |
| | | AUCGCCGGCUUCUGCGCCACAGCCAUGAGCUCUCCUGUGGACGU |
| | | CGUGAAGACCAGAUUCAUCAAUAGCCCUCCUGGACAGUACAAGU |
| | | CCGUGCCCAACUGUGCCAUGAAGGUGUUCACCAACGAGGGACCU |
| | | ACAGCAUUUUUCAAGGGCCUGGUGCCAUCAUUCCUGAGACUGGG |
| | | CUCCUGGAACGUGAUCAUGUUUGUGUGCUUCGAGCAGCUGAAGC |
| | | GGGAACUGAGCAAGAGCAGACAGACCAUGGACUGCGCCACCUGA |
| 23 | mol_type = | AUGGGCGGCCUGACCGCAUCUGAUGUGCAUCCAACACUGGGCGU |
| | RNA | UCAACUGUUCAGCGCCGGAAUCGCCGCCUGCCUGGCUGAUGUGA |
| | origin = | UCACAUUCCCCCUGGACACCGCCAAGGUGCGGCUGCAGGUGCAG |
| | synthetic | GGCGAGUGCCCUACAAGCAGCGUGAUCAGAUACAAGGGCGUGCU |
| | construct | GGGAACAAUCACCGCCGUGGUCAAGACCGAAGGGAGAAUGAAAC |
| | | UGUACAGCGGCCUGCCUGCCGGACUCCAGAGACAGAUUAGCUCC |
| | | GCUUCUCUGCGGAUCGGCCUGUACGAUACCGUGCAGGAGUUCCU |
| | | GACCGCUGGCAAGGAGACAGCCCCUUCUCUGGGAAGCAAGAUCC |
| | | UGGCCGGCCUCACAACCGGCGGAGUCGCCGUGUUCAUCGGCCAG |
| | | CCUACCGAGGUGGUGAAGGUGCGCCUGCAGGCCCAGAGCCACCU |
| | | GCACGGCAUCAAGCCUAGAUAUACAGGCACCUACAACGCCUACA |
| | | GGAUCAUCGCUACCACCGAGGGCCUGACUGGACUGUGGAAGGGC |
| | | ACAACCCCUAAUCUGAUGCGGAGCGUGAUUAUCAACUGCACCGA |
| | | ACUGGUGACCUACGACCUGAUGAAAGAAGCUUUUGUGAAGAACA |
| | | ACAUCCUGGCCGACGACGUCCCCUGUCACCUGGUGUCCGCCCUG |
| | | AUCGCCGGCUUCUGCGCCACCGCCAUGAGCAGCCCCGUGGACGU |
| | | GGUGAAAACAAGAUUCAUCAACAGCCCUCCUGGCCAAUACAAGU |
| | | CCGUGCCCAACUGUGCCAUGAAAGUGUUCACCAACGAGGGCCCU |
| | | ACCGCCUUUUUUAAGGGCCUGGUUCCAAGCUUCCUGAGACUGGG |
| | | CAGCUGGAAUGUGAUCAUGUUCGUGUGCUUCGAGCAGCUGAAGC |
| | | GGGAACUUUCUAAGAGCAGACAGACCAUGGACUGCGCCACCUGA |
| 24 | mol_type = | AUGGGCGGACUGACCGCUUCUGAUGUUCAUCCUACAUUGGGCGU |
| | RNA | GCAGCUGUUCAGCGCCGGCAUCGCCGCUUGUCUGGCUGAUGUGA |
| | origin = | UCACAUUCCCUCUGGACACCGCAAAGGUGCGGCUGCAAGUGCAA |
| | synthetic | GGCGAGUGCCCCACCAGCUCCGUGAUCCGGUACAAGGGAGUCCU |
| | construct | GGGUACAAUCACCGCCGUGGUGAAAACCGAAGGCAGAAUGAAGC |
| | | UGUACAGCGGCCUGCCAGCCGGCCUGCAGAGACAGAUCAGCAGC |
| | | GCCAGCCUGCGGAUCGGCCUGUACGAUACCGUGCAGGAGUUCCU |
| | | GACCGCUGGCAAGGAAACCGCCCCUUCUCUGGGAUCUAAAAUCC |
| | | UGGCCGGGCUGACAACCGGCGGCGUGGCCGUGUUUAUCGGCCAG |
| | | CCUACAGAGGUGGUCAAGGUGCGGCUGCAGGCCCAGAGCCACCU |
| | | GCACGGCAUUAAGCCCAGAUACACCGGCACCUACAACGCCUAUA |
| | | GAAUCAUCGCCACCACAGAAGGCCUGACAGGCCUGUGGAAGGGC |
| | | ACAACCCCUAAUCUGAUGAGAUCUGUGAUCAUUAACUGCACCGA |
| | | GCUGGUGACCUACGACCUGAUGAAAGAAGCCUUUGUGAAGAACA |
| | | ACAUCCUGGCCGACGACGUGCCUUGCCACCUGGUGUCCGCCCUG |
| | | AUCGCCGGCUUCUGCGCCACAGCCAUGAGCAGCCCUGUGGACGU |
| | | GGUGAAGACCAGAUUCAUCAACAGCCCACCUGGACAGUACAAGU |
| | | CCGUGCCCAAUUGUGCCAUGAAAGUGUUCACCAACGAGGGCCCU |
| | | ACCGCUUUUUUCAAGGGACUCGUCCCCAGCUUCCUGAGGCUCGG |
| | | CAGCUGGAACGUGAUCAUGUUCGUGUGCUUCGAGCAGCUGAAGA |
| | | GAGAGCUGAGCAAGAGCAGACAGACCAUGGACUGCGCCACAUGA |
| 25 | mol_type = | AUGGGCGGACUGACCGCCUCUGAUGUGCAUCCUACCCUGGGCGU |
| | RNA | CCAGCUGUUCAGCGCCGGAAUCGCCGCUUGUCUGGCUGAUGUGA |
| | origin = | UCACCUUCCCCCUGGACACAGCGAAGGUCAGACUGCAGGUGCAG |
| | synthetic | GGCGAGUGUCCUACCAGCAGCGUGAUUAGAUACAAGGGCGUGCU |
| | construct | GGGAACAAUCACAGCUGUGGUGAAGACAGAGGGCAGAAUGAAA |
| | | CUGUACAGCGGCCUGCCUGCCGGCCUGCAAAGACAGAUCAGCUC |
| | | CGCCAGCCUGCGGAUCGGCCUGUACGACACCGUGCAGGAGUUCC |
| | | UGACCGCCGGCAAGGAAACCGCCCCUAGCCUGGGCUCCAAGAUC |
| | | CUGGCCGGCUUGACCACCGGAGGCGUGGCCGUGUUCAUCGGCCA |
| | | GCCUACAGAAGUGGUGAAAGUGCGGCUGCAGGCCCAGAGCCACC |
| | | UGCACGGCAUCAAGCCUAGAUACACCGGCACCUACAACGCCUAC |
| | | CGGAUCAUCGCCACAACAGAAGGCCUGACAGGCCUGUGGAAGGG |
| | | CACAACCCCUAAUCUGAUGAGAAGCGUGAUCAUCAACUGCACCG |
| | | AGCUCGUGACCUAUGAUCUGAUGAAAGAGGCCUUCGUGAAGAAC |
| | | AACAUCCUGGCCGACGACGUGCCAUGCCACCUGGUGUCCGCCCU |
| | | GAUUGCCGGCUUCUGCGCCACCGCCAUGAGCUCUCCUGUGGACG |
| | | UUGUGAAAACCCGCUUUAUCAACAGCCCCCCCGGCCAAUACAAG |
| | | AGCGUGCCCAAUUGCGCCAUGAAGGUCUUUACCAACGAGGGUCC |
| | | UACAGCUUUCUUCAAGGGACUGGUUCCAUCUUUUCUGAGACUCG |
| | | GCAGCUGGAACGUGAUCAUGUUCGUGUGCUUCGAGCAGCUGAAG |
| | | CGGGAACUGAGCAAGUCUAGACAGACCAUGGACUGCGCUACCUG |
| | | A |
| 26 | mol_type = | AUGGGCGGACUGACCGCUUCUGAUGUGCAUCCCACCCUGGGCGU |
| | RNA | UCAACUGUUCAGCGCCGGCAUCGCCGCUUGUCUGGCAGAUGUGA |
| | origin = | UCACCUUUCCACUGGACACAGCCAAGGUGCGGCUGCAGGUGCAG |
| | synthetic | GGCGAGUGCCCCACCUCCUCCGUGAUUAGAUACAAGGGCGUUCU |
| | construct | CGGAACCAUCACAGCCGUGGUGAAGACCGAAGGCAGAAUGAAGC |
| | | UGUACAGCGGCCUGCCUGCCGGACUGCAGAGACAGAUCAGCAGC |
| | | GCCUCUCUGCGGAUCGGCCUGUAUGAUACAGUGCAGGAGUUCCU |
| | | GACAGCCGGUAAGGAAACCGCCCCUAGCCUGGGAUCUAAGAUCC |
| | | UGGCCGGACUGACCACAGGCGGCGUCGCCGUGUUCAUCGGCCAG |
| | | CCUACAGAGGUGGUGAAGGUGCGGCUUCAAGCCCAGAGCCACCU |
| | | GCACGGCAUCAAGCCUAGAUACACCGGCACAUACAACGCCUACA |
| | | GAAUCAUCGCUACCACCGAGGGCCUGACAGGCCUGUGGAAGGGC |
| | | ACCACCCCUAAUCUGAUGAGAAGCGUGAUCAUCAACUGUACAGA |
| | | ACUGGUGACCUACGACCUGAUGAAAGAGGCCUUUGUGAAAAACA |
| | | ACAUCCUCGCUGACGACGUGCCCUGCCACCUGGUCAGCGCCCUG |
| | | AUCGCCGGCUUCUGCGCCACCGCCAUGAGCUCUCCUGUGGACGU |
| | | GGUCAAAACCAGAUUCAUCAAUAGCCCCCCUGGACAGUACAAGA |
| | | GCGUGCCUAACUGCGCCAUGAAAGUGUUCACCAACGAGGGCCCU |
| | | ACAGCUUUUUUCAAGGGCCUGGUGCCAAGCUUCCUGAGACUGGG |
| | | CAGCUGGAACGUGAUUAUGUUCGUGUGCUUCGAGCAGCUGAAGC |
| | | GGGAACUGAGCAAGUCCCGCCAGACCAUGGACUGCGCCACCUGA |
| 27 | mol_type = | AUGGGCGGCCUGACCGCCAGCGACGUGCACCCCACACUGGGCGU |
| | RNA | GCAGCUGUUCAGCGCCGGCAUCGCCGCCUGCCUGGCUGAUGUGA |
| | origin = | UUACAUUCCCUCUGGAUACAGCCAAGGUGCGGCUGCAGGUGCAG |
| | synthetic | GGCGAGUGCCCUACCAGCAGCGUGAUCCGGUACAAGGGCGUGCU |
| | construct | GGGCACCAUCACCGCAGUGGUCAAGACCGAGGGCAGAAUGAAGC |
| | | UGUACAGCGGCCUCCCCGCCGGACUGCAAAGACAGAUCAGCUCU |
| | | GCUUCUCUGAGAAUCGGACUCUAUGAUACCGUGCAGGAGUUCCU |
| | | GACCGCUGGCAAGGAAACCGCCCCUUCCCUGGGAUCUAAGAUCC |
| | | UGGCCGGCCUGACAACCGGCGGAGUGGCCGUGUUCAUCGGCCAG |
| | | CCUACCGAAGUGGUGAAGGUCAGGCUGCAGGCCCAGAGCCAUCU |
| | | GCACGGCAUCAAACCCAGAUACACCGGCACCUACAACGCCUACA |
| | | GAAUCAUCGCCACCACCGAAGGCCUGACAGGCCUGUGGAAGGGC |
| | | ACCACACCUAAUCUGAUGAGAAGCGUGAUCAUCAACUGCACCGA |
| | | GCUGGUUACAUACGACCUGAUGAAAGAGGCCUUUGUGAAGAACA |
| | | ACAUCCUGGCCGACGACGUGCCCUGUCACCUGGUGUCCGCCCUG |
| | | AUCGCUGGCUUCUGCGCCACAGCCAUGAGCUCUCCUGUGGACGU |
| | | GGUGAAAACCAGAUUCAUCAACAGCCCUCCUGGCCAGUACAAGU |
| | | CCGUGCCAAAUUGUGCCAUGAAAGUUUUCACCAACGAGGGACCU |
| | | ACAGCUUUUUUCAAGGGACUGGUCCCAAGCUUCCUGCGGCUGGG |
| | | CAGCUGGAACGUGAUCAUGUUCGUGUGCUUUGAGCAGCUGAAGC |
| | | GGGAACUGAGCAAGAGCAGACAAACAAUGGACUGCGCCACCUGA |
| 28 | mol_type = | AUGGGCGGACUGACCGCUAGCGACGUGCACCCCACACUGGGAGU |
| | RNA | GCAGCUGUUCAGCGCCGGCAUCGCCGCCUGCCUGGCUGAUGUGA |
| | origin = | UCACAUUCCCCCUGGACACCGCCAAGGUGCGGCUGCAAGUGCAG |
| | synthetic | GGCGAGUGCCCUACCAGCUCCGUGAUCAGAUACAAGGGCGUGCU |
| | construct | GGGCACCAUCACCGCUGUGGUCAAGACCGAAGGCAGAAUGAAAC |
| | | UGUACAGCGGCCUGCCUGCCGGCCUGCAGAGACAGAUCAGCUCC |
| | | GCCAGCCUGCGGAUCGGCCUGUAUGAUACCGUUCAGGAGUUCCU |
| | | CACCGCCGGAAAAGAGACAGCCCCUUCUCUGGGCUCUAAGAUCC |
| | | UGGCCGGACUCACCACAGGCGGCGUGGCCGUGUUCAUCGGACAG |
| | | CCUACAGAAGUGGUGAAGGUGCGGCUUCAGGCCCAGAGCCAUCU |
| | | GCACGGCAUCAAGCCUAGAUACACCGGCACAUACAACGCCUACA |
| | | GAAUCAUCGCCACCACCGAGGGCCUGACAGGCCUGUGGAAGGGC |
| | | ACCACCCCUAAUCUGAUGAGAAGCGUGAUUAUCAACUGCACCGA |
| | | GCUGGUGACCUACGACCUGAUGAAGGAAGCUUUUGUGAAAAACA |
| | | ACAUCCUGGCCGAUGACGUCCCAUGUCACCUGGUCAGCGCCCUG |
| | | AUCGCCGGCUUCUGCGCCACAGCUAUGAGCUCUCCAGUGGACGU |
| | | GGUGAAGACCAGAUUCAUCAACAGCCCUCCUGGCCAGUACAAGU |
| | | CCGUGCCCAAUUGUGCCAUGAAAGUGUUUACCAACGAGGGUCCU |
| | | ACUGCCUUCUUCAAGGGACUGGUGCCCAGCUUUCUGAGACUGGG |
| | | CAGCUGGAACGUGAUCAUGUUCGUGUGCUUCGAGCAGCUGAAGC |
| | | GGGAACUGAGCAAGUCUAGGCAAACAAUGGACUGCGCCACAUGA |
| 29 | mol_type = | AUCGCCGCUUGUCUGGCUGAUGUCAUCACCUUCCCUCUGGACAC |
| | RNA | CGCCAAGGUGCGGCUGCAGGUGCAGGGCGAGUGCCCCACCAGCU |
| | origin = | CUGUGAUUCGGUACAAGGGCGUCCUGGGCACCAUCACAGCUGUU |
| | synthetic | GUGAAGACCGAGGGCAGAAUGAAACUGUACAGCGGCCUGCCUGC |
| | construct | CGGACUGCAAAGACAGAUCUCUUCUGCUUCCCUGAGAAUCGGCC |
| | | UGUAUGAUACCGUGCAGGAGUUCCUCACAGCCGGCAAGGAGACA |
| | | GCCCCUAGCCUGGGCUCCAAGAUCCUGGCCGGCCUGACCACAGG |
| | | CGGCGUCGCCGUGUUCAUCGGCCAGCCUACCGAGGUGGUGAAGG |
| | | UGCGGCUGCAGGCCCAGAGCCAUCUGCACGGCAUUAAGCCUAGA |
| | | UACACCGGCACAUACAACGCCUACAGAAUCAUCGCCACAACAGA |
| | | AGGCCUGACAGGCCUGUGGAAGGGCACCACCCCUAACCUGAUGA |
| | | GAAGCGUGAUCAUCAAUUGCACCGAACUGGUGACCUACGACCUG |
| | | AUGAAGGAAGCCUUUGUGAAGAACAACAUCCUGGCCGACGACGU |
| | | UCCAUGUCACCUGGUGUCUGCCCUGAUCGCCGGAUUUUGCGCCA |
| | | CAGCCAUGAGCUCCCCCGUGGACGUGGUGAAAACCAGAUUCAUC |
| | | AACAGCCCUCCAGGCCAGUACAAAAGCGUGCCUAAUUGCGCCAU |
| | | GAAAGUGUUCACCAACGAGGGACCUACCGCUUUUUUCAAGGGCC |
| | | UUGUGCCCAGCUUCCUGAGGCUGGGCAGCUGGAACGUGAUCAUG |
| | | UUCGUGUGCUUCGAGCAGCUGAAGCGGGAACUGAGCAAGAGCAG |
| | | ACAGACCAUGGACUGCGCCACCUGA |
| 30 | mol_type = | AUGGGCGGCCUGACUGCUUCUGAUGUGCACCCCACACUGGGCGU |
| | RNA | CCAACUGUUCAGCGCCGGCAUCGCCGCCUGCUUGGCUGACGUGA |
| | origin = | UCACCUUUCCUCUGGACACCGCAAAGGUGCGGCUGCAGGUGCAG |
| | synthetic | GGCGAGUGCCCUACAAGCUCUGUGAUCCGGUACAAGGGCGUUCU |
| | construct | GGGAACCAUCACAGCUGUGGUCAAGACCGAGGGAAGAAUGAAAC |
| | | UGUACAGCGGCCUGCCUGCCGGCCUGCAAAGACAGAUCAGCUCC |
| | | GCCAGCCUGCGGAUCGGCCUUUAUGAUACCGUGCAGGAGUUCCU |
| | | GACCGCCGGCAAGGAAACCGCCCCUAGCCUGGGCAGCAAGAUCC |
| | | UGGCCGGCCUGACAACAGGAGGCGUGGCCGUGUUCAUCGGACAG |
| | | CCUACCGAAGUGGUGAAGGUGCGCCUGCAGGCCCAGAGCCACCU |
| | | GCACGGCAUCAAGCCUAGAUACACAGGCACAUACAACGCUUACA |
| | | GAAUCAUCGCCACCACAGAGGGCCUGACCGGCCUGUGGAAGGGC |
| | | ACCACCCCUAAUCUGAUGAGAAGCGUGAUUAUCAACUGCACCGA |
| | | ACUGGUGACCUACGACCUGAUGAAAGAGGCCUUUGUGAAGAACA |
| | | ACAUCCUGGCCGACGACGUGCCAUGUCAUCUGGUGUCCGCCCUG |
| | | AUCGCCGGAUUCUGCGCUACCGCCAUGAGCUCCCCUGUGGACGU |
| | | GGUGAAAACCAGAUUCAUCAACAGCCCCCCCGGCCAGUACAAGA |
| | | GCGUGCCCAAUUGCGCCAUGAAAGUGUUCACCAACGAGGGCCCU |
| | | ACCGCCUUCUUCAAGGGCCUCGUUCCAAGCUUCCUGAGACUGGG |
| | | AUCUUGGAACGUGAUUAUGUUCGUGUGUUUUGAGCAGCUGAAG |
| | | CGGGAACUGUCUAAGUCAAGACAGACAAUGGAUUGCGCCACAUG |
| | | A |
| 31 | mol_type = | AUGGGCGGCCUGACCGCUAGCGACGUGCAUCCUACACUGGGCGU |
| | RNA | GCAGCUGUUCAGCGCUGGCAUCGCCGCCUGCCUGGCCGAUGUUA |
| | origin = | UCACCUUCCCCCUGGAUACCGCCAAGGUCAGACUGCAAGUGCAG |
| | synthetic | GGCGAAUGUCCUACAAGCAGCGUGAUCAGAUACAAGGGCGUGCU |
| | construct | GGGAACCAUCACAGCCGUGGUGAAAACAGAGGGCAGAAUGAAGC |
| | | UGUACAGCGGCUUGCCUGCCGGACUUCAAAGACAGAUCAGCUCU |
| | | GCUUCUCUGCGGAUCGGACUGUACGAUACAGUGCAGGAGUUCCU |
| | | GACCGCCGGCAAGGAAACCGCCCCUAGCCUGGGCUCCAAGAUCC |
| | | UGGCUGGACUGACAACUGGAGGAGUGGCCGUGUUCAUCGGCCAG |
| | | CCUACCGAGGUGGUGAAGGUGCGGCUGCAGGCCCAGAGCCACCU |
| | | GCACGGCAUCAAGCCCAGAUAUACAGGCACCUACAACGCCUACC |
| | | GGAUUAUCGCCACCACCGAAGGCCUGACCGGCCUGUGGAAGGGC |
| | | ACCACACCUAAUCUGAUGAGGUCCGUGAUUAUCAAUUGCACCGA |
| | | GCUGGUGACCUACGACCUGAUGAAAGAGGCCUUUGUGAAGAACA |
| | | ACAUCCUGGCUGACGACGUGCCUUGCCACCUGGUGUCCGCCCUG |
| | | AUCGCCGGCUUCUGCGCCACAGCCAUGAGCUCUCCAGUGGACGU |
| | | UGUGAAGACAAGAUUCAUCAACAGCCCCCCCGGCCAGUACAAAA |
| | | GCGUGCCUAACUGUGCCAUGAAAGUCUUUACCAACGAGGGCCCU |
| | | ACCGCCUUCUUUAAGGGCCUCGUGCCAAGCUUCCUGAGACUGGG |
| | | CAGCUGGAACGUGAUCAUGUUCGUGUGCUUCGAGCAGCUGAAGC |
| | | GGGAACUGUCUAAGAGCAGACAGACCAUGGACUGCGCCACCUGA |
| 32 | mol_type = | AUGGGCGGCCUGACAGCCAGCGACGUCCACCCCACCCUGGGCGU |
| | RNA | GCAGCUGUUCAGCGCCGGCAUCGCUGCUUGUCUGGCUGAUGUGA |
| | origin = | UCACUUUUCCACUGGAUACCGCAAAGGUGCGGCUGCAGGUGCAG |
| | synthetic | GGCGAGUGCCCUACCAGCUCUGUGAUCAGAUACAAGGGCGUGCU |
| | construct | GGGAACAAUCACCGCCGUUGUUAAGACAGAAGGCAGAAUGAAGC |
| | | UGUACAGCGGCCUGCCUGCUGGCCUUCAGAGACAGAUCAGCAGC |
| | | GCCUCUCUGCGGAUCGGCCUGUACGACACCGUGCAGGAGUUCCU |
| | | GACCGCCGGCAAGGAAACCGCCCCUAGCCUGGGAUCUAAGAUCC |
| | | UGGCCGGCCUGACAACCGGAGGAGUCGCCGUGUUCAUCGGCCAG |
| | | CCUACCGAGGUGGUGAAAGUGCGCCUGCAGGCCCAGAGCCACCU |
| | | GCACGGCAUCAAGCCUAGAUAUACAGGCACAUACAACGCCUACA |
| | | GGAUCAUCGCCACCACCGAGGGCCUGACCGGACUCUGGAAGGGC |
| | | ACCACCCCUAACCUGAUGCGGUCCGUGAUUAUCAAUUGUACCGA |
| | | GCUGGUGACCUACGACCUGAUGAAGGAAGCCUUCGUGAAAAACA |
| | | ACAUCCUGGCCGACGACGUGCCCUGCCAUCUGGUGUCCGCCCUG |
| | | AUCGCCGGCUUCUGCGCCACAGCCAUGAGCUCUCCAGUGGAUGU |
| | | GGUGAAAACCAGAUUCAUCAACAGCCCCCCCGGACAAUACAAGU |
| | | CCGUGCCUAAUUGCGCCAUGAAAGUGUUCACCAACGAGGGCCCU |
| | | ACAGCUUUUUUCAAGGGCCUGGUCCCUAGCUUUCUGAGACUGGG |
| | | CAGCUGGAACGUGAUUAUGUUCGUGUGCUUCGAGCAGCUGAAGC |
| | | GGGAACUGAGCAAGAGCAGACAAACAAUGGACUGCGCCACAUGA |
| 33 | mol_type = | AUGGGAGGCCUGACAGCUUCUGAUGUGCACCCCACCCUGGGCGU |
| | RNA | GCAACUGUUCAGCGCCGGCAUCGCCGCUUGCCUGGCCGACGUGA |
| | origin = | UCACAUUCCCCCUGGACACCGCCAAGGUGCGGCUUCAGGUGCAG |
| | synthetic | GGAGAAUGUCCUACCAGCAGCGUGAUCAGAUACAAGGGCGUGCU |
| | construct | GGGCACAAUCACAGCUGUGGUCAAGACCGAAGGCAGAAUGAAAC |
| | | UGUACAGCGGCCUGCCUGCCGGCCUGCAGAGACAGAUCAGCUCC |
| | | GCCAGCCUGAGGAUCGGCCUGUACGACACCGUGCAGGAGUUCCU |
| | | GACCGCAGGCAAGGAGACAGCCCCUAGCCUGGGCAGCAAGAUCC |
| | | UGGCCGGCCUCACAACCGGAGGAGUGGCCGUGUUCAUCGGCCAG |
| | | CCUACCGAGGUGGUGAAGGUCAGACUGCAGGCCCAGUCCCACCU |
| | | GCACGGCAUUAAGCCUAGAUACACCGGCACCUAUAAUGCCUACC |
| | | GGAUCAUCGCCACCACCGAGGGACUGACCGGCCUCUGGAAGGGC |
| | | ACAACACCUAACCUGAUGCGGAGCGUGAUCAUCAACUGCACCGA |
| | | GCUGGUUACAUACGACCUGAUGAAGGAAGCCUUUGUGAAAAACA |
| | | ACAUCCUGGCUGAUGAUGUUCCAUGUCAUCUGGUGUCCGCCCUG |
| | | AUCGCCGGCUUCUGCGCCACUGCCAUGAGCUCUCCUGUGGACGU |
| | | GGUGAAAACCAGAUUCAUUAACAGCCCUCCAGGCCAAUACAAGA |
| | | GCGUGCCCAAUUGCGCCAUGAAGGUUUUCACCAACGAGGGCCCC |
| | | ACCGCUUUUUUCAAAGGCCUGGUGCCUUCUUUUCUGAGACUGGG |
| | | AUCUUGGAACGUGAUCAUGUUCGUGUGCUUCGAGCAGCUGAAGC |
| | | GGGAACUGUCUAAGAGCAGACAGACCAUGGACUGCGCCACAUGA |
| 34 | mol_type = | AUGGGCGGACUUACAGCCAGUGAUGUGCAUCCUACCCUGGGCGU |
| | RNA | UCAACUGUUCAGCGCCGGAAUCGCCGCUUGUCUGGCUGAUGUGA |
| | origin = | UCACCUUCCCCCUGGACACCGCCAAGGUGCGGCUGCAGGUGCAG |
| | synthetic | GGCGAGUGCCCUACAAGCAGCGUGAUUAGAUACAAGGGCGUGCU |
| | construct | GGGCACCAUCACAGCCGUGGUGAAAACCGAAGGUAGAAUGAAGC |
| | | UGUACAGCGGCCUGCCUGCCGGCCUGCAAAGGCAGAUCAGCUCU |
| | | GCCUCUCUGCGGAUCGGCCUCUAUGAUACAGUGCAGGAGUUCCU |
| | | GACCGCUGGCAAGGAAACCGCUCCUUCUCUGGGCAGCAAGAUCC |
| | | UGGCCGGACUGACAACCGGCGGCGUGGCCGUGUUUAUCGGACAG |
| | | CCAACAGAAGUGGUCAAAGUGCGGCUGCAGGCCCAGAGCCACCU |
| | | GCACGGCAUCAAGCCUAGAUACACCGGCACAUACAACGCCUACA |
| | | GAAUCAUCGCCACCACUGAGGGCCUCACCGGCCUGUGGAAGGGC |
| | | ACCACACCUAAUCUGAUGAGAAGCGUGAUUAUCAACUGUACCGA |
| | | GCUGGUCACCUACGACCUGAUGAAAGAGGCCUUUGUGAAGAACA |
| | | ACAUCCUGGCCGACGACGUCCCCUGCCACCUGGUGUCCGCCCUG |
| | | AUCGCCGGCUUCUGCGCCACAGCUAUGAGCUCCCCUGUGGACGU |
| | | GGUGAAGACCAGAUUCAUCAAUAGCCCCCCCGGCCAGUACAAGA |
| | | GCGUGCCUAACUGCGCCAUGAAAGUGUUCACCAACGAGGGCCCU |
| | | ACCGCCUUUUUCAAGGGCCUGGUUCCAAGCUUCCUGAGACUGGG |
| | | AUCUUGGAACGUGAUCAUGUUCGUGUGCUUCGAGCAGCUGAAGC |
| | | GGGAACUGAGCAAGUCCAGACAGACCAUGGACUGCGCCACAUGA |
| 35 | mol_type = | AUGGGUGGCCUGACAGCCUCCGACGUGCAUCCUACCCUGGGAGU |
| | RNA | GCAACUGUUCAGCGCCGGCAUCGCCGCUUGUCUGGCUGAUGUGA |
| | origin = | UUACCUUCCCCCUGGACACAGCCAAGGUGAGACUGCAAGUGCAG |
| | synthetic | GGCGAGUGCCCCACAAGCAGCGUGAUCCGGUACAAGGGCGUGCU |
| | construct | GGGCACCAUCACCGCUGUGGUUAAGACAGAAGGCAGAAUGAAGC |
| | | UGUACAGCGGCCUGCCUGCCGGCCUGCAGAGACAGAUCAGCUCU |
| | | GCCUCUCUGCGGAUCGGCCUCUAUGAUACCGUGCAGGAGUUCCU |
| | | GACCGCCGGAAAGGAGACAGCCCCUUCUCUGGGAAGCAAGAUCC |
| | | UGGCCGGCCUGACAACCGGCGGAGUGGCCGUCUUCAUCGGCCAG |
| | | CCUACAGAAGUGGUGAAGGUGCGGCUGCAGGCCCAGAGCCACCU |
| | | GCACGGCAUCAAGCCCAGAUACACCGGAACCUACAACGCCUACA |
| | | GAAUCAUCGCCACCACCGAGGGCCUCACAGGCCUGUGGAAAGGC |
| | | ACCACCCCUAAUCUGAUGAGGUCCGUGAUCAUCAACUGCACCGA |
| | | ACUGGUGACCUACGACCUGAUGAAAGAGGCCUUUGUGAAAAACA |
| | | ACAUCCUGGCCGACGAUGUGCCAUGCCACCUGGUCAGCGCCCUG |
| | | AUCGCUGGCUUUUGCGCAACCGCCAUGAGCUCCCCAGUGGACGU |
| | | GGUGAAGACAAGAUUCAUUAACAGCCCUCCUGGCCAGUACAAGA |
| | | GCGUCCCCAAUUGCGCCAUGAAAGUGUUCACCAACGAGGGACCU |
| | | ACAGCUUUCUUCAAGGGCCUGGUGCCUAGCUUCCUGAGACUGGG |
| | | CAGCUGGAACGUGAUCAUGUUCGUUUGUUUUGAGCAGCUGAAGC |
| | | GGGAACUGAGCAAGUCUAGACAGACCAUGGACUGCGCCACCUGA |
| 36 | mol_type = | AUGGGCGGACUGACCGCCUCUGAUGUGCACCCCACCCUGGGAGU |
| | RNA | GCAGCUGUUCAGCGCCGGUAUCGCCGCUUGUCUGGCCGACGUGA |
| | | UUACAUUCCCCCUGGACACCGCGAAGGUGCGGCUGCAAGUGCAG |
| | origin = | GGCGAGUGCCCUACAAGCUCUGUUAUUAGAUAUAAGGGCGUGCU |
| | synthetic | GGGCACCAUCACCGCCGUGGUGAAAACCGAGGGCAGAAUGAAGC |
| | construct | UGUACAGCGGCCUGCCUGCCGGACUGCAGAGACAGAUCAGCAGC |
| | | GCCAGCCUGAGGAUCGGCCUCUACGAUACCGUGCAGGAGUUCCU |
| | | GACCGCCGGCAAGGAGACAGCCCCUAGCCUGGGCUCCAAGAUCC |
| | | UCGCCGGCCUGACCACAGGCGGCGUCGCCGUGUUCAUCGGCCAA |
| | | CCUACCGAAGUGGUCAAAGUGCGGCUGCAGGCCCAGAGCCACCU |
| | | GCACGGCAUCAAGCCCAGAUACACCGGAACCUACAACGCCUACA |
| | | GAAUCAUCGCCACAACAGAAGGCCUGACAGGCCUGUGGAAGGGC |
| | | ACCACACCUAACCUGAUGCGGAGCGUGAUCAUCAAUUGCACUGA |
| | | GCUGGUUACAUACGACCUGAUGAAGGAAGCCUUCGUGAAAAACA |
| | | ACAUCCUGGCUGAUGACGUGCCUUGUCAUCUGGUGUCCGCCCUG |
| | | AUCGCCGGAUUUUGCGCCACCGCUAUGAGCUCUCCAGUGGACGU |
| | | GGUCAAGACCAGAUUCAUCAAUUCUCCUCCUGGCCAGUACAAGU |
| | | CCGUGCCAAACUGCGCUAUGAAAGUGUUUACCAACGAGGGACCU |
| | | ACCGCUUUUUUCAAGGGCCUGGUGCCCAGCUUCCUGAGACUGGG |
| | | CAGCUGGAACGUGAUCAUGUUCGUGUGCUUCGAGCAGCUGAAGC |
| | | GGGAACUGAGCAAGAGCAGACAGACCAUGGACUGCGCCACCUGA |
| 37 | mol_type = | AUGGGCGGCCUGACCGCUAGCGACGUGCACCCCACACUGGGCGU |
| | RNA | UCAGCUGUUCAGCGCCGGCAUCGCCGCCUGUCUGGCUGAUGUGA |
| | origin = | UUACCUUCCCCCUGGACACAGCCAAGGUGCGGCUGCAAGUGCAG |
| | synthetic | GGCGAGUGCCCUACCAGCAGCGUGAUCAGAUAUAAGGGCGUGCU |
| | construct | GGGCACCAUCACCGCCGUGGUGAAGACCGAAGGCAGAAUGAAGC |
| | | UGUACAGCGGCCUGCCUGCCGGACUUCAAAGACAGAUCAGCUCC |
| | | GCUUCUCUGCGGAUUGGACUCUACGACACCGUGCAGGAGUUCCU |
| | | GACCGCCGGAAAAGAGACAGCCCCUUCUCUGGGAUCUAAGAUCC |
| | | UGGCCGGCCUGACAACCGGAGGCGUGGCCGUGUUUAUCGGACAG |
| | | CCUACAGAAGUGGUGAAGGUGAGGCUGCAGGCCCAGAGCCAUCU |
| | | GCACGGCAUCAAGCCCAGAUACACCGGCACAUACAACGCCUACC |
| | | GGAUCAUCGCCACCACCGAGGGCCUCACAGGCCUGUGGAAGGGC |
| | | ACCACCCCUAAUCUGAUGAGAUCUGUGAUCAUCAAUUGCACCGA |
| | | GCUGGUCACCUACGAUCUGAUGAAAGAAGCCUUCGUCAAGAACA |
| | | ACAUCCUGGCGGAUGACGUUCCAUGUCACCUGGUGUCCGCCCUG |
| | | AUCGCCGGCUUCUGCGCCACAGCCAUGAGCUCCCCUGUGGACGU |
| | | CGUGAAAACGAGAUUCAUCAACAGCCCUCCAGGCCAGUACAAGA |
| | | GCGUGCCUAACUGCGCCAUGAAAGUGUUCACCAACGAGGGUCCU |
| | | ACCGCUUUUUUCAAGGGCCUGGUGCCCAGCUUUCUGAGACUGGG |
| | | CAGCUGGAACGUGAUCAUGUUCGUGUGCUUCGAGCAGCUGAAGC |
| | | GGGAACUGAGCAAGUCUAGACAGACCAUGGACUGCGCUACAUGA |
| 38 | mol_type = | AUGGGCGGACUGACCGCCUCUGAUGUCCAUCCUACCCUGGGCGU |
| | RNA | UCAGCUCUUUAGCGCCGGAAUCGCCGCUUGUCUGGCCGAUGUCA |
| | origin = | UCACCUUCCCCCUGGACACCGCCAAGGUGCGGCUGCAAGUGCAG |
| | synthetic | GGCGAGUGUCCUACCAGCAGCGUGAUCCGGUACAAGGGCGUGCU |
| | construct | GGGCACUAUCACAGCUGUGGUGAAGACCGAGGGCAGAAUGAAAC |
| | | UGUACAGCGGCCUGCCAGCCGGCCUGCAGAGACAGAUUUCCAGC |
| | | GCUAGCCUGAGAAUCGGCCUGUAUGAUACCGUGCAGGAGUUCCU |
| | | GACCGCAGGCAAGGAAACCGCCCCUAGCCUGGGCAGCAAGAUCC |
| | | UGGCCGGCCUGACAACCGGCGGCGUGGCCGUGUUUAUUGGACAG |
| | | CCUACAGAGGUGGUGAAGGUGAGGCUGCAGGCCCAGAGCCACCU |
| | | GCACGGCAUCAAGCCCAGAUACACCGGCACCUACAACGCCUACA |
| | | GAAUCAUCGCCACAACCGAAGGCCUGACAGGACUGUGGAAGGGC |
| | | ACCACACCUAAUCUGAUGAGAAGCGUGAUCAUCAAUUGCACCGA |
| | | GCUGGUGACCUACGACCUGAUGAAGGAAGCCUUUGUGAAGAACA |
| | | ACAUCCUGGCCGACGACGUGCCCUGCCACCUGGUGUCCGCCCUG |
| | | AUCGCUGGCUUCUGCGCCACAGCCAUGAGCUCUCCUGUGGACGU |
| | | GGUCAAAACCAGAUUCAUCAACAGCCCUCCCGGCCAGUACAAAA |
| | | GCGUCCCAAACUGCGCCAUGAAAGUGUUCACCAACGAGGGACCU |
| | | ACAGCUUUCUUCAAGGGACUUGUGCCUUCCUUCCUGCGGCUGGG |
| | | CAGCUGGAACGUGAUCAUGUUCGUGUGCUUCGAGCAGCUGAAGC |
| | | GGGAACUGUCUAAGUCUAGACAAACAAUGGACUGCGCCACCUGA |
| 39 | mol_type = | AUGGGCGGGCUCACAGCUUCUGAUGUGCAUCCUACACUGGGCGU |
| | RNA | CCAGCUGUUCAGCGCCGGAAUCGCCGCUUGUCUGGCCGAUGUGA |
| | origin = | UCACCUUCCCCCUGGACACCGCCAAGGUGCGGCUGCAGGUGCAG |
| | synthetic | GGCGAGUGCCCUACCAGCUCUGUGAUCCGCUACAAGGGCGUGCU |
| | construct | GGGCACCAUCACAGCUGUGGUGAAGACCGAAGGCAGAAUGAAAC |
| | | UGUACAGCGGCCUGCCUGCCGGCCUGCAAAGACAAAUCAGCAGC |
| | | GCCAGCCUGAGAAUCGGACUGUAUGAUACAGUGCAGGAGUUCCU |
| | | GACUGCUGGCAAGGAGACAGCCCCUAGCCUGGGAAGCAAGAUCC |
| | | UUGCCGGACUGACCACCGGCGGCGUGGCCGUGUUUAUCGGCCAG |
| | | CCUACCGAAGUGGUUAAGGUGCGGCUGCAGGCCCAGAGCCACCU |
| | | GCACGGCAUCAAGCCUAGAUACACCGGCACCUACAACGCUUACA |
| | | GAAUUAUCGCCACAACCGAAGGUCUGACAGGACUGUGGAAGGGC |
| | | ACCACCCCUAACCUGAUGCGGAGCGUCAUCAUUAACUGCACCGA |
| | | GCUGGUUACAUACGACCUGAUGAAAGAGGCCUUUGUGAAGAACA |
| | | ACAUCCUGGCCGACGACGUCCCAUGUCACCUGGUGUCCGCCCUG |
| | | AUCGCCGGCUUCUGCGCCACAGCCAUGAGCUCCCCUGUGGACGU |
| | | GGUGAAGACAAGAUUCAUCAACAGCCCCCCCGGCCAGUACAAGU |
| | | CUGUGCCAAAUUGCGCCAUGAAAGUGUUCACCAACGAGGGCCCU |
| | | ACCGCUUUUUUCAAGGGCCUGGUGCCCUCUUUCCUCAGACUGGG |
| | | CAGCUGGAAUGUGAUCAUGUUCGUGUGCUUCGAGCAGCUGAAGC |
| | | GGGAACUGAGCAAAUCCAGACAGACCAUGGACUGCGCCACCUGA |
| 40 | mol_type = | AUGGGCGGCCUGACCGCCAGCGACGUGCAUCCUACACUAGGCGU |
| | RNA | GCAGCUGUUCAGCGCCGGCAUCGCCGCCUGCCUGGCUGAUGUGA |
| | origin = | UUACCUUCCCCCUGGACACCGCCAAGGUGCGGCUGCAAGUGCAG |
| | synthetic | GGCGAGUGUCCUACCAGCAGCGUGAUCAGAUACAAGGGAGUGCU |
| | construct | GGGAACAAUCACCGCCGUGGUGAAAACCGAGGGAAGAAUGAAGC |
| | | UGUACAGCGGCCUCCCCGCCGGCCUGCAGAGACAGAUCAGCUCU |
| | | GCUUCUCUGCGGAUCGGCCUGUAUGAUACCGUGCAGGAGUUCCU |
| | | GACAGCUGGCAAGGAGACAGCCCCUAGCCUGGGCAGCAAGAUCC |
| | | UGGCCGGACUCACAACCGGCGGAGUGGCCGUGUUUAUUGGCCAG |
| | | CCUACAGAAGUGGUCAAAGUGCGGCUGCAGGCCCAGAGCCACCU |
| | | GCACGGCAUCAAGCCCAGAUACACCGGCACCUACAACGCCUACA |
| | | GAAUCAUCGCCACAACAGAAGGCCUGACCGGACUGUGGAAGGGC |
| | | ACCACCCCUAAUCUGAUGAGGUCCGUGAUCAUCAACUGCACCGA |
| | | ACUGGUCACCUACGACCUGAUGAAAGAGGCCUUUGUCAAGAACA |
| | | ACAUCCUGGCCGAUGACGUGCCUUGUCACCUGGUGUCCGCCCUG |
| | | AUCGCCGGCUUCUGCGCCACAGCUAUGAGCUCUCCUGUGGACGU |
| | | GGUUAAGACCAGAUUCAUCAACAGCCCUCCAGGCCAAUACAAGU |
| | | CCGUGCCAAAUUGCGCCAUGAAGGUGUUCACCAACGAGGGUCCU |
| | | ACCGCUUUUUUCAAGGGCCUGGUGCCCAGCUUCCUGAGACUGGG |
| | | CUCUUGGAACGUGAUCAUGUUCGUGUGCUUCGAGCAGCUGAAGC |
| | | GGGAACUGAGCAAAAGCAGACAGACCAUGGACUGCGCUACAUGA |
| 41 | mol_type = | AUGGGCGGCCUGACCGCUUCCGAUGUGCACCCCACCCUGGGCGU |
| | RNA | GCAGCUGUUCAGCGCCGGCAUCGCCGCUUGUCUGGCCGACGUGA |
| | origin = | UCACUUUUCCACUGGACACAGCCAAGGUGCGGCUGCAAGUGCAG |
| | synthetic | GGCGAGUGCCCCACCAGCUCCGUGAUCCGGUACAAAGGCGUGCU |
| | construct | GGGCACCAUCACCGCCGUGGUGAAGACAGAAGGCAGAAUGAAAC |
| | | UGUACAGCGGCCUGCCUGCCGGCCUGCAGAGGCAGAUCAGCAGC |
| | | GCCUCUCUGCGGAUCGGACUGUACGACACCGUGCAGGAGUUCCU |
| | | GACGGCCGGAAAAGAGACAGCCCCUAGCCUCGGCAGCAAGAUCC |
| | | UGGCCGGCCUCACCACCGGCGGUGUGGCCGUGUUCAUUGGACAA |
| | | CCUACCGAAGUGGUGAAGGUCAGACUGCAGGCCCAGAGCCACCU |
| | | GCAUGGCAUCAAGCCUAGAUACACCGGAACAUACAACGCCUACA |
| | | GAAUCAUUGCCACCACCGAGGGACUGACAGGCCUGUGGAAGGGC |
| | | ACCACACCUAAUCUGAUGAGAAGCGUGAUCAUCAAUUGUACCGA |
| | | GCUGGUUACAUAUGACCUGAUGAAGGAAGCCUUCGUGAAAAACA |
| | | ACAUCCUUGCUGAUGAUGUGCCCUGCCACCUGGUGUCCGCCCUG |
| | | AUCGCCGGCUUCUGCGCCACCGCAAUGAGCAGCCCUGUGGACGU |
| | | CGUGAAGACCAGAUUCAUCAACAGCCCCCCUGGCCAGUACAAGA |
| | | GCGUGCCUAACUGCGCCAUGAAGGUGUUCACCAACGAGGGCCCU |
| | | ACAGCUUUUUUCAAGGGCCUGGUCCCAUCUUUUCUGAGACUGGG |
| | | AUCUUGGAACGUGAUCAUGUUCGUGUGCUUCGAGCAGCUGAAGC |
| | | GGGAACUGAGCAAGUCUAGACAGACCAUGGACUGCGCUACAUGA |
| 42 | mol_type = | AUGGGCGGCCUGACCGCUAGCGACGUGCAUCCUACCCUGGGAGU |
| | RNA | GCAGCUGUUCAGUGCCGGCAUCGCUGCUUGUCUGGCUGAUGUGA |
| | origin = | UCACCUUCCCCCUGGACACCGCAAAGGUGCGGCUGCAAGUGCAG |
| | synthetic | GGAGAAUGUCCUACAAGCUCCGUGAUUAGAUAUAAGGGCGUGCU |
| | construct | GGGUACCAUUACAGCCGUGGUCAAAACCGAAGGCAGAAUGAAAC |
| | | UGUACAGCGGCCUCCCUGCCGGCCUGCAGCGCCAGAUCAGCAGC |
| | | GCCAGCCUGCGGAUCGGACUGUACGAUACCGUGCAGGAGUUCCU |
| | | GACCGCCGGCAAGGAAACCGCCCCUAGCCUGGGCUCCAAGAUCC |
| | | UGGCCGGCCUGACAACAGGCGGAGUGGCCGUCUUUAUCGGCCAG |
| | | CCUACAGAGGUGGUGAAGGUUAGACUGCAGGCCCAGAGCCACCU |
| | | GCACGGAAUCAAGCCCAGAUACACCGGCACCUACAACGCCUACA |
| | | GAAUCAUCGCCACCACAGAGGGCCUGACAGGCCUGUGGAAGGGC |
| | | ACAACCCCUAAUCUGAUGCGGAGCGUGAUCAUCAACUGCACCGA |
| | | GCUGGUGACCUACGAUCUGAUGAAAGAGGCCUUUGUGAAGAACA |
| | | ACAUCCUGGCCGACGACGUGCCGUGCCACCUGGUGUCUGCCCUG |
| | | AUCGCCGGCUUCUGCGCCACCGCCAUGAGCUCUCCAGUGGACGU |
| | | GGUGAAAACCAGAUUCAUCAACAGCCCUCCAGGCCAAUACAAGU |
| | | CCGUGCCCAAUUGCGCCAUGAAGGUGUUCACCAACGAGGGCCCU |
| | | ACAGCUUUUUUCAAGGGCCUGGUCCCCAGCUUCCUGAGACUGGG |
| | | AUCUUGGAACGUGAUCAUGUUCGUGUGCUUCGAGCAGCUCAAGC |
| | | GGGAACUGUCUAAGAGCAGACAGACCAUGGACUGCGCCACAUGA |
| 43 | mol_type = | AUGGGAGGCCUGACCGCAUCUGAUGUGCACCCUACACUGGGAGU |
| | RNA | GCAGCUGUUCAGCGCCGGAAUCGCCGCUUGUCUGGCCGAUGUGA |
| | origin = | UUACCUUCCCCCUGGACACCGCCAAGGUGCGCCUGCAGGUGCAG |
| | synthetic | GGCGAGUGCCCUACCAGCUCUGUGAUCAGAUACAAAGGCGUGCU |
| | construct | GGGCACCAUCACCGCUGUGGUCAAGACAGAGGGCAGAAUGAAAC |
| | | UGUACAGCGGCCUCCCCGCCGGCCUGCAAAGACAGAUCAGCAGC |
| | | GCCAGCCUGAGAAUCGGCCUGUAUGACACCGUGCAGGAGUUCCU |
| | | GACAGCUGGCAAGGAAACCGCCCCUAGCCUGGGCAGCAAGAUCC |
| | | UGGCCGGUCUUACAACCGGCGGCGUGGCCGUGUUCAUCGGCCAA |
| | | CCUACAGAAGUGGUGAAGGUGCGGCUGCAGGCCCAGAGCCACCU |
| | | GCAUGGCAUCAAGCCUAGAUACACUGGAACAUACAACGCCUACC |
| | | GGAUCAUCGCCACCACCGAGGGCCUGACAGGCCUCUGGAAGGGC |
| | | ACCACCCCUAAUCUGAUGAGAUCCGUGAUCAUCAACUGCACCGA |
| | | GCUGGUCACCUACGACCUGAUGAAAGAAGCCUUUGUGAAGAACA |
| | | ACAUCCUGGCUGAUGACGUUCCAUGCCACCUGGUGUCUGCCCUG |
| | | AUCGCCGGCUUCUGCGCCACAGCCAUGAGCUCCCCUGUGGACGU |
| | | GGUGAAGACCAGAUUCAUCAAUAGCCCUCCAGGACAGUACAAGU |
| | | CCGUCCCUAACUGCGCCAUGAAAGUGUUCACCAACGAGGGCCCC |
| | | ACAGCUUUUUUCAAGGGCCUGGUGCCCAGCUUCCUGCGGCUGGG |
| | | AUCUUGGAACGUGAUUAUGUUCGUUUGUUUUGAGCAGCUGAAG |
| | | CGGGAACUGAGCAAGAGCAGACAGACCAUGGACUGCGCCACCUG |
| | | A |
| 44 | mol_type = | AUGGGCGGACUGACCGCUUCUGAUGUGCAUCCCACACUGGGCGU |
| | RNA | GCAACUGUUCAGCGCCGGAAUCGCCGCAUGCCUGGCUGAUGUUA |
| | origin = | UUACCUUCCCUCUGGAUACCGCCAAGGUGCGGCUGCAAGUGCAG |
| | synthetic | GGCGAGUGUCCUACCAGCAGCGUGAUCAGAUACAAGGGCGUGCU |
| | construct | GGGAACCAUCACCGCCGUGGUGAAGACAGAGGGCAGAAUGAAAC |
| | | UGUAUUCUGGCCUGCCUGCCGGCCUGCAGAGACAGAUCUCCAGC |
| | | GCCAGCCUGCGGAUCGGCCUGUACGACACCGUGCAGGAGUUCCU |
| | | GACAGCUGGCAAGGAAACCGCCCCUAGCCUGGGCUCUAAGAUCC |
| | | UGGCUGGCCUGACCACAGGCGGCGUGGCCGUUUUUAUUGGCCAG |
| | | CCUACCGAGGUGGUGAAGGUGCGCCUCCAGGCCCAGAGCCACCU |
| | | GCACGGCAUCAAGCCCAGAUACACCGGCACAUACAACGCCUACA |
| | | GAAUCAUCGCCACAACAGAGGGCCUGACCGGACUGUGGAAGGGA |
| | | ACAACCCCUAACCUGAUGCGGAGCGUGAUCAUCAAUUGCACCGA |
| | | ACUGGUGACCUACGACCUGAUGAAAGAAGCCUUCGUGAAAAACA |
| | | ACAUCCUGGCCGACGACGUGCCAUGCCACCUGGUGUCCGCCCUG |
| | | AUCGCCGGCUUCUGCGCCACAGCCAUGAGCAGCCCUGUGGACGU |
| | | GGUCAAGACCAGAUUCAUCAACAGCCCCCCCGGCCAGUACAAGU |
| | | CCGUGCCUAAUUGUGCCAUGAAAGUGUUCACCAACGAGGGUCCU |
| | | ACAGCUUUUUUCAAGGGCCUUGUGCCAUCUUUUCUGAGACUGGG |
| | | CAGCUGGAACGUGAUCAUGUUCGUCUGCUUCGAGCAGCUGAAGC |
| | | GGGAACUGAGCAAGAGCAGACAGACCAUGGACUGCGCCACCUGA |
| 45 | mol_type = | AUGGGAGGCCUGACCGCUUCUGAUGUGCAUCCCACCCUGGGCGU |
| | RNA | CCAGCUGUUCAGCGCCGGCAUCGCCGCUUGUCUGGCUGAUGUGA |
| | origin = | UUACAUUCCCCCUGGACACCGCCAAGGUGCGGCUGCAGGUGCAG |
| | synthetic | GGCGAGUGCCCCACCAGCAGCGUGAUCAGAUAUAAGGGCGUUCU |
| | construct | GGGCACCAUCACCGCCGUGGUCAAGACCGAAGGCAGAAUGAAAC |
| | | UGUACAGCGGCCUGCCUGCCGGCCUGCAAAGACAGAUCAGCAGC |
| | | GCCAGCCUCAGGAUCGGCCUGUACGACACAGUGCAGGAGUUCCU |
| | | GACAGCUGGCAAGGAAACCGCCCCUUCUCUGGGCAGCAAGAUCC |
| | | UGGCCGGACUGACCACCGGCGGCGUGGCCGUCUUUAUCGGACAG |
| | | CCUACAGAAGUGGUGAAGGUGCGGCUGCAGGCCCAGAGCCACCU |
| | | GCACGGCAUUAAGCCUAGAUACACCGGCACAUACAACGCCUACA |
| | | GAAUCAUCGCCACCACAGAGGGCCUGACAGGCCUGUGGAAGGGC |
| | | ACCACCCCUAAUCUGAUGCGGAGCGUGAUCAUCAAUUGUACCGA |
| | | GCUGGUGACCUACGAUCUGAUGAAAGAGGCCUUCGUGAAAAACA |
| | | ACAUCCUGGCCGACGACGUGCCCUGCCACCUCGUGUCCGCCCUG |
| | | AUCGCCGGCUUCUGCGCCACAGCCAUGAGCUCUCCUGUGGACGU |
| | | CGUGAAGACCAGAUUCAUCAACAGCCCUCCUGGACAGUACAAGU |
| | | CCGUGCCAAACUGCGCCAUGAAAGUGUUCACCAACGAGGGUCCU |
| | | ACCGCUUUUUUCAAGGGCCUGGUGCCAUCUUUUCUGAGACUGGG |
| | | AUCCUGGAACGUGAUCAUGUUCGUGUGCUUCGAGCAGCUCAAGC |
| | | GGGAACUGAGCAAGAGCAGACAAACAAUGGACUGCGCUACAUGA |
| 46 | mol_type = | AUGGGCGGCCUUACAGCUAGCGACGUGCACCCUACCCUGGGAGU |
| | RNA | GCAGCUGUUCAGCGCCGGAAUCGCCGCCUGCCUGGCUGAUGUGA |
| | origin = | UCACAUUCCCCCUGGACACCGCCAAGGUGCGGCUGCAAGUGCAG |
| | synthetic | GGCGAGUGCCCCACCAGCAGCGUGAUCCGGUACAAAGGCGUGCU |
| | construct | GGGAACCAUCACCGCCGUGGUGAAGACAGAAGGAAGAAUGAAAC |
| | | UGUACUCAGGCCUGCCUGCCGGCCUGCAACGCCAGAUCAGCUCU |
| | | GCUUCUCUGAGAAUCGGACUGUAUGAUACCGUGCAGGAGUUCCU |
| | | GACCGCUGGCAAGGAAACCGCCCCUAGCCUGGGCAGCAAGAUCC |
| | | UGGCCGGCCUGACAACAGGUGGCGUCGCCGUGUUCAUCGGCCAG |
| | | CCUACUGAGGUGGUGAAGGUUAGACUGCAGGCCCAGAGCCAUCU |
| | | GCACGGCAUCAAGCCCAGAUACACCGGCACCUACAACGCCUACA |
| | | GAAUCAUCGCCACAACCGAGGGCCUGACCGGCCUCUGGAAGGGC |
| | | ACAACCCCUAAUCUGAUGAGAAGCGUGAUCAUCAACUGCACCGA |
| | | GCUGGUGACCUACGAUCUGAUGAAGGAAGCCUUUGUGAAGAACA |
| | | ACAUCCUGGCCGACGACGUGCCUUGCCACCUCGUGUCCGCCCUG |
| | | AUUGCCGGCUUCUGCGCCACCGCCAUGAGCAGCCCUGUGGACGU |
| | | GGUGAAAACCAGAUUCAUCAACAGCCCACCUGGCCAGUACAAGU |
| | | CCGUGCCCAACUGCGCCAUGAAGGUGUUCACCAAUGAGGGACCU |
| | | ACAGCAUUCUUCAAGGGCCUGGUCCCAUCUUUUCUGCGGCUGGG |
| | | CUCCUGGAACGUGAUCAUGUUCGUUUGUUUUGAGCAGCUGAAAC |
| | | GGGAACUGAGCAAGUCUAGACAGACCAUGGACUGUGCUACAUGA |
| 47 | mol_type = | AUGGGCGGCCUGACCGCUUCUGAUGUGCACCCCACACUGGGAGU |
| | RNA | CCAGCUGUUCAGCGCCGGCAUCGCCGCUUGUCUGGCCGAUGUGA |
| | origin = | UUACUUUUCCACUGGACACCGCCAAGGUGCGGCUGCAGGUGCAA |
| | synthetic | GGCGAGUGCCCUACCAGCAGCGUGAUCAGAUACAAGGGCGUGCU |
| | construct | GGGCACCAUCACCGCUGUUGUGAAGACCGAAGGCAGAAUGAAAC |
| | | UGUACAGCGGCCUGCCUGCCGGACUGCAAAGACAGAUCAGCUCC |
| | | GCCAGCCUGCGCAUCGGACUGUACGAUACCGUGCAGGAGUUCCU |
| | | GACAGCCGGCAAGGAGACAGCCCCUAGCCUGGGCUCUAAGAUCC |
| | | UGGCUGGCCUGACCACAGGCGGAGUGGCCGUCUUCAUCGGCCAG |
| | | CCUACCGAGGUGGUCAAAGUGCGGCUGCAGGCCCAGAGCCACCU |
| | | GCAUGGCAUCAAGCCCCGGUAUACAGGCACCUACAACGCCUACA |
| | | GAAUCAUUGCCACAACAGAGGGCCUGACCGGCCUGUGGAAGGGA |
| | | ACCACCCCCAAUCUGAUGAGAAGCGUGAUCAUCAACUGCACCGA |
| | | ACUGGUGACCUACGACCUGAUGAAGGAAGCCUUCGUGAAAAACA |
| | | ACAUCCUCGCCGACGACGUGCCUUGUCACCUGGUGUCCGCCCUG |
| | | AUCGCCGGCUUCUGCGCCACCGCCAUGAGCUCCCCUGUGGACGU |
| | | GGUGAAGACAAGAUUCAUCAACAGCCCUCCUGGCCAGUACAAGA |
| | | GCGUGCCAAAUUGCGCCAUGAAGGUGUUUACCAACGAGGGCCCU |
| | | ACAGCUUUUUUCAAGGGCCUUGUGCCCAGCUUCCUGAGACUCGG |
| | | AUCUUGGAACGUGAUCAUGUUCGUCUGCUUCGAGCAGCUGAAGC |
| | | GGGAACUGUCUAAAAGCAGACAGACCAUGGACUGCGCAACAUGA |
| 48 | mol_type = | AUGGGCGGACUCACCGCCAGCGACGUGCACCCAACCCUGGGCGU |
| | RNA | GCAGCUGUUCAGCGCCGGAAUCGCCGCUUGUCUGGCCGACGUGA |
| | origin = | UUACCUUCCCUCUGGAUACAGCCAAGGUGCGGCUGCAGGUGCAG |
| | synthetic | GGCGAGUGCCCCACCAGCAGCGUCAUCCGGUAUAAGGGCGUUCU |
| | construct | GGGCACCAUCACAGCUGUGGUGAAGACCGAAGGUAGAAUGAAGC |
| | | UGUACAGCGGCCUCCCUGCCGGCCUGCAAAGACAGAUCAGCUCU |
| | | GCUUCUCUGCGGAUCGGACUGUACGACACCGUCCAGGAGUUCCU |
| | | GACAGCUGGCAAGGAGACAGCCCCUUCUCUGGGAUCCAAGAUCC |
| | | UGGCCGGCCUGACAACAGGCGGCGUGGCCGUCUUUAUUGGCCAG |
| | | CCUACCGAAGUGGUGAAGGUGAGACUGCAGGCCCAGUCCCACCU |
| | | GCAUGGCAUCAAGCCUAGAUACACCGGCACUUACAACGCCUACA |
| | | GAAUCAUCGCAACCACCGAAGGCCUGACCGGCCUGUGGAAGGGA |
| | | ACAACCCCUAACCUGAUGAGAAGCGUGAUCAUCAAUUGCACCGA |
| | | GCUGGUGACCUACGACCUGAUGAAAGAGGCCUUUGUGAAAAACA |
| | | ACAUCCUGGCUGAUGAUGUGCCCUGCCACCUUGUGUCCGCCCUG |
| | | AUCGCCGGAUUUUGUGCCACCGCCAUGAGCUCUCCUGUGGACGU |
| | | GGUGAAAACCAGAUUCAUCAACAGCCCCCCCGGCCAGUACAAGA |
| | | GCGUGCCUAACUGCGCCAUGAAAGUGUUCACCAACGAGGGCCCU |
| | | ACAGCCUUCUUCAAGGGCCUGGUGCCAAGCUUCCUGAGGCUGGG |
| | | CAGCUGGAAUGUGAUCAUGUUCGUGUGCUUCGAGCAGCUGAAGC |
| | | GGGAACUGAGCAAGUCAAGACAAACAAUGGACUGCGCCACCUGA |
| 49 | mol_type = | AUGGGCGGCCUCACCGCCUCUGAUGUGCACCCUACACUGGGCGU |
| | RNA | GCAGCUGUUCAGCGCCGGAAUCGCCGCUUGUCUGGCUGAUGUGA |
| | origin = | UUACCUUCCCCCUGGACACAGCAAAGGUGCGGCUGCAAGUGCAA |
| | synthetic | GGCGAGUGCCCUACCAGCUCCGUGAUCAGAUACAAAGGCGUCCU |
| | construct | GGGCACCAUCACCGCUGUGGUCAAGACCGAAGGCAGAAUGAAGC |
| | | UGUACAGCGGCCUGCCUGCUGGCCUGCAGAGACAGAUCAGCAGC |
| | | GCCAGCCUGAGAAUCGGCCUGUAUGAUACCGUGCAGGAGUUCCU |
| | | GACCGCCGGCAAGGAAACCGCCCCUUCUCUGGGAAGCAAGAUCC |
| | | UGGCCGGACUGACAACCGGCGGAGUGGCCGUGUUUAUCGGACAG |
| | | CCUACAGAAGUGGUGAAAGUGCGGCUGCAGGCCCAGAGCCACCU |
| | | GCACGGCAUCAAGCCCAGAUACACCGGCACCUACAACGCCUACA |
| | | GAAUCAUCGCCACCACAGAGGGCCUGACCGGCCUGUGGAAGGGG |
| | | ACAACCCCUAAUCUGAUGCGGAGCGUGAUCAUCAACUGCACUGA |
| | | GCUGGUCACAUACGACCUGAUGAAAGAGGCCUUUGUGAAGAACA |
| | | ACAUCCUGGCCGACGACGUGCCAUGUCAUCUCGUGUCCGCCCUG |
| | | AUUGCCGGCUUCUGCGCCACCGCCAUGAGCAGCCCUGUGGACGU |
| | | CGUGAAGACAAGAUUCAUCAACAGCCCCCCCGGCCAGUACAAGU |
| | | CCGUGCCAAAUUGCGCCAUGAAAGUGUUCACCAACGAGGGCCCU |
| | | ACAGCUUUUUUCAAGGGCCUGGUUCCUAGCUUCCUGAGGCUGGG |
| | | AUCUUGGAACGUGAUCAUGUUCGUGUGCUUCGAGCAGCUGAAGC |
| | | GGGAACUGUCUAAGUCAAGACAGACCAUGGACUGCGCCACAUGA |
| 50 | mol_type = | AUGGGCGGCUUGACAGCUAGCGACGUGCACCCAACCCUGGGCGU |
| | RNA | CCAGCUGUUCAGCGCUGGAAUCGCCGCUUGUCUGGCCGACGUGA |
| | origin = | UCACAUUUCCUCUGGACACCGCCAAGGUUAGACUGCAAGUGCAG |
| | synthetic | GGCGAGUGCCCUACCAGCUCCGUGAUCAGAUACAAGGGCGUGCU |
| | construct | GGGAACCAUCACCGCCGUGGUCAAGACCGAAGGUAGAAUGAAGC |
| | | UGUACAGCGGCCUGCCUGCCGGCCUUCAGCGGCAGAUCAGCUCU |
| | | GCCUCUCUGCGCAUCGGCCUGUACGAUACCGUGCAGGAGUUUCU |
| | | GACCGCAGGCAAGGAAACAGCCCCUAGCCUGGGCAGCAAGAUUC |
| | | UGGCCGGACUGACUACAGGCGGAGUGGCCGUGUUCAUCGGGCAG |
| | | CCCACAGAGGUGGUGAAGGUGCGGCUGCAGGCCCAGAGCCACCU |
| | | GCAUGGCAUCAAGCCCAGAUACACAGGCACAUACAACGCCUACA |
| | | GAAUCAUUGCUACAACCGAGGGCCUGACCGGCCUGUGGAAGGGC |
| | | ACCACACCUAAUCUGAUGCGGUCCGUGAUCAUCAACUGCACCGA |
| | | ACUGGUGACCUAUGAUCUGAUGAAAGAGGCCUUUGUGAAGAAC |
| | | AACAUCCUCGCCGAUGACGUCCCUUGCCACCUGGUGUCCGCCCU |
| | | GAUCGCCGGCUUCUGCGCCACCGCCAUGAGCAGCCCUGUGGACG |
| | | UGGUGAAAACCAGAUUCAUCAAUAGCCCCCCCGGCCAAUACAAA |
| | | AGCGUGCCUAACUGUGCCAUGAAAGUGUUCACCAACGAGGGCCC |
| | | UACCGCUUUCUUCAAGGGCCUGGUGCCAAGUUUCCUGAGACUGG |
| | | GAUCUUGGAACGUGAUCAUGUUCGUGUGCUUCGAGCAGCUGAAG |
| | | CGGGAACUGAGCAAGUCUAGACAGACCAUGGACUGCGCCACAUG |
| | | A |
| 51 | mol_type = | AUGGGCGGACUGACAGCCUCUGAUGUGCACCCCACACUGGGCGU |
| | RNA | CCAGCUGUUCAGCGCCGGCAUCGCCGCUUGUCUGGCCGACGUGA |
| | origin = | UCACCUUCCCACUGGACACCGCAAAGGUGCGGCUGCAGGUGCAA |
| | synthetic | GGCGAGUGUCCUACCAGCAGCGUGAUCCGCUACAAGGGCGUGCU |
| | construct | GGGCACCAUCACCGCCGUUGUGAAGACCGAAGGCAGAAUGAAGC |
| | | UGUACAGCGGCCUUCCUGCCGGACUGCAAAGACAGAUCAGCUCC |
| | | GCUUCUCUGCGGAUCGGACUGUAUGAUACAGUGCAGGAGUUCCU |
| | | GACAGCUGGGAAAGAGACAGCCCCUAGCCUGGGCAGCAAGAUCC |
| | | UGGCUGGACUGACCACCGGCGGCGUGGCCGUGUUUAUCGGCCAG |
| | | CCUACCGAGGUGGUGAAGGUGAGACUGCAGGCCCAGAGCCACCU |
| | | GCACGGCAUCAAGCCUAGAUACACCGGCACAUACAACGCCUACA |
| | | GAAUCAUCGCCACAACAGAAGGCCUGACCGGCCUGUGGAAGGGC |
| | | ACCACACCUAAUCUGAUGCGGAGCGUGAUUAUCAAUUGCACCGA |
| | | GCUGGUGACCUACGACCUGAUGAAAGAAGCCUUCGUGAAGAACA |
| | | ACAUCCUGGCCGAUGACGUUCCAUGCCAUCUGGUGUCCGCCCUG |
| | | AUCGCCGGCUUCUGCGCCACCGCCAUGUCCAGCCCCGUGGACGU |
| | | GGUGAAAACCAGAUUCAUCAACAGCCCUCCUGGACAGUACAAGU |
| | | CCGUGCCUAACUGCGCCAUGAAGGUGUUCACCAACGAGGGCCCC |
| | | ACCGCUUUUUUUAAGGGCCUGGUCCCCAGCUUCCUGAGACUCGG |
| | | AUCUUGGAACGUGAUUAUGUUCGUGUGCUUCGAGCAGCUGAAGC |
| | | GGGAAUUGUCUAAAAGCAGACAGACCAUGGACUGCGCCACAUGA |
| 52 | mol_type = | AUGGGAGGCCUGACCGCUAGCGACGUGCACCCCACACUGGGCGU |
| | RNA | UCAGCUGUUCAGCGCCGGCAUCGCCGCUUGUCUGGCUGAUGUUA |
| | origin = | UUACCUUCCCCCUGGACACCGCCAAGGUGAGACUGCAAGUGCAG |
| | synthetic | GGCGAAUGUCCUACCAGCUCUGUGAUCCGGUACAAGGGCGUGCU |
| | construct | GGGCACAAUCACCGCAGUGGUCAAGACAGAAGGCAGAAUGAAGC |
| | | UGUACAGCGGCCUGCCUGCCGGCCUGCAGAGACAAAUCAGCUCU |
| | | GCCUCCCUGAGAAUCGGCCUGUAUGAUACCGUGCAGGAGUUCCU |
| | | GACCGCCGGAAAAGAGACAGCCCCUAGCCUGGGCAGCAAGAUCC |
| | | UGGCCGGACUUACCACAGGCGGCGUGGCCGUGUUCAUUGGACAG |
| | | CCUACCGAGGUGGUGAAAGUGCGGCUGCAGGCCCAGAGCCACCU |
| | | GCACGGCAUCAAGCCUAGAUACACCGGAACUUACAACGCCUACA |
| | | GAAUCAUCGCCACCACCGAAGGCCUGACCGGCCUGUGGAAGGGU |
| | | ACAACCCCUAAUCUGAUGCGGAGCGUGAUCAUCAACUGCACCGA |
| | | GCUGGUGACCUACGAUCUGAUGAAAGAGGCCUUUGUCAAGAACA |
| | | ACAUCCUGGCCGACGACGUGCCUUGCCAUCUGGUGUCCGCCCUG |
| | | AUCGCCGGCUUCUGCGCCACAGCUAUGAGCUCCCCUGUGGACGU |
| | | GGUGAAGACCAGAUUCAUCAACAGCCCCCCCGGCCAGUACAAGA |
| | | GCGUGCCAAAUUGCGCCAUGAAAGUCUUUACCAACGAGGGCCCU |
| | | ACAGCUUUUUUCAAGGGACUCGUGCCAUCAUUCCUGCGGCUGGG |
| | | CUCUUGGAACGUGAUCAUGUUCGUGUGCUUCGAGCAGCUGAAGA |
| | | GGGAACUGAGCAAGUCUAGACAGACCAUGGACUGCGCCACAUGA |
| 53 | mol_type = | AUGGGCGGACUGACAGCUAGCGACGUGCACCCUACCCUGGGCGU |
| | RNA | CCAGCUGUUCAGCGCCGGAAUCGCCGCUUGUCUGGCCGAUGUGA |
| | origin = | UUACAUUCCCCCUGGAUACCGCCAAGGUGCGGCUGCAAGUGCAG |
| | synthetic | GGCGAGUGCCCUACCUCCAGCGUGAUCCGGUACAAGGGAGUGCU |
| | construct | GGGAACAAUCACCGCCGUGGUGAAGACCGAAGGCAGAAUGAAAC |
| | | UGUACAGCGGCCUCCCCGCCGGCCUUCAAAGACAGAUCAGCUCU |
| | | GCCAGCCUGCGGAUCGGCCUGUACGACACAGUGCAGGAGUUCCU |
| | | GACCGCCGGCAAGGAAACCGCCCCUUCUCUGGGUUCUAAGAUCC |
| | | UGGCUGGCCUGACCACCGGCGGCGUGGCCGUGUUUAUUGGACAG |
| | | CCUACCGAGGUGGUGAAGGUGCGGCUGCAGGCCCAGAGCCAUCU |
| | | GCACGGCAUCAAGCCCAGAUACACCGGCACCUACAACGCCUACA |
| | | GAAUCAUCGCUACCACCGAGGGCCUGACCGGCCUGUGGAAGGGC |
| | | ACAACACCUAAUCUGAUGAGAAGCGUGAUCAUCAACUGUACAGA |
| | | ACUGGUGACUUAUGAUCUGAUGAAAGAAGCCUUUGUGAAGAAC |
| | | AACAUCCUGGCCGACGACGUGCCAUGCCACCUGGUGUCCGCCCU |
| | | GAUCGCCGGCUUCUGCGCCACCGCCAUGAGCAGCCCCGUGGACG |
| | | UGGUCAAAACCAGAUUCAUCAACAGCCCUCCUGGCCAGUACAAG |
| | | UCCGUUCCAAAUUGCGCCAUGAAAGUGUUCACCAACGAGGGCCC |
| | | UACAGCUUUUUUCAAGGGCCUGGUCCCUAGCUUCCUGAGACUCG |
| | | GAUCUUGGAACGUGAUCAUGUUCGUGUGCUUCGAGCAGCUGAAG |
| | | AGGGAGCUGAGCAAGAGCAGACAGACCAUGGACUGCGCAACAUG |
| | | A |
| 54 | mol_type = | AUCGCUGCUUGUCUGGCCGACGUGAUUACAUUCCCUCUGGACAC |
| | RNA | CGCCAAGGUGCGGCUCCAGGUGCAAGGCGAAUGUCCUACCAGCU |
| | origin = | CCGUGAUCCGGUACAAAGGCGUCCUGGGAACCAUCACCGCCGUG |
| | synthetic | GUGAAAACCGAGGGCAGAAUGAAACUGUACAGCGGCCUGCCUGC |
| | construct | CGGACUGCAAAGACAGAUCAGCAGCGCCAGCCUGCGGAUCGGCC |
| | | UUUAUGAUACCGUGCAGGAGUUCCUGACCGCCGGCAAGGAGACU |
| | | GCCCCUAGCCUGGGCUCUAAGAUCCUGGCUGGCCUGACAACCGG |
| | | CGGCGUCGCCGUGUUCAUCGGACAGCCUACCGAGGUGGUGAAGG |
| | | UUAGACUGCAGGCCCAGAGCCACCUGCACGGCAUCAAGCCAAGA |
| | | UACACCGGCACCUACAACGCCUACAGAAUCAUCGCCACAACCGA |
| | | GGGCCUCACAGGGCUGUGGAAGGGCACCACACCUAAUCUGAUGA |
| | | GAAGCGUGAUCAUCAAUUGCACCGAACUGGUUACAUACGACCUG |
| | | AUGAAGGAAGCCUUUGUGAAGAACAACAUCCUGGCGGAUGACGU |
| | | GCCCUGCCAUCUGGUGUCCGCCCUGAUCGCCGGCUUCUGCGCCA |
| | | CAGCUAUGAGCUCCCCUGUGGACGUGGUGAAGACCAGAUUCAUU |
| | | AACAGCCCCCCCGGCCAGUACAAGAGCGUGCCAAACUGCGCCAU |
| | | GAAAGUGUUCACCAACGAGGGUCCUACAGCUUUUUUCAAGGGCC |
| | | UGGUGCCUAGCUUUCUGAGGCUGGGCUCUUGGAACGUGAUCAUG |
| | | UUCGUCUGCUUCGAGCAGCUGAAGCGGGAACUGAGCAAGUCUAG |
| | | ACAGACCAUGGACUGCGCCACAUGA |
| 55 | mol_type = | AUGGGCGGACUGACAGCCAGCGACGUGCACCCCACCCUGGGAGU |
| | RNA | GCAGCUGUUCAGCGCCGGCAUCGCCGCUUGCCUGGCUGAUGUGA |
| | origin = | UCACUUUUCCACUGGACACCGCAAAGGUGAGACUGCAGGUGCAG |
| | synthetic | GGCGAAUGUCCUACAAGCAGCGUGAUUAGAUACAAGGGCGUGCU |
| | construct | GGGCACAAUCACGGCUGUGGUCAAGACCGAGGGCAGAAUGAAGC |
| | | UGUACAGCGGCUUGCCUGCCGGCCUCCAGAGGCAGAUCAGCUCC |
| | | GCCUCUCUGCGGAUCGGCCUCUACGACACCGUGCAGGAGUUCCU |
| | | GACAGCCGGGAAGGAGACAGCCCCUUCUCUGGGCAGCAAGAUCC |
| | | UGGCCGGACUGACCACCGGCGGCGUGGCCGUGUUCAUUGGACAG |
| | | CCCACCGAAGUGGUGAAAGUUCGGCUGCAAGCCCAGAGCCAUCU |
| | | GCACGGCAUCAAGCCCAGAUACACCGGCACCUACAACGCCUACA |
| | | GAAUCAUCGCCACAACAGAAGGCCUGACCGGCCUGUGGAAGGGC |
| | | ACCACCCCUAACCUGAUGCGGUCCGUGAUCAUCAAUUGCACCGA |
| | | GCUGGUCACCUAUGAUCUGAUGAAAGAGGCCUUCGUGAAGAACA |
| | | ACAUCCUGGCCGACGACGUCCCAUGCCACCUGGUGUCCGCCCUG |
| | | AUCGCCGGCUUCUGCGCUACAGCCAUGAGCUCUCCUGUUGAUGU |
| | | GGUGAAAACCAGAUUCAUCAACAGCCCUCCUGGCCAGUACAAAA |
| | | GCGUGCCUAAUUGUGCCAUGAAGGUGUUCACCAACGAGGGACCU |
| | | ACCGCCUUUUUCAAGGGCCUGGUGCCCAGCUUUCUGAGACUGGG |
| | | AUCUUGGAACGUGAUCAUGUUCGUGUGCUUCGAGCAGCUGAAGC |
| | | GGGAACUGAGCAAGAGCAGACAAACAAUGGACUGCGCUACCUGA |
| 56 | mol_type = | AUGGGCGGACUGACCGCCUCUGAUGUGCACCCUACCCUGGGCGU |
| | RNA | GCAACUGUUCAGCGCCGGCAUCGCCGCUUGUCUGGCCGACGUGA |
| | origin = | UUACAUUCCCUCUGGACACCGCCAAGGUGCGGCUGCAAGUGCAG |
| | synthetic | GGCGAAUGCCCUACCUCCAGCGUUAUCCGGUACAAGGGAGUUCU |
| | construct | GGGCACCAUCACAGCUGUGGUGAAAACCGAGGGCAGAAUGAAGC |
| | | UGUACAGCGGCCUCCCCGCCGGACUGCAGAGACAGAUCAGCAGC |
| | | GCCUCUCUGAGGAUCGGCCUAUAUGACACCGUGCAGGAGUUCCU |
| | | GACAGCCGGCAAGGAAACAGCCCCUAGCCUGGGCAGCAAGAUCC |
| | | UGGCCGGCCUGACCACCGGCGGCGUGGCCGUCUUUAUCGGCCAG |
| | | CCUACAGAGGUGGUGAAAGUGCGGCUGCAGGCCCAGAGCCACCU |
| | | GCACGGCAUUAAGCCUAGAUACACCGGCACCUACAACGCCUACA |
| | | GAAUCAUCGCCACAACCGAAGGCCUGACAGGCCUGUGGAAGGGC |
| | | ACAACACCUAACCUGAUGCGGAGCGUGAUCAUCAAUUGCACCGA |
| | | ACUGGUCACCUACGACCUGAUGAAAGAGGCCUUUGUGAAGAACA |
| | | ACAUCCUGGCUGAUGAUGUGCCAUGUCAUCUGGUCUCCGCCCUG |
| | | AUCGCCGGUUUCUGCGCCACCGCUAUGAGCUCCCCUGUGGACGU |
| | | GGUGAAGACCAGAUUCAUCAACAGCCCCCCCGGCCAGUACAAGU |
| | | CUGUGCCAAAUUGCGCUAUGAAAGUGUUCACCAACGAGGGACCU |
| | | ACAGCUUUUUUCAAGGGCCUGGUGCCCAGCUUCCUCAGACUGGG |
| | | AUCUUGGAACGUGAUCAUGUUCGUGUGCUUCGAGCAGCUGAAGA |
| | | GAGAGCUGAGCAAGAGCAGACAGACCAUGGACUGCGCCACCUGA |
| 57 | mol_type = | AUGGGAGGACUGACCGCCAGCGACGUGCACCCUACCCUGGGAGU |
| | RNA | GCAGCUGUUCAGCGCCGGAAUCGCCGCCUGCCUGGCCGAUGUCA |
| | origin = | UCACCUUCCCCCUGGAUACAGCUAAGGUGCGGCUGCAGGUGCAG |
| | synthetic | GGCGAGUGCCCCACAAGCAGCGUGAUUAGAUACAAGGGCGUGCU |
| | construct | GGGCACAAUCACAGCUGUGGUGAAGACCGAGGGCAGAAUGAAAC |
| | | UGUACAGCGGCCUGCCUGCCGGCCUGCAAAGACAGAUCAGCAGC |
| | | GCCUCUCUGCGGAUCGGCCUCUACGACACCGUGCAGGAGUUCCU |
| | | GACCGCCGGCAAGGAAACCGCCCCUAGCCUGGGCUCCAAGAUCC |
| | | UGGCUGGACUGACCACCGGCGGCGUGGCCGUGUUCAUCGGCCAA |
| | | CCUACAGAGGUGGUCAAAGUGCGGCUGCAGGCCCAGAGCCACCU |
| | | GCAUGGCAUCAAGCCUAGAUACACCGGCACCUACAACGCCUACA |
| | | GAAUCAUCGCCACCACAGAAGGCCUGACCGGCCUGUGGAAGGGC |
| | | ACGACUCCUAAUCUGAUGCGGAGCGUGAUCAUCAACUGCACCGA |
| | | ACUGGUGACCUAUGACCUGAUGAAAGAGGCCUUUGUGAAAAACA |
| | | ACAUCCUUGCUGAUGACGUUCCAUGUCACCUGGUGUCCGCCCUG |
| | | AUCGCCGGCUUCUGCGCCACAGCCAUGUCUUCUCCUGUGGACGU |
| | | GGUGAAGACAAGAUUUAUCAACAGCCCUCCAGGCCAGUACAAGA |
| | | GCGUGCCCAAUUGCGCCAUGAAGGUGUUCACCAACGAAGGCCCU |
| | | ACCGCUUUUUUCAAGGGACUGGUCCCCAGCUUCCUGAGACUGGG |
| | | UUCUUGGAACGUGAUUAUGUUCGUGUGCUUCGAGCAGCUGAAGC |
| | | GCGAGCUGAGCAAGUCCAGACAGACCAUGGACUGUGCCACAUGA |
| 58 | mol_type = | AUGGGCGGACUGACCGCUUCUGAUGUGCACCCUACCCUGGGCGU |
| | RNA | GCAGCUGUUCUCCGCCGGCAUCGCCGCUUGUCUGGCCGACGUGA |
| | origin = | UUACUUUUCCACUGGACACCGCAAAGGUCAGACUGCAGGUGCAG |
| | synthetic | GGCGAGUGCCCCACAAGCAGCGUGAUCAGAUAUAAGGGCGUGCU |
| | construct | GGGAACCAUCACCGCCGUGGUGAAGACCGAGGGCAGAAUGAAGC |
| | | UGUACAGCGGCCUCCCCGCCGGCCUGCAGAGACAGAUCAGCUCU |
| | | GCCAGCCUGCGGAUCGGACUCUACGAUACAGUGCAGGAGUUCCU |
| | | GACCGCUGGCAAGGAAACCGCCCCUAGCCUGGGUUCUAAGAUCC |
| | | UGGCCGGACUGACCACCGGAGGAGUGGCCGUGUUCAUCGGCCAA |
| | | CCUACCGAGGUGGUCAAGGUGCGGCUGCAAGCCCAGAGCCAUCU |
| | | GCACGGCAUCAAGCCUAGAUACACCGGCACAUACAACGCCUACA |
| | | GAAUUAUCGCCACCACAGAAGGCCUGACAGGCCUGUGGAAGGGC |
| | | ACCACCCCUAAUCUGAUGCGGAGCGUUAUCAUCAACUGCACCGA |
| | | ACUGGUGACCUACGACCUGAUGAAAGAGGCCUUCGUGAAGAACA |
| | | ACAUCCUGGCUGAUGACGUGCCCUGCCACCUGGUGUCCGCCCUG |
| | | AUCGCCGGCUUCUGCGCCACAGCCAUGAGCAGCCCUGUGGACGU |
| | | GGUGAAAACAAGAUUCAUCAACAGCCCUCCUGGCCAGUACAAGA |
| | | GCGUGCCAAACUGUGCCAUGAAAGUCUUUACCAACGAGGGCCCU |
| | | ACAGCUUUUUUCAAGGGCCUAGUGCCCUCCUUCCUGAGGCUGGG |
| | | CUCUUGGAAUGUGAUCAUGUUCGUGUGCUUCGAGCAGCUGAAGC |
| | | GGGAACUGAGCAAAAGCAGACAGACAAUGGACUGCGCCACAUGA |
| 59 | mol_type = | AUGGGCGGCCUGACCGCCAGUGAUGUGCACCCCACCCUGGGUGU |
| | RNA | UCAACUGUUCUCUGCCGGAAUCGCCGCCUGCCUGGCCGACGUGA |
| | origin = | UCACCUUUCCACUGGACACCGCCAAGGUGAGGCUGCAGGUGCAG |
| | synthetic | GGCGAGUGUCCUACAAGCAGCGUGAUCAGAUACAAGGGCGUGCU |
| | construct | GGGCACCAUCACCGCUGUGGUCAAGACAGAGGGCAGAAUGAAAC |
| | | UGUACAGCGGCCUGCCUGCCGGCCUCCAGCGGCAGAUCAGCUCC |
| | | GCCUCUCUUCGGAUCGGCCUCUACGACACCGUGCAGGAGUUCCU |
| | | GACAGCUGGCAAGGAAACCGCCCCUUCUCUGGGAUCUAAGAUCC |
| | | UGGCUGGCCUGACCACAGGAGGAGUGGCCGUGUUUAUCGGCCAG |
| | | CCUACCGAAGUGGUGAAGGUGCGGCUGCAGGCCCAGAGCCAUCU |
| | | GCACGGCAUCAAGCCUAGAUAUACAGGCACCUACAACGCCUACA |
| | | GAAUCAUCGCGACAACCGAGGGCCUGACCGGCCUGUGGAAGGGC |
| | | ACAACACCUAAUCUGAUGAGAAGCGUGAUUAUUAACUGCACCGA |
| | | GCUGGUUACAUACGAUCUGAUGAAAGAAGCCUUCGUGAAGAACA |
| | | ACAUCCUGGCCGAUGACGUGCCUUGUCACCUGGUGUCCGCUCUG |
| | | AUCGCCGGCUUCUGCGCCACGGCCAUGAGCUCCCCUGUGGACGU |
| | | GGUCAAAACCAGAUUCAUCAACAGCCCCCCCGGCCAGUACAAGA |
| | | GCGUGCCCAAUUGCGCCAUGAAAGUCUUUACCAACGAGGGACCU |
| | | ACCGCUUUCUUCAAGGGCCUGGUGCCAAGCUUCCUGAGACUGGG |
| | | CAGCUGGAACGUGAUCAUGUUCGUGUGCUUCGAGCAGCUGAAGC |
| | | GGGAACUGAGCAAGAGCAGACAAACAAUGGACUGCGCCACCUGA |
| 60 | mol_type = | AUGGGCGGCCUGACAGCCAGUGAUGUGCACCCCACCCUGGGCGU |
| | RNA | GCAACUGUUCAGCGCCGGCAUCGCAGCCUGCCUGGCCGACGUGA |
| | origin = | UCACAUUUCCAUUGGACACAGCCAAGGUGAGACUGCAGGUGCAG |
| | synthetic | GGCGAGUGCCCCACAAGCAGCGUGAUUCGGUACAAGGGCGUGCU |
| | construct | GGGCACCAUCACCGCCGUGGUCAAGACCGAAGGCAGGAUGAAAC |
| | | UGUACAGCGGCCUGCCUGCCGGCCUGCAGAGACAGAUCAGCUCU |
| | | GCUAGCCUGAGAAUCGGACUCUACGACACCGUCCAGGAGUUCCU |
| | | GACUGCUGGCAAGGAAACCGCCCCUUCUCUGGGAAGCAAGAUCC |
| | | UGGCCGGACUCACUACCGGCGGAGUGGCCGUGUUCAUCGGCCAG |
| | | CCUACAGAGGUGGUGAAAGUGCGGCUGCAAGCCCAGAGCCAUCU |
| | | GCACGGCAUCAAGCCUAGAUACACCGGCACCUACAACGCCUACA |
| | | GAAUCAUCGCCACCACAGAAGGCCUGACCGGCCUGUGGAAGGGC |
| | | ACAACCCCUAAUCUGAUGCGGUCUGUGAUCAUCAAUUGCACAGA |
| | | GCUGGUGACCUAUGACCUGAUGAAAGAAGCCUUCGUGAAGAACA |
| | | ACAUCCUGGCUGAUGAUGUGCCCUGUCACCUGGUCUCCGCCCUG |
| | | AUCGCCGGCUUCUGCGCCACCGCCAUGUCCAGCCCUGUGGACGU |
| | | GGUGAAGACAAGAUUCAUCAACAGCCCACCUGGCCAGUACAAGU |
| | | CUGUUCCCAACUGUGCUAUGAAAGUGUUUACCAACGAGGGACCU |
| | | ACCGCUUUUUUCAAGGGACUGGUGCCUAGCUUCCUGCGGCUGGG |
| | | CUCCUGGAACGUGAUCAUGUUCGUGUGCUUCGAGCAGCUGAAGA |
| | | GAGAGCUGAGCAAGAGCAGACAGACCAUGGACUGCGCCACAUGA |
| 61 | mol_type = | AUGGGCGGCCUGACCGCCUCUGAUGUGCACCCUACCCUGGGCGU |
| | RNA | GCAGCUGUUCAGCGCCGGAAUCGCCGCCUGUCUGGCCGACGUGA |
| | origin = | UCACCUUUCCACUGGACACCGCUAAAGUGCGGCUGCAGGUGCAA |
| | synthetic | GGCGAGUGCCCUACAAGCUCUGUGAUCAGAUACAAGGGAGUGCU |
| | construct | GGGCACCAUCACAGCCGUGGUGAAGACCGAAGGCAGAAUGAAAC |
| | | UGUACAGCGGCCUGCCUGCUGGACUGCAAAGACAGAUCAGCUCU |
| | | GCUAGCCUGAGGAUCGGACUUUAUGAUACCGUGCAGGAGUUCCU |
| | | GACAGCCGGCAAAGAGACAGCCCCUAGCCUGGGCUCCAAGAUCC |
| | | UGGCCGGCCUGACCACAGGAGGCGUCGCCGUGUUCAUCGGCCAG |
| | | CCUACAGAAGUGGUCAAGGUGCGGCUACAGGCCCAGAGCCACCU |
| | | GCACGGCAUCAAGCCCAGAUACACCGGCACCUACAACGCCUACC |
| | | GGAUCAUCGCCACAACCGAGGGCCUGACCGGCCUCUGGAAGGGC |
| | | ACAACCCCUAACCUGAUGCGCAGCGUGAUCAUCAAUUGCACCGA |
| | | ACUGGUCACCUACGACCUGAUGAAAGAAGCCUUCGUGAAGAACA |
| | | ACAUCCUGGCUGAUGACGUGCCCUGCCAUCUGGUGUCCGCCCUG |
| | | AUCGCCGGCUUCUGCGCCACCGCCAUGAGCAGCCCCGUGGACGU |
| | | GGUUAAGACAAGAUUCAUUAACUCCCCUCCAGGCCAGUACAAGA |
| | | GCGUGCCUAAUUGCGCCAUGAAGGUGUUCACCAACGAGGGACCU |
| | | ACAGCUUUUUUCAAGGGCCUGGUGCCCAGCUUCCUGAGACUGGG |
| | | CAGCUGGAACGUGAUUAUGUUCGUGUGUUUUGAGCAGCUGAAG |
| | | CGGGAGCUGUCUAAGUCCAGACAGACCAUGGACUGCGCAACAUG |
| | | A |
| 62 | mol_type = | AUGGGCGGACUGACAGCCAGUGAUGUGCACCCCACCCUGGGAGU |
| | RNA | UCAGCUGUUCAGCGCUGGAAUCGCCGCCUGCCUGGCCGACGUGA |
| | origin = | UUACAUUUCCUCUGGACACCGCCAAGGUGCGGCUGCAAGUGCAG |
| | synthetic | GGCGAGUGUCCUACAAGCAGCGUGAUCCGGUACAAGGGCGUGCU |
| | construct | GGGAACCAUCACAGCUGUGGUGAAAACCGAGGGAAGAAUGAAGC |
| | | UGUACAGCGGCCUGCCUGCUGGCCUGCAGAGACAGAUCAGCUCC |
| | | GCUUCUCUGCGGAUCGGCCUUUAUGAUACCGUGCAGGAGUUCCU |
| | | GACAGCUGGCAAGGAAACCGCCCCUAGCCUGGGCUCUAAAAUCC |
| | | UGGCUGGCCUGACCACCGGUGGCGUGGCCGUGUUCAUCGGCCAG |
| | | CCUACAGAAGUGGUCAAGGUGCGGCUGCAGGCCCAGAGCCACCU |
| | | GCACGGCAUCAAGCCCAGAUACACCGGCACAUACAACGCCUACA |
| | | GAAUCAUCGCCACCACCGAGGGCCUAACCGGCCUGUGGAAGGGC |
| | | ACCACCCCUAAUCUGAUGAGAAGCGUGAUCAUCAACUGCACCGA |
| | | ACUGGUGACCUACGACCUCAUGAAGGAAGCCUUUGUGAAGAACA |
| | | ACAUCCUGGCCGAUGACGUGCCCUGCCAUCUGGUCUCCGCCCUG |
| | | AUCGCCGGCUUCUGCGCCACAGCCAUGAGCAGCCCCGUGGACGU |
| | | GGUGAAGACAAGAUUCAUCAACAGCCCUCCUGGCCAGUACAAGA |
| | | GCGUGCCAAAUUGUGCCAUGAAAGUGUUUACCAACGAGGGCCCU |
| | | ACCGCCUUCUUCAAGGGACUGGUUCCAUCCUUCCUGAGGCUGGG |
| | | CAGCUGGAACGUGAUCAUGUUCGUGUGCUUCGAGCAGCUGAAGA |
| | | GAGAGCUGUCUAAAUCUAGACAAACAAUGGACUGCGCCACCUGA |
| 63 | mol_type = | AUGGGAGGCCUGACCGCUAGCGACGUGCACCCUACACUGGGCGU |
| | RNA | GCAGCUGUUCAGCGCCGGCAUCGCCGCAUGCCUGGCUGAUGUGA |
| | origin = | UCACCUUCCCCCUGGAUACCGCCAAGGUGCGGCUGCAAGUGCAG |
| | synthetic | GGCGAGUGCCCUACAAGCUCCGUGAUCAGAUACAAGGGAGUGCU |
| | construct | GGGCACAAUCACAGCUGUGGUGAAAACCGAGGGCAGAAUGAAGC |
| | | UGUACAGCGGCCUGCCUGCCGGACUUCAGAGACAGAUUAGCAGU |
| | | GCCUCUCUGAGAAUCGGCCUGUAUGAUACAGUGCAGGAGUUCCU |
| | | GACAGCCGGCAAAGAGACAGCUCCUUCCCUGGGCAGCAAGAUCC |
| | | UGGCCGGCCUGACCACCGGCGGCGUGGCCGUGUUCAUCGGACAG |
| | | CCUACCGAAGUGGUCAAAGUGCGGCUGCAGGCCCAGAGCCACCU |
| | | GCAUGGCAUCAAGCCUCGGUACACCGGCACCUACAACGCCUACA |
| | | GAAUCAUCGCCACCACAGAAGGCCUGACCGGACUGUGGAAGGGA |
| | | ACCACCCCUAACCUGAUGAGAAGCGUGAUCAUCAAUUGCACUGA |
| | | GCUGGUGACCUACGACCUGAUGAAGGAAGCCUUCGUUAAGAACA |
| | | ACAUCCUCGCCGACGACGUCCCCUGUCACCUGGUCAGCGCCCUG |
| | | AUCGCCGGCUUCUGCGCCACCGCCAUGAGCUCUCCAGUGGACGU |
| | | GGUGAAGACCAGAUUCAUCAACAGCCCCCCCGGCCAGUACAAAA |
| | | GCGUGCCUAAUUGCGCCAUGAAGGUGUUUACCAACGAGGGCCCU |
| | | ACAGCUUUUUUCAAGGGCCUGGUGCCAUCUUUCCUGCGCCUCGG |
| | | AAGCUGGAACGUGAUUAUGUUCGUUUGUUUUGAGCAGCUGAAG |
| | | CGGGAACUGUCUAAGUCCAGACAAACAAUGGACUGCGCCACCUG |
| | | A |
| 64 | mol_type = | AUGGGCGGCCUGACAGCCUCUGAUGUGCACCCUACCCUGGGAGU |
| | RNA | GCAGCUGUUCAGCGCCGGCAUCGCCGCUUGUCUGGCCGAUGUGA |
| | origin = | UCACCUUUCCUCUGGACACCGCUAAGGUGCGCCUGCAGGUGCAG |
| | synthetic | GGCGAGUGUCCUACCAGCAGCGUGAUCAGAUACAAGGGCGUGCU |
| | construct | GGGCACCAUCACAGCAGUGGUCAAAACCGAGGGCAGAAUGAAAC |
| | | UGUACAGCGGCCUCCCCGCCGGCCUGCAAAGACAAAUCAGCUCU |
| | | GCCAGCCUGCGGAUUGGCCUGUAUGACACCGUACAGGAGUUCCU |
| | | GACAGCCGGAAAGGAAACCGCCCCUUCUCUGGGAUCCAAGAUCC |
| | | UGGCCGGCCUGACCACUGGCGGAGUGGCCGUGUUCAUCGGCCAG |
| | | CCUACCGAGGUGGUGAAGGUGCGGCUGCAGGCCCAGAGCCACCU |
| | | GCAUGGCAUCAAGCCUAGAUACACAGGCACGUACAACGCCUACA |
| | | GAAUCAUCGCCACCACCGAAGGACUGACCGGCCUGUGGAAGGGC |
| | | ACAACCCCUAACCUGAUGAGAAGCGUCAUCAUCAACUGCACCGA |
| | | ACUGGUCACAUACGACCUGAUGAAAGAGGCCUUUGUGAAGAACA |
| | | AUAUCCUGGCCGACGAUGUGCCAUGCCACCUGGUGUCCGCCCUC |
| | | AUCGCCGGCUUCUGCGCUACCGCUAUGAGCUCCCCUGUGGACGU |
| | | GGUGAAGACCCGGUUCAUCAACAGCCCCCCCGGCCAGUACAAAA |
| | | GCGUGCCAAAUUGCGCCAUGAAGGUGUUCACAAACGAGGGACCU |
| | | ACAGCUUUUUUCAAGGGCCUGGUUCCCAGCUUCCUGAGACUGGG |
| | | CUCUUGGAACGUGAUCAUGUUCGUGUGCUUCGAGCAGCUGAAGC |
| | | GGGAACUGAGCAAGAGCAGACAGACCAUGGACUGCGCCACAUGA |
| 65 | mol_type = | AUGGGCGGACUGACCGCUUCUGAUGUGCACCCUACACUGGGCGU |
| | RNA | CCAACUGUUCUCUGCCGGCAUCGCCGCUUGUCUGGCUGAUGUGA |
| | origin = | UCACAUUCCCCCUGGACACCGCCAAGGUGCGCCUGCAGGUGCAG |
| | synthetic | GGCGAGUGCCCUACCUCUAGCGUGAUUCGGUACAAGGGCGUGCU |
| | construct | GGGCACCAUCACAGCCGUUGUGAAGACCGAAGGCAGAAUGAAAC |
| | | UGUACUCCGGCCUCCCUGCCGGCCUGCAAAGACAGAUCAGCAGC |
| | | GCCAGCCUCAGAAUUGGCCUGUAUGAUACCGUGCAGGAGUUCCU |
| | | GACCGCCGGCAAGGAGACAGCCCCUAGCCUGGGCAGCAAGAUCC |
| | | UGGCCGGCCUGACAACCGGCGGAGUCGCCGUGUUUAUCGGACAG |
| | | CCUACCGAGGUGGUGAAAGUGCGGCUGCAGGCCCAGAGCCACCU |
| | | GCACGGCAUCAAGCCCAGAUACACCGGCACCUACAACGCCUACC |
| | | GGAUCAUCGCCACCACCGAAGGCCUGACCGGACUGUGGAAGGGC |
| | | ACAACCCCUAAUCUGAUGCGGAGCGUGAUCAUCAACUGCACAGA |
| | | ACUGGUCACCUACGACCUGAUGAAGGAAGCCUUCGUGAAGAACA |
| | | ACAUCCUGGCCGACGACGUUCCAUGUCAUCUGGUGUCCGCCCUG |
| | | AUCGCCGGCUUCUGCGCGACCGCUAUGAGCAGCCCUGUGGACGU |
| | | GGUGAAGACAAGAUUCAUCAACAGCCCCCCCGGCCAGUACAAGU |
| | | CCGUGCCUAAUUGCGCCAUGAAAGUGUUCACCAACGAGGGCCCU |
| | | ACAGCUUUUUUCAAGGGACUGGUGCCAUCUUUUCUGAGACUGGG |
| | | AAGCUGGAACGUGAUCAUGUUCGUGUGCUUCGAGCAGCUGAAGA |
| | | GAGAGCUGAGCAAAAGCAGACAGACGAUGGACUGCGCCACAUGA |
| 66 | mol_type = | AUGGGCGGACUGACCGCUUCUGAUGUGCAUCCCACCCUGGGAGU |
| | RNA | GCAGCUGUUCAGCGCCGGCAUCGCUGCUUGUCUGGCCGACGUGA |
| | origin = | UCACCUUCCCCCUGGACACCGCCAAGGUGCGCCUGCAAGUGCAG |
| | synthetic | GGCGAGUGCCCUACCAGCAGCGUGAUCAGAUACAAGGGCGUGCU |
| | construct | GGGCACAAUCACCGCCGUGGUGAAGACCGAGGGCAGAAUGAAAC |
| | | UGUAUAGUGGCCUGCCUGCCGGUCUCCAGCGGCAGAUUAGCUCU |
| | | GCCAGCCUGCGGAUCGGCCUGUACGACACCGUGCAGGAGUUUCU |
| | | GACAGCAGGCAAAGAGACAGCCCCUUCUCUCGGAAGCAAGAUCC |
| | | UGGCCGGCCUGACCACCGGCGGCGUGGCCGUGUUUAUCGGACAA |
| | | CCUACAGAAGUGGUGAAAGUGCGGCUGCAGGCCCAGAGCCACCU |
| | | GCACGGCAUCAAGCCUAGAUACACCGGAACAUACAACGCCUACA |
| | | GAAUCAUCGCCACCACCGAAGGCCUGACCGGCUUAUGGAAGGGC |
| | | ACCACACCUAAUCUGAUGAGAAGCGUGAUUAUCAACUGUACAGA |
| | | GCUGGUGACCUACGACCUGAUGAAGGAAGCCUUCGUGAAGAACA |
| | | ACAUCCUGGCUGAUGAUGUGCCAUGCCACCUGGUGUCCGCCCUG |
| | | AUCGCCGGCUUCUGCGCCACCGCCAUGAGCAGCCCUGUCGACGU |
| | | CGUGAAGACAAGAUUCAUCAACAGCCCCCCCGGCCAGUACAAGU |
| | | CCGUGCCAAAUUGCGCCAUGAAAGUUUUCACCAACGAGGGCCCU |
| | | ACAGCUUUUUUCAAGGGACUGGUCCCUAGCUUCCUGAGACUGGG |
| | | CUCCUGGAACGUGAUCAUGUUCGUGUGCUUCGAGCAGCUGAAGC |
| | | GGGAACUGUCUAAGAGCAGACAGACCAUGGACUGCGCCACGUGA |
| 67 | mol_type = | AUGGGCGGACUGACAGCCUCUGAUGUGCACCCCACCCUGGGAGU |
| | RNA | GCAGCUGUUCAGCGCCGGCAUCGCCGCUUGUCUGGCCGACGUGA |
| | origin = | UUACCUUCCCUCUGGACACCGCAAAGGUGAGGCUGCAGGUUCAG |
| | synthetic | GGCGAGUGCCCUACCAGCAGUGUGAUCCGGUACAAGGGCGUACU |
| | construct | GGGCACCAUCACCGCUGUGGUGAAGACAGAAGGCAGAAUGAAAC |
| | | UGUACAGCGGCCUUCCCGCCGGCCUGCAAAGACAGAUCAGCAGC |
| | | GCCUCUCUGCGGAUCGGCCUGUAUGAUACAGUGCAGGAGUUCCU |
| | | GACAGCCGGCAAGGAAACCGCCCCUAGCCUGGGAUCUAAGAUCC |
| | | UGGCUGGACUGACCACCGGCGGCGUGGCCGUGUUCAUCGGCCAA |
| | | CCUACCGAGGUGGUCAAGGUGCGGCUGCAGGCCCAGAGCCACCU |
| | | GCAUGGCAUCAAGCCUAGAUACACCGGCACAUACAACGCCUACA |
| | | GAAUCAUCGCCACCACCGAGGGCCUCACCGGCCUGUGGAAGGGA |
| | | ACAACCCCUAAUCUGAUGAGAAGCGUGAUCAUCAAUUGCACGGA |
| | | GCUGGUGACCUACGACCUGAUGAAGGAAGCCUUUGUGAAAAACA |
| | | ACAUCCUGGCCGAUGACGUGCCAUGUCACCUGGUGUCCGCCCUG |
| | | AUCGCCGGCUUUUGCGCCACCGCUAUGAGCUCUCCUGUGGACGU |
| | | GGUGAAAACAAGAUUCAUCAACAGCCCUCCAGGCCAGUACAAGU |
| | | CCGUCCCCAACUGCGCCAUGAAAGUGUUCACCAACGAGGGCCCU |
| | | ACAGCUUUUUUCAAGGGACUGGUCCCCAGCUUCCUGAGACUGGG |
| | | CAGCUGGAACGUGAUUAUGUUCGUGUGCUUCGAGCAGCUGAAGC |
| | | GGGAACUGAGCAAGUCCAGACAGACCAUGGACUGCGCCACAUGA |
| 68 | mol_type = | AUGGGAGGACUGACAGCCUCUGAUGUGCAUCCCACCCUGGGCGU |
| | RNA | GCAGCUGUUCAGCGCCGGCAUCGCCGCUUGUCUGGCCGAUGUGA |
| | origin = | UUACCUUCCCCCUGGACACCGCCAAGGUGCGGCUGCAGGUUCAG |
| | synthetic | GGCGAGUGCCCCACAAGCAGCGUGAUCAGAUACAAGGGCGUGCU |
| | construct | GGGCACCAUCACCGCAGUGGUGAAGACAGAAGGCAGAAUGAAAC |
| | | UGUACAGCGGCCUGCCUGCCGGCCUGCAAAGACAGAUCAGCUCC |
| | | GCCAGCCUGAGAAUCGGCCUGUAUGAUACCGUGCAGGAGUUCCU |
| | | CACAGCCGGCAAGGAAACCGCCCCUUCUCUGGGGUCCAAGAUCC |
| | | UGGCUGGCCUGACCACAGGAGGCGUCGCCGUGUUUAUCGGACAG |
| | | CCUACAGAGGUGGUGAAGGUGCGGCUGCAGGCCCAGAGCCACCU |
| | | GCACGGCAUCAAGCCUAGAUACACCGGCACCUACAACGCCUACA |
| | | GAAUCAUCGCCACCACCGAAGGCCUGACCGGCCUCUGGAAGGGA |
| | | ACCACCCCUAAUCUGAUGCGGAGCGUGAUCAUCAACUGCACCGA |
| | | GCUGGUGACCUACGACCUGAUGAAAGAGGCCUUUGUGAAGAACA |
| | | ACAUCCUGGCCGACGACGUGCCUUGUCACCUGGUCAGCGCCCUG |
| | | AUCGCCGGCUUCUGCGCUACAGCUAUGAGCUCUCCUGUGGACGU |
| | | GGUGAAGACCAGAUUCAUUAACAGCCCACCUGGCCAGUACAAGU |
| | | CCGUGCCAAAUUGCGCCAUGAAAGUCUUCACCAACGAGGGACCU |
| | | ACAGCUUUUUUCAAGGGCCUGGUGCCCAGCUUCCUGAGGCUGGG |
| | | CUCUUGGAACGUGAUCAUGUUCGUGUGCUUCGAGCAGCUGAAGC |
| | | GGGAACUGUCUAAAAGCAGACAAACAAUGGACUGCGCCACAUGA |
| 69 | mol_type = | AUGGGCGGACUCACAGCCAGCGACGUACAUCCUACACUGGGCGU |
| | RNA | GCAGCUGUUCAGCGCCGGCAUCGCUGCCUGCCUGGCUGAUGUGA |
| | origin = | UCACCUUCCCCCUGGAUACAGCAAAGGUUAGACUGCAAGUGCAG |
| | synthetic | GGCGAGUGCCCCACAAGCAGCGUGAUCCGGUACAAAGGCGUGCU |
| | construct | GGGAACCAUUACAGCUGUGGUGAAGACAGAGGGCAGAAUGAAG |
| | | CUGUAUUCUGGCCUGCCUGCCGGCCUGCAGAGACAGAUCAGCUC |
| | | UGCUAGCCUGCGGAUCGGCCUGUACGACACCGUGCAGGAGUUCC |
| | | UCACAGCCGGAAAGGAAACCGCCCCUUCUCUGGGCAGCAAGAUC |
| | | CUGGCCGGCCUGACCACAGGCGGCGUGGCCGUGUUUAUUGGACA |
| | | ACCUACAGAGGUGGUCAAGGUCAGACUGCAGGCCCAGAGCCACC |
| | | UGCACGGCAUCAAACCUAGAUACACCGGCACCUACAACGCCUAC |
| | | AGAAUCAUCGCCACCACCGAGGGCCUGACCGGCCUUUGGAAGGG |
| | | CACCACCCCUAAUCUGAUGCGCAGCGUGAUCAUCAAUUGUACCG |
| | | AACUGGUGACAUACGACCUGAUGAAAGAAGCCUUUGUGAAAAAC |
| | | AACAUCCUGGCCGACGAUGUGCCAUGCCACCUGGUGUCCGCCCU |
| | | GAUCGCCGGCUUCUGCGCCACCGCUAUGAGCUCCCCUGUGGACG |
| | | UGGUGAAGACCAGAUUCAUCAACAGCCCCCCCGGCCAGUACAAG |
| | | UCCGUGCCCAACUGUGCCAUGAAGGUCUUCACUAACGAGGGUCC |
| | | UACCGCCUUUUUCAAGGGACUGGUUCCAAGCUUCCUGAGGCUGG |
| | | GCUCUUGGAACGUGAUCAUGUUCGUGUGCUUCGAGCAGCUGAAG |
| | | CGGGAACUGAGCAAGAGCCGGCAGACCAUGGACUGCGCCACCUG |
| | | A |
| 70 | mol_type = | AUGGGAGGUCUCACAGCCAGCGACGUACACCCCACAUUAGGGGU |
| | RNA | UCAGCUCUUUAGUGCAGGCAUAGCCGCUUGCCUCGCUGAUGUUA |
| | origin = | UUACUUUUCCCCUCGACACUGCUAAGGUGCGACUGCAAGUACAG |
| | synthetic | GGCGAAUGCCCUACUUCAUCAGUGAUUCGAUACAAGGGCGUAUU |
| | construct | GGGCACUAUUACUGCCGUCGUGAAAACAGAAGGCCGUAUGAAGC |
| | | UGUAUAGUGGGCUGCCAGCGGGAUUGCAGAGGCAGAUCAGCUCU |
| | | GCAUCUCUGCGAAUCGGACUGUACGACACCGUUCAAGAGUUCCU |
| | | UACCGCAGGAAAGGAGACAGCACCUAGCCUAGGGUCCAAAAUCU |
| | | UGGCAGGUCUCACUACAGGGGGCGUAGCUGUUUUCAUCGGACAA |
| | | CCCACAGAAGUUGUAAAGGUCCGACUCCAAGCACAGAGCCAUCU |
| | | UCACGGGAUCAAACCUCGCUACACUGGCACGUACAACGCUUACA |
| | | GGAUCAUCGCCACUACAGAGGGUUUAACAGGGCUUUGGAAGGGG |
| | | ACAACACCUAAUCUGAUGCGAUCCGUGAUUAUUAACUGCACCGA |
| | | AUUGGUGACUUACGAUCUCAUGAAGGAGGCCUUCGUGAAGAACA |
| | | ACAUCUUGGCGGACGACGUGCCUUGUCAUUUAGUCUCUGCACUG |
| | | AUAGCCGGAUUCUGUGCCACCGCCAUGUCGAGCCCUGUUGACGU |
| | | CGUGAAGACGCGAUUUAUCAAUUCUCCUCCUGGUCAAUAUAAGA |
| | | GUGUCCCUAAUUGUGCCAUGAAGGUCUUCACAAACGAGGGCCCU |
| | | ACAGCCUUUUUCAAGGGACUCGUCCCAUCCUUUCUACGGCUCGG |
| | | UUCUUGGAACGUCAUCAUGUUCGUGUGUUUCGAACAACUGAAAC |
| | | GAGAGCUGUCCAAAAGUAGGCAGACCAUGGACUGCGCUACUUGA |
| 71 | mol_type = | AUGGGCGGUCUAACGGCCUCUGACGUCCAUCCCACACUUGGGGU |
| | RNA | GCAGCUCUUCAGUGCGGGGAUCGCCGCCUGCUUGGCCGAUGUCA |
| | origin = | UUACUUUUCCUCUGGACACAGCAAAGGUGCGAUUGCAGGUUCAA |
| | synthetic | GGAGAAUGCCCCACCUCCUCCGUCAUUCGAUAUAAAGGCGUUUU |
| | construct | GGGGACGAUCACUGCCGUGGUGAAGACGGAGGGCCGUAUGAAAU |
| | | UGUACAGCGGGCUGCCAGCAGGCCUGCAGAGGCAGAUAAGCUCC |
| | | GCAUCAUUGCGAAUCGGCCUGUACGACACAGUGCAAGAAUUUCU |
| | | UACUGCCGGAAAGGAGACAGCACCCAGUUUGGGGAGCAAAAUCC |
| | | UCGCCGGGCUGACCACAGGCGGCGUCGCUGUAUUUAUUGGACAG |
| | | CCUACUGAAGUGGUAAAGGUACGACUCCAGGCCCAGUCUCAUCU |
| | | GCACGGAAUCAAGCCUAGAUACACUGGGACAUAUAACGCUUACA |
| | | GGAUCAUCGCUACAACAGAAGGCCUCACUGGACUUUGGAAGGGC |
| | | ACAACUCCUAAUUUAAUGCGAUCAGUCAUCAUCAAUUGCACCGA |
| | | GUUGGUCACAUACGACCUAAUGAAGGAGGCCUUCGUGAAAAACA |
| | | AUAUACUCGCAGAUGACGUUCCUUGUCACUUAGUGUCUGCGCUG |
| | | AUCGCCGGAUUUUGUGCCACCGCCAUGUCUAGCCCUGUUGAUGU |
| | | CGUGAAGACAAGGUUUAUCAAUUCACCACCUGGCCAGUAUAAAU |
| | | CUGUUCCUAAUUGUGCAAUGAAGGUGUUCACGAAUGAAGGUCCA |
| | | ACAGCAUUCUUCAAGGGGCUUGUCCCUUCAUUUCUGCGACUGGG |
| | | AUCAUGGAACGUGAUCAUGUUUGUGUGUUUUGAACAACUGAAA |
| | | AGAGAGCUGUCGAAGUCCAGGCAGACAAUGGACUGCGCCACCUG |
| | | A |
| 72 | mol_type = | AUGGGCGGACUGACCGCUUCCGACGUACACCCUACCUUAGGAGU |
| | RNA | GCAGCUCUUCAGUGCAGGAAUCGCCGCUUGCCUUGCUGAUGUCA |
| | origin = | UUACUUUUCCAUUAGACACCGCCAAGGUGCGGCUACAGGUUCAG |
| | synthetic | GGCGAAUGUCCCACUUCUUCCGUCAUUCGGUACAAAGGCGUUCU |
| | construct | GGGAACGAUCACAGCUGUCGUGAAAACCGAAGGCCGUAUGAAGC |
| | | UGUACAGCGGGCUUCCAGCAGGACUUCAGAGGCAGAUCAGCUCG |
| | | GCGUCCUUACGCAUCGGACUCUACGACACAGUCCAAGAAUUCCU |
| | | CACAGCUGGAAAGGAAACUGCCCCUAGCCUGGGGAGCAAAAUAC |
| | | UGGCCGGGCUCACAACAGGCGGCGUUGCUGUGUUUAUUGGACAG |
| | | CCCACUGAGGUUGUCAAGGUCCGCCUGCAAGCCCAAAGCCACCU |
| | | UCACGGGAUUAAGCCUCGCUACACUGGAACAUACAAUGCAUAUA |
| | | GAAUCAUCGCCACCACCGAAGGCCUAACUGGACUGUGGAAAGGG |
| | | ACAACACCUAACUUAAUGCGGUCAGUUAUUAUUAACUGCACUGA |
| | | GUUAGUGACUUACGAUCUCAUGAAGGAAGCAUUCGUGAAGAAC |
| | | AACAUUUUGGCAGAUGACGUUCCUUGUCAUUUGGUUUCCGCAUU |
| | | AAUAGCCGGGUUUUGUGCUACCGCCAUGUCUUCCCCAGUCGACG |
| | | UCGUGAAGACAAGGUUUAUAAAUUCCCCACCUGGCCAGUAUAAA |
| | | AGUGUCCCUAACUGUGCAAUGAAAGUCUUCACAAACGAGGGCCC |
| | | UACCGCCUUCUUCAAGGGGCUUGUUCCCUCUUUUCUGCGACUCG |
| | | GCUCAUGGAACGUCAUCAUGUUUGUCUGUUUUGAACAGCUGAAA |
| | | AGGGAGCUAUCAAAGUCAAGACAGACCAUGGACUGCGCCACCUG |
| | | A |
| 73 | mol_type = | AUGGGCGGCCUGACCGCUUCAGACGUUCACCCGACGUUGGGAGU |
| | RNA | GCAGCUAUUCAGCGCAGGAAUAGCCGCUUGCUUAGCAGAUGUGA |
| | origin = | UUACUUUCCCUCUCGAUACUGCUAAGGUCCGCCUUCAAGUUCAG |
| | synthetic | GGCGAAUGCCCUACUUCUUCCGUGAUUCGCUAUAAGGGCGUACU |
| | construct | GGGAACUAUUACAGCUGUCGUGAAAACAGAAGGCCGCAUGAAAC |
| | | UCUACAGCGGACUCCCUGCCGGGUUACAAAGACAAAUCAGCUCA |
| | | GCUUCCCUGCGGAUCGGCUUGUACGACACCGUUCAGGAAUUCCU |
| | | CACAGCCGGAAAGGAGACUGCACCUAGUUUAGGAAGCAAAAUUC |
| | | UCGCAGGGCUUACCACAGGUGGCGUCGCCGUUUUCAUUGGACAA |
| | | CCAACCGAAGUAGUUAAGGUCCGACUCCAAGCUCAAUCUCACCU |
| | | ACACGGAAUUAAGCCUCGCUACACUGGCACGUAUAACGCUUAUA |
| | | GGAUUAUCGCUACAACAGAAGGAUUAACGGGCCUGUGGAAGGGC |
| | | ACCACCCCCAAUUUAAUGCGAUCAGUCAUCAUCAACUGCACCGA |
| | | GUUGGUAACAUACGAUCUCAUGAAGGAAGCCUUUGUGAAAAAC |
| | | AACAUACUGGCAGACGACGUCCCUUGUCAUUUGGUUUCUGCGCU |
| | | CAUCGCAGGGUUUUGUGCUACCGCCAUGUCUAGCCCUGUUGACG |
| | | UUGUCAAGACACGAUUUAUAAACUCUCCACCAGGCCAGUAUAAA |
| | | UCUGUCCCUAAUUGUGCCAUGAAGGUCUUCACAAACGAGGGUCC |
| | | CACUGCUUUCUUCAAGGGGCUUGUUCCCUCGUUUCUGCGGCUUG |
| | | GAUCCUGGAAUGUUAUCAUGUUUGUGUGCUUUGAACAGUUGAA |
| | | AAGAGAGUUGUCCAAAUCCAGGCAGACUAUGGACUGCGCCACAU |
| | | AG |
| 74 | mol_type = | AUGGGAGGGCUCACAGCAAGCGACGUGCAUCCAACCUUAGGAGU |
| | RNA | ACAGCUCUUCAGUGCAGGAAUCGCCGCUUGCCUAGCCGAUGUCA |
| | origin = | UUACCUUUCCACUGGACACCGCUAAGGUUCGCCUGCAAGUUCAG |
| | synthetic | GGCGAGUGUCCUACCUCUUCUGUAAUUCGAUAUAAAGGCGUGUU |
| | construct | GGGGACAAUCACAGCUGUCGUGAAAACAGAAGGCCGCAUGAAGC |
| | | UCUACAGCGGACUGCCAGCAGGUUUGCAACGGCAGAUAAGCUCA |
| | | GCAUCCCUCCGUAUCGGGCUGUACGACACAGUUCAGGAAUUCCU |
| | | CACUGCCGGGAAAGAGACAGCCCCUAGCUUGGGGAGCAAAAUUU |
| | | UGGCCGGGCUCACUACAGGUGGCGUCGCUGUGUUCAUUGGACAA |
| | | CCCACUGAGGUGGUGAAGGUACGACUCCAGGCUCAAAGCCAUUU |
| | | ACACGGGAUUAAACCACGCUACACUGGAACUUACAACGCAUACA |
| | | GAAUCAUCGCUACCACAGAAGGUUUGACGGGACUUUGGAAAGGG |
| | | ACAACACCUAAUUUAAUGCGAUCUGUCAUCAUCAAUUGCACUGA |
| | | AUUGGUGACUUAUGACCUGAUGAAAGAAGCAUUUGUUAAAAAU |
| | | AACAUCCUGGCGGAUGAUGUCCCUUGUCAUUUGGUAUCAGCACU |
| | | CAUUGCAGGGUUUUGUGCCACCGCCAUGUCUUCCCCAGUUGAUG |
| | | UUGUAAAGACACGGUUCAUAAACUCACCUCCAGGCCAGUAUAAG |
| | | AGUGUCCCCAAUUGUGCAAUGAAAGUGUUCACAAACGAAGGUCC |
| | | AACUGCAUUCUUCAAGGGGCUUGUUCCCUCUUUCCUUCGUCUCG |
| | | GAUCCUGGAACGUGAUAAUGUUUGUGUGUUUUGAACAACUUAA |
| | | ACGAGAGCUUUCUAAAAGUAGGCAGACUAUGGACUGUGCAACGU |
| | | AG |
| 75 | mol_type = | AUGGGCGGCCUCACCGCAUCAGACGUCCAUCCAACAUUGGGGGU |
| | RNA | GCAGCUCUUCAGUGCCGGCAUCGCUGCCUGUCUCGCCGACGUCA |
| | origin = | UUACCUUUCCGUUAGAUACUGCAAAGGUCCGCCUGCAGGUUCAA |
| | synthetic | GGCGAGUGCCCAACUUCCUCCGUUAUUCGCUACAAGGGCGUGCU |
| | construct | GGGAACAAUCACCGCAGUGGUGAAAACAGAGGGCCGAAUGAAGC |
| | | UCUACUCCGGGCUACCAGCAGGCUUGCAGAGGCAAAUCAGUUCU |
| | | GCAUCCCUGCGCAUCGGUCUAUACGACACUGUUCAGGAGUUCCU |
| | | CACCGCCGGGAAGGAGACAGCCCCUAGUUUGGGCAGCAAAAUAC |
| | | UCGCCGGGCUCACAACAGGUGGCGUUGCCGUUUUCAUCGGACAA |
| | | CCAACCGAGGUCGUGAAGGUGCGACUGCAAGCACAAAGCCAUCU |
| | | CCACGGGAUCAAGCCUAGAUACACUGGAACUUACAACGCCUACA |
| | | GGAUCAUCGCUACAACCGAAGGCUUAACUGGACUCUGGAAAGGG |
| | | ACAACCCCUAACUUAAUGCGAAGCGUCAUAAUCAACUGUACAGA |
| | | ACUAGUCACAUACGAUCUCAUGAAAGAAGCCUUUGUGAAAAAUA |
| | | AUAUCCUGGCAGAUGACGUUCCUUGUCACCUGGUCUCAGCACUG |
| | | AUCGCCGGAUUUUGCGCUACUGCAAUGUCUUCUCCUGUUGAUGU |
| | | UGUGAAAACACGGUUCAUAAAUUCUCCUCCAGGUCAGUAUAAGA |
| | | GUGUUCCUAAUUGUGCCAUGAAAGUCUUCACUAACGAAGGUCCU |
| | | ACAGCAUUCUUUAAGGGACUCGUUCCCUCUUUUCUCCGUCUUGG |
| | | CUCAUGGAACGUUAUCAUGUUUGUGUGUUUUGAACAGCUGAAG |
| | | AGGGAGCUGUCCAAAUCAAGACAAACCAUGGACUGCGCCACAUG |
| | | A |
| 76 | mol_type = | AUGGGAGGGCUUACUGCUAGUGACGUCCACCCUACCCUCGGAGU |
| | RNA | UCAGCUCUUCAGUGCCGGAAUCGCCGCGUGCCUCGCCGACGUGA |
| | origin = | UUACCUUUCCACUAGACACCGCUAAGGUGCGGCUGCAAGUCCAG |
| | synthetic | GGCGAGUGUCCUACAUCCUCCGUCAUUCGCUAUAAAGGCGUUUU |
| | construct | GGGCACCAUCACGGCUGUAGUCAAAACAGAGGGCCGUAUGAAGC |
| | | UCUACAGCGGGCUGCCCGCCGGGUUGCAGAGACAAAUAAGCUCU |
| | | GCUUCUCUGCGGAUCGGUCUGUACGACACUGUUCAGGAGUUCCU |
| | | CACUGCUGGUAAGGAGACAGCACCUAGCUUGGGUAGUAAAAUUC |
| | | UCGCAGGGCUCACUACAGGCGGCGUCGCCGUAUUCAUUGGACAG |
| | | CCCACCGAAGUUGUGAAGGUACGACUGCAAGCUCAGUCCCAUCU |
| | | GCACGGGAUAAAACCUCGUUACACUGGCACUUACAAUGCUUACA |
| | | GGAUAAUAGCUACAACAGAAGGCUUAACGGGACUCUGGAAGGG |
| | | GACAACACCAAAUUUAAUGCGAUCUGUUAUCAUCAACUGCACUG |
| | | AACUCGUGACCUAUGAUCUGAUGAAGGAAGCCUUUGUGAAAAAC |
| | | AACAUCCUGGCCGACGACGUUCCCUGUCACUUAGUGUCUGCACU |
| | | CAUCGCCGGCUUCUGUGCCACCGCUAUGUCCUCCCCUGUGGACG |
| | | UUGUGAAGACACGCUUCAUAAAUUCCCCUCCUGGCCAAUACAAA |
| | | AGCGUGCCUAACUGUGCCAUGAAGGUUUUCACAAACGAGGGCCC |
| | | UACUGCUUUUUUCAAAGGCCUCGUUCCUUCCUUUCUUCGGCUCG |
| | | GGUCCUGGAAUGUCAUCAUGUUCGUGUGUUUUGAGCAGUUAAA |
| | | GAGAGAGCUUAGCAAAUCCAGGCAGACCAUGGACUGCGCCACCU |
| | | GA |
| 77 | mol_type = | AUGGGGGGACUGACCGCCAGCGACGUGCACCCUACUCUGGGAGU |
| | RNA | CCAGCUCUUCAGUGCCGGUAUCGCCGCCUGUCUUGCCGAUGUUA |
| | origin = | UAACAUUUCCCCUGGACACAGCUAAGGUCCGACUUCAAGUUCAA |
| | synthetic | GGCGAAUGUCCCACUUCUUCUGUCAUCCGGUACAAAGGCGUACU |
| | construct | GGGAACCAUCACAGCAGUGGUGAAAACCGAAGGCCGUAUGAAGC |
| | | UUUACAGCGGACUCCCAGCAGGACUGCAGAGGCAAAUAAGCUCG |
| | | GCAUCACUGCGGAUCGGCCUCUACGAUACCGUCCAGGAGUUUCU |
| | | GACCGCCGGAAAAGAAACAGCUCCUAGUUUGGGCAGUAAAAUAC |
| | | UCGCCGGGCUCACUACAGGUGGCGUUGCCGUUUUCAUAGGACAA |
| | | CCAACAGAAGUGGUCAAGGUCAGACUACAGGCUCAAAGUCACCU |
| | | UCACGGGAUUAAACCUCGCUAUACCGGAACUUAUAACGCUUAUA |
| | | GGAUCAUAGCCACGACAGAAGGCUUGACUGGGCUCUGGAAGGGG |
| | | ACAACACCUAACUUAAUGCGAUCCGUGAUCAUAAAUUGUACUGA |
| | | GCUCGUGACUUACGAUCUCAUGAAAGAGGCGUUCGUAAAAAAUA |
| | | ACAUAUUGGCAGAUGAUGUUCCCUGUCACUUAGUUUCCGCACUG |
| | | AUCGCCGGGUUUUGUGCAACAGCUAUGUCUUCUCCUGUCGACGU |
| | | UGUCAAAACACGGUUCAUAAAUUCUCCUCCGGGCCAAUAUAAAA |
| | | GUGUCCCUAACUGUGCCAUGAAAGUCUUCACGAAUGAGGGGCCU |
| | | ACAGCCUUCUUUAAGGGACUGGUCCCCUCGUUUCUUCGCCUCGG |
| | | UUCAUGGAAUGUUAUCAUGUUCGUGUGUUUUGAGCAGUUAAAG |
| | | CGGGAGCUGAGCAAGAGUAGACAAACAAUGGACUGCGCCACCUG |
| | | A |
| 78 | mol_type = | AUGGGCGGAUUGACCGCCAGCGAUGUGCAUCCCACGUUAGGUGU |
| | RNA | GCAGCUCUUUAGCGCAGGUAUUGCCGCGUGCUUAGCGGAUGUGA |
| | origin = | UUACGUUUCCCCUCGACACGGCUAAGGUACGGCUUCAAGUCCAG |
| | synthetic | GGCGAGUGCCCAACUUCUUCCGUAAUUCGCUACAAAGGCGUUCU |
| | construct | CGGAACCAUCACAGCCGUGGUGAAGACAGAAGGCCGCAUGAAAC |
| | | UUUACAGCGGACUGCCAGCCGGCUUGCAGAGGCAGAUAAGCUCG |
| | | GCCUCUCUCCGCAUCGGUUUGUACGACACGGUUCAGGAGUUCCU |
| | | UACUGCUGGAAAGGAGACAGCACCCAGUCUGGGGAGCAAAAUCC |
| | | UAGCCGGGCUUACAACAGGUGGUGUGGCUGUCUUCAUUGGACAG |
| | | CCCACUGAGGUUGUUAAGGUACGGCUGCAGGCUCAAAGCCACCU |
| | | CCACGGCAUCAAACCACGCUAUACUGGUACAUAUAACGCCUACA |
| | | GGAUCAUUGCGACUACAGAAGGACUCACAGGACUGUGGAAGGGG |
| | | ACGACUCCUAAUUUAAUGCGGUCUGUGAUCAUCAACUGCACCGA |
| | | ACUGGUCACUUACGAUCUAAUGAAAGAGGCUUUCGUGAAAAAU |
| | | AAUAUCUUGGCCGAUGACGUGCCUUGUCACCUCGUUUCUGCGCU |
| | | CAUCGCCGGAUUCUGUGCAACCGCCAUGUCUUCUCCGGUUGACG |
| | | UUGUCAAAACACGAUUUAUAAACUCACCACCAGGCCAAUACAAA |
| | | AGCGUCCCCAACUGUGCAAUGAAAGUCUUCACAAACGAAGGCCC |
| | | GACAGCGUUCUUUAAGGGGCUCGUCCCCUCCUUUCUUCGGCUCG |
| | | GUUCCUGGAAUGUUAUAAUGUUUGUGUGUUUUGAACAGCUAAA |
| | | AAGGGAGCUGUCAAAAUCCAGGCAAACAAUGGACUGUGCAACAU |
| | | GA |
| 79 | mol_type = | AUGGGGGGACUGACAGCCAGUGAUGUGCAUCCCACUCUGGGUGU |
| | RNA | UCAGCUUUUCAGCGCCGGGAUUGCCGCAUGUCUCGCUGAUGUAA |
| | origin = | UUACUUUUCCUCUCGACACAGCUAAAGUCCGCCUGCAGGUUCAG |
| | synthetic | GGAGAGUGUCCAACGUCCUCCGUGAUUCGGUAUAAAGGAGUUUU |
| | construct | AGGAACAAUCACAGCAGUGGUGAAGACCGAGGGCCGGAUGAAGC |
| | | UCUACAGCGGGCUCCCGGCAGGAUUGCAGAGGCAAAUCAGUUCU |
| | | GCCUCUCUCCGUAUCGGCCUGUACGAUACUGUACAAGAAUUCCU |
| | | CACCGCCGGAAAGGAGACAGCACCCAGUUUGGGGAGUAAAAUUC |
| | | UGGCAGGGCUGACCACAGGCGGCGUGGCUGUUUUCAUUGGACAG |
| | | CCUACCGAGGUGGUCAAGGUCCGAUUGCAGGCUCAAUCUCAUCU |
| | | UCACGGCAUAAAACCUAGAUAUACUGGGACAUACAACGCUUACA |
| | | GGAUCAUCGCAACCACAGAAGGCCUGACUGGACUAUGGAAAGGG |
| | | ACAACACCCAAUUUAAUGCGAUCAGUCAUUAUCAACUGCACUGA |
| | | AUUGGUAACAUACGAUCUCAUGAAGGAAGCUUUCGUGAAGAAU |
| | | AACAUCCUCGCAGAUGACGUUCCCUGUCACCUUGUGUCUGCACU |
| | | GAUCGCCGGAUUUUGUGCCACCGCGAUGUCUUCCCCGGUCGAUG |
| | | UUGUGAAGACACGAUUCAUAAAUUCUCCACCAGGCCAAUACAAA |
| | | AGCGUUCCCAACUGUGCCAUGAAAGUAUUUACAAACGAAGGCCC |
| | | GACAGCUUUCUUUAAGGGGCUCGUCCCCUCAUUUCUUCGACUCG |
| | | GUUCCUGGAAUGUAAUCAUGUUUGUGUGUUUCGAACAAUUAAA |
| | | AAGGGAGCUGAGUAAAAGUAGACAAACCAUGGACUGCGCCACCU |
| | | AG |
| 80 | mol_type = | AUGGGUGGUCUCACAGCCAGUGACGUUCAUCCCACUCUGGGGGU |
| | RNA | UCAGCUUUUCAGUGCAGGAAUCGCCGCCUGCCUCGCCGACGUGA |
| | origin = | UCACCUUUCCGUUGGACACUGCCAAAGUACGCCUACAGGUCCAG |
| | synthetic | GGCGAGUGUCCUACUUCUUCCGUGAUUCGGUAUAAGGGUGUGCU |
| | construct | AGGAACCAUCACUGCCGUGGUGAAAACAGAGGGCCGUAUGAAAC |
| | | UCUACAGCGGGUUGCCUGCCGGGCUACAGAGGCAGAUCAGCUCA |
| | | GCUUCUUUGAGAAUCGGUCUCUACGACACGGUCCAGGAAUUUCU |
| | | UACUGCCGGGAAGGAGACUGCACCUAGUCUGGGCAGCAAAAUUU |
| | | UAGCGGGGCUCACAACAGGCGGCGUGGCUGUAUUCAUUGGACAA |
| | | CCCACAGAGGUUGUGAAGGUCCGACUGCAGGCUCAAAGCCAUCU |
| | | GCACGGCAUUAAACCUCGCUACACUGGCACUUACAAUGCUUACC |
| | | GAAUCAUUGCUACAACUGAGGGCCUUACUGGACUCUGGAAAGGC |
| | | ACAACUCCCAAUUUAAUGCGAUCCGUAAUCAUCAAUUGCACUGA |
| | | ACUCGUGACUUACGACCUCAUGAAGGAAGCUUUUGUGAAAAAUA |
| | | ACAUCCUGGCAGAUGACGUCCCUUGUCACUUGGUUUCUGCUCUU |
| | | AUCGCAGGAUUCUGCGCAACCGCCAUGUCAUCUCCAGUGGACGU |
| | | UGUGAAGACAAGGUUCAUAAAUUCCCCUCCCGGUCAAUAUAAAA |
| | | GUGUCCCCAAUUGUGCCAUGAAGGUGUUCACAAACGAAGGCCCA |
| | | ACCGCCUUUUUUAAGGGGCUUGUUCCAUCUUUUCUGCGUCUCGG |
| | | AUCCUGGAACGUCAUCAUGUUCGUGUGUUUUGAACAAUUGAAA |
| | | AGGGAGUUGUCGAAGUCCAGGCAAACAAUGGAUUGUGCAACGU |
| | | AA |
| 81 | mol_type = | AUGGGCGGACUCACGGCUUCCGACGUCCAUCCCACCCUGGGGGU |
| | RNA | GCAGCUCUUCAGCGCAGGCAUUGCCGCGUGUCUCGCCGAUGUUA |
| | origin = | UUACUUUUCCACUUGACACCGCUAAGGUGCGAUUACAGGUCCAG |
| | synthetic | GGCGAAUGCCCAACAUCUUCCGUGAUCCGGUAUAAAGGUGUCCU |
| | construct | GGGAACCAUUACCGCGGUGGUGAAGACAGAAGGCCGCAUGAAAC |
| | | UGUACAGCGGGCUGCCCGCAGGGCUGCAGAGACAAAUAAGCUCC |
| | | GCAUCCCUCCGCAUCGGACUAUACGACACAGUUCAGGAAUUCCU |
| | | CACGGCCGGGAAGGAGACAGCUCCUAGUCUGGGAAGCAAGAUUC |
| | | UUGCUGGGCUCACUACAGGCGGCGUCGCUGUCUUCAUUGGACAA |
| | | CCUACUGAGGUGGUGAAGGUCCGACUGCAAGCCCAAUCCCACCU |
| | | UCAUGGAAUCAAGCCUCGCUACACUGGGACAUAUAACGCCUAUA |
| | | GAAUCAUUGCUACUACAGAAGGCUUGACUGGCCUUUGGAAAGGG |
| | | ACAACUCCCAAUUUAAUGCGAAGCGUGAUUAUCAAUUGCACUGA |
| | | ACUCGUGACUUACGAUCUCAUGAAGGAAGCAUUUGUGAAAAACA |
| | | ACAUCCUGGCUGAUGACGUUCCAUGUCAUCUGGUAUCUGCACUG |
| | | AUCGCCGGAUUUUGCGCUACUGCCAUGUCUAGCCCUGUAGAUGU |
| | | CGUCAAGACACGGUUCAUUAAUUCUCCUCCAGGCCAAUACAAAA |
| | | GUGUCCCCAAUUGUGCUAUGAAAGUGUUCACAAAUGAGGGCCCC |
| | | ACAGCCUUCUUUAAGGGGCUCGUCCCCUCUUUUCUGCGCCUCGG |
| | | UUCCUGGAAUGUCAUCAUGUUUGUGUGCUUCGAGCAAUUGAAGC |
| | | GAGAGUUGUCAAAGUCCCGGCAGACUAUGGAUUGUGCCACGUAA |
| 82 | mol_type = | AUGGGAGGCCUCACCGCUAGUGACGUGCACCCUACUCUGGGUGU |
| | RNA | ACAGCUCUUCAGUGCAGGAAUCGCCGCGUGCUUGGCCGAUGUUA |
| | origin = | UUACUUUUCCCCUGGACACAGCUAAGGUGCGCCUGCAGGUUCAG |
| | synthetic | GGGGAGUGUCCCACCUCCUCCGUCAUCAGAUACAAAGGCGUUUU |
| | construct | AGGAACCAUCACAGCCGUGGUAAAAACUGAGGGCCGCAUGAAGC |
| | | UCUACAGCGGUCUCCCGGCAGGACUUCAGAGGCAGAUCAGCUCU |
| | | GCAUCACUCCGUAUCGGGCUCUACGACACCGUUCAAGAGUUUCU |
| | | UACAGCAGGAAAGGAGACUGCACCCAGUUUGGGAAGCAAAAUUC |
| | | UGGCAGGUCUUACAACCGGCGGCGUGGCUGUGUUCAUCGGACAG |
| | | CCCACUGAGGUGGUCAAGGUGCGGCUCCAGGCUCAAUCUCACUU |
| | | GCACGGCAUCAAGCCUCGCUACACUGGCACGUACAAUGCUUACA |
| | | GAAUCAUUGCAACUACAGAAGGCCUGACAGGACUUUGGAAGGGA |
| | | ACAACUCCUAACCUUAUGCGAUCCGUUAUCAUUAACUGCACAGA |
| | | GCUCGUCACAUAUGACCUCAUGAAGGAAGCGUUCGUGAAAAAUA |
| | | ACAUCCUUGCAGAUGACGUUCCUUGCCACUUGGUGUCCGCGCUG |
| | | AUCGCCGGAUUCUGUGCUACCGCUAUGUCAAGCCCUGUCGACGU |
| | | CGUGAAGACAAGGUUCAUCAAUUCCCCUCCAGGACAAUAUAAGA |
| | | GUGUUCCUAACUGUGCAAUGAAAGUAUUCACAAACGAAGGCCCC |
| | | ACCGCCUUCUUCAAGGGACUGGUUCCUUCUUUUCUUCGACUCGG |
| | | UUCCUGGAACGUUAUCAUGUUCGUGUGUUUUGAGCAAUUAAAA |
| | | AGGGAGCUGUCAAAGUCCCGGCAGACAAUGGACUGUGCCACAUG |
| | | A |
| 83 | mol_type = | AUGGGUGGUUUAACCGCAAGCGACGUCCACCCCACCCUAGGGGU |
| | RNA | GCAGCUCUUCAGUGCAGGAAUCGCCGCAUGUCUCGCCGACGUCA |
| | origin = | UUACAUUUCCCUUAGACACCGCUAAGGUGCGCCUCCAGGUCCAG |
| | synthetic | GGCGAAUGUCCCACAUCUUCUGUAAUCCGCUAUAAAGGCGUACU |
| | construct | UGGAACAAUCACAGCUGUAGUGAAAACCGAGGGCCGGAUGAAAC |
| | | UGUACAGUGGGCUGCCCGCAGGACUGCAGAGGCAGAUCAGCUCC |
| | | GCAUCACUGCGAAUCGGGCUUUACGACACCGUGCAGGAAUUUCU |
| | | CACUGCCGGAAAAGAGACUGCUCCCAGUCUGGGUAGCAAAAUUC |
| | | UGGCCGGACUCACAACAGGCGGCGUAGCUGUCUUCAUUGGCCAG |
| | | CCCACCGAGGUGGUUAAGGUCCGGCUGCAAGCCCAAUCCCACCU |
| | | UCACGGUAUCAAGCCGCGUUACACUGGAACUUACAACGCUUACA |
| | | GAAUUAUCGCUACCACCGAAGGCUUAACGGGACUCUGGAAGGGG |
| | | ACAACUCCCAAUUUAAUGCGAUCCGUUAUCAUCAACUGCACCGA |
| | | ACUAGUGACCUAUGACCUCAUGAAGGAAGCCUUCGUGAAAAACA |
| | | ACAUCCUGGCAGACGACGUGCCCUGUCACCUGGUCUCUGCACUC |
| | | AUCGCUGGAUUCUGUGCUACAGCAAUGUCAUCUCCUGUCGACGU |
| | | UGUGAAGACACGAUUCAUCAACUCUCCACCUGGUCAGUAUAAAA |
| | | GUGUCCCUAACUGUGCCAUGAAGGUCUUCACAAAUGAAGGCCCU |
| | | ACCGCAUUCUUCAAGGGGCUCGUCCCCUCCUUUUUACGCCUCGG |
| | | CUCCUGGAACGUUAUUAUGUUUGUAUGUUUUGAGCAACUCAAA |
| | | AGGGAGCUGUCAAAAUCCAGACAGACUAUGGACUGCGCCACAUG |
| | | A |
| 84 | mol_type = | AUGGGUGGGCUGACCGCCAGCGAUGUUCAUCCGACUUUAGGUGU |
| | RNA | GCAACUCUUCAGUGCCGGCAUCGCCGCCUGCCUCGCUGAUGUGA |
| | origin = | UUACCUUUCCUCUUGAUACCGCUAAGGUACGGCUGCAGGUCCAA |
| | synthetic | GGCGAAUGCCCCACCUCUUCCGUCAUUCGGUACAAAGGCGUGUU |
| | construct | GGGAACAAUUACGGCAGUCGUGAAGACUGAAGGCCGAAUGAAAC |
| | | UCUACAGUGGGCUGCCUGCUGGUUUGCAGAGACAGAUAAGCUCA |
| | | GCGUCUCUGCGAAUUGGGCUCUAUGACACGGUUCAGGAGUUCCU |
| | | CACCGCUGGGAAGGAGACAGCUCCUAGUCUGGGAAGCAAAAUAC |
| | | UCGCCGGACUAACAACCGGUGGCGUUGCUGUAUUCAUCGGACAG |
| | | CCUACCGAAGUCGUUAAGGUCAGACUCCAGGCUCAGAGCCACCU |
| | | GCAUGGCAUUAAGCCUCGCUACACUGGUACCUACAACGCUUACA |
| | | GGAUUAUCGCUACCACAGAAGGCCUGACUGGACUGUGGAAAGGG |
| | | ACAACUCCUAAUUUAAUGCGCUCUGUUAUCAUCAACUGCACCGA |
| | | AUUGGUCACUUACGAUCUCAUGAAGGAAGCGUUUGUGAAAAAC |
| | | AACAUCUUGGCUGAUGACGUUCCUUGUCACCUAGUUUCGGCACU |
| | | GAUCGCUGGGUUUUGUGCCACCGCCAUGUCUUCUCCUGUCGACG |
| | | UUGUCAAAACAAGGUUUAUAAACUCACCACCAGGUCAGUAUAAA |
| | | AGUGUCCCUAACUGUGCCAUGAAAGUCUUCACCAACGAAGGGCC |
| | | CACAGCUUUCUUCAAGGGGCUUGUCCCUUCGUUUCUACGACUCG |
| | | GAUCUUGGAACGUGAUCAUGUUUGUCUGUUUCGAGCAAUUGAA |
| | | GCGAGAGCUGUCUAAGUCUAGGCAGACAAUGGACUGCGCAACCU |
| | | GA |
| 85 | mol_type = | AUGGGGGGGCUGACAGCUUCUGACGUACAUCCUACUCUGGGGGU |
| | RNA | CCAACUCUUCAGUGCCGGAAUCGCCGCUUGCCUCGCUGACGUUA |
| | origin = | UCACCUUCCCUUUAGACACAGCUAAAGUCCGACUGCAAGUUCAA |
| | synthetic | GGCGAGUGCCCUACUUCUUCCGUGAUCAGAUACAAGGGCGUGCU |
| | construct | GGGUACAAUUACUGCGGUGGUGAAAACUGAAGGCCGCAUGAAGC |
| | | UCUAUAGCGGGCUGCCUGCAGGCUUGCAGAGGCAGAUAAGCUCA |
| | | GCCUCCCUGCGGAUUGGUCUGUACGAUACUGUUCAGGAGUUCCU |
| | | UACCGCUGGAAAGGAGACAGCUCCCAGUUUAGGGAGCAAAAUCC |
| | | UGGCCGGACUCACUACAGGCGGCGUCGCUGUCUUCAUUGGACAG |
| | | CCCACAGAGGUUGUAAAGGUAAGGCUGCAAGCCCAAAGCCACCU |
| | | CCACGGCAUCAAACCUCGCUAUACUGGGACUUACAACGCUUACC |
| | | GAAUCAUAGCUACAACCGAAGGCUUAACUGGACUGUGGAAGGGC |
| | | ACAACACCAAACUUAAUGCGAUCAGUUAUUAUCAACUGCACCGA |
| | | ACUGGUUACUUAUGAUCUCAUGAAAGAAGCUUUUGUGAAAAAU |
| | | AACAUCCUCGCUGAUGACGUCCCUUGUCAUUUGGUGUCUGCAUU |
| | | AAUCGCCGGAUUUUGUGCGACCGCUAUGUCGAGUCCUGUCGACG |
| | | UUGUGAAAACACGAUUCAUAAAUUCACCUCCCGGGCAGUAUAAA |
| | | AGCGUUCCCAAUUGUGCCAUGAAGGUAUUCACCAACGAAGGUCC |
| | | UACGGCAUUCUUCAAGGGGCUGGUUCCUUCCUUUCUUCGGCUCG |
| | | GUUCCUGGAAUGUUAUCAUGUUUGUCUGUUUUGAACAGCUGAA |
| | | AAGGGAGCUAUCAAAGUCCAGGCAGACCAUGGACUGCGCUACUU |
| | | GA |
| 86 | mol_type = | AUGGGCGGACUCACAGCCUCGGACGUGCACCCCACACUGGGAGU |
| | RNA | UCAGCUUUUCAGCGCAGGCAUAGCCGCCUGCCUCGCAGAUGUGA |
| | origin = | UUACCUUUCCUCUAGACACUGCUAAGGUCCGCUUGCAAGUCCAG |
| | synthetic | GGCGAGUGCCCAACUUCUAGUGUGAUCCGAUAUAAGGGCGUGUU |
| | construct | GGGGACCAUUACGGCGGUCGUUAAAACAGAGGGCCGCAUGAAGC |
| | | UAUAUAGCGGGCUGCCAGCCGGCCUGCAGAGGCAGAUCAGCUCU |
| | | GCCUCCCUACGUAUCGGCCUGUAUGACACCGUUCAGGAGUUCCU |
| | | UACUGCAGGAAAAGAAACUGCUCCUAGCUUGGGCAGCAAAAUUC |
| | | UCGCCGGGCUCACUACAGGCGGCGUGGCUGUGUUCAUUGGACAG |
| | | CCUACUGAAGUGGUCAAGGUCCGACUGCAGGCUCAAAGCCACCU |
| | | CCAUGGCAUCAAACCUCGCUACACUGGCACAUAUAACGCUUACA |
| | | GAAUCAUAGCCACAACUGAAGGGCUCACAGGACUUUGGAAGGGG |
| | | ACAACACCUAACUUAAUGCGAUCUGUUAUCAUAAACUGCACCGA |
| | | ACUCGUAACGUAUGAUCUCAUGAAGGAGGCCUUCGUGAAAAACA |
| | | ACAUCCUGGCCGACGACGUUCCAUGUCAUUUGGUUUCCGCUCUC |
| | | AUCGCCGGGUUUUGUGCUACUGCUAUGUCAAGCCCUGUCGAUGU |
| | | UGUUAAGACACGAUUUAUAAACUCCCCACCUGGCCAGUAUAAGA |
| | | GCGUCCCCAACUGCGCCAUGAAGGUGUUCACAAACGAAGGGCCC |
| | | ACAGCGUUCUUUAAGGGGCUGGUCCCUUCUUUUCUACGGCUCGG |
| | | AUCCUGGAACGUUAUCAUGUUCGUGUGUUUCGAACAAUUAAAA |
| | | AGGGAGCUCUCCAAGUCACGGCAAACAAUGGACUGCGCUACAUA |
| | | G |
| 87 | mol_type = | AUGGGCGGUCUUACUGCUAGUGACGUGCACCCAACGCUGGGGGU |
| | RNA | GCAACUCUUCAGUGCUGGCAUCGCCGCUUGCCUUGCCGACGUUA |
| | origin = | UUACUUUUCCCUUAGAUACCGCUAAGGUGCGAUUGCAGGUCCAA |
| | synthetic | GGCGAAUGCCCUACAUCAUCAGUUAUUCGAUAUAAAGGUGUUCU |
| | construct | AGGUACCAUUACCGCCGUGGUGAAAACCGAGGGCCGCAUGAAGC |
| | | UUUACAGCGGGCUUCCUGCCGGCCUACAGCGCCAGAUAAGCUCA |
| | | GCAUCUCUACGGAUCGGACUGUACGACACCGUGCAGGAGUUCCU |
| | | UACAGCCGGAAAGGAAACAGCUCCUAGUCUGGGCAGCAAAAUCC |
| | | UCGCCGGGCUCACCACAGGCGGUGUCGCUGUAUUCAUUGGUCAA |
| | | CCCACUGAGGUCGUAAAGGUCCGGCUGCAGGCUCAAAGUCACCU |
| | | GCACGGUAUAAAACCGCGGUACACUGGUACUUACAACGCCUAUA |
| | | GGAUCAUCGCUACAACGGAGGGCUUAACUGGAUUGUGGAAGGG |
| | | GACGACUCCUAACUUAAUGCGCUCUGUCAUCAUAAAUUGCACCG |
| | | AACUGGUUACUUACGACCUCAUGAAGGAAGCCUUCGUGAAAAAC |
| | | AACAUCCUGGCAGAUGACGUUCCAUGCCACCUCGUGUCUGCACU |
| | | CAUCGCAGGAUUCUGUGCUACCGCUAUGUCUAGUCCAGUCGAUG |
| | | UUGUCAAGACACGGUUUAUCAAUUCACCACCAGGACAAUAUAAA |
| | | AGUGUCCCAAACUGUGCCAUGAAAGUCUUCACAAACGAGGGGCC |
| | | UACAGCAUUCUUCAAGGGGCUCGUCCCCUCUUUUCUGCGGCUCG |
| | | GGUCUUGGAACGUUAUCAUGUUUGUGUGUUUCGAACAGCUAAA |
| | | AAGGGAACUGUCCAAGUCAAGGCAAACAAUGGACUGCGCCACCU |
| | | AA |
| 88 | mol_type = | AUGGGGGGUCUGACCGCGAGCGACGUCCAUCCUACCCUGGGUGU |
| | RNA | UCAGCUCUUCAGUGCGGGCAUCGCCGCGUGCCUGGCCGACGUUA |
| | origin = | UUACCUUUCCCUUAGACACAGCAAAGGUACGACUACAGGUACAA |
| | synthetic | GGCGAAUGUCCUACUUCUUCCGUUAUUCGCUAUAAGGGCGUCUU |
| | construct | GGGAACCAUUACUGCUGUGGUAAAAACGGAAGGCCGCAUGAAGC |
| | | UGUAUAGCGGGCUGCCCGCGGGCCUUCAGAGGCAGAUCAGCUCG |
| | | GCCUCCCUGCGUAUUGGAUUGUACGACACAGUCCAAGAAUUCCU |
| | | UACCGCCGGGAAAGAAACAGCACCUAGCCUGGGCAGCAAAAUAC |
| | | UCGCCGGACUCACUACAGGCGGCGUGGCCGUGUUUAUUGGACAG |
| | | CCUACUGAGGUGGUCAAGGUCCGACUGCAGGCCCAGAGUCACUU |
| | | GCACGGCAUCAAACCACGGUAUACUGGCACGUACAACGCUUACA |
| | | GGAUCAUCGCAACCACCGAAGGCUUAACAGGACUCUGGAAGGGU |
| | | ACAACGCCUAAUUUAAUGCGCUCCGUCAUCAUAAAUUGCACCGA |
| | | GCUCGUGACUUAUGAUCUCAUGAAGGAAGCCUUUGUGAAGAAU |
| | | AACAUCCUGGCAGACGACGUUCCCUGUCACCUAGUAUCUGCACU |
| | | CAUCGCAGGAUUCUGCGCCACCGCUAUGUCUUCUCCCGUAGAUG |
| | | UCGUUAAGACGCGGUUUAUUAAUUCGCCACCCGGUCAAUAUAAA |
| | | AGUGUCCCUAAUUGUGCCAUGAAGGUCUUUACAAACGAAGGCCC |
| | | UACCGCCUUUUUUAAGGGGCUGGUUCCUUCUUUUUUACGGCUCG |
| | | GGUCGUGGAACGUCAUCAUGUUCGUGUGUUUUGAACAGCUGAA |
| | | ACGGGAACUUUCCAAAUCCAGGCAGACAAUGGACUGCGCUACUU |
| | | GA |
| 89 | mol_type = | AUGGGUGGUCUGACCGCAAGUGACGUGCAUCCUACACUGGGAGU |
| | RNA | GCAGCUCUUCAGUGCGGGCAUUGCCGCUUGCCUUGCCGAUGUAA |
| | origin = | UUACCUUUCCAUUAGACACAGCCAAGGUGCGCCUGCAGGUUCAG |
| | synthetic | GGAGAAUGUCCAACGUCUUCCGUGAUUCGGUAUAAGGGCGUUCU |
| | construct | GGGCACCAUUACGGCUGUGGUGAAAACAGAAGGCCGUAUGAAAC |
| | | UUUACAGCGGACUCCCUGCAGGGCUACAGCGACAGAUCAGCUCU |
| | | GCUUCUCUGCGUAUCGGUUUGUAUGACACAGUACAGGAGUUCCU |
| | | CACUGCCGGAAAGGAGACUGCACCCAGUUUAGGCAGCAAAAUCC |
| | | UUGCAGGGCUCACAACAGGUGGCGUUGCCGUUUUUAUCGGACAG |
| | | CCCACAGAAGUGGUGAAGGUACGACUGCAAGCUCAGAGUCACCU |
| | | GCACGGGAUCAAGCCUCGCUAUACUGGAACUUAUAACGCUUAUA |
| | | GAAUCAUUGCUACUACAGAAGGUCUAACUGGACUCUGGAAAGGG |
| | | ACAACACCUAAUUUAAUGCGAUCUGUUAUCAUCAACUGCACAGA |
| | | ACUGGUGACUUAUGACCUCAUGAAGGAAGCUUUUGUGAAAAAC |
| | | AACAUCCUCGCAGACGACGUUCCUUGUCACCUGGUGUCUGCACU |
| | | CAUUGCAGGGUUUUGUGCAACCGCCAUGUCUUCUCCUGUUGACG |
| | | UUGUCAAAACACGUUUUAUAAACUCACCUCCUGGACAGUACAAG |
| | | AGUGUCCCCAAUUGUGCCAUGAAGGUAUUCACAAACGAAGGGCC |
| | | CACCGCAUUCUUUAAGGGGCUGGUUCCCUCUUUUCUGCGCCUCG |
| | | GUUCUUGGAACGUGAUAAUGUUUGUGUGCUUUGAACAGUUAAA |
| | | AAGAGAACUAUCCAAAUCAAGGCAGACAAUGGACUGUGCCACAU |
| | | GA |
| 90 | mol_type = | AUGGGAGGACUGACAGCCAGCGACGUUCACCCUACACUGGGGGU |
| | RNA | UCAGCUCUUCAGCGCGGGAAUCGCUGCUUGCCUCGCUGAUGUCA |
| | origin = | UUACUUUUCCUCUUGAUACAGCUAAGGUCCGACUGCAAGUUCAG |
| | synthetic | GGCGAAUGCCCUACUUCUUCCGUCAUCCGCUACAAGGGCGUGCU |
| | construct | GGGCACCAUCACAGCUGUCGUAAAAACAGAGGGCCGCAUGAAAU |
| | | UAUAUAGCGGCUUGCCCGCAGGGCUGCAAAGGCAGAUCAGCUCU |
| | | GCGUCCCUCCGGAUCGGACUGUACGACACAGUGCAGGAAUUUCU |
| | | UACGGCCGGAAAGGAGACUGCUCCUAGCUUGGGUAGCAAAAUUC |
| | | UCGCCGGGCUGACCACAGGCGGUGUGGCUGUGUUCAUCGGGCAG |
| | | CCUACCGAGGUUGUUAAGGUGCGACUGCAGGCUCAAAGUCACCU |
| | | GCAUGGAAUCAAGCCUCGCUACACUGGUACAUAUAACGCUUAUC |
| | | GGAUUAUUGCAACCACUGAAGGCCUCACAGGCCUCUGGAAGGGC |
| | | ACCACUCCUAAUUUAAUGCGAUCAGUCAUCAUAAAUUGCACCGA |
| | | ACUCGUGACUUAUGAUCUCAUGAAGGAAGCUUUUGUAAAAAAC |
| | | AACAUUCUGGCUGACGACGUUCCCUGCCAUCUGGUGUCGGCUCU |
| | | CAUCGCAGGAUUUUGUGCAACCGCCAUGUCUUCUCCUGUCGACG |
| | | UUGUGAAAACAAGGUUUAUAAACUCACCACCUGGCCAAUAUAAG |
| | | UCUGUUCCCAAUUGUGCCAUGAAGGUGUUCACAAAUGAAGGUCC |
| | | CACAGCAUUCUUCAAAGGGCUUGUUCCCUCCUUUCUUCGGCUCG |
| | | GGUCCUGGAACGUCAUCAUGUUCGUGUGCUUUGAACAGUUAAAA |
| | | AGGGAACUGAGUAAGUCCAGGCAGACAAUGGACUGUGCUACAUG |
| | | A |
| 91 | mol_type = | AUGGGUGGCCUAACUGCCAGUGACGUUCAUCCCACUUUGGGAGU |
| | RNA | GCAGCUUUUCAGCGCAGGAAUUGCUGCUUGCCUUGCUGACGUUA |
| | origin = | UUACCUUUCCCUUAGAUACCGCGAAGGUCCGCUUGCAAGUCCAA |
| | synthetic | GGCGAGUGCCCAACUUCAUCCGUCAUUCGCUACAAAGGCGUUCU |
| | construct | GGGUACCAUCACAGCCGUCGUGAAGACCGAAGGCCGCAUGAAAC |
| | | UCUAUAGCGGGCUGCCCGCGGGCCUCCAGAGGCAAAUCAGCUCA |
| | | GCUUCCUUGCGCAUCGGAUUAUACGACACUGUUCAAGAGUUUCU |
| | | UACAGCCGGAAAGGAGACAGCGCCUAGUCUGGGGAGUAAAAUCU |
| | | UGGCCGGGCUCACAACAGGCGGCGUCGCCGUAUUCAUCGGACAG |
| | | CCCACCGAAGUAGUCAAGGUCCGACUGCAGGCUCAAUCUCAUCU |
| | | ACAUGGUAUCAAGCCCCGCUACACUGGCACAUAUAACGCUUAUA |
| | | GAAUUAUUGCUACAACAGAAGGCUUAACUGGACUAUGGAAGGG |
| | | CACUACACCUAAUUUGAUGCGGUCCGUCAUCAUCAAUUGCACCG |
| | | AGCUCGUAACUUACGAUCUAAUGAAGGAGGCCUUCGUGAAAAAC |
| | | AACAUUCUGGCCGAUGAUGUCCCUUGUCACUUGGUCUCUGCACU |
| | | GAUCGCCGGAUUCUGUGCCACCGCCAUGUCUAGCCCUGUCGACG |
| | | UCGUGAAAACGCGAUUCAUCAAUUCUCCCCCCGGUCAGUAUAAA |
| | | UCUGUUCCCAAUUGUGCUAUGAAGGUGUUCACAAACGAAGGCCC |
| | | AACUGCAUUCUUUAAAGGGCUAGUACCCUCUUUUCUGCGGCUUG |
| | | GCUCCUGGAAUGUCAUCAUGUUCGUGUGUUUCGAACAACUGAAA |
| | | AGGGAGCUGUCCAAAUCCAGGCAGACUAUGGACUGCGCCACCUG |
| | | A |
| 92 | mol_type = | AUGGGUGGACUCACAGCUUCAGACGUCCAUCCAACCCUGGGAGU |
| | RNA | GCAGCUCUUCAGUGCCGGAAUCGCCGCAUGCCUGGCAGACGUCA |
| | origin = | UUACCUUUCCCCUGGAUACAGCCAAGGUGCGACUACAGGUUCAG |
| | synthetic | GGGGAAUGCCCUACAUCCUCUGUGAUCCGGUAUAAGGGCGUGCU |
| | construct | GGGGACCAUUACAGCCGUGGUAAAGACCGAGGGCCGCAUGAAGC |
| | | UUUAUAGCGGGCUCCCCGCCGGCUUGCAGAGGCAGAUCAGCUCC |
| | | GCAUCACUGCGAAUCGGGUUGUACGACACAGUGCAGGAAUUUCU |
| | | UACUGCUGGAAAAGAGACUGCCCCUAGUCUGGGCAGCAAAAUCC |
| | | UGGCCGGGCUGACUACAGGUGGCGUUGCUGUCUUUAUUGGACAG |
| | | CCCACUGAGGUUGUAAAGGUGCGACUGCAAGCUCAAUCCCACCU |
| | | CCACGGAAUUAAGCCACGCUAUACUGGAACGUAUAACGCCUAUA |
| | | GGAUCAUUGCUACAACAGAAGGUUUGACUGGGCUUUGGAAGGG |
| | | UACAACACCCAAUUUAAUGCGAUCCGUCAUUAUCAACUGCACCG |
| | | AACUAGUGACAUACGAUCUCAUGAAGGAAGCAUUUGUGAAAAA |
| | | UAACAUCCUGGCUGACGACGUUCCUUGUCACUUAGUAUCUGCAC |
| | | UGAUCGCUGGGUUUUGUGCAACCGCAAUGUCUUCCCCCGUCGAU |
| | | GUCGUCAAAACAAGAUUUAUAAAUUCUCCACCUGGACAAUAUAA |
| | | AAGUGUUCCCAACUGCGCUAUGAAGGUCUUCACAAACGAAGGCC |
| | | CAACCGCAUUCUUUAAGGGGCUAGUUCCUUCUUUUCUUCGGCUU |
| | | GGUUCUUGGAACGUCAUCAUGUUCGUGUGUUUCGAACAGUUGA |
| | | AGCGGGAGCUGUCAAAAUCGAGGCAGACCAUGGACUGCGCUACG |
| | | UAA |
| 93 | mol_type = | AUGGGGGGACUGACUGCCUCGGACGUCCAUCCGACACUGGGUGU |
| | RNA | GCAGCUCUUCAGUGCAGGGAUCGCUGCUUGUCUCGCCGACGUUA |
| | origin = | UAACCUUUCCCCUAGACACGGCCAAGGUACGUCUACAGGUUCAG |
| | synthetic | GGCGAAUGCCCUACUUCUUCUGUCAUUCGUUACAAAGGUGUGUU |
| | construct | AGGGACCAUAACAGCGGUAGUGAAGACCGAAGGCCGCAUGAAAC |
| | | UCUACAGCGGGCUUCCUGCCGGACUGCAACGGCAGAUUAGCUCU |
| | | GCAUCCCUGCGCAUUGGGCUGUACGACACUGUGCAGGAAUUCCU |
| | | CACAGCAGGAAAGGAGACAGCGCCUAGUCUGGGGAGUAAAAUCC |
| | | UCGCAGGACUAACGACAGGUGGCGUCGCUGUGUUCAUUGGACAG |
| | | CCUACCGAGGUUGUGAAGGUAAGGCUGCAAGCCCAAUCACACCU |
| | | GCACGGAAUUAAGCCUCGCUACACUGGAACUUACAACGCCUACA |
| | | GGAUUAUUGCCACAACCGAGGGCCUUACUGGACUAUGGAAGGGG |
| | | ACAACACCUAAUCUUAUGCGAUCAGUUAUUAUCAACUGCACUGA |
| | | GUUGGUAACCUACGAUCUCAUGAAGGAAGCGUUCGUGAAAAACA |
| | | AUAUCCUGGCCGACGACGUGCCCUGCCAUUUGGUGUCUGCACUG |
| | | AUCGCAGGAUUUUGUGCUACCGCAAUGUCUUCUCCUGUCGAUGU |
| | | UGUGAAGACACGGUUUAUCAAUUCCCCACCGGGCCAGUAUAAAA |
| | | GUGUUCCUAACUGUGCCAUGAAAGUAUUCACAAACGAGGGCCCC |
| | | ACAGCCUUCUUCAAGGGGCUAGUUCCCUCAUUCCUUCGGCUCGG |
| | | GUCCUGGAAUGUCAUCAUGUUUGUGUGUUUUGAACAGUUAAAA |
| | | CGAGAGCUCUCGAAGUCCAGGCAGACAAUGGACUGUGCCACGUG |
| | | A |
| 94 | mol_type = | AUGGGCGGUCUCACGGCCUCAGACGUUCAUCCAACGCUGGGCGU |
| | RNA | GCAGCUCUUCAGUGCAGGAAUCGCCGCUUGCCUGGCUGAUGUCA |
| | origin = | UUACCUUUCCUCUUGACACUGCCAAGGUGCGGUUGCAGGUCCAG |
| | synthetic | GGUGAAUGCCCAACUUCUUCUGUUAUUCGCUACAAAGGCGUUUU |
| | construct | GGGAACCAUCACCGCUGUGGUGAAAACAGAGGGUCGCAUGAAGC |
| | | UGUACAGCGGGCUGCCAGCGGGACUACAGCGACAGAUAAGCUCA |
| | | GCAUCACUUCGGAUUGGUCUGUAUGACACAGUUCAGGAGUUCCU |
| | | CACUGCUGGAAAGGAGACAGCACCUAGUCUGGGUAGCAAAAUCC |
| | | UGGCUGGGCUAACAACAGGCGGCGUAGCUGUAUUUAUUGGACAA |
| | | CCCACUGAGGUGGUCAAGGUCCGACUGCAGGCCCAAUCUCACCU |
| | | GCAUGGCAUCAAGCCUCGCUACACUGGUACUUACAACGCUUACA |
| | | GGAUCAUAGCUACAACCGAAGGCCUAACUGGGCUGUGGAAGGGC |
| | | ACGACACCCAAUUUAAUGCGAUCCGUCAUCAUAAACUGCACCGA |
| | | ACUCGUUACAUAUGAUCUUAUGAAGGAAGCAUUUGUGAAAAAU |
| | | AACAUCCUGGCAGAUGACGUUCCUUGUCAUCUGGUUUCUGCACU |
| | | CAUCGCGGGAUUUUGUGCUACCGCUAUGUCAAGUCCCGUCGACG |
| | | UCGUGAAGACACGGUUCAUAAAUUCCCCACCCGGCCAGUACAAA |
| | | AGUGUCCCCAACUGUGCAAUGAAAGUCUUCACAAACGAAGGCCC |
| | | CACAGCAUUCUUCAAGGGACUAGUUCCCUCCUUUCUACGCCUCG |
| | | GCUCUUGGAACGUAAUCAUGUUCGUGUGUUUUGAACAAUUAAA |
| | | AAGGGAGCUGUCUAAAUCACGUCAAACUAUGGACUGCGCCACAU |
| | | GA |
| 95 | mol_type = | AUGGGAGGCCUAACAGCUUCUGAUGUCCACCCCACCCUUGGAGU |
| | RNA | GCAGCUCUUCAGUGCAGGUAUCGCUGCUUGCCUGGCCGAUGUUA |
| | origin = | UAACCUUUCCUCUGGAUACAGCCAAGGUGCGAUUGCAGGUCCAG |
| | synthetic | GGCGAGUGCCCGACCUCAAGCGUGAUCCGAUAUAAAGGCGUGUU |
| | construct | GGGAACUAUCACAGCCGUCGUGAAGACCGAAGGCCGAAUGAAGC |
| | | UUUACAGCGGACUUCCUGCCGGCCUGCAGAGGCAGAUAAGCUCU |
| | | GCAUCUCUCCGGAUCGGACUGUACGACACAGUCCAAGAGUUCCU |
| | | UACUGCAGGAAAGGAGACUGCACCUAGUUUAGGGAGCAAGAUCU |
| | | UGGCCGGACUCACAACAGGUGGCGUUGCUGUUUUUAUAGGACAG |
| | | CCUACUGAAGUGGUGAAGGUGAGACUGCAGGCUCAAAGCCAUCU |
| | | GCACGGCAUCAAACCUCGCUAUACUGGAACAUAUAAUGCUUAUA |
| | | GGAUCAUUGCGACCACAGAGGGCCUCACAGGGCUGUGGAAAGGG |
| | | ACAACCCCUAAUUUGAUGCGGUCAGUCAUCAUCAACUGCACUGA |
| | | GCUAGUUACUUAUGAUCUCAUGAAAGAAGCAUUUGUCAAAAAU |
| | | AACAUUCUGGCAGAUGAUGUGCCUUGUCACCUGGUGUCUGCAUU |
| | | AAUCGCAGGAUUCUGCGCCACCGCUAUGUCAUCUCCUGUCGAUG |
| | | UUGUGAAGACACGAUUCAUAAACUCCCCUCCUGGUCAAUAUAAG |
| | | AGCGUUCCCAACUGCGCUAUGAAAGUCUUCACCAACGAGGGCCC |
| | | AACAGCUUUCUUCAAGGGGCUCGUGCCCUCCUUUCUCCGACUGG |
| | | GAUCCUGGAAUGUGAUCAUGUUUGUCUGCUUUGAGCAGUUGAA |
| | | ACGGGAAUUGUCUAAAUCGAGGCAAACUAUGGACUGCGCCACAU |
| | | GA |
| 96 | mol_type = | AUGGGCGGCCUGACAGCCUCAGACGUUCAUCCCACAUUGGGUGU |
| | RNA | UCAGCUCUUCAGUGCAGGUAUUGCCGCCUGUCUCGCCGAUGUAA |
| | origin = | UAACCUUUCCACUCGACACCGCUAAGGUCCGCCUUCAGGUUCAG |
| | synthetic | GGCGAGUGCCCCACCAGUUCCGUUAUCAGAUACAAAGGUGUCCU |
| | construct | AGGGACCAUUACAGCGGUGGUGAAGACCGAGGGCCGUAUGAAGC |
| | | UCUAUAGCGGGCUGCCAGCAGGCCUCCAGCGCCAGAUAAGCUCU |
| | | GCCUCCCUGCGGAUCGGCCUCUAUGACACAGUCCAAGAAUUCCU |
| | | CACAGCUGGAAAGGAGACUGCGCCUAGUCUGGGUAGCAAGAUCC |
| | | UUGCCGGACUCACCACAGGCGGCGUUGCUGUAUUCAUUGGACAG |
| | | CCUACUGAAGUAGUCAAGGUUCGACUGCAGGCCCAAAGUCACCU |
| | | CCACGGCAUUAAGCCUCGCUACACUGGUACGUAUAACGCUUAUA |
| | | GAAUAAUAGCCACAACAGAAGGCUUAACGGGACUCUGGAAGGGG |
| | | ACUACACCUAAUUUAAUGCGAUCCGUCAUCAUCAACUGCACUGA |
| | | GCUUGUAACUUACGACCUCAUGAAGGAAGCCUUCGUGAAAAAUA |
| | | ACAUUUUGGCUGAUGACGUUCCGUGUCACCUGGUGUCUGCACUC |
| | | AUUGCAGGGUUCUGUGCCACCGCCAUGUCUUCUCCGGUCGACGU |
| | | UGUGAAGACGCGAUUCAUCAAUUCCCCUCCCGGUCAAUAUAAAU |
| | | CUGUCCCCAAUUGUGCCAUGAAAGUAUUCACUAACGAGGGCCCA |
| | | ACAGCCUUUUUCAAGGGGCUUGUUCCUUCCUUUUUGCGGCUCGG |
| | | AUCUUGGAACGUUAUCAUGUUCGUGUGUUUUGAACAAUUGAAA |
| | | CGGGAGUUGUCUAAGUCCCGCCAAACGAUGGACUGCGCUACCUG |
| | | A |
| 97 | mol_type = | AUGGGUGGGCUGACCGCAAGUGACGUGCACCCCACGUUAGGGGU |
| | RNA | GCAACUCUUCAGUGCCGGAAUUGCCGCCUGCCUCGCUGAUGUUA |
| | origin = | UUACUUUUCCUCUGGACACCGCGAAGGUACGGUUGCAAGUCCAA |
| | synthetic | GGCGAAUGUCCAACCUCUUCCGUCAUUCGGUAUAAAGGCGUCCU |
| | construct | UGGGACCAUCACUGCUGUCGUGAAAACGGAGGGCCGUAUGAAAC |
| | | UCUACAGCGGGCUUCCUGCAGGCCUGCAAAGGCAGAUCAGUUCA |
| | | GCUUCCCUGCGGAUCGGGCUGUAUGACACCGUGCAGGAAUUCCU |
| | | UACUGCAGGUAAGGAGACCGCACCCAGUUUGGGGUCCAAAAUCU |
| | | UGGCAGGACUCACAACAGGAGGCGUUGCUGUAUUCAUCGGACAG |
| | | CCCACCGAAGUGGUAAAGGUAAGACUGCAGGCCCAGUCUCACCU |
| | | GCACGGCAUCAAGCCUCGCUACACUGGAACCUACAACGCCUACA |
| | | GGAUCAUUGCCACCACUGAAGGUCUCACUGGAUUAUGGAAGGGG |
| | | ACCACUCCCAAUUUAAUGCGAUCAGUCAUCAUAAAUUGCACCGA |
| | | AUUGGUUACUUACGAUCUCAUGAAAGAAGCGUUUGUGAAAAAU |
| | | AAUAUCCUUGCAGACGACGUUCCUUGUCACCUGGUAUCUGCGCU |
| | | CAUCGCCGGAUUUUGCGCGACCGCCAUGUCUUCUCCUGUCGAUG |
| | | UUGUCAAAACACGGUUUAUCAACUCUCCUCCUGGUCAAUAUAAA |
| | | AGUGUUCCCAAUUGUGCCAUGAAGGUCUUCACCAACGAAGGCCC |
| | | UACAGCAUUCUUUAAGGGGCUUGUUCCCUCCUUUCUGCGCCUUG |
| | | GUUCUUGGAAUGUCAUCAUGUUUGUCUGUUUCGAACAACUAAA |
| | | AAGGGAGCUGAGCAAGAGUAGACAAACCAUGGACUGUGCAACAU |
| | | GA |
| 98 | mol_type = | AUGGGUGGCCUUACCGCUUCGGACGUUCAUCCCACCCUGGGCGU |
| | RNA | UCAGCUGUUUAGCGCCGGAAUAGCCGCAUGUCUCGCCGAUGUAA |
| | origin = | UCACCUUUCCUCUGGAUACCGCCAAGGUGCGCCUACAAGUCCAA |
| | synthetic | GGCGAAUGCCCGACCUCUUCUGUUAUUCGGUAUAAAGGUGUCUU |
| | construct | GGGAACCAUCACUGCAGUCGUAAAAACAGAAGGCCGUAUGAAAC |
| | | UCUACAGCGGACUUCCUGCUGGGCUCCAGAGGCAGAUAAGCUCU |
| | | GCCUCCUUGAGGAUUGGUCUGUACGACACCGUUCAGGAGUUCCU |
| | | UACAGCGGGAAAGGAGACAGCUCCCAGCUUAGGCAGCAAAAUAC |
| | | UUGCCGGGCUUACAACAGGUGGCGUGGCGGUGUUCAUUGGACAG |
| | | CCUACCGAGGUGGUAAAGGUGCGACUGCAGGCCCAGUCUCAUUU |
| | | GCAUGGGAUCAAGCCGCGCUACACUGGGACUUAUAACGCCUAUA |
| | | GAAUCAUUGCUACGACGGAGGGCUUAACUGGACUUUGGAAGGG |
| | | GACAACACCUAAUUUGAUGCGGUCGGUCAUAAUCAAUUGCACCG |
| | | AACUGGUGACUUAUGACCUCAUGAAAGAGGCUUUUGUGAAGAA |
| | | CAACAUCCUGGCUGACGACGUUCCUUGUCAUCUUGUAUCAGCAC |
| | | UUAUCGCAGGAUUCUGUGCAACAGCAAUGUCCAGCCCUGUCGAU |
| | | GUUGUCAAAACACGGUUCAUAAAUUCUCCACCUGGCCAAUAUAA |
| | | AAGCGUCCCCAAUUGUGCCAUGAAAGUAUUCACAAACGAAGGGC |
| | | CAACCGCCUUCUUCAAGGGACUCGUCCCCUCCUUUCUCCGUCUC |
| | | GGAUCCUGGAAUGUUAUCAUGUUCGUGUGCUUUGAGCAAUUAA |
| | | AGCGAGAGCUCUCCAAGUCAAGGCAAACAAUGGACUGCGCUACA |
| | | UAG |
| 99 | mol_type = | AUGGGAGGUCUCACCGCAUCUGAUGUGCAUCCAACACUGGGGGU |
| | RNA | UCAGCUCUUCAGUGCGGGAAUAGCCGCCUGUCUGGCUGAUGUUA |
| | origin = | UUACCUUUCCCCUGGACACCGCAAAGGUACGACUCCAAGUCCAG |
| | synthetic | GGCGAAUGUCCGACCUCAUCGGUGAUUAGAUACAAAGGCGUGUU |
| | construct | GGGGACCAUCACCGCAGUCGUGAAAACAGAAGGCAGGAUGAAAC |
| | | UUUACAGUGGGCUGCCCGCAGGCCUGCAGAGGCAGAUCAGCUCU |
| | | GCAUCUCUACGCAUCGGCUUGUACGAUACCGUCCAGGAAUUCCU |
| | | GACAGCAGGCAAAGAAACCGCCCCCAGUCUGGGUAGUAAGAUUU |
| | | UGGCAGGGCUGACUACAGGUGGUGUCGCUGUCUUCAUCGGGCAA |
| | | CCCACUGAAGUGGUGAAGGUGCGACUGCAAGCUCAGAGUCACCU |
| | | GCACGGAAUAAAACCUCGCUAUACUGGUACCUACAACGCUUAUA |
| | | GAAUUAUCGCGACCACCGAAGGCCUCACUGGACUCUGGAAAGGG |
| | | ACGACACCUAACUUGAUGCGAUCCGUAAUCAUCAAUUGCACUGA |
| | | GCUAGUUACAUACGAUCUCAUGAAGGAGGCAUUCGUGAAAAACA |
| | | AUAUCCUGGCCGAUGACGUCCCUUGUCACUUGGUUUCCGCGCUC |
| | | AUCGCAGGAUUCUGUGCUACCGCCAUGUCAUCCCCCGUCGAUGU |
| | | CGUUAAAACUCGGUUCAUUAACUCACCCCCUGGCCAGUACAAAA |
| | | GUGUUCCUAACUGUGCCAUGAAGGUGUUCACAAAUGAGGGCCCC |
| | | ACAGCAUUCUUCAAGGGACUAGUCCCUUCUUUUCUUCGGCUCGG |
| | | CAGCUGGAAUGUCAUCAUGUUCGUGUGCUUCGAACAGCUAAAAC |
| | | GAGAGCUGUCCAAGUCAAGGCAAACCAUGGACUGUGCCACAUAG |
| 100 | mol_type = | AUGGGGGGGCUCACCGCCUCCGAUGUGCACCCCACCCUUGGAGU |
| | RNA | UCAGCUCUUCAGUGCAGGAAUCGCCGCUUGCCUCGCCGAUGUUA |
| | origin = | UUACUUUUCCCUUAGACACAGCGAAGGUACGCCUGCAGGUUCAA |
| | synthetic | GGCGAAUGUCCAACUUCAAGCGUGAUUCGAUACAAAGGCGUGCU |
| | construct | CGGAACAAUCACAGCUGUGGUGAAGACAGAGGGCCGAAUGAAGC |
| | | UGUACUCUGGUCUCCCAGCAGGCCUCCAGAGGCAGAUCAGCUCC |
| | | GCAUCCUUGCGUAUCGGACUGUACGACACAGUCCAAGAAUUUCU |
| | | UACAGCUGGAAAGGAAACAGCUCCUAGUCUGGGAAGCAAAAUCC |
| | | UUGCAGGUCUUACCACAGGCGGCGUCGCCGUGUUCAUUGGACAG |
| | | CCUACUGAGGUUGUGAAGGUCCGACUGCAGGCUCAAUCCCAUCU |
| | | GCAUGGUAUUAAGCCACGCUACACUGGAACAUAUAACGCUUACA |
| | | GAAUCAUCGCAACCACAGAGGGCUUGACAGGACUCUGGAAAGGC |
| | | ACGACGCCCAAUUUAAUGCGAUCCGUCAUCAUCAACUGCACAGA |
| | | ACUGGUAACGUAUGAUCUCAUGAAAGAAGCUUUUGUGAAGAAU |
| | | AACAUCUUGGCAGACGACGUUCCAUGUCACCUGGUUUCUGCAUU |
| | | GAUCGCCGGAUUCUGUGCUACUGCUAUGUCAUCGCCUGUUGACG |
| | | UCGUAAAGACACGCUUUAUAAACUCCCCACCUGGUCAAUAUAAA |
| | | AGUGUCCCCAACUGUGCAAUGAAAGUCUUCACAAACGAAGGCCC |
| | | UACAGCAUUUUUUAAGGGACUGGUUCCCUCCUUUCUUCGUCUCG |
| | | GAUCCUGGAAUGUCAUUAUGUUUGUAUGCUUUGAACAGCUGAA |
| | | AAGAGAGCUGUCCAAAUCCAGACAAACAAUGGACUGUGCAACCU |
| | | GA |
| 101 | mol_type = | AUGGGCGGUCUGACAGCCAGCGACGUGCAUCCCACACUGGGUGU |
| | RNA | GCAGCUCUUCAGUGCAGGAAUUGCCGCUUGCCUCGCUGAUGUUA |
| | origin = | UUACAUUUCCUCUAGAUACUGCCAAGGUACGAUUGCAGGUUCAA |
| | synthetic | GGCGAAUGCCCAACAAGUAGCGUUAUCCGAUAUAAAGGCGUGUU |
| | construct | GGGAACCAUCACCGCUGUAGUGAAAACCGAAGGCAGGAUGAAGC |
| | | UCUACAGCGGGCUCCCAGCCGGCCUACAAAGGCAGAUAAGCUCC |
| | | GCCUCCCUGCGUAUCGGAUUGUACGACACGGUCCAAGAGUUCCU |
| | | UACAGCGGGAAAAGAGACAGCGCCUAGCUUGGGGUCCAAAAUUC |
| | | UGGCCGGACUCACAACAGGAGGUGUGGCUGUGUUCAUUGGACAG |
| | | CCUACAGAAGUUGUCAAGGUGCGACUACAGGCUCAAUCUCACCU |
| | | GCAUGGGAUAAAACCUCGCUACACUGGGACUUACAACGCUUACA |
| | | GGAUUAUUGCCACAACAGAAGGCCUGACGGGACUUUGGAAAGGU |
| | | ACAACACCUAAUUUAAUGCGUUCCGUUAUCAUCAAUUGCACUGA |
| | | GCUGGUCACUUACGAUCUCAUGAAGGAAGCAUUCGUGAAAAAUA |
| | | ACAUCUUGGCAGAUGACGUUCCCUGUCACUUGGUGUCCGCACUG |
| | | AUCGCAGGAUUCUGUGCCACUGCAAUGUCAAGCCCUGUCGAUGU |
| | | CGUGAAAACAAGGUUUAUCAAUUCCCCACCUGGUCAGUAUAAAU |
| | | CUGUUCCUAAUUGUGCCAUGAAGGUAUUCACCAACGAAGGCCCC |
| | | ACAGCCUUCUUUAAGGGACUGGUUCCCUCUUUUCUCCGGCUUGG |
| | | AUCCUGGAACGUCAUAAUGUUCGUAUGUUUUGAGCAGUUAAAA |
| | | AGAGAGCUGUCUAAAUCCAGGCAGACAAUGGACUGUGCUACAUA |
| | | G |
| 102 | mol_type = | AUGGGCGGACUUACAGCAUCUGACGUGCACCCUACAUUAGGUGU |
| | RNA | UCAGCUCUUCAGUGCAGGCAUCGCUGCUUGCCUCGCCGAUGUCA |
| | origin = | UUACCUUUCCUCUGGAUACCGCUAAGGUACGCCUCCAGGUUCAG |
| | synthetic | GGCGAAUGCCCAACUUCCUCCGUGAUUCGAUAUAAGGGAGUUUU |
| | construct | GGGAACCAUUACGGCUGUGGUGAAGACUGAGGGCCGAAUGAAAC |
| | | UUUACAGCGGGCUGCCUGCGGGACUGCAGCGGCAGAUCAGCUCA |
| | | GCUUCGUUGCGGAUCGGGCUAUAUGACACCGUUCAGGAAUUCCU |
| | | UACGGCCGGAAAGGAAACCGCACCUAGUCUGGGUAGUAAAAUUC |
| | | UUGCCGGGCUUACGACAGGCGGCGUCGCUGUGUUCAUUGGCCAA |
| | | CCCACUGAGGUGGUAAAGGUCCGGCUUCAGGCUCAAAGCCACCU |
| | | GCACGGAAUCAAGCCUCGCUACACUGGAACAUACAACGCUUACA |
| | | GGAUCAUAGCCACUACAGAAGGCCUGACAGGGCUUUGGAAGGGC |
| | | ACGACACCCAAUUUAAUGCGGUCUGUGAUCAUCAAUUGCACUGA |
| | | ACUGGUGACUUACGACCUCAUGAAGGAAGCUUUCGUGAAAAACA |
| | | ACAUACUGGCCGAUGACGUCCCAUGUCAUCUGGUGUCCGCAUUG |
| | | AUCGCCGGAUUUUGUGCUACAGCUAUGUCUUCCCCUGUCGACGU |
| | | CGUUAAGACACGAUUUAUAAACUCUCCACCAGGACAAUACAAGA |
| | | GUGUCCCCAAUUGCGCCAUGAAAGUCUUCACAAACGAAGGCCCU |
| | | ACAGCCUUCUUUAAGGGGCUCGUUCCCUCCUUUCUUCGGCUCGG |
| | | CUCUUGGAACGUCAUCAUGUUCGUGUGCUUCGAGCAACUGAAAC |
| | | GGGAGUUAUCCAAAAGUAGACAGACCAUGGACUGCGCUACUUGA |
| 103 | mol_type = | AUGGGCGGAUUAACUGCCAGUGACGUCCACCCCACUCUGGGGGU |
| | RNA | ACAACUCUUCAGUGCCGGAAUCGCCGCUUGUCUCGCCGAUGUUA |
| | origin = | UUACCUUUCCCUUAGACACCGCUAAGGUGCGCCUGCAAGUUCAA |
| | synthetic | GGCGAAUGCCCAACAUCAAGCGUUAUACGAUAUAAGGGCGUUCU |
| | construct | CGGGACCAUCACAGCUGUUGUAAAAACAGAAGGCCGUAUGAAAC |
| | | UCUACAGCGGACUGCCUGCCGGCCUGCAGAGGCAGAUCAGUUCG |
| | | GCGUCCCUGCGCAUCGGUUUGUACGAUACCGUGCAAGAAUUCCU |
| | | GACCGCCGGAAAAGAGACAGCUCCCAGUUUGGGUAGUAAAAUUC |
| | | UGGCGGGGCUCACAACAGGUGGCGUUGCUGUGUUCAUCGGACAG |
| | | CCUACAGAGGUAGUCAAGGUCCGACUCCAGGCUCAAUCACACCU |
| | | GCACGGAAUCAAACCUCGCUACACUGGGACAUACAACGCUUACA |
| | | GGAUUAUCGCCACCACAGAAGGCUUGACUGGGUUGUGGAAGGGC |
| | | ACAACACCUAAUUUAAUGCGAUCUGUCAUAAUCAAUUGCACGGA |
| | | ACUAGUAACGUACGAUCUCAUGAAAGAAGCUUUUGUGAAAAAU |
| | | AACAUUCUGGCAGAUGACGUCCCCUGUCACUUGGUGUCUGCACU |
| | | CAUAGCUGGAUUUUGUGCAACUGCUAUGUCAUCCCCCGUCGAUG |
| | | UCGUGAAAACACGGUUUAUCAAUUCACCACCAGGCCAAUACAAA |
| | | UCUGUACCUAAUUGUGCCAUGAAGGUGUUCACGAACGAAGGGCC |
| | | AACAGCCUUCUUCAAGGGGCUUGUUCCAUCCUUUCUCCGACUCG |
| | | GGUCAUGGAAUGUGAUCAUGUUCGUGUGUUUCGAGCAACUGAA |
| | | AAGAGAGUUGUCAAAGAGUAGACAAACGAUGGACUGCGCCACAU |
| | | GA |
| 104 | mol_type = | AUGGGUGGAUUGACUGCCUCCGAUGUCCAUCCUACAUUGGGGGU |
| | RNA | UCAGCUCUUCAGUGCGGGCAUCGCCGCUUGCCUUGCCGAUGUCA |
| | origin = | UUACCUUUCCACUAGACACAGCUAAGGUACGUCUACAAGUCCAA |
| | synthetic | GGAGAAUGCCCCACCUCUUCUGUGAUUAGAUACAAGGGCGUACU |
| | construct | GGGAACCAUCACAGCUGUCGUGAAGACCGAGGGACGCAUGAAGC |
| | | UUUACAGCGGGCUGCCAGCAGGACUGCAGAGGCAGAUAAGCUCA |
| | | GCAUCCCUGCGAAUCGGACUAUACGACACAGUCCAGGAAUUUCU |
| | | UACUGCGGGAAAAGAGACAGCUCCCAGUUUAGGGAGUAAAAUAC |
| | | UCGCCGGGCUGACAACAGGCGGCGUUGCCGUUUUCAUAGGACAG |
| | | CCGACUGAAGUGGUUAAGGUGCGACUCCAAGCUCAGUCUCACCU |
| | | GCAUGGUAUCAAACCUCGCUAUACUGGUACAUAUAACGCCUACA |
| | | GGAUUAUAGCAACAACAGAAGGUUUGACUGGACUCUGGAAGGG |
| | | GACAACACCCAAUUUGAUGCGAUCCGUCAUUAUUAACUGCACCG |
| | | AGCUCGUAACCUAUGAUCUCAUGAAGGAGGCCUUUGUAAAAAAC |
| | | AACAUCCUGGCAGACGACGUUCCUUGCCACUUGGUUUCAGCACU |
| | | CAUCGCCGGGUUCUGUGCUACCGCUAUGUCUAGCCCUGUGGAUG |
| | | UUGUCAAAACACGAUUCAUCAAUUCACCACCGGGCCAAUAUAAA |
| | | UCUGUCCCCAAUUGCGCCAUGAAGGUCUUCACAAAUGAAGGCCC |
| | | GACUGCCUUCUUCAAGGGGCUCGUUCCUUCUUUUCUCCGGCUCG |
| | | GGUCCUGGAAUGUGAUAAUGUUCGUGUGUUUCGAACAGCUGAA |
| | | AAGGGAGCUGUCAAAAUCAAGGCAAACCAUGGACUGCGCCACCU |
| | | GA |
| 105 | mol_type = | AUGGGAGGUCUGACAGCUUCAGACGUACACCCCACAUUAGGCGU |
| | RNA | GCAACUCUUCAGUGCCGGCAUAGCCGCUUGCCUCGCCGAUGUUA |
| | origin = | UUACCUUUCCUCUGGACACCGCUAAGGUGCGACUUCAGGUUCAG |
| | synthetic | GGCGAGUGUCCAACCUCAUCCGUUAUUCGAUACAAAGGCGUGCU |
| | construct | AGGAACCAUAACCGCCGUGGUCAAGACGGAAGGCCGUAUGAAAC |
| | | UUUACAGCGGACUCCCAGCGGGACUCCAGAGACAAAUAAGCUCU |
| | | GCAUCUCUUCGAAUCGGACUGUACGAUACCGUCCAGGAGUUUCU |
| | | AACAGCUGGAAAGGAGACAGCACCUAGUUUGGGCAGCAAAAUUC |
| | | UUGCCGGGCUCACUACAGGAGGCGUCGCCGUGUUUAUUGGACAG |
| | | CCCACAGAGGUAGUGAAGGUGCGACUGCAGGCCCAAUCUCAUCU |
| | | GCACGGAAUUAAACCGCGCUACACCGGUACAUAUAACGCCUACA |
| | | GAAUCAUCGCUACCACAGAAGGUCUGACUGGGUUGUGGAAGGGG |
| | | ACAACCCCCAAUUUAAUGCGAUCUGUGAUCAUCAACUGCACUGA |
| | | GCUGGUCACUUACGAUCUCAUGAAAGAAGCAUUCGUGAAAAACA |
| | | ACAUUUUGGCUGACGACGUCCCAUGUCAUUUAGUUUCUGCGCUC |
| | | AUCGCUGGGUUUUGUGCUACAGCAAUGUCUUCUCCCGUUGAUGU |
| | | CGUCAAAACACGAUUCAUAAACUCUCCACCUGGCCAAUACAAAA |
| | | GUGUUCCUAACUGUGCCAUGAAAGUCUUCACGAACGAAGGCCCC |
| | | ACAGCCUUCUUCAAGGGGCUGGUUCCCUCCUUUCUUCGGCUCGG |
| | | AUCCUGGAAUGUAAUCAUGUUUGUGUGCUUCGAACAGUUAAAA |
| | | AGAGAGCUCUCAAAAUCAAGGCAGACAAUGGAUUGUGCCACUUG |
| | | A |
| 106 | mol_type = | AUGGGUGGACUGACAGCUAGCGAUGUGCACCCCACACUGGGCGU |
| | RNA | GCAACUCUUCAGUGCUGGUAUUGCCGCUUGCCUCGCCGACGUCA |
| | origin = | UAACGUUUCCCUUAGACACUGCUAAAGUCCGGCUUCAGGUCCAG |
| | synthetic | GGUGAAUGCCCAACCUCUUCAGUCAUCCGUUAUAAAGGCGUGCU |
| | construct | GGGAACCAUCACAGCGGUGGUAAAAACAGAGGGCCGCAUGAAGC |
| | | UGUAUAGCGGGCUCCCCGCAGGCCUCCAGAGGCAGAUAAGCUCU |
| | | GCUUCUCUGCGGAUCGGUUUGUAUGACACUGUUCAAGAAUUCCU |
| | | UACCGCCGGCAAAGAAACCGCACCUAGUCUGGGUAGUAAAAUCC |
| | | UCGCCGGGCUCACUACAGGCGGUGUCGCCGUAUUCAUCGGACAA |
| | | CCUACCGAGGUGGUCAAGGUAAGACUGCAGGCCCAAAGCCACCU |
| | | GCACGGCAUCAAACCUCGCUACACCGGCACUUACAACGCCUACA |
| | | GGAUUAUAGCUACCACCGAAGGCUUGACGGGACUGUGGAAAGGC |
| | | ACAACCCCAAAUUUAAUGCGGUCCGUUAUUAUCAACUGCACCGA |
| | | GCUGGUAACUUAUGAUCUCAUGAAGGAAGCAUUUGUGAAAAAU |
| | | AACAUCUUGGCAGACGACGUUCCAUGUCACUUGGUUUCGGCGCU |
| | | AAUCGCCGGAUUUUGUGCUACAGCCAUGUCAUCCCCUGUCGAUG |
| | | UCGUGAAGACAAGGUUCAUCAAUUCACCACCAGGUCAGUACAAA |
| | | AGUGUCCCCAAUUGCGCAAUGAAAGUAUUCACAAACGAAGGCCC |
| | | AACCGCGUUUUUUAAGGGGCUUGUUCCCUCCUUUCUGCGCCUCG |
| | | GUUCUUGGAAUGUCAUCAUGUUUGUAUGCUUCGAGCAGCUGAA |
| | | ACGGGAGCUGUCCAAGUCCAGACAGACUAUGGACUGUGCGACAU |
| | | GA |
| 107 | mol_type = | AUGGGGGGGCUGACAGCUUCAGACGUUCACCCCACCCUGGGAGU |
| | RNA | UCAACUCUUUAGUGCUGGAAUCGCCGCCUGCCUCGCAGAUGUCA |
| | origin = | UCACCUUUCCUCUGGAUACAGCAAAGGUGCGGCUUCAGGUUCAG |
| | synthetic | GGCGAAUGUCCAACUUCUUCAGUGAUCCGCUAUAAAGGCGUCUU |
| | construct | GGGAACCAUCACCGCAGUCGUGAAGACCGAAGGCCGAAUGAAGC |
| | | UUUACAGCGGGCUGCCAGCCGGGCUGCAACGACAGAUAAGCUCA |
| | | GCAUCCCUGCGUAUAGGCCUAUACGACACCGUCCAAGAGUUUCU |
| | | CACUGCCGGAAAGGAGACAGCUCCCAGUCUGGGAAGCAAGAUCU |
| | | UAGCCGGACUCACCACAGGCGGCGUGGCCGUGUUCAUUGGACAA |
| | | CCUACCGAGGUUGUUAAGGUGCGACUACAGGCUCAGAGCCAUCU |
| | | GCAUGGGAUUAAACCACGCUAUACUGGAACUUACAACGCUUAUA |
| | | GGAUUAUCGCCACCACUGAAGGAUUAACUGGACUUUGGAAGGGC |
| | | ACAACACCCAACUUGAUGCGAUCAGUGAUCAUAAAUUGCACCGA |
| | | ACUGGUGACCUACGAUCUCAUGAAGGAGGCUUUCGUGAAGAACA |
| | | ACAUACUUGCCGACGACGUUCCUUGUCACCUCGUCUCUGCGUUG |
| | | AUCGCCGGGUUCUGCGCUACUGCUAUGUCAUCUCCCGUCGAUGU |
| | | UGUGAAAACAAGGUUUAUCAAUUCGCCUCCAGGCCAGUAUAAAA |
| | | GUGUCCCUAACUGUGCCAUGAAAGUGUUCACAAAUGAAGGCCCC |
| | | ACAGCAUUUUUCAAGGGACUCGUUCCUUCCUUUCUUCGUCUCGG |
| | | AUCCUGGAAUGUCAUCAUGUUUGUGUGCUUCGAGCAAUUGAAAC |
| | | GAGAGCUGUCGAAGUCACGACAAACCAUGGACUGCGCAACCUGA |
| 108 | mol_type = | AUGGGUGGUCUCACUGCAAGCGACGUGCAUCCAACACUGGGGGU |
| | RNA | UCAGCUCUUCAGCGCCGGAAUCGCCGCUUGCCUUGCCGACGUCA |
| | origin = | UAACUUUUCCUUUAGAUACCGCUAAGGUACGCCUGCAAGUCCAG |
| | synthetic | GGCGAGUGCCCCACAUCAAGUGUGAUUCGCUACAAAGGCGUUUU |
| | construct | GGGGACCAUCACAGCAGUGGUGAAGACGGAAGGCCGAAUGAAAC |
| | | UUUAUAGCGGACUGCCAGCAGGACUGCAGCGCCAAAUAAGCUCU |
| | | GCUUCACUGCGUAUCGGUCUAUAUGAUACCGUGCAGGAAUUUCU |
| | | UACUGCAGGAAAGGAGACGGCACCCAGUUUGGGGAGCAAAAUCU |
| | | UGGCCGGCCUGACUACAGGCGGUGUCGCUGUAUUCAUUGGACAG |
| | | CCAACUGAAGUGGUGAAGGUGCGACUCCAGGCUCAAUCUCACCU |
| | | GCACGGUAUCAAACCCCGCUACACUGGCACAUAUAACGCUUACA |
| | | GGAUUAUCGCUACGACAGAAGGCUUAACGGGCCUCUGGAAAGGC |
| | | ACCACCCCUAAUUUAAUGCGAUCUGUCAUCAUCAAUUGCACUGA |
| | | ACUCGUAACGUAUGAUCUCAUGAAAGAAGCGUUUGUGAAAAAU |
| | | AAUAUCCUGGCGGACGACGUCCCUUGUCACCUGGUGUCUGCACU |
| | | UAUCGCCGGAUUUUGUGCCACCGCCAUGUCCAGUCCUGUUGACG |
| | | UUGUCAAAACAAGGUUCAUCAAUUCACCUCCUGGUCAAUAUAAA |
| | | AGUGUUCCCAACUGUGCCAUGAAAGUCUUCACAAAUGAAGGUCC |
| | | CACAGCUUUUUUUAAAGGACUAGUUCCUUCCUUCCUGCGGCUUG |
| | | GAUCCUGGAAUGUCAUCAUGUUUGUGUGUUUUGAACAGCUGAA |
| | | ACGGGAACUUAGUAAAUCCAGACAGACCAUGGACUGUGCCACCU |
| | | GA |
| 109 | mol_type = | AUGGGUGGUCUAACCGCUUCAGACGUGCAUCCUACUCUGGGAGU |
| | RNA | GCAGCUCUUCAGUGCAGGUAUCGCCGCUUGCCUUGCCGACGUGA |
| | origin = | UCACCUUUCCCCUGGACACCGCUAAGGUGCGCCUGCAGGUCCAG |
| | synthetic | GGCGAAUGUCCUACAUCAAGCGUGAUUCGGUAUAAAGGCGUAUU |
| | construct | GGGAACCAUCACUGCUGUGGUAAAGACUGAAGGAAGAAUGAAG |
| | | CUAUACAGCGGGCUUCCAGCUGGACUUCAGAGGCAGAUCAGCUC |
| | | CGCUUCUUUGCGUAUCGGCCUGUACGACACCGUUCAGGAAUUUC |
| | | UCACUGCAGGAAAGGAGACAGCACCCAGCCUGGGCAGCAAAAUU |
| | | CUAGCCGGGCUCACCACAGGUGGCGUUGCUGUAUUUAUAGGGCA |
| | | ACCAACAGAGGUGGUCAAGGUGAGGCUGCAAGCUCAAUCCCACU |
| | | UACACGGUAUUAAGCCACGCUAUACUGGAACUUACAACGCCUAC |
| | | AGGAUUAUUGCUACAACAGAAGGUCUUACUGGACUUUGGAAGG |
| | | GGACAACACCUAAUUUAAUGCGGUCCGUGAUCAUUAACUGCACC |
| | | GAAUUGGUUACCUACGAUCUCAUGAAAGAAGCCUUUGUGAAGA |
| | | AUAACAUCUUGGCCGAUGACGUUCCUUGUCACCUCGUGUCUGCA |
| | | CUAAUAGCCGGAUUCUGUGCUACAGCUAUGUCAUCUCCUGUCGA |
| | | UGUUGUCAAAACGAGGUUUAUCAAUUCUCCUCCGGGUCAGUAUA |
| | | AAAGUGUCCCUAAUUGUGCCAUGAAAGUAUUCACGAACGAAGGA |
| | | CCCACCGCCUUCUUCAAGGGACUUGUUCCUUCUUUUCUCCGUCU |
| | | CGGUUCCUGGAAUGUCAUCAUGUUCGUGUGUUUUGAACAGCUGA |
| | | AAAGGGAACUUAGCAAAUCAAGACAAACUAUGGACUGUGCUACG |
| | | UGA |
| 110 | mol_type = | AUGGGGGGUCUGACUGCCUCAGACGUUCAUCCGACACUGGGGGU |
| | RNA | GCAGCUCUUCAGUGCCGGCAUUGCCGCUUGCCUCGCCGAUGUCA |
| | origin = | UUACUUUUCCUCUGGACACCGCGAAGGUCCGCCUGCAAGUUCAG |
| | synthetic | GGCGAGUGUCCUACAUCUUCCGUCAUCCGGUAUAAAGGCGUUCU |
| | construct | GGGAACCAUCACAGCCGUCGUGAAGACCGAGGGCCGAAUGAAGC |
| | | UUUACAGCGGGCUGCCUGCUGGGCUGCAGCGACAGAUAAGCUCC |
| | | GCUUCUCUUCGGAUCGGGCUGUACGACACAGUACAGGAGUUUCU |
| | | CACAGCUGGAAAGGAGACUGCACCCAGUUUGGGUAGUAAAAUUC |
| | | UCGCGGGGCUCACCACUGGCGGUGUCGCUGUGUUUAUUGGUCAG |
| | | CCUACUGAGGUCGUCAAGGUCAGGCUGCAGGCUCAGAGCCAUCU |
| | | GCAUGGGAUCAAACCUCGCUACACUGGCACAUAUAAUGCCUACA |
| | | GGAUAAUUGCAACAACAGAAGGCCUGACUGGACUGUGGAAAGG |
| | | GACAACACCCAAUUUAAUGCGAUCCGUCAUCAUUAAUUGCACCG |
| | | AGCUCGUAACAUACGAUCUCAUGAAGGAGGCAUUUGUGAAAAA |
| | | UAACAUUUUAGCAGAUGAUGUUCCAUGUCACCUGGUAUCAGCAC |
| | | UCAUCGCAGGAUUCUGUGCUACUGCUAUGUCUUCUCCAGUGGAC |
| | | GUGGUGAAGACUCGGUUUAUCAAUUCUCCACCUGGCCAAUACAA |
| | | AAGUGUCCCUAACUGUGCCAUGAAGGUAUUCACUAACGAAGGCC |
| | | CGACUGCCUUCUUUAAGGGACUCGUCCCUUCUUUUCUGCGGCUU |
| | | GGCUCGUGGAACGUGAUUAUGUUUGUGUGUUUUGAACAGCUGA |
| | | AAAGGGAGCUAAGUAAGAGCAGGCAAACAAUGGACUGUGCCACU |
| | | UGA |
| 111 | mol_type = | AUGGGUGGGCUCACCGCAUCAGACGUUCACCCCACACUGGGAGU |
| | RNA | UCAACUCUUCAGUGCGGGCAUCGCCGCCUGCCUGGCUGACGUUA |
| | origin = | UUACUUUUCCUCUGGACACAGCUAAGGUACGUUUACAGGUUCAA |
| | synthetic | GGAGAAUGUCCCACUUCUUCCGUGAUUCGGUAUAAAGGCGUUUU |
| | construct | GGGGACUAUCACAGCCGUAGUGAAGACAGAAGGCCGCAUGAAGC |
| | | UCUACAGUGGUCUGCCAGCAGGCCUGCAGCGACAAAUCAGCUCU |
| | | GCUUCACUGCGUAUCGGUUUGUACGACACUGUGCAAGAAUUCCU |
| | | CACCGCAGGAAAGGAGACCGCACCCAGCUUAGGGAGCAAAAUCC |
| | | UUGCCGGGCUCACAACAGGCGGCGUCGCUGUGUUCAUCGGACAG |
| | | CCUACAGAGGUGGUUAAGGUAAGACUUCAGGCCCAGUCACACCU |
| | | UCAUGGUAUAAAACCUCGCUAUACUGGAACGUACAAUGCUUACA |
| | | GGAUCAUCGCCACCACAGAAGGCCUCACAGGGCUGUGGAAAGGU |
| | | ACAACCCCUAAUUUGAUGCGAUCAGUCAUCAUCAAUUGCACUGA |
| | | GCUGGUGACUUACGAUCUCAUGAAGGAAGCUUUUGUCAAAAACA |
| | | ACAUACUGGCCGAUGACGUCCCAUGCCACCUGGUUUCCGCACUC |
| | | AUCGCAGGAUUCUGUGCCACCGCAAUGUCUUCUCCUGUCGAUGU |
| | | CGUGAAGACACGAUUUAUCAAUUCACCUCCUGGACAGUACAAAU |
| | | CUGUCCCCAAUUGUGCCAUGAAAGUGUUCACAAACGAAGGGCCA |
| | | ACAGCCUUUUUUAAGGGGCUCGUGCCCUCCUUUCUUCGACUCGG |
| | | AUCUUGGAACGUCAUUAUGUUUGUGUGUUUUGAACAGUUAAAA |
| | | AGAGAGCUCUCCAAAUCUAGGCAGACAAUGGAUUGCGCCACUUA |
| | | G′ |
| 112 | mol_type = | AUGGGCGGUCUCACAGCUAGCGAUGUGCAUCCCACGCUGGGGGU |
| | RNA | GCAACUUUUCAGCGCGGGCAUCGCCGCUUGCUUAGCUGAUGUCA |
| | origin = | UUACCUUUCCCUUAGAUACGGCUAAGGUCCGCCUUCAGGUCCAG |
| | synthetic | GGCGAAUGUCCCACCUCAUCCGUGAUUCGCUAUAAGGGCGUAUU |
| | construct | AGGGACAAUCACAGCCGUCGUGAAGACCGAGGGCCGUAUGAAAC |
| | | UCUAUAGCGGGCUGCCCGCCGGAUUGCAGCGACAGAUCAGCUCU |
| | | GCAUCCCUGCGAAUUGGCCUGUAUGACACUGUGCAGGAAUUCCU |
| | | CACCGCCGGGAAAGAGACGGCGCCCAGUCUGGGCAGCAAGAUAC |
| | | UGGCCGGACUCACUACAGGUGGCGUGGCGGUGUUCAUUGGACAG |
| | | CCUACAGAGGUGGUUAAGGUUAGACUGCAGGCUCAAUCCCAUCU |
| | | GCACGGGAUCAAACCUCGCUAUACUGGCACUUAUAAUGCUUACA |
| | | GGAUCAUUGCCACGACAGAGGGCCUCACUGGACUUUGGAAAGGG |
| | | ACAACACCCAAUUUAAUGCGAAGCGUUAUCAUCAACUGCACCGA |
| | | GCUCGUGACCUAUGAUCUGAUGAAGGAAGCCUUUGUAAAAAACA |
| | | ACAUCCUGGCAGAUGACGUCCCAUGUCACUUGGUAUCUGCACUC |
| | | AUAGCUGGCUUUUGUGCUACCGCCAUGUCUUCCCCUGUGGAUGU |
| | | GGUAAAGACACGAUUCAUCAAUUCUCCACCUGGUCAAUAUAAAA |
| | | GUGUUCCUAAUUGUGCAAUGAAAGUAUUCACUAACGAAGGCCCA |
| | | ACAGCGUUCUUCAAGGGGCUCGUGCCAUCUUUUCUGCGGCUCGG |
| | | GUCCUGGAAUGUCAUCAUGUUUGUGUGCUUCGAACAGUUAAAA |
| | | AGGGAACUGUCAAAAUCAAGGCAGACAAUGGAUUGCGCUACUUG |
| | | A |
| 113 | mol_type = | AUGGGCGGUCUGACCGCAUCUGACGUACAUCCUACCCUUGGGGU |
| | RNA | GCAGCUCUUUAGUGCUGGAAUCGCCGCUUGCCUUGCGGACGUUA |
| | origin = | UUACUUUUCCCUUAGAUACUGCUAAGGUGCGCCUCCAGGUCCAG |
| | synthetic | GGCGAGUGUCCUACAUCAUCCGUUAUUCGCUACAAGGGCGUAUU |
| | construct | AGGCACUAUUACAGCCGUGGUGAAAACCGAAGGCCGCAUGAAAU |
| | | UGUAUAGCGGGCUCCCUGCUGGUUUGCAAAGGCAGAUCAGCUCC |
| | | GCUUCUCUGCGGAUCGGUUUAUACGACACCGUUCAGGAGUUUCU |
| | | UACCGCAGGAAAGGAGACGGCACCUAGUUUAGGGAGCAAAAUUC |
| | | UCGCCGGGCUCACUACAGGUGGCGUCGCCGUAUUCAUCGGACAG |
| | | CCUACAGAGGUGGUAAAGGUACGCCUGCAAGCUCAGUCUCAUCU |
| | | GCACGGCAUUAAACCUCGCUACACUGGGACAUAUAACGCUUAUA |
| | | GGAUUAUCGCCACAACAGAAGGCCUAACGGGACUAUGGAAAGGC |
| | | ACAACCCCCAACUUAAUGCGGUCUGUCAUUAUCAACUGCACUGA |
| | | ACUCGUGACUUACGAUCUUAUGAAAGAAGCUUUCGUGAAGAACA |
| | | ACAUCCUGGCCGAUGAUGUUCCUUGUCACUUGGUGUCCGCACUU |
| | | AUCGCUGGAUUCUGUGCUACCGCUAUGUCAUCCCCUGUCGACGU |
| | | CGUUAAAACACGCUUCAUAAAUUCUCCACCUGGUCAAUAUAAAA |
| | | GUGUUCCCAAUUGUGCCAUGAAGGUGUUUACAAAUGAGGGACCC |
| | | ACAGCCUUUUUCAAGGGCCUGGUUCCAUCGUUUCUACGGCUCGG |
| | | AUCCUGGAACGUCAUCAUGUUCGUCUGUUUUGAGCAACUGAAAC |
| | | GGGAGCUUUCCAAAUCCAGACAGACGAUGGACUGUGCUACUUGA |
| | | ′ |
| 114 | mol_type = | AUGGGGGGUUUAACCGCUUCAGAUGUGCAUCCAACGCUGGGGGU |
| | RNA | GCAACUUUUCAGUGCAGGGAUUGCCGCUUGCCUCGCAGACGUCA |
| | origin = | UUACUUUUCCAUUGGACACCGCAAAGGUGCGCCUGCAGGUCCAG |
| | synthetic | GGCGAAUGCCCAACUUCAUCGGUUAUCAGAUAUAAGGGCGUCUU |
| | construct | GGGAACCAUCACAGCCGUGGUGAAGACUGAAGGCAGGAUGAAAC |
| | | UCUACAGCGGGCUCCCAGCCGGCCUACAGCGGCAAAUCAGCUCC |
| | | GCAUCGCUGAGAAUCGGACUGUAUGACACCGUUCAAGAAUUCCU |
| | | AACCGCAGGAAAGGAGACAGCACCCAGUCUGGGAAGUAAAAUUC |
| | | UCGCUGGGCUGACUACUGGCGGCGUGGCCGUAUUCAUCGGACAA |
| | | CCUACUGAAGUGGUUAAGGUACGGCUACAAGCUCAAAGCCACCU |
| | | GCACGGCAUUAAACCACGCUAUACUGGCACUUACAAUGCUUAUA |
| | | GGAUAAUUGCAACAACAGAAGGCCUCACUGGACUUUGGAAGGGC |
| | | ACAACACCUAAUCUGAUGCGAUCUGUUAUUAUUAAUUGCACUGA |
| | | GCUCGUGACUUACGAUCUCAUGAAAGAGGCCUUUGUGAAGAAUA |
| | | AUAUCCUGGCAGACGACGUUCCUUGCCACCUUGUGUCCGCACUA |
| | | AUCGCGGGAUUCUGUGCCACCGCAAUGUCUAGCCCUGUGGAUGU |
| | | UGUCAAAACACGGUUUAUAAACUCCCCUCCCGGCCAGUAUAAAA |
| | | GUGUCCCCAAUUGUGCAAUGAAGGUCUUCACUAAUGAAGGCCCC |
| | | ACAGCAUUUUUCAAAGGGCUGGUGCCCUCCUUUCUUCGGCUCGG |
| | | AUCGUGGAACGUGAUCAUGUUCGUGUGUUUCGAACAGCUGAAA |
| | | AGGGAGUUGUCAAAGAGCAGACAAACCAUGGACUGUGCUACCUG |
| | | A |
| 115 | mol_type = | AUGGGAGGUUUAACUGCCAGCGACGUUCAUCCAACUCUGGGGGU |
| | RNA | GCAGCUCUUCAGUGCAGGCAUCGCCGCAUGCCUCGCUGACGUGA |
| | origin = | UUACGUUUCCUUUAGACACCGCUAAGGUCCGUCUUCAAGUCCAA |
| | synthetic | GGCGAAUGUCCUACUUCUUCCGUCAUUCGUUACAAGGGCGUGUU |
| | construct | GGGGACCAUCACAGCCGUGGUGAAGACUGAAGGCAGGAUGAAAC |
| | | UUUAUAGCGGGCUCCCUGCUGGCUUACAGAGGCAAAUCAGCUCU |
| | | GCUUCACUCCGGAUUGGGCUCUACGACACUGUCCAGGAAUUUCU |
| | | UACCGCCGGAAAGGAGACAGCUCCCAGCUUGGGGAGUAAAAUCC |
| | | UCGCGGGACUCACUACAGGCGGCGUCGCCGUCUUCAUUGGGCAA |
| | | CCCACUGAGGUGGUGAAGGUCCGGUUGCAGGCUCAAUCCCACCU |
| | | UCACGGCAUUAAGCCUCGCUACACAGGCACAUACAACGCCUACA |
| | | GAAUCAUUGCCACCACAGAAGGGUUAACCGGACUUUGGAAGGGG |
| | | ACAACACCCAACUUAAUGCGAUCUGUCAUCAUCAAUUGCACUGA |
| | | ACUCGUGACUUACGACCUCAUGAAGGAAGCCUUUGUGAAGAAUA |
| | | ACAUCUUGGCCGACGACGUUCCUUGUCACCUGGUUUCUGCAUUA |
| | | AUUGCUGGUUUUUGUGCUACUGCCAUGUCAUCUCCUGUUGAUGU |
| | | CGUCAAAACACGGUUCAUCAACUCCCCUCCCGGGCAGUACAAAU |
| | | CUGUCCCCAAUUGUGCCAUGAAAGUCUUCACAAACGAGGGUCCA |
| | | ACCGCCUUCUUCAAAGGGCUUGUUCCCUCUUUUCUUCGUCUCGG |
| | | CUCCUGGAACGUGAUCAUGUUCGUGUGCUUCGAACAGCUGAAGA |
| | | GGGAACUGUCCAAAUCCAGGCAAACGAUGGAUUGUGCCACAUGA |
| 116 | mol_type = | AUGGGCGGUUUAACGGCAAGCGACGUGCAUCCCACGCUGGGCGU |
| | RNA | GCAGCUUUUCAGUGCCGGAAUCGCUGCUUGCUUAGCAGACGUUA |
| | origin = | UAACUUUUCCCCUUGACACGGCCAAGGUCCGCCUGCAAGUCCAA |
| | synthetic | GGCGAAUGUCCAACCUCAUCGGUUAUCCGCUAUAAAGGCGUUUU |
| | construct | GGGAACUAUUACAGCCGUCGUGAAGACCGAGGGCAGGAUGAAAC |
| | | UUUACAGCGGGCUCCCUGCUGGUUUGCAGAGGCAGAUAAGCUCA |
| | | GCAUCCCUUCGAAUUGGUCUGUACGACACCGUUCAGGAGUUCCU |
| | | UACUGCGGGAAAGGAAACAGCCCCUAGUUUGGGCAGCAAAAUUC |
| | | UGGCCGGGCUGACUACAGGUGGUGUCGCCGUAUUUAUUGGACAA |
| | | CCCACAGAAGUAGUAAAGGUUCGACUGCAGGCCCAGAGCCAUUU |
| | | GCAUGGAAUCAAGCCGCGCUACACUGGUACUUACAACGCCUAUA |
| | | GAAUCAUCGCCACCACAGAGGGAUUAACUGGACUUUGGAAGGGG |
| | | ACAACCCCUAAUUUAAUGCGAUCAGUUAUCAUCAAUUGCACCGA |
| | | GCUAGUGACGUACGAUCUCAUGAAAGAAGCAUUUGUCAAAAACA |
| | | ACAUUUUGGCAGAUGACGUUCCAUGUCACUUGGUCUCUGCGUUA |
| | | AUCGCCGGAUUUUGUGCAACCGCUAUGUCUUCUCCUGUCGACGU |
| | | UGUGAAGACAAGGUUCAUAAACUCUCCACCUGGCCAAUAUAAAA |
| | | GUGUCCCUAAUUGUGCCAUGAAAGUCUUUACUAAUGAAGGGCCU |
| | | ACCGCAUUUUUUAAGGGGCUCGUUCCGUCCUUUCUGCGGCUCGG |
| | | AUCCUGGAACGUGAUCAUGUUCGUGUGUUUUGAGCAGUUGAAA |
| | | AGGGAGCUGUCUAAGUCAAGGCAAACGAUGGAUUGUGCCACCUG |
| | | A′ |
| 117 | mol_type = | AUGGGGGGUCUUACUGCAUCAGACGUUCAUCCCACACUGGGGGU |
| | RNA | GCAGCUCUUCAGUGCGGGCAUCGCCGCGUGCCUCGCAGAUGUCA |
| | origin = | UUACGUUUCCCCUGGAUACAGCUAAGGUGCGCCUGCAGGUCCAG |
| | synthetic | GGCGAAUGCCCAACUUCCUCCGUAAUCCGUUACAAAGGCGUGUU |
| | construct | GGGCACCAUCACAGCCGUCGUGAAGACCGAGGGACGUAUGAAAC |
| | | UUUACAGCGGGCUCCCUGCCGGGCUGCAACGGCAGAUCAGCUCC |
| | | GCUUCCCUCCGUAUCGGACUAUACGACACAGUGCAGGAAUUCCU |
| | | UACGGCUGGAAAAGAGACGGCACCUAGCUUGGGCAGCAAAAUCU |
| | | UGGCAGGGCUCACUACAGGUGGCGUUGCUGUCUUUAUCGGACAG |
| | | CCUACUGAGGUAGUGAAGGUCCGUCUCCAAGCUCAAUCCCACCU |
| | | GCACGGCAUCAAACCUCGAUACACCGGCACUUAUAACGCCUACA |
| | | GGAUUAUCGCUACUACAGAAGGCCUUACUGGGCUGUGGAAGGGG |
| | | ACAACACCUAAUUUAAUGCGGUCGGUUAUCAUCAAUUGCACUGA |
| | | GCUCGUGACCUACGAUCUCAUGAAGGAAGCGUUUGUGAAAAACA |
| | | ACAUUCUGGCAGAUGACGUCCCCUGCCACUUGGUGUCUGCUCUC |
| | | AUCGCCGGAUUUUGUGCCACCGCCAUGUCUUCCCCUGUUGACGU |
| | | UGUGAAAACAAGAUUUAUCAAUUCUCCUCCAGGUCAAUAUAAAA |
| | | GUGUCCCCAAUUGUGCCAUGAAGGUGUUCACAAAUGAGGGCCCA |
| | | ACGGCAUUCUUCAAGGGGCUCGUCCCUUCCUUUCUUCGACUGGG |
| | | GUCGUGGAACGUCAUCAUGUUUGUAUGCUUUGAACAAUUGAAA |
| | | CGGGAGCUGUCCAAGUCCCGGCAGACAAUGGACUGUGCCACUUA |
| | | G |
| 118 | mol_type = | AUGGGCGGCCUGACAGCUUCUGAUGUGCAUCCUACACUGGGUGU |
| | RNA | GCAACUUUUCAGUGCGGGAAUCGCUGCUUGCCUCGCCGACGUCA |
| | origin = | UUACCUUUCCCUUAGACACAGCAAAGGUCCGCCUGCAGGUCCAA |
| | synthetic | GGCGAGUGUCCUACUUCCUCCGUCAUUCGAUACAAGGGUGUGCU |
| | construct | GGGAACCAUCACCGCUGUUGUAAAAACAGAAGGCCGUAUGAAGC |
| | | UUUACAGCGGACUGCCAGCCGGUUUACAGAGGCAGAUCAGCUCU |
| | | GCGUCGCUGCGUAUCGGGCUCUAUGACACCGUCCAAGAGUUCCU |
| | | AACAGCAGGGAAGGAGACAGCGCCCAGCCUGGGGAGUAAAAUCC |
| | | UCGCAGGACUCACAACAGGUGGUGUCGCCGUGUUCAUUGGACAG |
| | | CCAACUGAGGUGGUGAAGGUCCGACUGCAGGCCCAGUCCCACCU |
| | | CCACGGGAUCAAACCUCGCUACACCGGGACGUACAACGCUUAUA |
| | | GAAUCAUCGCCACCACAGAAGGGCUCACAGGACUCUGGAAGGGG |
| | | ACAACACCUAAUUUGAUGCGAUCUGUUAUUAUUAAUUGUACUG |
| | | AACUCGUGACUUAUGAUCUCAUGAAGGAGGCCUUUGUGAAAAAC |
| | | AACAUCCUGGCCGACGACGUCCCAUGUCACCUGGUAUCUGCACU |
| | | GAUCGCCGGAUUCUGUGCAACCGCCAUGUCAUCUCCUGUUGACG |
| | | UUGUCAAGACACGCUUCAUAAACUCUCCUCCGGGUCAGUAUAAA |
| | | AGUGUCCCCAAUUGUGCCAUGAAAGUAUUUACCAACGAAGGCCC |
| | | AACCGCAUUCUUUAAGGGGCUUGUGCCAUCUUUUCUGCGGCUGG |
| | | GCUCCUGGAAUGUCAUCAUGUUCGUGUGUUUCGAACAAUUAAAA |
| | | AGAGAGCUGUCGAAGUCCCGGCAGACGAUGGACUGCGCCACUUA |
| | | G |
| 119 | mol_type = | AUGGGGGGUCUCACAGCUAGUGACGUUCAUCCUACACUCGGAGU |
| | RNA | GCAGCUCUUCAGUGCAGGAAUCGCCGCUUGCCUUGCAGACGUUA |
| | origin = | UCACCUUUCCUCUUGACACCGCUAAGGUCCGACUGCAGGUCCAA |
| | synthetic | GGCGAGUGUCCCACCUCCUCCGUAAUCAGAUACAAGGGUGUCCU |
| | construct | GGGGACAAUCACCGCAGUCGUAAAGACAGAGGGCCGUAUGAAAC |
| | | UUUACAGCGGGCUCCCGGCAGGAUUGCAGCGCCAGAUCAGCUCC |
| | | GCAUCUCUGAGAAUUGGUCUCUACGACACUGUUCAAGAAUUCCU |
| | | AACAGCCGGAAAGGAGACUGCACCCAGUCUGGGAAGUAAAAUAC |
| | | UAGCCGGGCUCACCACAGGCGGCGUUGCUGUAUUCAUAGGACAG |
| | | CCUACCGAAGUAGUCAAGGUCAGACUUCAGGCUCAGUCUCAUCU |
| | | GCACGGAAUCAAGCCCCGUUAUACCGGAACUUAUAAUGCUUACA |
| | | GGAUCAUCGCCACCACAGAAGGCUUAACUGGACUUUGGAAGGGG |
| | | ACCACACCUAAUUUAAUGCGUUCCGUUAUAAUCAACUGCACCGA |
| | | GCUAGUGACUUACGACCUCAUGAAGGAGGCAUUUGUGAAAAACA |
| | | ACAUCCUGGCAGAUGACGUUCCUUGUCACUUAGUGUCUGCACUC |
| | | AUCGCCGGAUUUUGUGCAACCGCCAUGUCCAGUCCCGUCGAUGU |
| | | CGUUAAGACUCGGUUCAUUAACUCCCCUCCGGGCCAAUACAAAU |
| | | CUGUCCCUAACUGUGCCAUGAAAGUAUUUACAAACGAGGGUCCU |
| | | ACAGCCUUCUUUAAGGGGCUCGUCCCGUCUUUUCUCCGGCUAGG |
| | | CUCGUGGAACGUUAUCAUGUUUGUGUGUUUCGAACAACUAAAA |
| | | AGAGAGCUUUCCAAAUCCAGGCAAACCAUGGAUUGCGCAACUUG |
| | | A |
| 120 | mol_type = | AUGGGGGGCCUGACAGCUUCCGACGUCCACCCCACAUUGGGAGU |
| | RNA | UCAGCUCUUCAGUGCAGGAAUCGCCGCUUGCCUUGCCGAUGUCA |
| | origin = | UUACUUUUCCUUUGGAUACCGCUAAGGUGCGCCUGCAAGUCCAG |
| | synthetic | GGCGAGUGUCCUACUUCUUCUGUGAUUCGAUACAAGGGCGUCUU |
| | construct | AGGAACUAUUACAGCCGUCGUGAAAACCGAAGGUCGUAUGAAGC |
| | | UCUACAGUGGGCUGCCAGCCGGACUGCAGCGGCAGAUAAGCUCA |
| | | GCUUCUCUCCGAAUCGGACUGUACGACACCGUUCAGGAGUUUCU |
| | | UACUGCCGGGAAGGAAACCGCACCCAGUCUGGGGAGCAAAAUUC |
| | | UUGCCGGGCUCACCACAGGCGGCGUCGCCGUCUUCAUCGGACAG |
| | | CCUACCGAAGUCGUGAAGGUGCGACUGCAAGCCCAGUCACACCU |
| | | GCAUGGCAUCAAGCCACGUUAUACUGGAACUUACAACGCUUACA |
| | | GGAUUAUCGCCACCACUGAAGGCCUUACUGGACUCUGGAAGGGG |
| | | ACAACUCCUAAUUUAAUGCGAUCAGUUAUCAUUAACUGCACAGA |
| | | GCUCGUUACUUAUGACCUUAUGAAGGAAGCAUUCGUGAAAAACA |
| | | ACAUCUUAGCAGACGACGUCCCCUGUCACCUGGUAUCUGCACUA |
| | | AUCGCCGGAUUUUGUGCUACCGCCAUGUCAUCCCCUGUGGACGU |
| | | CGUAAAGACGCGAUUCAUAAAUUCUCCUCCCGGUCAAUACAAAU |
| | | CGGUCCCCAAUUGUGCCAUGAAAGUGUUCACAAACGAGGGGCCA |
| | | ACCGCCUUUUUCAAAGGGCUUGUGCCCUCUUUUCUGCGACUCGG |
| | | CUCCUGGAAUGUCAUCAUGUUCGUGUGCUUCGAACAGCUAAAAA |
| | | GGGAGCUGUCUAAAUCCAGGCAAACAAUGGACUGUGCUACUUAA |
| 121 | mol_type = | AUGGGCGGUCUUACAGCAAGCGACGUUCAUCCCACACUCGGGGU |
| | RNA | GCAGCUCUUUAGUGCGGGAAUCGCCGCUUGCCUCGCCGACGUGA |
| | origin = | UAACCUUUCCGUUGGACACAGCCAAAGUACGCCUUCAAGUCCAG |
| | synthetic | GGCGAGUGCCCAACUUCUUCCGUGAUUCGAUAUAAAGGCGUGUU |
| | construct | GGGAACCAUUACUGCCGUCGUGAAGACCGAAGGCCGCAUGAAGU |
| | | UAUACAGCGGGCUGCCCGCUGGGUUGCAGAGGCAGAUAAGCUCU |
| | | GCCUCCCUGCGUAUCGGCUUGUAUGACACGGUUCAAGAAUUCCU |
| | | CACUGCCGGGAAGGAGACCGCCCCCAGCUUGGGCAGCAAAAUCC |
| | | UGGCCGGGCUCACUACAGGCGGCGUGGCUGUAUUUAUUGGACAA |
| | | CCUACCGAAGUUGUUAAGGUACGACUUCAAGCUCAAUCUCACCU |
| | | GCACGGAAUUAAACCGCGCUACACUGGAACUUACAACGCUUAUC |
| | | GAAUUAUUGCCACUACAGAAGGCUUAACAGGACUUUGGAAGGG |
| | | GACGACCCCUAAUUUAAUGCGGUCGGUUAUCAUCAAUUGCACCG |
| | | AAUUGGUGACAUACGAUCUUAUGAAAGAGGCUUUCGUGAAAAA |
| | | CAACAUCUUGGCAGAUGACGUUCCUUGUCACUUAGUAUCCGCAU |
| | | UAAUCGCCGGAUUCUGUGCUACUGCCAUGUCCUCCCCUGUUGAU |
| | | GUUGUCAAGACAAGGUUCAUAAAUUCGCCUCCUGGCCAAUAUAA |
| | | AAGUGUCCCCAAUUGUGCCAUGAAAGUCUUCACCAACGAAGGGC |
| | | CUACCGCCUUCUUUAAGGGGCUCGUCCCGUCUUUUCUGCGUCUG |
| | | GGGUCCUGGAACGUGAUCAUGUUUGUGUGUUUUGAGCAAUUGA |
| | | AAAGAGAGCUGUCGAAGUCCAGACAGACGAUGGACUGUGCUACA |
| | | UGA |
| 122 | mol_type = | AUGGGUGGACUGACCGCUUCGGACGUUCACCCUACCCUGGGAGU |
| | RNA | GCAGCUCUUCAGUGCAGGAAUCGCCGCGUGCCUCGCCGAUGUCA |
| | origin = | UUACUUUUCCUUUAGAUACAGCUAAAGUGCGGCUGCAAGUCCAA |
| | synthetic | GGAGAAUGCCCCACAUCCUCAGUCAUUCGAUACAAAGGCGUUUU |
| | construct | GGGGACCAUCACUGCCGUGGUGAAAACAGAGGGCCGAAUGAAAC |
| | | UCUACAGCGGGCUGCCAGCCGGAUUGCAGAGGCAGAUCAGCUCC |
| | | GCAUCUCUGCGAAUCGGGUUGUACGACACAGUUCAGGAGUUUCU |
| | | GACUGCCGGAAAGGAGACCGCACCUAGUUUGGGCAGUAAAAUCC |
| | | UGGCCGGACUCACUACAGGUGGCGUCGCCGUAUUUAUCGGGCAG |
| | | CCCACCGAAGUUGUGAAGGUCCGGCUGCAGGCUCAGUCUCACCU |
| | | ACACGGGAUUAAGCCACGCUACACUGGGACAUACAACGCUUAUA |
| | | GGAUCAUCGCCACCACUGAAGGUCUUACGGGACUUUGGAAAGGC |
| | | ACAACACCCAACCUAAUGCGAUCUGUUAUCAUCAAUUGUACUGA |
| | | GCUGGUCACUUACGAUCUGAUGAAAGAAGCGUUUGUGAAAAAC |
| | | AACAUUCUCGCAGAUGACGUUCCAUGUCAUCUAGUGUCCGCACU |
| | | CAUCGCCGGGUUCUGUGCAACCGCCAUGUCCAGCCCUGUUGACG |
| | | UCGUUAAGACAAGAUUUAUUAAUUCUCCACCAGGCCAAUACAAA |
| | | AGCGUCCCUAACUGUGCCAUGAAAGUGUUCACAAACGAGGGCCC |
| | | UACUGCCUUCUUUAAGGGGCUGGUUCCCUCCUUUCUGCGACUCG |
| | | GAUCCUGGAAUGUCAUCAUGUUCGUAUGCUUUGAGCAAUUGAA |
| | | AAGAGAGUUGUCUAAGUCAAGACAAACCAUGGACUGCGCCACAU |
| | | GA |
| 123 | mol_type = | AUGGGCGGCCUAACCGCGAGUGACGUUCACCCUACUUUAGGGGU |
| | RNA | GCAGCUUUUCAGCGCAGGCAUCGCAGCCUGCCUCGCUGACGUCA |
| | origin = | UUACAUUUCCCCUCGACACCGCCAAGGUCCGCCUGCAAGUUCAG |
| | synthetic | GGUGAAUGUCCGACUUCCUCCGUCAUUCGUUAUAAAGGCGUGCU |
| | construct | CGGGACCAUUACCGCUGUGGUGAAGACCGAAGGCCGCAUGAAAC |
| | | UGUAUAGCGGGCUGCCUGCGGGUCUCCAGAGACAAAUCAGCUCA |
| | | GCCUCUCUCCGUAUCGGCCUAUACGAUACAGUUCAAGAAUUCUU |
| | | AACGGCCGGGAAAGAAACCGCACCUUCUCUGGGAAGCAAAAUUC |
| | | UCGCCGGGCUCACUACAGGCGGCGUCGCCGUCUUCAUCGGCCAA |
| | | CCAACUGAAGUUGUUAAGGUACGACUGCAAGCUCAAAGUCACCU |
| | | UCACGGCAUCAAGCCUCGCUACACUGGCACUUACAAUGCUUACA |
| | | GGAUUAUCGCUACAACCGAAGGCCUAACUGGACUCUGGAAGGGG |
| | | ACAACACCCAAUUUGAUGCGGUCUGUUAUUAUCAACUGCACAGA |
| | | ACUCGUGACAUACGAUCUCAUGAAGGAAGCUUUCGUGAAGAAUA |
| | | ACAUCCUGGCUGAUGAUGUCCCUUGCCACCUCGUAUCUGCUCUG |
| | | AUCGCAGGAUUUUGUGCUACCGCUAUGUCAAGCCCUGUCGACGU |
| | | CGUAAAAACACGGUUUAUAAACUCCCCCCCUGGCCAGUACAAGU |
| | | CUGUCCCUAAUUGUGCCAUGAAAGUCUUCACCAAUGAAGGCCCC |
| | | ACUGCCUUUUUCAAGGGGCUCGUGCCCUCCUUUCUGCGGCUCGG |
| | | GUCCUGGAAUGUCAUCAUGUUUGUGUGUUUUGAGCAACUGAAA |
| | | AGGGAGUUAAGUAAAUCCAGGCAGACCAUGGACUGCGCCACAUG |
| | | A |
| 124 | mol_type = | AUGGGCGGUCUGACAGCAUCCGACGUGCAUCCUACUCUGGGGGU |
| | RNA | GCAGCUCUUCAGCGCCGGGAUUGCCGCGUGCCUCGCUGAUGUUA |
| | origin = | UAACUUUUCCCCUGGACACCGCUAAGGUCCGUCUGCAAGUUCAG |
| | synthetic | GGCGAGUGCCCCACCUCUUCUGUAAUUCGCUAUAAAGGCGUCCU |
| | construct | UGGAACCAUAACUGCUGUUGUGAAGACUGAAGGCCGUAUGAAGC |
| | | UCUAUAGCGGGCUGCCUGCGGGCCUGCAGAGGCAGAUCAGCUCC |
| | | GCCUCCCUCCGUAUCGGCCUGUACGACACUGUCCAAGAAUUCCU |
| | | CACUGCUGGAAAAGAAACUGCACCCAGUCUGGGGAGUAAGAUCC |
| | | UCGCCGGGCUUACUACAGGUGGCGUCGCUGUCUUUAUUGGACAG |
| | | CCAACAGAGGUCGUGAAGGUGCGACUGCAGGCUCAAAGCCACCU |
| | | GCAUGGAAUUAAACCUCGCUACACUGGAACCUAUAACGCCUAUA |
| | | GGAUUAUCGCAACCACCGAAGGUUUAACUGGGCUCUGGAAGGGA |
| | | ACAACUCCUAAUCUAAUGCGAUCAGUUAUUAUCAAUUGCACUGA |
| | | ACUCGUUACUUAUGAUCUCAUGAAGGAAGCCUUUGUGAAAAAU |
| | | AAUAUAUUGGCGGACGACGUCCCUUGUCACCUGGUAUCCGCACU |
| | | UAUCGCCGGAUUUUGUGCUACCGCUAUGUCCAGCCCUGUCGAUG |
| | | UUGUUAAAACAAGAUUUAUAAACUCUCCCCCUGGUCAAUAUAAA |
| | | AGUGUUCCUAACUGUGCCAUGAAGGUCUUCACUAACGAAGGCCC |
| | | CACAGCUUUUUUUAAGGGGCUUGUUCCCUCCUUUUUGCGGCUGG |
| | | GUUCUUGGAAUGUGAUCAUGUUCGUCUGUUUUGAACAGCUGAA |
| | | AAGGGAGCUAUCCAAAUCCAGACAGACGAUGGACUGUGCCACCU |
| | | GA |
| 125 | mol_type = | AUGGGUGGUCUGACUGCUAGUGAUGUGCAUCCAACACUGGGGGU |
| | RNA | GCAACUUUUCAGUGCUGGGAUCGCCGCCUGCCUCGCCGACGUUA |
| | origin = | UAACUUUUCCGCUGGAUACCGCCAAGGUGCGCCUACAGGUUCAG |
| | synthetic | GGCGAGUGUCCUACCUCUUCAGUGAUUCGAUAUAAAGGCGUCUU |
| | construct | GGGUACCAUCACUGCUGUGGUGAAGACUGAAGGCCGCAUGAAGC |
| | | UCUACAGCGGCCUGCCAGCGGGCUUACAGAGGCAGAUCAGCUCC |
| | | GCUUCCCUGCGCAUCGGCCUGUACGACACAGUCCAAGAAUUUCU |
| | | UACUGCCGGGAAGGAAACCGCUCCAAGUUUGGGGUCCAAAAUUC |
| | | UCGCCGGGCUCACCACAGGUGGUGUCGCUGUCUUUAUUGGCCAG |
| | | CCUACCGAAGUGGUGAAGGUCCGACUCCAAGCUCAGUCCCAUCU |
| | | CCACGGAAUUAAGCCUCGGUACACCGGGACUUACAAUGCUUAUA |
| | | GAAUCAUCGCUACCACAGAAGGAUUAACUGGACUUUGGAAGGGC |
| | | ACGACACCCAAUUUGAUGCGGUCAGUCAUUAUCAACUGCACAGA |
| | | GUUGGUUACCUAUGAUCUCAUGAAGGAAGCCUUUGUGAAAAAU |
| | | AACAUUCUGGCAGACGACGUUCCAUGUCACCUGGUUUCUGCAUU |
| | | AAUCGCUGGGUUUUGUGCUACCGCCAUGUCAUCUCCUGUCGACG |
| | | UUGUGAAAACCCGGUUUAUAAAUUCACCACCUGGCCAGUAUAAA |
| | | AGCGUUCCCAAUUGUGCCAUGAAGGUGUUCACAAAUGAGGGCCC |
| | | UACCGCAUUCUUCAAAGGGCUCGUCCCAUCCUUUCUCCGUCUAG |
| | | GAUCCUGGAAUGUCAUAAUGUUCGUCUGUUUUGAACAGUUAAA |
| | | AAGGGAACUGAGUAAAUCCAGGCAAACAAUGGACUGCGCUACCU |
| | | GA |
| 126 | mol_type = | AUGGGAGGUCUGACUGCAAGCGACGUACACCCAACUCUAGGAGU |
| | RNA | GCAGCUCUUUAGUGCAGGAAUUGCCGCUUGUCUCGCUGAUGUCA |
| | origin = | UUACUUUUCCCUUGGAUACCGCUAAGGUCCGUCUGCAGGUCCAG |
| | synthetic | GGCGAGUGUCCUACUUCAUCUGUCAUUCGUUAUAAAGGCGUAUU |
| | construct | GGGAACCAUUACAGCAGUCGUGAAAACUGAGGGCCGCAUGAAGC |
| | | UCUACAGUGGGCUUCCAGCUGGCCUGCAGCGACAGAUCAGCUCG |
| | | GCGUCCCUGCGUAUUGGCCUAUACGACACAGUCCAAGAGUUCCU |
| | | CACAGCUGGGAAGGAGACCGCGCCAAGUUUGGGCAGCAAAAUAC |
| | | UCGCCGGUCUCACAACAGGCGGCGUGGCCGUAUUCAUUGGACAG |
| | | CCCACUGAGGUCGUUAAGGUACGACUACAGGCUCAGUCUCAUCU |
| | | CCAUGGGAUAAAACCUCGCUAUACUGGCACAUACAAUGCUUAUC |
| | | GAAUCAUCGCAACAACCGAAGGCCUCACAGGGCUGUGGAAAGGG |
| | | ACGACACCUAACUUAAUGCGGUCAGUGAUCAUCAACUGCACUGA |
| | | ACUGGUGACCUACGAUCUGAUGAAGGAAGCUUUCGUAAAGAAU |
| | | AACAUUCUGGCAGACGACGUCCCAUGUCAUCUAGUAUCCGCGUU |
| | | AAUCGCCGGAUUUUGUGCCACUGCUAUGUCUUCCCCUGUCGAUG |
| | | UUGUGAAGACACGAUUCAUAAACUCUCCUCCCGGCCAAUACAAA |
| | | UCUGUCCCUAAUUGUGCCAUGAAGGUCUUCACAAACGAGGGUCC |
| | | AACAGCCUUUUUUAAGGGGCUCGUUCCUUCGUUUCUUCGGCUUG |
| | | GGUCCUGGAAUGUGAUCAUGUUUGUGUGUUUUGAACAAUUGAA |
| | | GAGAGAGCUCUCUAAGUCCAGGCAGACAAUGGAUUGUGCCACCU |
| | | AG |
| 127 | mol_type = | AUGGGUGGACUGACAGCCAGCGAUGUGCACCCUACGCUCGGAGU |
| | RNA | UCAGCUCUUUAGCGCCGGCAUCGCCGCCUGCCUUGCCGAUGUCA |
| | origin = | UUACAUUUCCUCUAGACACUGCUAAGGUACGCCUGCAGGUCCAA |
| | synthetic | GGAGAAUGUCCAACAUCAUCCGUAAUUCGGUACAAAGGUGUAUU |
| | construct | GGGGACCAUCACCGCUGUCGUGAAGACUGAGGGCCGCAUGAAAC |
| | | UUUACAGCGGGCUGCCAGCGGGCCUGCAGAGGCAAAUAAGCUCU |
| | | GCAUCCCUGCGCAUCGGCCUGUACGACACAGUUCAGGAAUUUCU |
| | | UACUGCCGGAAAGGAGACUGCACCCAGUCUGGGGAGCAAAAUUC |
| | | UCGCCGGCCUCACUACAGGCGGCGUAGCCGUAUUCAUAGGACAA |
| | | CCUACCGAAGUCGUCAAGGUAAGACUGCAGGCCCAAUCACAUCU |
| | | GCAUGGGAUCAAGCCACGCUACACCGGGACAUACAAUGCCUAUA |
| | | GGAUUAUCGCCACAACCGAAGGCUUAACUGGGCUCUGGAAGGGG |
| | | ACAACCCCUAAUUUAAUGCGAUCAGUUAUCAUUAAUUGUACGGA |
| | | ACUGGUGACAUACGACCUCAUGAAGGAAGCUUUUGUAAAAAAU |
| | | AACAUCCUGGCAGACGACGUCCCAUGUCAUUUGGUCUCUGCAUU |
| | | AAUCGCCGGAUUUUGUGCCACCGCUAUGUCAAGCCCUGUCGAUG |
| | | UUGUCAAGACGCGGUUCAUAAAUUCCCCUCCCGGCCAGUACAAA |
| | | AGUGUUCCUAACUGUGCCAUGAAGGUGUUCACCAACGAAGGCCC |
| | | CACUGCAUUCUUCAAGGGGUUGGUUCCUUCCUUUCUGCGGCUCG |
| | | GAUCCUGGAAUGUCAUCAUGUUUGUGUGUUUCGAGCAGCUGAA |
| | | AAGAGAGCUCUCUAAGUCUAGACAAACAAUGGACUGCGCAACCU |
| | | GA |
| 128 | mol_type = | AUGGGGGGUCUCACAGCUUCUGACGUUCAUCCAACACUGGGAGU |
| | RNA | GCAGCUCUUUAGCGCAGGAAUUGCCGCUUGUCUCGCCGACGUCA |
| | origin = | UAACUUUUCCCUUAGACACCGCUAAGGUCCGCCUGCAGGUCCAG |
| | synthetic | GGCGAAUGUCCUACAUCUUCAGUGAUCCGCUAUAAAGGCGUCUU |
| | construct | GGGAACCAUCACUGCAGUCGUGAAGACCGAAGGCCGUAUGAAGC |
| | | UGUACAGCGGACUGCCGGCAGGGCUCCAGAGGCAAAUAAGUUCC |
| | | GCAUCACUGCGAAUUGGUCUGUAUGACACCGUUCAGGAAUUCCU |
| | | GACAGCCGGAAAAGAAACAGCACCUAGCUUGGGUAGCAAAAUUC |
| | | UGGCCGGUCUCACCACAGGCGGCGUUGCUGUUUUCAUAGGACAG |
| | | CCCACCGAGGUAGUAAAGGUGCGACUUCAGGCACAGAGUCAUCU |
| | | GCACGGCAUCAAACCGCGCUACACGGGCACUUACAACGCUUAUA |
| | | GGAUCAUCGCCACUACAGAAGGCCUAACUGGUCUGUGGAAGGGC |
| | | ACAACACCUAAUUUAAUGCGAUCUGUUAUUAUCAAUUGUACUGA |
| | | ACUCGUAACAUAUGACCUCAUGAAGGAAGCCUUUGUAAAAAACA |
| | | ACAUCCUCGCAGAUGACGUCCCUUGCCAUUUGGUUUCAGCUCUA |
| | | AUAGCAGGAUUUUGUGCUACUGCCAUGUCUUCUCCCGUCGACGU |
| | | UGUAAAGACACGAUUCAUUAAUUCUCCACCUGGUCAGUAUAAAU |
| | | CUGUCCCUAAUUGUGCAAUGAAAGUCUUCACAAACGAAGGCCCA |
| | | ACCGCAUUCUUCAAAGGGCUUGUGCCUUCCUUUCUGCGACUCGG |
| | | CUCCUGGAACGUUAUCAUGUUCGUGUGCUUCGAACAACUGAAGC |
| | | GGGAGCUGUCAAAAUCCAGGCAGACAAUGGACUGUGCCACAUGA |
| 129 | mol_type = | AUGGGCGGGCUCACAGCAAGCGACGUCCAUCCUACACUGGGUGU |
| | RNA | GCAGCUCUUCAGUGCAGGAAUAGCCGCCUGUCUCGCCGAUGUUA |
| | origin = | UUACUUUUCCCUUAGAUACGGCAAAGGUCCGCCUUCAAGUCCAG |
| | synthetic | GGCGAGUGCCCAACAUCAUCUGUCAUACGUUAUAAGGGCGUAUU |
| | construct | GGGCACCAUCACAGCCGUGGUGAAAACCGAAGGACGGAUGAAGC |
| | | UCUAUAGCGGACUGCCAGCAGGGCUACAAAGGCAGAUCAGCUCA |
| | | GCCUCUCUACGGAUCGGAUUGUACGACACUGUCCAAGAAUUCCU |
| | | CACUGCGGGGAAGGAAACAGCACCCAGUUUGGGGAGUAAAAUCC |
| | | UGGCCGGUCUCACCACAGGCGGCGUCGCCGUCUUCAUUGGACAG |
| | | CCUACAGAGGUGGUGAAGGUUCGACUGCAAGCCCAAAGUCACCU |
| | | GCACGGAAUCAAGCCACGCUACACUGGAACUUAUAACGCUUACA |
| | | GGAUUAUCGCCACCACCGAAGGGCUCACUGGGCUGUGGAAGGGG |
| | | ACAACUCCUAACUUGAUGCGGUCUGUCAUUAUCAACUGCACCGA |
| | | AUUGGUGACUUACGAUCUCAUGAAAGAAGCUUUUGUGAAAAAC |
| | | AACAUCCUUGCAGACGACGUUCCUUGUCACCUGGUGUCCGCACU |
| | | CAUCGCCGGAUUCUGUGCCACCGCUAUGUCUAGCCCUGUCGACG |
| | | UUGUCAAAACACGUUUUAUAAACUCUCCGCCCGGCCAGUACAAA |
| | | AGCGUCCCCAACUGUGCCAUGAAGGUGUUCACCAACGAGGGUCC |
| | | CACUGCCUUCUUCAAGGGUCUGGUUCCUUCUUUUCUGCGGCUCG |
| | | GAUCUUGGAAUGUGAUCAUGUUCGUCUGCUUUGAACAAUUAAA |
| | | ACGGGAGCUGUCCAAGAGUAGACAAACGAUGGACUGCGCCACGU |
| | | GA |

### EQUIVALENTS

Those skilled in the art will recognize or be able to ascertain using no more than routine experimentation, many equivalents to the specific embodiments of the present invention described herein. Such equivalents are intended to be encompassed by the following claim.

All publications, patents, and patent applications mentioned in this specification are herein incorporated by reference into the specification to the same extent as if each individual publication, patent or patent application was specifically indicated to be incorporated herein by reference.

1. A pharmaceutical composition comprising:
a synthetic mRNA comprising the nucleotide sequence set forth in SEQ ID NO: 6;
wherein the synthetic mRNA is packaged in a lipid nanoparticle (LNP).

2. The pharmaceutical composition of claim 1, wherein the LNP comprises (C14-4: non-cationic lipids (NCL):Cholesterol:PEG/Lipid Conjugate) at a molar ratio of (35:16:46.5:2.5), (50:10:38.5:1.5) or (56.5:10:31.8:1.7).

3. The pharmaceutical composition of claim 1, wherein the LNP comprises (SM-102:NCL:Cholesterol :PEG/Lipid Conjugate) at a molar ratio of (50:10:38.5:1.5), (48.3:9.1:40.5:2.1) or (46.6:10.4:41.2:1.8).

4. The pharmaceutical composition of claim 1, wherein all uridine nucleotides of the synthetic mRNA are substituted with pseudouridines.

5. The pharmaceutical composition of claim 1, wherein all uridine nucleotides of the synthetic mRNA are substituted with N1-Methylpseudouridines.

6. A pharmaceutical composition comprising:
a synthetic mRNA comprising the nucleotide sequence set forth in SEQ ID NO: 3;
wherein the synthetic mRNA is packaged in a lipid nanoparticle (LNP).

7. The pharmaceutical composition of claim 6, wherein the LNP comprises (C14-4: non-cationic lipids (NCL):Cholesterol:PEG/Lipid Conjugate) at a molar ratio of (35:16:46.5:2.5), (50:10:38.5:1.5) or (56.5:10:31.8:1.7).

8. The pharmaceutical composition of claim 6, wherein the LNP comprises (SM-102:NCL:Cholesterol :PEG/Lipid Conjugate) at a molar ratio of (50:10:38.5:1.5), (48.3:9.1:40.5:2.1) or (46.6:10.4:41.2:1.8).

9. The pharmaceutical composition of claim 6, wherein all uridine nucleotides of the synthetic mRNA are substituted with pseudouridines.

10. The pharmaceutical composition of claim 6, wherein all uridine nucleotides of the synthetic mRNA are substituted with N1-Methylpseudouridines.

11. A pharmaceutical composition comprising:
a synthetic mRNA comprising the nucleotide sequence set forth in SEQ ID NO: 4;
wherein the synthetic mRNA is packaged in a lipid nanoparticle (LNP).

12. The pharmaceutical composition of claim 11, wherein the LNP comprises (C14-4:non-cationic lipids (NCL):Cholesterol:PEG/Lipid Conjugate) at a molar ratio of (35:16:46.5:2.5), (50:10:38.5:1.5) or (56.5:10:31.8:1.7).

13. The pharmaceutical composition of claim 11, wherein the LNP comprises (SM-102:NCL :Cholesterol:PEG/Lipid Conjugate) at a molar ratio of (50:10:38.5:1.5), (48.3:9.1:40.5:2.1) or (46.6:10.4:41.2:1.8).

14. The pharmaceutical composition of claim 11, wherein all uridine nucleotides of the synthetic mRNA are substituted with pseudouridines.

15. The pharmaceutical composition of claim 11, wherein all uridine nucleotides of the synthetic mRNA are substituted with N1-Methylpseudouridines.


SEQUENCE LISTING (130 sequences)

SEQ ID NO: 1 - RNA, 2837 nt, synthetic construct
  source 1..2837: mol_type = other RNA
ggagaattcgtcgactggatccggtaccgaggagatctgccgccgcgatcgccatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacccggggatccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctagagtcgacctgcaggcatgcaagcttctcgagatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcctaggcgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccataaagtcgatagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 2 - RNA, 928 nt, synthetic construct
  source 1..928: mol_type = other RNA
atgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgac

SEQ ID NO: 3 - RNA, 932 nt, synthetic construct
  source 1..932: mol_type = other RNA
cgccatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgac

SEQ ID NO: 4 - RNA, 932 nt, synthetic construct
  source 1..932: mol_type = other RNA
caagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgac

SEQ ID NO: 5 - RNA, 1160 nt, synthetic construct
  source 1..1160: mol_type = other RNA
agagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagtgaagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgac

SEQ ID NO: 6 - RNA, 994 nt, synthetic construct
  source 1..994: mol_type = other RNA
acagcaccctcctgaaaactgcagcttccttctcaccttgaagaataatcctagaaaactcacaaaatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgac

SEQ ID NO: 7 - RNA, 1393 nt, synthetic construct
  source 1..1393: mol_type = other RNA
atgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgactcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaa

SEQ ID NO: 8 - RNA, 1397 nt, synthetic construct
  source 1..1397: mol_type = other RNA
cgccatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgactcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaa

SEQ ID NO: 9 - RNA, 1397 nt, synthetic construct
  source 1..1397: mol_type = other RNA
caagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgactcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaa

SEQ ID NO: 10 - RNA, 1625 nt, synthetic construct
  source 1..1625: mol_type = other RNA
agagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagtgaagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgactcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaa

SEQ ID NO: 11 - RNA, 1459 nt, synthetic construct
  source 1..1459: mol_type = other RNA
acagcaccctcctgaaaactgcagcttccttctcaccttgaagaataatcctagaaaactcacaaaatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgactcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaa

SEQ ID NO: 12 - RNA, 1223 nt, synthetic construct
  source 1..1223: mol_type = other RNA
atgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 13 - RNA, 1227 nt, synthetic construct
  source 1..1227: mol_type = other RNA
cgccatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 14 - RNA, 1227 nt, synthetic construct
  source 1..1227: mol_type = other RNA
caagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 15 - RNA, 1455 nt, synthetic construct
  source 1..1455: mol_type = other RNA
agagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagtgaagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 16 - RNA, 1289 nt, synthetic construct
  source 1..1289: mol_type = other RNA
acagcaccctcctgaaaactgcagcttccttctcaccttgaagaataatcctagaaaactcacaaaatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 17 - RNA, 1142 nt, synthetic construct
  source 1..1142: mol_type = other RNA
atgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacgctcgctttcttgctgtccaatttctattaaaggttcctttgttccctaagtccaactactaaactgggggatattatgaagggccttgagcatctggattctgcctgctcgctttcttgctgtccaatttctattaaaggttcctttgttccctaagtccaactactaaactgggggatattatgaagggccttgagcatctggattctgcct

SEQ ID NO: 18 - RNA, 1146 nt, synthetic construct
  source 1..1146: mol_type = other RNA
cgccatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacgctcgctttcttgctgtccaatttctattaaaggttcctttgttccctaagtccaactactaaactgggggatattatgaagggccttgagcatctggattctgcctgctcgctttcttgctgtccaatttctattaaaggttcctttgttccctaagtccaactactaaactgggggatattatgaagggccttgagcatctggattctgcct

SEQ ID NO: 19 - RNA, 1146 nt, synthetic construct
  source 1..1146: mol_type = other RNA
caagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacgctcgctttcttgctgtccaatttctattaaaggttcctttgttccctaagtccaactactaaactgggggatattatgaagggccttgagcatctggattctgcctgctcgctttcttgctgtccaatttctattaaaggttcctttgttccctaagtccaactactaaactgggggatattatgaagggccttgagcatctggattctgcct

SEQ ID NO: 20 - RNA, 1373 nt, synthetic construct
  source 1..1373: mol_type = other RNA
agagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagtgaagatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacgctcgctttcttgctgtccaatttctattaaaggttcctttgttccctaagtccaactactaaactgggggatattatgaagggccttgagcatctggattctgcctgctcgctttcttgctgtccaatttctattaaaggttcctttgttccctaagtccaactactaaactgggggatattatgaagggccttgagcatctggattctgcc

SEQ ID NO: 21 - RNA, 1208 nt, synthetic construct
  source 1..1208: mol_type = other RNA
acagcaccctcctgaaaactgcagcttccttctcaccttgaagaataatcctagaaaactcacaaaatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacgctcgctttcttgctgtccaatttctattaaaggttcctttgttccctaagtccaactactaaactgggggatattatgaagggccttgagcatctggattctgcctgctcgctttcttgctgtccaatttctattaaaggttcctttgttccctaagtccaactactaaactgggggatattatgaagggccttgagcatctggattctgcct

SEQ ID NO: 22 - RNA, 924 nt, synthetic construct
  source 1..924: mol_type = other RNA
atgggcggcctgactgccagcgacgtgcaccccaccctcggcgtgcagctgtttagcgccggaatcgccgcctgtctggctgatgtgattaccttccctctggacaccgccaaggtgcggctgcaggtgcaaggcgagtgccctaccagcagcgtgattagatacaagggagttctgggcaccatcacagctgtggtgaagacagaaggcagaatgaaactgtacagcggcctgccagccggcctgcagagacagatctctagcgccagcctgaggatcggcctgtatgataccgtgcaggagttcctgacagccggaaaggaaaccgctcctagcctgggctctaagatcctggctggtctgaccacaggcggagtggccgtgttcatcggccagcctacagaggtggtcaaagtccggctgcaagcccagtctcacctgcatggcatcaagcccagatacaccggcacctacaacgcctacagaatcatcgccaccaccgagggcttgacaggcctgtggaagggcacaacacctaatctgatgcggagcgtgatcatcaactgcaccgaactggtgacctacgacctgatgaaagaggccttcgtgaaaaacaacatcctggctgatgacgtgccctgccacctggtgtccgctctgatcgccggcttctgcgccacagccatgagctctcctgtggacgtcgtgaagaccagattcatcaatagccctcctggacagtacaagtccgtgcccaactgtgccatgaaggtgttcaccaacgagggacctacagcatttttcaagggcctggtgccatcattcctgagactgggctcctggaacgtgatcatgtttgtgtgcttcgagcagctgaagcgggaactgagcaagagcagacagaccatggactgcgccacctga

SEQ ID NO: 23 - RNA, 924 nt, synthetic construct
  source 1..924: mol_type = other RNA
atgggcggcctgaccgcatctgatgtgcatccaacactgggcgttcaactgttcagcgccggaatcgccgcctgcctggctgatgtgatcacattccccctggacaccgccaaggtgcggctgcaggtgcagggcgagtgccctacaagcagcgtgatcagatacaagggcgtgctgggaacaatcaccgccgtggtcaagaccgaagggagaatgaaactgtacagcggcctgcctgccggactccagagacagattagctccgcttctctgcggatcggcctgtacgataccgtgcaggagttcctgaccgctggcaaggagacagccccttctctgggaagcaagatcctggccggcctcacaaccggcggagtcgccgtgttcatcggccagcctaccgaggtggtgaaggtgcgcctgcaggcccagagccacctgcacggcatcaagcctagatatacaggcacctacaacgcctacaggatcatcgctaccaccgagggcctgactggactgtggaagggcacaacccctaatctgatgcggagcgtgattatcaactgcaccgaactggtgacctacgacctgatgaaagaagcttttgtgaagaacaacatcctggccgacgacgtcccctgtcacctggtgtccgccctgatcgccggcttctgcgccaccgccatgagcagccccgtggacgtggtgaaaacaagattcatcaacagccctcctggccaatacaagtccgtgcccaactgtgccatgaaagtgttcaccaacgagggccctaccgccttttttaagggcctggttccaagcttcctgagactgggcagctggaatgtgatcatgttcgtgtgcttcgagcagctgaagcgggaactttctaagagcagacagaccatggactgcgccacctga

SEQ ID NO: 24 - RNA, 924 nt, synthetic construct
  source 1..924: mol_type = other RNA
atgggcggactgaccgcttctgatgttcatcctacattgggcgtgcagctgttcagcgccggcatcgccgcttgtctggctgatgtgatcacattccctctggacaccgcaaaggtgcggctgcaagtgcaaggcgagtgccccaccagctccgtgatccggtacaagggagtcctgggtacaatcaccgccgtggtgaaaaccgaaggcagaatgaagctgtacagcggcctgccagccggcctgcagagacagatcagcagcgccagcctgcggatcggcctgtacgataccgtgcaggagttcctgaccgctggcaaggaaaccgccccttctctgggatctaaaatcctggccgggctgacaaccggcggcgtggccgtgtttatcggccagcctacagaggtggtcaaggtgcggctgcaggcccagagccacctgcacggcattaagcccagatacaccggcacctacaacgcctatagaatcatcgccaccacagaaggcctgacaggcctgtggaagggcacaacccctaatctgatgagatctgtgatcattaactgcaccgagctggtgacctacgacctgatgaaagaagcctttgtgaagaacaacatcctggccgacgacgtgccttgccacctggtgtccgccctgatcgccggcttctgcgccacagccatgagcagccctgtggacgtggtgaagaccagattcatcaacagcccacctggacagtacaagtccgtgcccaattgtgccatgaaagtgttcaccaacgagggccctaccgcttttttcaagggactcgtccccagcttcctgaggctcggcagctggaacgtgatcatgttcgtgtgcttcgagcagctgaagagagagctgagcaagagcagacagaccatggactgcgccacatga

SEQ ID NO: 25 - RNA, 924 nt, synthetic construct
  source 1..924: mol_type = other RNA
atgggcggactgaccgcctctgatgtgcatcctaccctgggcgtccagctgttcagcgccggaatcgccgcttgtctggctgatgtgatcaccttccccctggacacagcgaaggtcagactgcaggtgcagggcgagtgtcctaccagcagcgtgattagatacaagggcgtgctgggaacaatcacagctgtggtgaagacagagggcagaatgaaactgtacagcggcctgcctgccggcctgcaaagacagatcagctccgccagcctgcggatcggcctgtacgacaccgtgcaggagttcctgaccgccggcaaggaaaccgcccctagcctgggctccaagatcctggccggcttgaccaccggaggcgtggccgtgttcatcggccagcctacagaagtggtgaaagtgcggctgcaggcccagagccacctgcacggcatcaagcctagatacaccggcacctacaacgcctaccggatcatcgccacaacagaaggcctgacaggcctgtggaagggcacaacccctaatctgatgagaagcgtgatcatcaactgcaccgagctcgtgacctatgatctgatgaaagaggccttcgtgaagaacaacatcctggccgacgacgtgccatgccacctggtgtccgccctgattgccggcttctgcgccaccgccatgagctctcctgtggacgttgtgaaaacccgctttatcaacagcccccccggccaatacaagagcgtgcccaattgcgccatgaaggtctttaccaacgagggtcctacagctttcttcaagggactggttccatcttttctgagactcggcagctggaacgtgatcatgttcgtgtgcttcgagcagctgaagcgggaactgagcaagtctagacagaccatggactgcgctacctga

SEQ ID NO: 26 - RNA, 924 nt, synthetic construct
  source 1..924: mol_type = other RNA
atgggcggactgaccgcttctgatgtgcatcccaccctgggcgttcaactgttcagcgccggcatcgccgcttgtctggcagatgtgatcacctttccactggacacagccaaggtgcggctgcaggtgcagggcgagtgccccacctcctccgtgattagatacaagggcgttctcggaaccatcacagccgtggtgaagaccgaaggcagaatgaagctgtacagcggcctgcctgccggactgcagagacagatcagcagcgcctctctgcggatcggcctgtatgatacagtgcaggagttcctgacagccggtaaggaaaccgcccctagcctgggatctaagatcctggccggactgaccacaggcggcgtcgccgtgttcatcggccagcctacagaggtggtgaaggtgcggcttcaagcccagagccacctgcacggcatcaagcctagatacaccggcacatacaacgcctacagaatcatcgctaccaccgagggcctgacaggcctgtggaagggcaccacccctaatctgatgagaagcgtgatcatcaactgtacagaactggtgacctacgacctgatgaaagaggcctttgtgaaaaacaacatcctcgctgacgacgtgccctgccacctggtcagcgccctgatcgccggcttctgcgccaccgccatgagctctcctgtggacgtggtcaaaaccagattcatcaatagcccccctggacagtacaagagcgtgcctaactgcgccatgaaagtgttcaccaacgagggccctacagcttttttcaagggcctggtgccaagcttcctgagactgggcagctggaacgtgattatgttcgtgtgcttcgagcagctgaagcgggaactgagcaagtcccgccagaccatggactgcgccacctga

SEQ ID NO: 27 - RNA, 924 nt, synthetic construct
  source 1..924: mol_type = other RNA
atgggcggcctgaccgccagcgacgtgcaccccacactgggcgtgcagctgttcagcgccggcatcgccgcctgcctggctgatgtgattacattccctctggatacagccaaggtgcggctgcaggtgcagggcgagtgccctaccagcagcgtgatccggtacaagggcgtgctgggcaccatcaccgcagtggtcaagaccgagggcagaatgaagctgtacagcggcctccccgccggactgcaaagacagatcagctctgcttctctgagaatcggactctatgataccgtgcaggagttcctgaccgctggcaaggaaaccgccccttccctgggatctaagatcctggccggcctgacaaccggcggagtggccgtgttcatcggccagcctaccgaagtggtgaaggtcaggctgcaggcccagagccatctgcacggcatcaaacccagatacaccggcacctacaacgcctacagaatcatcgccaccaccgaaggcctgacaggcctgtggaagggcaccacacctaatctgatgagaagcgtgatcatcaactgcaccgagctggttacatacgacctgatgaaagaggcctttgtgaagaacaacatcctggccgacgacgtgccctgtcacctggtgtccgccctgatcgctggcttctgcgccacagccatgagctctcctgtggacgtggtgaaaaccagattcatcaacagccctcctggccagtacaagtccgtgccaaattgtgccatgaaagttttcaccaacgagggacctacagcttttttcaagggactggtcccaagcttcctgcggctgggcagctggaacgtgatcatgttcgtgtgctttgagcagctgaagcgggaactgagcaagagcagacaaacaatggactgcgccacctga

SEQ ID NO: 28 - RNA, 924 nt, synthetic construct
  source 1..924: mol_type = other RNA
atgggcggactgaccgctagcgacgtgcaccccacactgggagtgcagctgttcagcgccggcatcgccgcctgcctggctgatgtgatcacattccccctggacaccgccaaggtgcggctgcaagtgcagggcgagtgccctaccagctccgtgatcagatacaagggcgtgctgggcaccatcaccgctgtggtcaagaccgaaggcagaatgaaactgtacagcggcctgcctgccggcctgcagagacagatcagctccgccagcctgcggatcggcctgtatgataccgttcaggagttcctcaccgccggaaaagagacagccccttctctgggctctaagatcctggccggactcaccacaggcggcgtggccgtgttcatcggacagcctacagaagtggtgaaggtgcggcttcaggcccagagccatctgcacggcatcaagcctagatacaccggcacatacaacgcctacagaatcatcgccaccaccgagggcctgacaggcctgtggaagggcaccacccctaatctgatgagaagcgtgattatcaactgcaccgagctggtgacctacgacctgatgaaggaagcttttgtgaaaaacaacatcctggccgatgacgtcccatgtcacctggtcagcgccctgatcgccggcttctgcgccacagctatgagctctccagtggacgtggtgaagaccagattcatcaacagccctcctggccagtacaagtccgtgcccaattgtgccatgaaagtgtttaccaacgagggtcctactgccttcttcaagggactggtgcccagctttctgagactgggcagctggaacgtgatcatgttcgtgtgcttcgagcagctgaagcgggaactgagcaagtctaggcaaacaatggactgcgccacatga

SEQ ID NO: 29 - RNA, 861 nt, synthetic construct
  source 1..861: mol_type = other RNA
atcgccgcttgtctggctgatgtcatcaccttccctctggacaccgccaaggtgcggctgcaggtgcagggcgagtgccccaccagctctgtgattcggtacaagggcgtcctgggcaccatcacagctgttgtgaagaccgagggcagaatgaaactgtacagcggcctgcctgccggactgcaaagacagatctcttctgcttccctgagaatcggcctgtatgataccgtgcaggagttcctcacagccggcaaggagacagcccctagcctgggctccaagatcctggccggcctgaccacaggcggcgtcgccgtgttcatcggccagcctaccgaggtggtgaaggtgcggctgcaggcccagagccatctgcacggcattaagcctagatacaccggcacatacaacgcctacagaatcatcgccacaacagaaggcctgacaggcctgtggaagggcaccacccctaacctgatgagaagcgtgatcatcaattgcaccgaactggtgacctacgacctgatgaaggaagcctttgtgaagaacaacatcctggccgacgacgttccatgtcacctggtgtctgccctgatcgccggattttgcgccacagccatgagctcccccgtggacgtggtgaaaaccagattcatcaacagccctccaggccagtacaaaagcgtgcctaattgcgccatgaaagtgttcaccaacgagggacctaccgcttttttcaagggccttgtgcccagcttcctgaggctgggcagctggaacgtgatcatgttcgtgtgcttcgagcagctgaagcgggaactgagcaagagcagacagaccatggactgcgccacctga

SEQ ID NO: 30 - RNA, 924 nt, synthetic construct
  source 1..924: mol_type = other RNA
atgggcggcctgactgcttctgatgtgcaccccacactgggcgtccaactgttcagcgccggcatcgccgcctgcttggctgacgtgatcacctttcctctggacaccgcaaaggtgcggctgcaggtgcagggcgagtgccctacaagctctgtgatccggtacaagggcgttctgggaaccatcacagctgtggtcaagaccgagggaagaatgaaactgtacagcggcctgcctgccggcctgcaaagacagatcagctccgccagcctgcggatcggcctttatgataccgtgcaggagttcctgaccgccggcaaggaaaccgcccctagcctgggcagcaagatcctggccggcctgacaacaggaggcgtggccgtgttcatcggacagcctaccgaagtggtgaaggtgcgcctgcaggcccagagccacctgcacggcatcaagcctagatacacaggcacatacaacgcttacagaatcatcgccaccacagagggcctgaccggcctgtggaagggcaccacccctaatctgatgagaagcgtgattatcaactgcaccgaactggtgacctacgacctgatgaaagaggcctttgtgaagaacaacatcctggccgacgacgtgccatgtcatctggtgtccgccctgatcgccggattctgcgctaccgccatgagctcccctgtggacgtggtgaaaaccagattcatcaacagcccccccggccagtacaagagcgtgcccaattgcgccatgaaagtgttcaccaacgagggccctaccgccttcttcaagggcctcgttccaagcttcctgagactgggatcttggaacgtgattatgttcgtgtgttttgagcagctgaagcgggaactgtctaagtcaagacagacaatggattgcgccacatga

SEQ ID NO: 31 - RNA, 924 nt, synthetic construct
  source 1..924: mol_type = other RNA
atgggcggcctgaccgctagcgacgtgcatcctacactgggcgtgcagctgttcagcgctggcatcgccgcctgcctggccgatgttatcaccttccccctggataccgccaaggtcagactgcaagtgcagggcgaatgtcctacaagcagcgtgatcagatacaagggcgtgctgggaaccatcacagccgtggtgaaaacagagggcagaatgaagctgtacagcggcttgcctgccggacttcaaagacagatcagctctgcttctctgcggatcggactgtacgatacagtgcaggagttcctgaccgccggcaaggaaaccgcccctagcctgggctccaagatcctggctggactgacaactggaggagtggccgtgttcatcggccagcctaccgaggtggtgaaggtgcggctgcaggcccagagccacctgcacggcatcaagcccagatatacaggcacctacaacgcctaccggattatcgccaccaccgaaggcctgaccggcctgtggaagggcaccacacctaatctgatgaggtccgtgattatcaattgcaccgagctggtgacctacgacctgatgaaagaggcctttgtgaagaacaacatcctggctgacgacgtgccttgccacctggtgtccgccctgatcgccggcttctgcgccacagccatgagctctccagtggacgttgtgaagacaagattcatcaacagcccccccggccagtacaaaagcgtgcctaactgtgccatgaaagtctttaccaacgagggccctaccgccttctttaagggcctcgtgccaagcttcctgagactgggcagctggaacgtgatcatgttcgtgtgcttcgagcagctgaagcgggaactgtctaagagcagacagaccatggactgcgccacctga

SEQ ID NO: 32 - RNA, 924 nt, synthetic construct
  source 1..924: mol_type = other RNA
atgggcggcctgacagccagcgacgtccaccccaccctgggcgtgcagctgttcagcgccggcatcgctgcttgtctggctgatgtgatcacttttccactggataccgcaaaggtgcggctgcaggtgcagggcgagtgccctaccagctctgtgatcagatacaagggcgtgctgggaacaatcaccgccgttgttaagacagaaggcagaatgaagctgtacagcggcctgcctgctggccttcagagacagatcagcagcgcctctctgcggatcggcctgtacgacaccgtgcaggagttcctgaccgccggcaaggaaaccgcccctagcctgggatctaagatcctggccggcctgacaaccggaggagtcgccgtgttcatcggccagcctaccgaggtggtgaaagtgcgcctgcaggcccagagccacctgcacggcatcaagcctagatatacaggcacatacaacgcctacaggatcatcgccaccaccgagggcctgaccggactctggaagggcaccacccctaacctgatgcggtccgtgattatcaattgtaccgagctggtgacctacgacctgatgaaggaagccttcgtgaaaaacaacatcctggccgacgacgtgccctgccatctggtgtccgccctgatcgccggcttctgcgccacagccatgagctctccagtggatgtggtgaaaaccagattcatcaacagcccccccggacaatacaagtccgtgcctaattgcgccatgaaagtgttcaccaacgagggccctacagcttttttcaagggcctggtccctagctttctgagactgggcagctggaacgtgattatgttcgtgtgcttcgagcagctgaagcgggaactgagcaagagcagacaaacaatggactgcgccacatga

SEQ ID NO: 33 - RNA, 924 nt, synthetic construct
  source 1..924: mol_type = other RNA
atgggaggcctgacagcttctgatgtgcaccccaccctgggcgtgcaactgttcagcgccggcatcgccgcttgcctggccgacgtgatcacattccccctggacaccgccaaggtgcggcttcaggtgcagggagaatgtcctaccagcagcgtgatcagatacaagggcgtgctgggcacaatcacagctgtggtcaagaccgaaggcagaatgaaactgtacagcggcctgcctgccggcctgcagagacagatcagctccgccagcctgaggatcggcctgtacgacaccgtgcaggagttcctgaccgcaggcaaggagacagcccctagcctgggcagcaagatcctggccggcctcacaaccggaggagtggccgtgttcatcggccagcctaccgaggtggtgaaggtcagactgcaggcccagtcccacctgcacggcattaagcctagatacaccggcacctataatgcctaccggatcatcgccaccaccgagggactgaccggcctctggaagggcacaacacctaacctgatgcggagcgtgatcatcaactgcaccgagctggttacatacgacctgatgaaggaagcctttgtgaaaaacaacatcctggctgatgatgttccatgtcatctggtgtccgccctgatcgccggcttctgcgccactgccatgagctctcctgtggacgtggtgaaaaccagattcattaacagccctccaggccaatacaagagcgtgcccaattgcgccatgaaggttttcaccaacgagggccccaccgcttttttcaaaggcctggtgccttcttttctgagactgggatcttggaacgtgatcatgttcgtgtgcttcgagcagctgaagcgggaactgtctaagagcagacagaccatggactgcgccacatga

SEQ ID NO: 34 - RNA, 924 nt, synthetic construct
  source 1..924: mol_type = other RNA
atgggcggacttacagccagtgatgtgcatcctaccctgggcgttcaactgttcagcgccggaatcgccgcttgtctggctgatgtgatcaccttccccctggacaccgccaaggtgcggctgcaggtgcagggcgagtgccctacaagcagcgtgattagatacaagggcgtgctgggcaccatcacagccgtggtgaaaaccgaaggtagaatgaagctgtacagcggcctgcctgccggcctgcaaaggcagatcagctctgcctctctgcggatcggcctctatgatacagtgcaggagttcctgaccgctggcaaggaaaccgctccttctctgggcagcaagatcctggccggactgacaaccggcggcgtggccgtgtttatcggacagccaacagaagtggtcaaagtgcggctgcaggcccagagccacctgcacggcatcaagcctagatacaccggcacatacaacgcctacagaatcatcgccaccactgagggcctcaccggcctgtggaagggcaccacacctaatctgatgagaagcgtgattatcaactgtaccgagctggtcacctacgacctgatgaaagaggcctttgtgaagaacaacatcctggccgacgacgtcccctgccacctggtgtccgccctgatcgccggcttctgcgccacagctatgagctcccctgtggacgtggtgaagaccagattcatcaatagcccccccggccagtacaagagcgtgcctaactgcgccatgaaagtgttcaccaacgagggccctaccgcctttttcaagggcctggttccaagcttcctgagactgggatcttggaacgtgatcatgttcgtgtgcttcgagcagctgaagcgggaactgagcaagtccagacagaccatggactgcgccacatga

SEQ ID NO: 35 - RNA, 924 nt, synthetic construct
  source 1..924: mol_type = other RNA
atgggtggcctgacagcctccgacgtgcatcctaccctgggagtgcaactgttcagcgccggcatcgccgcttgtctggctgatgtgattaccttccccctggacacagccaaggtgagactgcaagtgcagggcgagtgccccacaagcagcgtgatccggtacaagggcgtgctgggcaccatcaccgctgtggttaagacagaaggcagaatgaagctgtacagcggcctgcctgccggcctgcagagacagatcagctctgcctctctgcggatcggcctctatgataccgtgcaggagttcctgaccgccggaaaggagacagccccttctctgggaagcaagatcctggccggcctgacaaccggcggagtggccgtcttcatcggccagcctacagaagtggtgaaggtgcggctgcaggcccagagccacctgcacggcatcaagcccagatacaccggaacctacaacgcctacagaatcatcgccaccaccgagggcctcacaggcctgtggaaaggcaccacccctaatctgatgaggtccgtgatcatcaactgcaccgaactggtgacctacgacctgatgaaagaggcctttgtgaaaaacaacatcctggccgacgatgtgccatgccacctggtcagcgccctgatcgctggcttttgcgcaaccgccatgagctccccagtggacgtggtgaagacaagattcattaacagccctcctggccagtacaagagcgtccccaattgcgccatgaaagtgttcaccaacgagggacctacagctttcttcaagggcctggtgcctagcttcctgagactgggcagctggaacgtgatcatgttcgtttgttttgagcagctgaagcgggaactgagcaagtctagacagaccatggactgcgccacctga

SEQ ID NO: 36 - RNA, 924 nt, synthetic construct
  source 1..924: mol_type = other RNA
atgggcggactgaccgcctctgatgtgcaccccaccctgggagtgcagctgttcagcgccggtatcgccgcttgtctggccgacgtgattacattccccctggacaccgcgaaggtgcggctgcaagtgcagggcgagtgccctacaagctctgttattagatataagggcgtgctgggcaccatcaccgccgtggtgaaaaccgagggcagaatgaagctgtacagcggcctgcctgccggactgcagagacagatcagcagcgccagcctgaggatcggcctctacgataccgtgcaggagttcctgaccgccggcaaggagacagcccctagcctgggctccaagatcctcgccggcctgaccacaggcggcgtcgccgtgttcatcggccaacctaccgaagtggtcaaagtgcggctgcaggcccagagccacctgcacggcatcaagcccagatacaccggaacctacaacgcctacagaatcatcgccacaacagaaggcctgacaggcctgtggaagggcaccacacctaacctgatgcggagcgtgatcatcaattgcactgagctggttacatacgacctgatgaaggaagccttcgtgaaaaacaacatcctggctgatgacgtgccttgtcatctggtgtccgccctgatcgccggattttgcgccaccgctatgagctctccagtggacgtggtcaagaccagattcatcaattctcctcctggccagtacaagtccgtgccaaactgcgctatgaaagtgtttaccaacgagggacctaccgcttttttcaagggcctggtgcccagcttcctgagactgggcagctggaacgtgatcatgttcgtgtgcttcgagcagctgaagcgggaactgagcaagagcagacagaccatggactgcgccacctga

SEQ ID NO: 37 - RNA, 924 nt, synthetic construct
  source 1..924: mol_type = other RNA
atgggcggcctgaccgctagcgacgtgcaccccacactgggcgttcagctgttcagcgccggcatcgccgcctgtctggctgatgtgattaccttccccctggacacagccaaggtgcggctgcaagtgcagggcgagtgccctaccagcagcgtgatcagatataagggcgtgctgggcaccatcaccgccgtggtgaagaccgaaggcagaatgaagctgtacagcggcctgcctgccggacttcaaagacagatcagctccgcttctctgcggattggactctacgacaccgtgcaggagttcctgaccgccggaaaagagacagccccttctctgggatctaagatcctggccggcctgacaaccggaggcgtggccgtgtttatcggacagcctacagaagtggtgaaggtgaggctgcaggcccagagccatctgcacggcatcaagcccagatacaccggcacatacaacgcctaccggatcatcgccaccaccgagggcctcacaggcctgtggaagggcaccacccctaatctgatgagatctgtgatcatcaattgcaccgagctggtcacctacgatctgatgaaagaagccttcgtcaagaacaacatcctggcggatgacgttccatgtcacctggtgtccgccctgatcgccggcttctgcgccacagccatgagctcccctgtggacgtcgtgaaaacgagattcatcaacagccctccaggccagtacaagagcgtgcctaactgcgccatgaaagtgttcaccaacgagggtcctaccgcttttttcaagggcctggtgcccagctttctgagactgggcagctggaacgtgatcatgttcgtgtgcttcgagcagctgaagcgggaactgagcaagtctagacagaccatggactgcgctacatga

SEQ ID NO: 38 - RNA, 924 nt, synthetic construct
  source 1..924: mol_type = other RNA
atgggcggactgaccgcctctgatgtccatcctaccctgggcgttcagctctttagcgccggaatcgccgcttgtctggccgatgtcatcaccttccccctggacaccgccaaggtgcggctgcaagtgcagggcgagtgtcctaccagcagcgtgatccggtacaagggcgtgctgggcactatcacagctgtggtgaagaccgagggcagaatgaaactgtacagcggcctgccagccggcctgcagagacagatttccagcgctagcctgagaatcggcctgtatgataccgtgcaggagttcctgaccgcaggcaaggaaaccgcccctagcctgggcagcaagatcctggccggcctgacaaccggcggcgtggccgtgtttattggacagcctacagaggtggtgaaggtgaggctgcaggcccagagccacctgcacggcatcaagcccagatacaccggcacctacaacgcctacagaatcatcgccacaaccgaaggcctgacaggactgtggaagggcaccacacctaatctgatgagaagcgtgatcatcaattgcaccgagctggtgacctacgacctgatgaaggaagcctttgtgaagaacaacatcctggccgacgacgtgccctgccacctggtgtccgccctgatcgctggcttctgcgccacagccatgagctctcctgtggacgtggtcaaaaccagattcatcaacagccctcccggccagtacaaaagcgtcccaaactgcgccatgaaagtgttcaccaacgagggacctacagctttcttcaagggacttgtgccttccttcctgcggctgggcagctggaacgtgatcatgttcgtgtgcttcgagcagctgaagcgggaactgtctaagtctagacaaacaatggactgcgccacctga

SEQ ID NO: 39 - RNA, 924 nt, synthetic construct
  source 1..924: mol_type = other RNA
atgggcgggctcacagcttctgatgtgcatcctacactgggcgtccagctgttcagcgccggaatcgccgcttgtctggccgatgtgatcaccttccccctggacaccgccaaggtgcggctgcaggtgcagggcgagtgccctaccagctctgtgatccgctacaagggcgtgctgggcaccatcacagctgtggtgaagaccgaaggcagaatgaaactgtacagcggcctgcctgccggcctgcaaagacaaatcagcagcgccagcctgagaatcggactgtatgatacagtgcaggagttcctgactgctggcaaggagacagcccctagcctgggaagcaagatccttgccggactgaccaccggcggcgtggccgtgtttatcggccagcctaccgaagtggttaaggtgcggctgcaggcccagagccacctgcacggcatcaagcctagatacaccggcacctacaacgcttacagaattatcgccacaaccgaaggtctgacaggactgtggaagggcaccacccctaacctgatgcggagcgtcatcattaactgcaccgagctggttacatacgacctgatgaaagaggcctttgtgaagaacaacatcctggccgacgacgtcccatgtcacctggtgtccgccctgatcgccggcttctgcgccacagccatgagctcccctgtggacgtggtgaagacaagattcatcaacagcccccccggccagtacaagtctgtgccaaattgcgccatgaaagtgttcaccaacgagggccctaccgcttttttcaagggcctggtgccctctttcctcagactgggcagctggaatgtgatcatgttcgtgtgcttcgagcagctgaagcgggaactgagcaaatccagacagaccatggactgcgccacctga

SEQ ID NO: 40 - RNA, 924 nt, synthetic construct
  source 1..924: mol_type = other RNA
atgggcggcctgaccgccagcgacgtgcatcctacactaggcgtgcagctgttcagcgccggcatcgccgcctgcctggctgatgtgattaccttccccctggacaccgccaaggtgcggctgcaagtgcagggcgagtgtcctaccagcagcgtgatcagatacaagggagtgctgggaacaatcaccgccgtggtgaaaaccgagggaagaatgaagctgtacagcggcctccccgccggcctgcagagacagatcagctctgcttctctgcggatcggcctgtatgataccgtgcaggagttcctgacagctggcaaggagacagcccctagcctgggcagcaagatcctggccggactcacaaccggcggagtggccgtgtttattggccagcctacagaagtggtcaaagtgcggctgcaggcccagagccacctgcacggcatcaagcccagatacaccggcacctacaacgcctacagaatcatcgccacaacagaaggcctgaccggactgtggaagggcaccacccctaatctgatgaggtccgtgatcatcaactgcaccgaactggtcacctacgacctgatgaaagaggcctttgtcaagaacaacatcctggccgatgacgtgccttgtcacctggtgtccgccctgatcgccggcttctgcgccacagctatgagctctcctgtggacgtggttaagaccagattcatcaacagccctccaggccaatacaagtccgtgccaaattgcgccatgaaggtgttcaccaacgagggtcctaccgcttttttcaagggcctggtgcccagcttcctgagactgggctcttggaacgtgatcatgttcgtgtgcttcgagcagctgaagcgggaactgagcaaaagcagacagaccatggactgcgctacatga

SEQ ID NO: 41 - RNA, 924 nt, synthetic construct
  source 1..924: mol_type = other RNA
atgggcggcctgaccgcttccgatgtgcaccccaccctgggcgtgcagctgttcagcgccggcatcgccgcttgtctggccgacgtgatcacttttccactggacacagccaaggtgcggctgcaagtgcagggcgagtgccccaccagctccgtgatccggtacaaaggcgtgctgggcaccatcaccgccgtggtgaagacagaaggcagaatgaaactgtacagcggcctgcctgccggcctgcagaggcagatcagcagcgcctctctgcggatcggactgtacgacaccgtgcaggagttcctgacggccggaaaagagacagcccctagcctcggcagcaagatcctggccggcctcaccaccggcggtgtggccgtgttcattggacaacctaccgaagtggtgaaggtcagactgcaggcccagagccacctgcatggcatcaagcctagatacaccggaacatacaacgcctacagaatcattgccaccaccgagggactgacaggcctgtggaagggcaccacacctaatctgatgagaagcgtgatcatcaattgtaccgagctggttacatatgacctgatgaaggaagccttcgtgaaaaacaacatccttgctgatgatgtgccctgccacctggtgtccgccctgatcgccggcttctgcgccaccgcaatgagcagccctgtggacgtcgtgaagaccagattcatcaacagcccccctggccagtacaagagcgtgcctaactgcgccatgaaggtgttcaccaacgagggccctacagcttttttcaagggcctggtcccatcttttctgagactgggatcttggaacgtgatcatgttcgtgtgcttcgagcagctgaagcgggaactgagcaagtctagacagaccatggactgcgctacatga

SEQ ID NO: 42 - RNA, 924 nt, synthetic construct
  source 1..924: mol_type = other RNA
atgggcggcctgaccgctagcgacgtgcatcctaccctgggagtgcagctgttcagtgccggcatcgctgcttgtctggctgatgtgatcaccttccccctggacaccgcaaaggtgcggctgcaagtgcagggagaatgtcctacaagctccgtgattagatataagggcgtgctgggtaccattacagccgtggtcaaaaccgaaggcagaatgaaactgtacagcggcctccctgccggcctgcagcgccagatcagcagcgccagcctgcggatcggactgtacgataccgtgcaggagttcctgaccgccggcaaggaaaccgcccctagcctgggctccaagatcctggccggcctgacaacaggcggagtggccgtctttatcggccagcctacagaggtggtgaaggttagactgcaggcccagagccacctgcacggaatcaagcccagatacaccggcacctacaacgcctacagaatcatcgccaccacagagggcctgacaggcctgtggaagggcacaacccctaatctgatgcggagcgtgatcatcaactgcaccgagctggtgacctacgatctgatgaaagaggcctttgtgaagaacaacatcctggccgacgacgtgccgtgccacctggtgtctgccctgatcgccggcttctgcgccaccgccatgagctctccagtggacgtggtgaaaaccagattcatcaacagccctccaggccaatacaagtccgtgcccaattgcgccatgaaggtgttcaccaacgagggccctacagcttttttcaagggcctggtccccagcttcctgagactgggatcttggaacgtgatcatgttcgtgtgcttcgagcagctcaagcgggaactgtctaagagcagacagaccatggactgcgccacatga

SEQ ID NO: 43 - RNA, 924 nt, synthetic construct
  source 1..924: mol_type = other RNA
atgggaggcctgaccgcatctgatgtgcaccctacactgggagtgcagctgttcagcgccggaatcgccgcttgtctggccgatgtgattaccttccccctggacaccgccaaggtgcgcctgcaggtgcagggcgagtgccctaccagctctgtgatcagatacaaaggcgtgctgggcaccatcaccgctgtggtcaagacagagggcagaatgaaactgtacagcggcctccccgccggcctgcaaagacagatcagcagcgccagcctgagaatcggcctgtatgacaccgtgcaggagttcctgacagctggcaaggaaaccgcccctagcctgggcagcaagatcctggccggtcttacaaccggcggcgtggccgtgttcatcggccaacctacagaagtggtgaaggtgcggctgcaggcccagagccacctgcatggcatcaagcctagatacactggaacatacaacgcctaccggatcatcgccaccaccgagggcctgacaggcctctggaagggcaccacccctaatctgatgagatccgtgatcatcaactgcaccgagctggtcacctacgacctgatgaaagaagcctttgtgaagaacaacatcctggctgatgacgttccatgccacctggtgtctgccctgatcgccggcttctgcgccacagccatgagctcccctgtggacgtggtgaagaccagattcatcaatagccctccaggacagtacaagtccgtccctaactgcgccatgaaagtgttcaccaacgagggccccacagcttttttcaagggcctggtgcccagcttcctgcggctgggatcttggaacgtgattatgttcgtttgttttgagcagctgaagcgggaactgagcaagagcagacagaccatggactgcgccacctga

SEQ ID NO: 44 - RNA, 924 nt, synthetic construct
  source 1..924: mol_type = other RNA
atgggcggactgaccgcttctgatgtgcatcccacactgggcgtgcaactgttcagcgccggaatcgccgcatgcctggctgatgttattaccttccctctggataccgccaaggtgcggctgcaagtgcagggcgagtgtcctaccagcagcgtgatcagatacaagggcgtgctgggaaccatcaccgccgtggtgaagacagagggcagaatgaaactgtattctggcctgcctgccggcctgcagagacagatctccagcgccagcctgcggatcggcctgtacgacaccgtgcaggagttcctgacagctggcaaggaaaccgcccctagcctgggctctaagatcctggctggcctgaccacaggcggcgtggccgtttttattggccagcctaccgaggtggtgaaggtgcgcctccaggcccagagccacctgcacggcatcaagcccagatacaccggcacatacaacgcctacagaatcatcgccacaacagagggcctgaccggactgtggaagggaacaacccctaacctgatgcggagcgtgatcatcaattgcaccgaactggtgacctacgacctgatgaaagaagccttcgtgaaaaacaacatcctggccgacgacgtgccatgccacctggtgtccgccctgatcgccggcttctgcgccacagccatgagcagccctgtggacgtggtcaagaccagattcatcaacagcccccccggccagtacaagtccgtgcctaattgtgccatgaaagtgttcaccaacgagggtcctacagcttttttcaagggccttgtgccatcttttctgagactgggcagctggaacgtgatcatgttcgtctgcttcgagcagctgaagcgggaactgagcaagagcagacagaccatggactgcgccacctga

SEQ ID NO: 45 - RNA, 924 nt, synthetic construct
  source 1..924: mol_type = other RNA
atgggaggcctgaccgcttctgatgtgcatcccaccctgggcgtccagctgttcagcgccggcatcgccgcttgtctggctgatgtgattacattccccctggacaccgccaaggtgcggctgcaggtgcagggcgagtgccccaccagcagcgtgatcagatataagggcgttctgggcaccatcaccgccgtggtcaagaccgaaggcagaatgaaactgtacagcggcctgcctgccggcctgcaaagacagatcagcagcgccagcctcaggatcggcctgtacgacacagtgcaggagttcctgacagctggcaaggaaaccgccccttctctgggcagcaagatcctggccggactgaccaccggcggcgtggccgtctttatcggacagcctacagaagtggtgaaggtgcggctgcaggcccagagccacctgcacggcattaagcctagatacaccggcacatacaacgcctacagaatcatcgccaccacagagggcctgacaggcctgtggaagggcaccacccctaatctgatgcggagcgtgatcatcaattgtaccgagctggtgacctacgatctgatgaaagaggccttcgtgaaaaacaacatcctggccgacgacgtgccctgccacctcgtgtccgccctgatcgccggcttctgcgccacagccatgagctctcctgtggacgtcgtgaagaccagattcatcaacagccctcctggacagtacaagtccgtgccaaactgcgccatgaaagtgttcaccaacgagggtcctaccgcttttttcaagggcctggtgccatcttttctgagactgggatcctggaacgtgatcatgttcgtgtgcttcgagcagctcaagcgggaactgagcaagagcagacaaacaatggactgcgctacatga

SEQ ID NO: 46 - RNA, 924 nt, synthetic construct
  source 1..924: mol_type = other RNA
atgggcggccttacagctagcgacgtgcaccctaccctgggagtgcagctgttcagcgccggaatcgccgcctgcctggctgatgtgatcacattccccctggacaccgccaaggtgcggctgcaagtgcagggcgagtgccccaccagcagcgtgatccggtacaaaggcgtgctgggaaccatcaccgccgtggtgaagacagaaggaagaatgaaactgtactcaggcctgcctgccggcctgcaacgccagatcagctctgcttctctgagaatcggactgtatgataccgtgcaggagttcctgaccgctggcaaggaaaccgcccctagcctgggcagcaagatcctggccggcctgacaacaggtggcgtcgccgtgttcatcggccagcctactgaggtggtgaaggttagactgcaggcccagagccatctgcacggcatcaagcccagatacaccggcacctacaacgcctacagaatcatcgccacaaccgagggcctgaccggcctctggaagggcacaacccctaatctgatgagaagcgtgatcatcaactgcaccgagctggtgacctacgatctgatgaaggaagcctttgtgaagaacaacatcctggccgacgacgtgccttgccacctcgtgtccgccctgattgccggcttctgcgccaccgccatgagcagccctgtggacgtggtgaaaaccagattcatcaacagcccacctggccagtacaagtccgtgcccaactgcgccatgaaggtgttcaccaatgagggacctacagcattcttcaagggcctggtcccatcttttctgcggctgggctcctggaacgtgatcatgttcgtttgttttgagcagctgaaacgggaactgagcaagtctagacagaccatggactgtgctacatga

SEQ ID NO: 47 - RNA, 924 nt, synthetic construct
  source 1..924: mol_type = other RNA
atgggcggcctgaccgcttctgatgtgcaccccacactgggagtccagctgttcagcgccggcatcgccgcttgtctggccgatgtgattacttttccactggacaccgccaaggtgcggctgcaggtgcaaggcgagtgccctaccagcagcgtgatcagatacaagggcgtgctgggcaccatcaccgctgttgtgaagaccgaaggcagaatgaaactgtacagcggcctgcctgccggactgcaaagacagatcagctccgccagcctgcgcatcggactgtacgataccgtgcaggagttcctgacagccggcaaggagacagcccctagcctgggctctaagatcctggctggcctgaccacaggcggagtggccgtcttcatcggccagcctaccgaggtggtcaaagtgcggctgcaggcccagagccacctgcatggcatcaagccccggtatacaggcacctacaacgcctacagaatcattgccacaacagagggcctgaccggcctgtggaagggaaccacccccaatctgatgagaagcgtgatcatcaactgcaccgaactggtgacctacgacctgatgaaggaagccttcgtgaaaaacaacatcctcgccgacgacgtgccttgtcacctggtgtccgccctgatcgccggcttctgcgccaccgccatgagctcccctgtggacgtggtgaagacaagattcatcaacagccctcctggccagtacaagagcgtgccaaattgcgccatgaaggtgtttaccaacgagggccctacagcttttttcaagggccttgtgcccagcttcctgagactcggatcttggaacgtgatcatgttcgtctgcttcgagcagctgaagcgggaactgtctaaaagcagacagaccatggactgcgcaacatga

SEQ ID NO: 48 - RNA, 924 nt, synthetic construct
  source 1..924: mol_type = other RNA
atgggcggactcaccgccagcgacgtgcacccaaccctgggcgtgcagctgttcagcgccggaatcgccgcttgtctggccgacgtgattaccttccctctggatacagccaaggtgcggctgcaggtgcagggcgagtgccccaccagcagcgtcatccggtataagggcgttctgggcaccatcacagctgtggtgaagaccgaaggtagaatgaagctgtacagcggcctccctgccggcctgcaaagacagatcagctctgcttctctgcggatcggactgtacgacaccgtccaggagttcctgacagctggcaaggagacagccccttctctgggatccaagatcctggccggcctgacaacaggcggcgtggccgtctttattggccagcctaccgaagtggtgaaggtgagactgcaggcccagtcccacctgcatggcatcaagcctagatacaccggcacttacaacgcctacagaatcatcgcaaccaccgaaggcctgaccggcctgtggaagggaacaacccctaacctgatgagaagcgtgatcatcaattgcaccgagctggtgacctacgacctgatgaaagaggcctttgtgaaaaacaacatcctggctgatgatgtgccctgccaccttgtgtccgccctgatcgccggattttgtgccaccgccatgagctctcctgtggacgtggtgaaaaccagattcatcaacagcccccccggccagtacaagagcgtgcctaactgcgccatgaaagtgttcaccaacgagggccctacagccttcttcaagggcctggtgccaagcttcctgaggctgggcagctggaatgtgatcatgttcgtgtgcttcgagcagctgaagcgggaactgagcaagtcaagacaaacaatggactgcgccacctga

SEQ ID NO: 49 - RNA, 924 nt, synthetic construct
  source 1..924: mol_type = other RNA
atgggcggcctcaccgcctctgatgtgcaccctacactgggcgtgcagctgttcagcgccggaatcgccgcttgtctggctgatgtgattaccttccccctggacacagcaaaggtgcggctgcaagtgcaaggcgagtgccctaccagctccgtgatcagatacaaaggcgtcctgggcaccatcaccgctgtggtcaagaccgaaggcagaatgaagctgtacagcggcctgcctgctggcctgcagagacagatcagcagcgccagcctgagaatcggcctgtatgataccgtgcaggagttcctgaccgccggcaaggaaaccgccccttctctgggaagcaagatcctggccggactgacaaccggcggagtggccgtgtttatcggacagcctacagaagtggtgaaagtgcggctgcaggcccagagccacctgcacggcatcaagcccagatacaccggcacctacaacgcctacagaatcatcgccaccacagagggcctgaccggcctgtggaaggggacaacccctaatctgatgcggagcgtgatcatcaactgcactgagctggtcacatacgacctgatgaaagaggcctttgtgaagaacaacatcctggccgacgacgtgccatgtcatctcgtgtccgccctgattgccggcttctgcgccaccgccatgagcagccctgtggacgtcgtgaagacaagattcatcaacagcccccccggccagtacaagtccgtgccaaattgcgccatgaaagtgttcaccaacgagggccctacagcttttttcaagggcctggttcctagcttcctgaggctgggatcttggaacgtgatcatgttcgtgtgcttcgagcagctgaagcgggaactgtctaagtcaagacagaccatggactgcgccacatga

SEQ ID NO: 50 - RNA, 924 nt, synthetic construct
  source 1..924: mol_type = other RNA
atgggcggcttgacagctagcgacgtgcacccaaccctgggcgtccagctgttcagcgctggaatcgccgcttgtctggccgacgtgatcacatttcctctggacaccgccaaggttagactgcaagtgcagggcgagtgccctaccagctccgtgatcagatacaagggcgtgctgggaaccatcaccgccgtggtcaagaccgaaggtagaatgaagctgtacagcggcctgcctgccggccttcagcggcagatcagctctgcctctctgcgcatcggcctgtacgataccgtgcaggagtttctgaccgcaggcaaggaaacagcccctagcctgggcagcaagattctggccggactgactacaggcggagtggccgtgttcatcgggcagcccacagaggtggtgaaggtgcggctgcaggcccagagccacctgcatggcatcaagcccagatacacaggcacatacaacgcctacagaatcattgctacaaccgagggcctgaccggcctgtggaagggcaccacacctaatctgatgcggtccgtgatcatcaactgcaccgaactggtgacctatgatctgatgaaagaggcctttgtgaagaacaacatcctcgccgatgacgtcccttgccacctggtgtccgccctgatcgccggcttctgcgccaccgccatgagcagccctgtggacgtggtgaaaaccagattcatcaatagcccccccggccaatacaaaagcgtgcctaactgtgccatgaaagtgttcaccaacgagggccctaccgctttcttcaagggcctggtgccaagtttcctgagactgggatcttggaacgtgatcatgttcgtgtgcttcgagcagctgaagcgggaactgagcaagtctagacagaccatggactgcgccacatga

SEQ ID NO: 51 - RNA, 924 nt, synthetic construct
  source 1..924: mol_type = other RNA
atgggcggactgacagcctctgatgtgcaccccacactgggcgtccagctgttcagcgccggcatcgccgcttgtctggccgacgtgatcaccttcccactggacaccgcaaaggtgcggctgcaggtgcaaggcgagtgtcctaccagcagcgtgatccgctacaagggcgtgctgggcaccatcaccgccgttgtgaagaccgaaggcagaatgaagctgtacagcggccttcctgccggactgcaaagacagatcagctccgcttctctgcggatcggactgtatgatacagtgcaggagttcctgacagctgggaaagagacagcccctagcctgggcagcaagatcctggctggactgaccaccggcggcgtggccgtgtttatcggccagcctaccgaggtggtgaaggtgagactgcaggcccagagccacctgcacggcatcaagcctagatacaccggcacatacaacgcctacagaatcatcgccacaacagaaggcctgaccggcctgtggaagggcaccacacctaatctgatgcggagcgtgattatcaattgcaccgagctggtgacctacgacctgatgaaagaagccttcgtgaagaacaacatcctggccgatgacgttccatgccatctggtgtccgccctgatcgccggcttctgcgccaccgccatgtccagccccgtggacgtggtgaaaaccagattcatcaacagccctcctggacagtacaagtccgtgcctaactgcgccatgaaggtgttcaccaacgagggccccaccgctttttttaagggcctggtccccagcttcctgagactcggatcttggaacgtgattatgttcgtgtgcttcgagcagctgaagcgggaattgtctaaaagcagacagaccatggactgcgccacatga

SEQ ID NO: 52 - RNA, 924 nt, synthetic construct
  source 1..924: mol_type = other RNA
atgggaggcctgaccgctagcgacgtgcaccccacactgggcgttcagctgttcagcgccggcatcgccgcttgtctggctgatgttattaccttccccctggacaccgccaaggtgagactgcaagtgcagggcgaatgtcctaccagctctgtgatccggtacaagggcgtgctgggcacaatcaccgcagtggtcaagacagaaggcagaatgaagctgtacagcggcctgcctgccggcctgcagagacaaatcagctctgcctccctgagaatcggcctgtatgataccgtgcaggagttcctgaccgccggaaaagagacagcccctagcctgggcagcaagatcctggccggacttaccacaggcggcgtggccgtgttcattggacagcctaccgaggtggtgaaagtgcggctgcaggcccagagccacctgcacggcatcaagcctagatacaccggaacttacaacgcctacagaatcatcgccaccaccgaaggcctgaccggcctgtggaagggtacaacccctaatctgatgcggagcgtgatcatcaactgcaccgagctggtgacctacgatctgatgaaagaggcctttgtcaagaacaacatcctggccgacgacgtgccttgccatctggtgtccgccctgatcgccggcttctgcgccacagctatgagctcccctgtggacgtggtgaagaccagattcatcaacagcccccccggccagtacaagagcgtgccaaattgcgccatgaaagtctttaccaacgagggccctacagcttttttcaagggactcgtgccatcattcctgcggctgggctcttggaacgtgatcatgttcgtgtgcttcgagcagctgaagagggaactgagcaagtctagacagaccatggactgcgccacatga

SEQ ID NO: 53 - RNA, 924 nt, synthetic construct
  source 1..924: mol_type = other RNA
atgggcggactgacagctagcgacgtgcaccctaccctgggcgtccagctgttcagcgccggaatcgccgcttgtctggccgatgtgattacattccccctggataccgccaaggtgcggctgcaagtgcagggcgagtgccctacctccagcgtgatccggtacaagggagtgctgggaacaatcaccgccgtggtgaagaccgaaggcagaatgaaactgtacagcggcctccccgccggccttcaaagacagatcagctctgccagcctgcggatcggcctgtacgacacagtgcaggagttcctgaccgccggcaaggaaaccgccccttctctgggttctaagatcctggctggcctgaccaccggcggcgtggccgtgtttattggacagcctaccgaggtggtgaaggtgcggctgcaggcccagagccatctgcacggcatcaagcccagatacaccggcacctacaacgcctacagaatcatcgctaccaccgagggcctgaccggcctgtggaagggcacaacacctaatctgatgagaagcgtgatcatcaactgtacagaactggtgacttatgatctgatgaaagaagcctttgtgaagaacaacatcctggccgacgacgtgccatgccacctggtgtccgccctgatcgccggcttctgcgccaccgccatgagcagccccgtggacgtggtcaaaaccagattcatcaacagccctcctggccagtacaagtccgttccaaattgcgccatgaaagtgttcaccaacgagggccctacagcttttttcaagggcctggtccctagcttcctgagactcggatcttggaacgtgatcatgttcgtgtgcttcgagcagctgaagagggagctgagcaagagcagacagaccatggactgcgcaacatga

SEQ ID NO: 54 - RNA, 861 nt, synthetic construct
  source 1..861: mol_type = other RNA
atcgctgcttgtctggccgacgtgattacattccctctggacaccgccaaggtgcggctccaggtgcaaggcgaatgtcctaccagctccgtgatccggtacaaaggcgtcctgggaaccatcaccgccgtggtgaaaaccgagggcagaatgaaactgtacagcggcctgcctgccggactgcaaagacagatcagcagcgccagcctgcggatcggcctttatgataccgtgcaggagttcctgaccgccggcaaggagactgcccctagcctgggctctaagatcctggctggcctgacaaccggcggcgtcgccgtgttcatcggacagcctaccgaggtggtgaaggttagactgcaggcccagagccacctgcacggcatcaagccaagatacaccggcacctacaacgcctacagaatcatcgccacaaccgagggcctcacagggctgtggaagggcaccacacctaatctgatgagaagcgtgatcatcaattgcaccgaactggttacatacgacctgatgaaggaagcctttgtgaagaacaacatcctggcggatgacgtgccctgccatctggtgtccgccctgatcgccggcttctgcgccacagctatgagctcccctgtggacgtggtgaagaccagattcattaacagcccccccggccagtacaagagcgtgccaaactgcgccatgaaagtgttcaccaacgagggtcctacagcttttttcaagggcctggtgcctagctttctgaggctgggctcttggaacgtgatcatgttcgtctgcttcgagcagctgaagcgggaactgagcaagtctagacagaccatggactgcgccacatga

SEQ ID NO: 55 - RNA, 924 nt, synthetic construct
  source 1..924: mol_type = other RNA
atgggcggactgacagccagcgacgtgcaccccaccctgggagtgcagctgttcagcgccggcatcgccgcttgcctggctgatgtgatcacttttccactggacaccgcaaaggtgagactgcaggtgcagggcgaatgtcctacaagcagcgtgattagatacaagggcgtgctgggcacaatcacggctgtggtcaagaccgagggcagaatgaagctgtacagcggcttgcctgccggcctccagaggcagatcagctccgcctctctgcggatcggcctctacgacaccgtgcaggagttcctgacagccgggaaggagacagccccttctctgggcagcaagatcctggccggactgaccaccggcggcgtggccgtgttcattggacagcccaccgaagtggtgaaagttcggctgcaagcccagagccatctgcacggcatcaagcccagatacaccggcacctacaacgcctacagaatcatcgccacaacagaaggcctgaccggcctgtggaagggcaccacccctaacctgatgcggtccgtgatcatcaattgcaccgagctggtcacctatgatctgatgaaagaggccttcgtgaagaacaacatcctggccgacgacgtcccatgccacctggtgtccgccctgatcgccggcttctgcgctacagccatgagctctcctgttgatgtggtgaaaaccagattcatcaacagccctcctggccagtacaaaagcgtgcctaattgtgccatgaaggtgttcaccaacgagggacctaccgcctttttcaagggcctggtgcccagctttctgagactgggatcttggaacgtgatcatgttcgtgtgcttcgagcagctgaagcgggaactgagcaagagcagacaaacaatggactgcgctacctga

SEQ ID NO: 56 - RNA, 924 nt, synthetic construct
  source 1..924: mol_type = other RNA
atgggcggactgaccgcctctgatgtgcaccctaccctgggcgtgcaactgttcagcgccggcatcgccgcttgtctggccgacgtgattacattccctctggacaccgccaaggtgcggctgcaagtgcagggcgaatgccctacctccagcgttatccggtacaagggagttctgggcaccatcacagctgtggtgaaaaccgagggcagaatgaagctgtacagcggcctccccgccggactgcagagacagatcagcagcgcctctctgaggatcggcctatatgacaccgtgcaggagttcctgacagccggcaaggaaacagcccctagcctgggcagcaagatcctggccggcctgaccaccggcggcgtggccgtctttatcggccagcctacagaggtggtgaaagtgcggctgcaggcccagagccacctgcacggcattaagcctagatacaccggcacctacaacgcctacagaatcatcgccacaaccgaaggcctgacaggcctgtggaagggcacaacacctaacctgatgcggagcgtgatcatcaattgcaccgaactggtcacctacgacctgatgaaagaggcctttgtgaagaacaacatcctggctgatgatgtgccatgtcatctggtctccgccctgatcgccggtttctgcgccaccgctatgagctcccctgtggacgtggtgaagaccagattcatcaacagcccccccggccagtacaagtctgtgccaaattgcgctatgaaagtgttcaccaacgagggacctacagcttttttcaagggcctggtgcccagcttcctcagactgggatcttggaacgtgatcatgttcgtgtgcttcgagcagctgaagagagagctgagcaagagcagacagaccatggactgcgccacctga

SEQ ID NO: 57 - RNA, 924 nt, synthetic construct
  source 1..924: mol_type = other RNA
atgggaggactgaccgccagcgacgtgcaccctaccctgggagtgcagctgttcagcgccggaatcgccgcctgcctggccgatgtcatcaccttccccctggatacagctaaggtgcggctgcaggtgcagggcgagtgccccacaagcagcgtgattagatacaagggcgtgctgggcacaatcacagctgtggtgaagaccgagggcagaatgaaactgtacagcggcctgcctgccggcctgcaaagacagatcagcagcgcctctctgcggatcggcctctacgacaccgtgcaggagttcctgaccgccggcaaggaaaccgcccctagcctgggctccaagatcctggctggactgaccaccggcggcgtggccgtgttcatcggccaacctacagaggtggtcaaagtgcggctgcaggcccagagccacctgcatggcatcaagcctagatacaccggcacctacaacgcctacagaatcatcgccaccacagaaggcctgaccggcctgtggaagggcacgactcctaatctgatgcggagcgtgatcatcaactgcaccgaactggtgacctatgacctgatgaaagaggcctttgtgaaaaacaacatccttgctgatgacgttccatgtcacctggtgtccgccctgatcgccggcttctgcgccacagccatgtcttctcctgtggacgtggtgaagacaagatttatcaacagccctccaggccagtacaagagcgtgcccaattgcgccatgaaggtgttcaccaacgaaggccctaccgcttttttcaagggactggtccccagcttcctgagactgggttcttggaacgtgattatgttcgtgtgcttcgagcagctgaagcgcgagctgagcaagtccagacagaccatggactgtgccacatga

SEQ ID NO: 58 - RNA, 924 nt, synthetic construct
  source 1..924: mol_type = other RNA
atgggcggactgaccgcttctgatgtgcaccctaccctgggcgtgcagctgttctccgccggcatcgccgcttgtctggccgacgtgattacttttccactggacaccgcaaaggtcagactgcaggtgcagggcgagtgccccacaagcagcgtgatcagatataagggcgtgctgggaaccatcaccgccgtggtgaagaccgagggcagaatgaagctgtacagcggcctccccgccggcctgcagagacagatcagctctgccagcctgcggatcggactctacgatacagtgcaggagttcctgaccgctggcaaggaaaccgcccctagcctgggttctaagatcctggccggactgaccaccggaggagtggccgtgttcatcggccaacctaccgaggtggtcaaggtgcggctgcaagcccagagccatctgcacggcatcaagcctagatacaccggcacatacaacgcctacagaattatcgccaccacagaaggcctgacaggcctgtggaagggcaccacccctaatctgatgcggagcgttatcatcaactgcaccgaactggtgacctacgacctgatgaaagaggccttcgtgaagaacaacatcctggctgatgacgtgccctgccacctggtgtccgccctgatcgccggcttctgcgccacagccatgagcagccctgtggacgtggtgaaaacaagattcatcaacagccctcctggccagtacaagagcgtgccaaactgtgccatgaaagtctttaccaacgagggccctacagcttttttcaagggcctagtgccctccttcctgaggctgggctcttggaatgtgatcatgttcgtgtgcttcgagcagctgaagcgggaactgagcaaaagcagacagacaatggactgcgccacatga

SEQ ID NO: 59 - RNA, 924 nt, synthetic construct
  source 1..924: mol_type = other RNA
atgggcggcctgaccgccagtgatgtgcaccccaccctgggtgttcaactgttctctgccggaatcgccgcctgcctggccgacgtgatcacctttccactggacaccgccaaggtgaggctgcaggtgcagggcgagtgtcctacaagcagcgtgatcagatacaagggcgtgctgggcaccatcaccgctgtggtcaagacagagggcagaatgaaactgtacagcggcctgcctgccggcctccagcggcagatcagctccgcctctcttcggatcggcctctacgacaccgtgcaggagttcctgacagctggcaaggaaaccgccccttctctgggatctaagatcctggctggcctgaccacaggaggagtggccgtgtttatcggccagcctaccgaagtggtgaaggtgcggctgcaggcccagagccatctgcacggcatcaagcctagatatacaggcacctacaacgcctacagaatcatcgcgacaaccgagggcctgaccggcctgtggaagggcacaacacctaatctgatgagaagcgtgattattaactgcaccgagctggttacatacgatctgatgaaagaagccttcgtgaagaacaacatcctggccgatgacgtgccttgtcacctggtgtccgctctgatcgccggcttctgcgccacggccatgagctcccctgtggacgtggtcaaaaccagattcatcaacagcccccccggccagtacaagagcgtgcccaattgcgccatgaaagtctttaccaacgagggacctaccgctttcttcaagggcctggtgccaagcttcctgagactgggcagctggaacgtgatcatgttcgtgtgcttcgagcagctgaagcgggaactgagcaagagcagacaaacaatggactgcgccacctga

SEQ ID NO: 60 - RNA, 924 nt, synthetic construct
  source 1..924: mol_type = other RNA
atgggcggcctgacagccagtgatgtgcaccccaccctgggcgtgcaactgttcagcgccggcatcgcagcctgcctggccgacgtgatcacatttccattggacacagccaaggtgagactgcaggtgcagggcgagtgccccacaagcagcgtgattcggtacaagggcgtgctgggcaccatcaccgccgtggtcaagaccgaaggcaggatgaaactgtacagcggcctgcctgccggcctgcagagacagatcagctctgctagcctgagaatcggactctacgacaccgtccaggagttcctgactgctggcaaggaaaccgccccttctctgggaagcaagatcctggccggactcactaccggcggagtggccgtgttcatcggccagcctacagaggtggtgaaagtgcggctgcaagcccagagccatctgcacggcatcaagcctagatacaccggcacctacaacgcctacagaatcatcgccaccacagaaggcctgaccggcctgtggaagggcacaacccctaatctgatgcggtctgtgatcatcaattgcacagagctggtgacctatgacctgatgaaagaagccttcgtgaagaacaacatcctggctgatgatgtgccctgtcacctggtctccgccctgatcgccggcttctgcgccaccgccatgtccagccctgtggacgtggtgaagacaagattcatcaacagcccacctggccagtacaagtctgttcccaactgtgctatgaaagtgtttaccaacgagggacctaccgcttttttcaagggactggtgcctagcttcctgcggctgggctcctggaacgtgatcatgttcgtgtgcttcgagcagctgaagagagagctgagcaagagcagacagaccatggactgcgccacatga

SEQ ID NO: 61 - RNA, 924 nt, synthetic construct
  source 1..924: mol_type = other RNA
atgggcggcctgaccgcctctgatgtgcaccctaccctgggcgtgcagctgttcagcgccggaatcgccgcctgtctggccgacgtgatcacctttccactggacaccgctaaagtgcggctgcaggtgcaaggcgagtgccctacaagctctgtgatcagatacaagggagtgctgggcaccatcacagccgtggtgaagaccgaaggcagaatgaaactgtacagcggcctgcctgctggactgcaaagacagatcagctctgctagcctgaggatcggactttatgataccgtgcaggagttcctgacagccggcaaagagacagcccctagcctgggctccaagatcctggccggcctgaccacaggaggcgtcgccgtgttcatcggccagcctacagaagtggtcaaggtgcggctacaggcccagagccacctgcacggcatcaagcccagatacaccggcacctacaacgcctaccggatcatcgccacaaccgagggcctgaccggcctctggaagggcacaacccctaacctgatgcgcagcgtgatcatcaattgcaccgaactggtcacctacgacctgatgaaagaagccttcgtgaagaacaacatcctggctgatgacgtgccctgccatctggtgtccgccctgatcgccggcttctgcgccaccgccatgagcagccccgtggacgtggttaagacaagattcattaactcccctccaggccagtacaagagcgtgcctaattgcgccatgaaggtgttcaccaacgagggacctacagcttttttcaagggcctggtgcccagcttcctgagactgggcagctggaacgtgattatgttcgtgtgttttgagcagctgaagcgggagctgtctaagtccagacagaccatggactgcgcaacatga

SEQ ID NO: 62 - RNA, 924 nt, synthetic construct
  source 1..924: mol_type = other RNA
atgggcggactgacagccagtgatgtgcaccccaccctgggagttcagctgttcagcgctggaatcgccgcctgcctggccgacgtgattacatttcctctggacaccgccaaggtgcggctgcaagtgcagggcgagtgtcctacaagcagcgtgatccggtacaagggcgtgctgggaaccatcacagctgtggtgaaaaccgagggaagaatgaagctgtacagcggcctgcctgctggcctgcagagacagatcagctccgcttctctgcggatcggcctttatgataccgtgcaggagttcctgacagctggcaaggaaaccgcccctagcctgggctctaaaatcctggctggcctgaccaccggtggcgtggccgtgttcatcggccagcctacagaagtggtcaaggtgcggctgcaggcccagagccacctgcacggcatcaagcccagatacaccggcacatacaacgcctacagaatcatcgccaccaccgagggcctaaccggcctgtggaagggcaccacccctaatctgatgagaagcgtgatcatcaactgcaccgaactggtgacctacgacctcatgaaggaagcctttgtgaagaacaacatcctggccgatgacgtgccctgccatctggtctccgccctgatcgccggcttctgcgccacagccatgagcagccccgtggacgtggtgaagacaagattcatcaacagccctcctggccagtacaagagcgtgccaaattgtgccatgaaagtgtttaccaacgagggccctaccgccttcttcaagggactggttccatccttcctgaggctgggcagctggaacgtgatcatgttcgtgtgcttcgagcagctgaagagagagctgtctaaatctagacaaacaatggactgcgccacctga

SEQ ID NO: 63 - RNA, 924 nt, synthetic construct
  source 1..924: mol_type = other RNA
atgggaggcctgaccgctagcgacgtgcaccctacactgggcgtgcagctgttcagcgccggcatcgccgcatgcctggctgatgtgatcaccttccccctggataccgccaaggtgcggctgcaagtgcagggcgagtgccctacaagctccgtgatcagatacaagggagtgctgggcacaatcacagctgtggtgaaaaccgagggcagaatgaagctgtacagcggcctgcctgccggacttcagagacagattagcagtgcctctctgagaatcggcctgtatgatacagtgcaggagttcctgacagccggcaaagagacagctccttccctgggcagcaagatcctggccggcctgaccaccggcggcgtggccgtgttcatcggacagcctaccgaagtggtcaaagtgcggctgcaggcccagagccacctgcatggcatcaagcctcggtacaccggcacctacaacgcctacagaatcatcgccaccacagaaggcctgaccggactgtggaagggaaccacccctaacctgatgagaagcgtgatcatcaattgcactgagctggtgacctacgacctgatgaaggaagccttcgttaagaacaacatcctcgccgacgacgtcccctgtcacctggtcagcgccctgatcgccggcttctgcgccaccgccatgagctctccagtggacgtggtgaagaccagattcatcaacagcccccccggccagtacaaaagcgtgcctaattgcgccatgaaggtgtttaccaacgagggccctacagcttttttcaagggcctggtgccatctttcctgcgcctcggaagctggaacgtgattatgttcgtttgttttgagcagctgaagcgggaactgtctaagtccagacaaacaatggactgcgccacctga

SEQ ID NO: 64 - RNA, 924 nt, synthetic construct
  source 1..924: mol_type = other RNA
atgggcggcctgacagcctctgatgtgcaccctaccctgggagtgcagctgttcagcgccggcatcgccgcttgtctggccgatgtgatcacctttcctctggacaccgctaaggtgcgcctgcaggtgcagggcgagtgtcctaccagcagcgtgatcagatacaagggcgtgctgggcaccatcacagcagtggtcaaaaccgagggcagaatgaaactgtacagcggcctccccgccggcctgcaaagacaaatcagctctgccagcctgcggattggcctgtatgacaccgtacaggagttcctgacagccggaaaggaaaccgccccttctctgggatccaagatcctggccggcctgaccactggcggagtggccgtgttcatcggccagcctaccgaggtggtgaaggtgcggctgcaggcccagagccacctgcatggcatcaagcctagatacacaggcacgtacaacgcctacagaatcatcgccaccaccgaaggactgaccggcctgtggaagggcacaacccctaacctgatgagaagcgtcatcatcaactgcaccgaactggtcacatacgacctgatgaaagaggcctttgtgaagaacaatatcctggccgacgatgtgccatgccacctggtgtccgccctcatcgccggcttctgcgctaccgctatgagctcccctgtggacgtggtgaagacccggttcatcaacagcccccccggccagtacaaaagcgtgccaaattgcgccatgaaggtgttcacaaacgagggacctacagcttttttcaagggcctggttcccagcttcctgagactgggctcttggaacgtgatcatgttcgtgtgcttcgagcagctgaagcgggaactgagcaagagcagacagaccatggactgcgccacatga

SEQ ID NO: 65 - RNA, 924 nt, synthetic construct
  source 1..924: mol_type = other RNA
atgggcggactgaccgcttctgatgtgcaccctacactgggcgtccaactgttctctgccggcatcgccgcttgtctggctgatgtgatcacattccccctggacaccgccaaggtgcgcctgcaggtgcagggcgagtgccctacctctagcgtgattcggtacaagggcgtgctgggcaccatcacagccgttgtgaagaccgaaggcagaatgaaactgtactccggcctccctgccggcctgcaaagacagatcagcagcgccagcctcagaattggcctgtatgataccgtgcaggagttcctgaccgccggcaaggagacagcccctagcctgggcagcaagatcctggccggcctgacaaccggcggagtcgccgtgtttatcggacagcctaccgaggtggtgaaagtgcggctgcaggcccagagccacctgcacggcatcaagcccagatacaccggcacctacaacgcctaccggatcatcgccaccaccgaaggcctgaccggactgtggaagggcacaacccctaatctgatgcggagcgtgatcatcaactgcacagaactggtcacctacgacctgatgaaggaagccttcgtgaagaacaacatcctggccgacgacgttccatgtcatctggtgtccgccctgatcgccggcttctgcgcgaccgctatgagcagccctgtggacgtggtgaagacaagattcatcaacagcccccccggccagtacaagtccgtgcctaattgcgccatgaaagtgttcaccaacgagggccctacagcttttttcaagggactggtgccatcttttctgagactgggaagctggaacgtgatcatgttcgtgtgcttcgagcagctgaagagagagctgagcaaaagcagacagacgatggactgcgccacatga

SEQ ID NO: 66 - RNA, 924 nt, synthetic construct
  source 1..924: mol_type = other RNA
atgggcggactgaccgcttctgatgtgcatcccaccctgggagtgcagctgttcagcgccggcatcgctgcttgtctggccgacgtgatcaccttccccctggacaccgccaaggtgcgcctgcaagtgcagggcgagtgccctaccagcagcgtgatcagatacaagggcgtgctgggcacaatcaccgccgtggtgaagaccgagggcagaatgaaactgtatagtggcctgcctgccggtctccagcggcagattagctctgccagcctgcggatcggcctgtacgacaccgtgcaggagtttctgacagcaggcaaagagacagccccttctctcggaagcaagatcctggccggcctgaccaccggcggcgtggccgtgtttatcggacaacctacagaagtggtgaaagtgcggctgcaggcccagagccacctgcacggcatcaagcctagatacaccggaacatacaacgcctacagaatcatcgccaccaccgaaggcctgaccggcttatggaagggcaccacacctaatctgatgagaagcgtgattatcaactgtacagagctggtgacctacgacctgatgaaggaagccttcgtgaagaacaacatcctggctgatgatgtgccatgccacctggtgtccgccctgatcgccggcttctgcgccaccgccatgagcagccctgtcgacgtcgtgaagacaagattcatcaacagcccccccggccagtacaagtccgtgccaaattgcgccatgaaagttttcaccaacgagggccctacagcttttttcaagggactggtccctagcttcctgagactgggctcctggaacgtgatcatgttcgtgtgcttcgagcagctgaagcgggaactgtctaagagcagacagaccatggactgcgccacgtga

SEQ ID NO: 67 - RNA, 924 nt, synthetic construct
  source 1..924: mol_type = other RNA
atgggcggactgacagcctctgatgtgcaccccaccctgggagtgcagctgttcagcgccggcatcgccgcttgtctggccgacgtgattaccttccctctggacaccgcaaaggtgaggctgcaggttcagggcgagtgccctaccagcagtgtgatccggtacaagggcgtactgggcaccatcaccgctgtggtgaagacagaaggcagaatgaaactgtacagcggccttcccgccggcctgcaaagacagatcagcagcgcctctctgcggatcggcctgtatgatacagtgcaggagttcctgacagccggcaaggaaaccgcccctagcctgggatctaagatcctggctggactgaccaccggcggcgtggccgtgttcatcggccaacctaccgaggtggtcaaggtgcggctgcaggcccagagccacctgcatggcatcaagcctagatacaccggcacatacaacgcctacagaatcatcgccaccaccgagggcctcaccggcctgtggaagggaacaacccctaatctgatgagaagcgtgatcatcaattgcacggagctggtgacctacgacctgatgaaggaagcctttgtgaaaaacaacatcctggccgatgacgtgccatgtcacctggtgtccgccctgatcgccggcttttgcgccaccgctatgagctctcctgtggacgtggtgaaaacaagattcatcaacagccctccaggccagtacaagtccgtccccaactgcgccatgaaagtgttcaccaacgagggccctacagcttttttcaagggactggtccccagcttcctgagactgggcagctggaacgtgattatgttcgtgtgcttcgagcagctgaagcgggaactgagcaagtccagacagaccatggactgcgccacatga

SEQ ID NO: 68 - RNA, 924 nt, synthetic construct
  source 1..924: mol_type = other RNA
atgggaggactgacagcctctgatgtgcatcccaccctgggcgtgcagctgttcagcgccggcatcgccgcttgtctggccgatgtgattaccttccccctggacaccgccaaggtgcggctgcaggttcagggcgagtgccccacaagcagcgtgatcagatacaagggcgtgctgggcaccatcaccgcagtggtgaagacagaaggcagaatgaaactgtacagcggcctgcctgccggcctgcaaagacagatcagctccgccagcctgagaatcggcctgtatgataccgtgcaggagttcctcacagccggcaaggaaaccgccccttctctggggtccaagatcctggctggcctgaccacaggaggcgtcgccgtgtttatcggacagcctacagaggtggtgaaggtgcggctgcaggcccagagccacctgcacggcatcaagcctagatacaccggcacctacaacgcctacagaatcatcgccaccaccgaaggcctgaccggcctctggaagggaaccacccctaatctgatgcggagcgtgatcatcaactgcaccgagctggtgacctacgacctgatgaaagaggcctttgtgaagaacaacatcctggccgacgacgtgccttgtcacctggtcagcgccctgatcgccggcttctgcgctacagctatgagctctcctgtggacgtggtgaagaccagattcattaacagcccacctggccagtacaagtccgtgccaaattgcgccatgaaagtcttcaccaacgagggacctacagcttttttcaagggcctggtgcccagcttcctgaggctgggctcttggaacgtgatcatgttcgtgtgcttcgagcagctgaagcgggaactgtctaaaagcagacaaacaatggactgcgccacatga

SEQ ID NO: 69 - RNA, 924 nt, synthetic construct
  source 1..924: mol_type = other RNA
atgggcggactcacagccagcgacgtacatcctacactgggcgtgcagctgttcagcgccggcatcgctgcctgcctggctgatgtgatcaccttccccctggatacagcaaaggttagactgcaagtgcagggcgagtgccccacaagcagcgtgatccggtacaaaggcgtgctgggaaccattacagctgtggtgaagacagagggcagaatgaagctgtattctggcctgcctgccggcctgcagagacagatcagctctgctagcctgcggatcggcctgtacgacaccgtgcaggagttcctcacagccggaaaggaaaccgccccttctctgggcagcaagatcctggccggcctgaccacaggcggcgtggccgtgtttattggacaacctacagaggtggtcaaggtcagactgcaggcccagagccacctgcacggcatcaaacctagatacaccggcacctacaacgcctacagaatcatcgccaccaccgagggcctgaccggcctttggaagggcaccacccctaatctgatgcgcagcgtgatcatcaattgtaccgaactggtgacatacgacctgatgaaagaagcctttgtgaaaaacaacatcctggccgacgatgtgccatgccacctggtgtccgccctgatcgccggcttctgcgccaccgctatgagctcccctgtggacgtggtgaagaccagattcatcaacagcccccccggccagtacaagtccgtgcccaactgtgccatgaaggtcttcactaacgagggtcctaccgcctttttcaagggactggttccaagcttcctgaggctgggctcttggaacgtgatcatgttcgtgtgcttcgagcagctgaagcgggaactgagcaagagccggcagaccatggactgcgccacctga

SEQ ID NO: 70 - RNA, 924 nt, synthetic construct
  source 1..924: mol_type = other RNA
atgggaggtctcacagccagcgacgtacaccccacattaggggttcagctctttagtgcaggcatagccgcttgcctcgctgatgttattacttttcccctcgacactgctaaggtgcgactgcaagtacagggcgaatgccctacttcatcagtgattcgatacaagggcgtattgggcactattactgccgtcgtgaaaacagaaggccgtatgaagctgtatagtgggctgccagcgggattgcagaggcagatcagctctgcatctctgcgaatcggactgtacgacaccgttcaagagttccttaccgcaggaaaggagacagcacctagcctagggtccaaaatcttggcaggtctcactacagggggcgtagctgttttcatcggacaacccacagaagttgtaaaggtccgactccaagcacagagccatcttcacgggatcaaacctcgctacactggcacgtacaacgcttacaggatcatcgccactacagagggtttaacagggctttggaaggggacaacacctaatctgatgcgatccgtgattattaactgcaccgaattggtgacttacgatctcatgaaggaggccttcgtgaagaacaacatcttggcggacgacgtgccttgtcatttagtctctgcactgatagccggattctgtgccaccgccatgtcgagccctgttgacgtcgtgaagacgcgatttatcaattctcctcctggtcaatataagagtgtccctaattgtgccatgaaggtcttcacaaacgagggccctacagcctttttcaagggactcgtcccatcctttctacggctcggttcttggaacgtcatcatgttcgtgtgtttcgaacaactgaaacgagagctgtccaaaagtaggcagaccatggactgcgctacttga

SEQ ID NO: 71 - RNA, 924 nt, synthetic construct
  source 1..924: mol_type = other RNA
atgggcggtctaacggcctctgacgtccatcccacacttggggtgcagctcttcagtgcggggatcgccgcctgcttggccgatgtcattacttttcctctggacacagcaaaggtgcgattgcaggttcaaggagaatgccccacctcctccgtcattcgatataaaggcgttttggggacgatcactgccgtggtgaagacggagggccgtatgaaattgtacagcgggctgccagcaggcctgcagaggcagataagctccgcatcattgcgaatcggcctgtacgacacagtgcaagaatttcttactgccggaaaggagacagcacccagtttggggagcaaaatcctcgccgggctgaccacaggcggcgtcgctgtatttattggacagcctactgaagtggtaaaggtacgactccaggcccagtctcatctgcacggaatcaagcctagatacactgggacatataacgcttacaggatcatcgctacaacagaaggcctcactggactttggaagggcacaactcctaatttaatgcgatcagtcatcatcaattgcaccgagttggtcacatacgacctaatgaaggaggccttcgtgaaaaacaatatactcgcagatgacgttccttgtcacttagtgtctgcgctgatcgccggattttgtgccaccgccatgtctagccctgttgatgtcgtgaagacaaggtttatcaattcaccacctggccagtataaatctgttcctaattgtgcaatgaaggtgttcacgaatgaaggtccaacagcattcttcaaggggcttgtcccttcatttctgcgactgggatcatggaacgtgatcatgtttgtgtgttttgaacaactgaaaagagagctgtcgaagtccaggcagacaatggactgcgccacctga

SEQ ID NO: 72 - RNA, 924 nt, synthetic construct
  source 1..924: mol_type = other RNA
atgggcggactgaccgcttccgacgtacaccctaccttaggagtgcagctcttcagtgcaggaatcgccgcttgccttgctgatgtcattacttttccattagacaccgccaaggtgcggctacaggttcagggcgaatgtcccacttcttccgtcattcggtacaaaggcgttctgggaacgatcacagctgtcgtgaaaaccgaaggccgtatgaagctgtacagcgggcttccagcaggacttcagaggcagatcagctcggcgtccttacgcatcggactctacgacacagtccaagaattcctcacagctggaaaggaaactgcccctagcctggggagcaaaatactggccgggctcacaacaggcggcgttgctgtgtttattggacagcccactgaggttgtcaaggtccgcctgcaagcccaaagccaccttcacgggattaagcctcgctacactggaacatacaatgcatatagaatcatcgccaccaccgaaggcctaactggactgtggaaagggacaacacctaacttaatgcggtcagttattattaactgcactgagttagtgacttacgatctcatgaaggaagcattcgtgaagaacaacattttggcagatgacgttccttgtcatttggtttccgcattaatagccgggttttgtgctaccgccatgtcttccccagtcgacgtcgtgaagacaaggtttataaattccccacctggccagtataaaagtgtccctaactgtgcaatgaaagtcttcacaaacgagggccctaccgccttcttcaaggggcttgttccctcttttctgcgactcggctcatggaacgtcatcatgtttgtctgttttgaacagctgaaaagggagctatcaaagtcaagacagaccatggactgcgccacctga

SEQ ID NO: 73 - RNA, 924 nt, synthetic construct
  source 1..924: mol_type = other RNA
atgggcggcctgaccgcttcagacgttcacccgacgttgggagtgcagctattcagcgcaggaatagccgcttgcttagcagatgtgattactttccctctcgatactgctaaggtccgccttcaagttcagggcgaatgccctacttcttccgtgattcgctataagggcgtactgggaactattacagctgtcgtgaaaacagaaggccgcatgaaactctacagcggactccctgccgggttacaaagacaaatcagctcagcttccctgcggatcggcttgtacgacaccgttcaggaattcctcacagccggaaaggagactgcacctagtttaggaagcaaaattctcgcagggcttaccacaggtggcgtcgccgttttcattggacaaccaaccgaagtagttaaggtccgactccaagctcaatctcacctacacggaattaagcctcgctacactggcacgtataacgcttataggattatcgctacaacagaaggattaacgggcctgtggaagggcaccacccccaatttaatgcgatcagtcatcatcaactgcaccgagttggtaacatacgatctcatgaaggaagcctttgtgaaaaacaacatactggcagacgacgtcccttgtcatttggtttctgcgctcatcgcagggttttgtgctaccgccatgtctagccctgttgacgttgtcaagacacgatttataaactctccaccaggccagtataaatctgtccctaattgtgccatgaaggtcttcacaaacgagggtcccactgctttcttcaaggggcttgttccctcgtttctgcggcttggatcctggaatgttatcatgtttgtgtgctttgaacagttgaaaagagagttgtccaaatccaggcagactatggactgcgccacatag

SEQ ID NO: 74 - RNA, 924 nt, synthetic construct
  source 1..924: mol_type = other RNA
atgggagggctcacagcaagcgacgtgcatccaaccttaggagtacagctcttcagtgcaggaatcgccgcttgcctagccgatgtcattacctttccactggacaccgctaaggttcgcctgcaagttcagggcgagtgtcctacctcttctgtaattcgatataaaggcgtgttggggacaatcacagctgtcgtgaaaacagaaggccgcatgaagctctacagcggactgccagcaggtttgcaacggcagataagctcagcatccctccgtatcgggctgtacgacacagttcaggaattcctcactgccgggaaagagacagcccctagcttggggagcaaaattttggccgggctcactacaggtggcgtcgctgtgttcattggacaacccactgaggtggtgaaggtacgactccaggctcaaagccatttacacgggattaaaccacgctacactggaacttacaacgcatacagaatcatcgctaccacagaaggtttgacgggactttggaaagggacaacacctaatttaatgcgatctgtcatcatcaattgcactgaattggtgacttatgacctgatgaaagaagcatttgttaaaaataacatcctggcggatgatgtcccttgtcatttggtatcagcactcattgcagggttttgtgccaccgccatgtcttccccagttgatgttgtaaagacacggttcataaactcacctccaggccagtataagagtgtccccaattgtgcaatgaaagtgttcacaaacgaaggtccaactgcattcttcaaggggcttgttccctctttccttcgtctcggatcctggaacgtgataatgtttgtgtgttttgaacaacttaaacgagagctttctaaaagtaggcagactatggactgtgcaacgtag

SEQ ID NO: 75 - RNA, 924 nt, synthetic construct
  source 1..924: mol_type = other RNA
atgggcggcctcaccgcatcagacgtccatccaacattgggggtgcagctcttcagtgccggcatcgctgcctgtctcgccgacgtcattacctttccgttagatactgcaaaggtccgcctgcaggttcaaggcgagtgcccaacttcctccgttattcgctacaagggcgtgctgggaacaatcaccgcagtggtgaaaacagagggccgaatgaagctctactccgggctaccagcaggcttgcagaggcaaatcagttctgcatccctgcgcatcggtctatacgacactgttcaggagttcctcaccgccgggaaggagacagcccctagtttgggcagcaaaatactcgccgggctcacaacaggtggcgttgccgttttcatcggacaaccaaccgaggtcgtgaaggtgcgactgcaagcacaaagccatctccacgggatcaagcctagatacactggaacttacaacgcctacaggatcatcgctacaaccgaaggcttaactggactctggaaagggacaacccctaacttaatgcgaagcgtcataatcaactgtacagaactagtcacatacgatctcatgaaagaagcctttgtgaaaaataatatcctggcagatgacgttccttgtcacctggtctcagcactgatcgccggattttgcgctactgcaatgtcttctcctgttgatgttgtgaaaacacggttcataaattctcctccaggtcagtataagagtgttcctaattgtgccatgaaagtcttcactaacgaaggtcctacagcattctttaagggactcgttccctcttttctccgtcttggctcatggaacgttatcatgtttgtgtgttttgaacagctgaagagggagctgtccaaatcaagacaaaccatggactgcgccacatga

SEQ ID NO: 76 - RNA, 924 nt, synthetic construct
  source 1..924: mol_type = other RNA
atgggagggcttactgctagtgacgtccaccctaccctcggagttcagctcttcagtgccggaatcgccgcgtgcctcgccgacgtgattacctttccactagacaccgctaaggtgcggctgcaagtccagggcgagtgtcctacatcctccgtcattcgctataaaggcgttttgggcaccatcacggctgtagtcaaaacagagggccgtatgaagctctacagcgggctgcccgccgggttgcagagacaaataagctctgcttctctgcggatcggtctgtacgacactgttcaggagttcctcactgctggtaaggagacagcacctagcttgggtagtaaaattctcgcagggctcactacaggcggcgtcgccgtattcattggacagcccaccgaagttgtgaaggtacgactgcaagctcagtcccatctgcacgggataaaacctcgttacactggcacttacaatgcttacaggataatagctacaacagaaggcttaacgggactctggaaggggacaacaccaaatttaatgcgatctgttatcatcaactgcactgaactcgtgacctatgatctgatgaaggaagcctttgtgaaaaacaacatcctggccgacgacgttccctgtcacttagtgtctgcactcatcgccggcttctgtgccaccgctatgtcctcccctgtggacgttgtgaagacacgcttcataaattcccctcctggccaatacaaaagcgtgcctaactgtgccatgaaggttttcacaaacgagggccctactgcttttttcaaaggcctcgttccttcctttcttcggctcgggtcctggaatgtcatcatgttcgtgtgttttgagcagttaaagagagagcttagcaaatccaggcagaccatggactgcgccacctga

SEQ ID NO: 77 - RNA, 924 nt, synthetic construct
  source 1..924: mol_type = other RNA
atggggggactgaccgccagcgacgtgcaccctactctgggagtccagctcttcagtgccggtatcgccgcctgtcttgccgatgttataacatttcccctggacacagctaaggtccgacttcaagttcaaggcgaatgtcccacttcttctgtcatccggtacaaaggcgtactgggaaccatcacagcagtggtgaaaaccgaaggccgtatgaagctttacagcggactcccagcaggactgcagaggcaaataagctcggcatcactgcggatcggcctctacgataccgtccaggagtttctgaccgccggaaaagaaacagctcctagtttgggcagtaaaatactcgccgggctcactacaggtggcgttgccgttttcataggacaaccaacagaagtggtcaaggtcagactacaggctcaaagtcaccttcacgggattaaacctcgctataccggaacttataacgcttataggatcatagccacgacagaaggcttgactgggctctggaaggggacaacacctaacttaatgcgatccgtgatcataaattgtactgagctcgtgacttacgatctcatgaaagaggcgttcgtaaaaaataacatattggcagatgatgttccctgtcacttagtttccgcactgatcgccgggttttgtgcaacagctatgtcttctcctgtcgacgttgtcaaaacacggttcataaattctcctccgggccaatataaaagtgtccctaactgtgccatgaaagtcttcacgaatgaggggcctacagccttctttaagggactggtcccctcgtttcttcgcctcggttcatggaatgttatcatgttcgtgtgttttgagcagttaaagcgggagctgagcaagagtagacaaacaatggactgcgccacctga

SEQ ID NO: 78 - RNA, 924 nt, synthetic construct
  source 1..924: mol_type = other RNA
atgggcggattgaccgccagcgatgtgcatcccacgttaggtgtgcagctctttagcgcaggtattgccgcgtgcttagcggatgtgattacgtttcccctcgacacggctaaggtacggcttcaagtccagggcgagtgcccaacttcttccgtaattcgctacaaaggcgttctcggaaccatcacagccgtggtgaagacagaaggccgcatgaaactttacagcggactgccagccggcttgcagaggcagataagctcggcctctctccgcatcggtttgtacgacacggttcaggagttccttactgctggaaaggagacagcacccagtctggggagcaaaatcctagccgggcttacaacaggtggtgtggctgtcttcattggacagcccactgaggttgttaaggtacggctgcaggctcaaagccacctccacggcatcaaaccacgctatactggtacatataacgcctacaggatcattgcgactacagaaggactcacaggactgtggaaggggacgactcctaatttaatgcggtctgtgatcatcaactgcaccgaactggtcacttacgatctaatgaaagaggctttcgtgaaaaataatatcttggccgatgacgtgccttgtcacctcgtttctgcgctcatcgccggattctgtgcaaccgccatgtcttctccggttgacgttgtcaaaacacgatttataaactcaccaccaggccaatacaaaagcgtccccaactgtgcaatgaaagtcttcacaaacgaaggcccgacagcgttctttaaggggctcgtcccctcctttcttcggctcggttcctggaatgttataatgtttgtgtgttttgaacagctaaaaagggagctgtcaaaatccaggcaaacaatggactgtgcaacatga

SEQ ID NO: 79 - RNA, 924 nt, synthetic construct
  source 1..924: mol_type = other RNA
atggggggactgacagccagtgatgtgcatcccactctgggtgttcagcttttcagcgccgggattgccgcatgtctcgctgatgtaattacttttcctctcgacacagctaaagtccgcctgcaggttcagggagagtgtccaacgtcctccgtgattcggtataaaggagttttaggaacaatcacagcagtggtgaagaccgagggccggatgaagctctacagcgggctcccggcaggattgcagaggcaaatcagttctgcctctctccgtatcggcctgtacgatactgtacaagaattcctcaccgccggaaaggagacagcacccagtttggggagtaaaattctggcagggctgaccacaggcggcgtggctgttttcattggacagcctaccgaggtggtcaaggtccgattgcaggctcaatctcatcttcacggcataaaacctagatatactgggacatacaacgcttacaggatcatcgcaaccacagaaggcctgactggactatggaaagggacaacacccaatttaatgcgatcagtcattatcaactgcactgaattggtaacatacgatctcatgaaggaagctttcgtgaagaataacatcctcgcagatgacgttccctgtcaccttgtgtctgcactgatcgccggattttgtgccaccgcgatgtcttccccggtcgatgttgtgaagacacgattcataaattctccaccaggccaatacaaaagcgttcccaactgtgccatgaaagtatttacaaacgaaggcccgacagctttctttaaggggctcgtcccctcatttcttcgactcggttcctggaatgtaatcatgtttgtgtgtttcgaacaattaaaaagggagctgagtaaaagtagacaaaccatggactgcgccacctag

SEQ ID NO: 80 - RNA, 924 nt, synthetic construct
  source 1..924: mol_type = other RNA
atgggtggtctcacagccagtgacgttcatcccactctgggggttcagcttttcagtgcaggaatcgccgcctgcctcgccgacgtgatcacctttccgttggacactgccaaagtacgcctacaggtccagggcgagtgtcctacttcttccgtgattcggtataagggtgtgctaggaaccatcactgccgtggtgaaaacagagggccgtatgaaactctacagcgggttgcctgccgggctacagaggcagatcagctcagcttctttgagaatcggtctctacgacacggtccaggaatttcttactgccgggaaggagactgcacctagtctgggcagcaaaattttagcggggctcacaacaggcggcgtggctgtattcattggacaacccacagaggttgtgaaggtccgactgcaggctcaaagccatctgcacggcattaaacctcgctacactggcacttacaatgcttaccgaatcattgctacaactgagggccttactggactctggaaaggcacaactcccaatttaatgcgatccgtaatcatcaattgcactgaactcgtgacttacgacctcatgaaggaagcttttgtgaaaaataacatcctggcagatgacgtcccttgtcacttggtttctgctcttatcgcaggattctgcgcaaccgccatgtcatctccagtggacgttgtgaagacaaggttcataaattcccctcccggtcaatataaaagtgtccccaattgtgccatgaaggtgttcacaaacgaaggcccaaccgccttttttaaggggcttgttccatcttttctgcgtctcggatcctggaacgtcatcatgttcgtgtgttttgaacaattgaaaagggagttgtcgaagtccaggcaaacaatggattgtgcaacgtaa

SEQ ID NO: 81 - RNA, 924 nt, synthetic construct
  source 1..924: mol_type = other RNA
atgggcggactcacggcttccgacgtccatcccaccctgggggtgcagctcttcagcgcaggcattgccgcgtgtctcgccgatgttattacttttccacttgacaccgctaaggtgcgattacaggtccagggcgaatgcccaacatcttccgtgatccggtataaaggtgtcctgggaaccattaccgcggtggtgaagacagaaggccgcatgaaactgtacagcgggctgcccgcagggctgcagagacaaataagctccgcatccctccgcatcggactatacgacacagttcaggaattcctcacggccgggaaggagacagctcctagtctgggaagcaagattcttgctgggctcactacaggcggcgtcgctgtcttcattggacaacctactgaggtggtgaaggtccgactgcaagcccaatcccaccttcatggaatcaagcctcgctacactgggacatataacgcctatagaatcattgctactacagaaggcttgactggcctttggaaagggacaactcccaatttaatgcgaagcgtgattatcaattgcactgaactcgtgacttacgatctcatgaaggaagcatttgtgaaaaacaacatcctggctgatgacgttccatgtcatctggtatctgcactgatcgccggattttgcgctactgccatgtctagccctgtagatgtcgtcaagacacggttcattaattctcctccaggccaatacaaaagtgtccccaattgtgctatgaaagtgttcacaaatgagggccccacagccttctttaaggggctcgtcccctcttttctgcgcctcggttcctggaatgtcatcatgtttgtgtgcttcgagcaattgaagcgagagttgtcaaagtcccggcagactatggattgtgccacgtaa

SEQ ID NO: 82 - RNA, 924 nt, synthetic construct
  source 1..924: mol_type = other RNA
atgggaggcctcaccgctagtgacgtgcaccctactctgggtgtacagctcttcagtgcaggaatcgccgcgtgcttggccgatgttattacttttcccctggacacagctaaggtgcgcctgcaggttcagggggagtgtcccacctcctccgtcatcagatacaaaggcgttttaggaaccatcacagccgtggtaaaaactgagggccgcatgaagctctacagcggtctcccggcaggacttcagaggcagatcagctctgcatcactccgtatcgggctctacgacaccgttcaagagtttcttacagcaggaaaggagactgcacccagtttgggaagcaaaattctggcaggtcttacaaccggcggcgtggctgtgttcatcggacagcccactgaggtggtcaaggtgcggctccaggctcaatctcacttgcacggcatcaagcctcgctacactggcacgtacaatgcttacagaatcattgcaactacagaaggcctgacaggactttggaagggaacaactcctaaccttatgcgatccgttatcattaactgcacagagctcgtcacatatgacctcatgaaggaagcgttcgtgaaaaataacatccttgcagatgacgttccttgccacttggtgtccgcgctgatcgccggattctgtgctaccgctatgtcaagccctgtcgacgtcgtgaagacaaggttcatcaattcccctccaggacaatataagagtgttcctaactgtgcaatgaaagtattcacaaacgaaggccccaccgccttcttcaagggactggttccttcttttcttcgactcggttcctggaacgttatcatgttcgtgtgttttgagcaattaaaaagggagctgtcaaagtcccggcagacaatggactgtgccacatga

SEQ ID NO: 83 - RNA, 924 nt, synthetic construct
  source 1..924: mol_type = other RNA
atgggtggtttaaccgcaagcgacgtccaccccaccctaggggtgcagctcttcagtgcaggaatcgccgcatgtctcgccgacgtcattacatttcccttagacaccgctaaggtgcgcctccaggtccagggcgaatgtcccacatcttctgtaatccgctataaaggcgtacttggaacaatcacagctgtagtgaaaaccgagggccggatgaaactgtacagtgggctgcccgcaggactgcagaggcagatcagctccgcatcactgcgaatcgggctttacgacaccgtgcaggaatttctcactgccggaaaagagactgctcccagtctgggtagcaaaattctggccggactcacaacaggcggcgtagctgtcttcattggccagcccaccgaggtggttaaggtccggctgcaagcccaatcccaccttcacggtatcaagccgcgttacactggaacttacaacgcttacagaattatcgctaccaccgaaggcttaacgggactctggaaggggacaactcccaatttaatgcgatccgttatcatcaactgcaccgaactagtgacctatgacctcatgaaggaagccttcgtgaaaaacaacatcctggcagacgacgtgccctgtcacctggtctctgcactcatcgctggattctgtgctacagcaatgtcatctcctgtcgacgttgtgaagacacgattcatcaactctccacctggtcagtataaaagtgtccctaactgtgccatgaaggtcttcacaaatgaaggccctaccgcattcttcaaggggctcgtcccctcctttttacgcctcggctcctggaacgttattatgtttgtatgttttgagcaactcaaaagggagctgtcaaaatccagacagactatggactgcgccacatga

SEQ ID NO: 84 - RNA, 924 nt, synthetic construct
  source 1..924: mol_type = other RNA
atgggtgggctgaccgccagcgatgttcatccgactttaggtgtgcaactcttcagtgccggcatcgccgcctgcctcgctgatgtgattacctttcctcttgataccgctaaggtacggctgcaggtccaaggcgaatgccccacctcttccgtcattcggtacaaaggcgtgttgggaacaattacggcagtcgtgaagactgaaggccgaatgaaactctacagtgggctgcctgctggtttgcagagacagataagctcagcgtctctgcgaattgggctctatgacacggttcaggagttcctcaccgctgggaaggagacagctcctagtctgggaagcaaaatactcgccggactaacaaccggtggcgttgctgtattcatcggacagcctaccgaagtcgttaaggtcagactccaggctcagagccacctgcatggcattaagcctcgctacactggtacctacaacgcttacaggattatcgctaccacagaaggcctgactggactgtggaaagggacaactcctaatttaatgcgctctgttatcatcaactgcaccgaattggtcacttacgatctcatgaaggaagcgtttgtgaaaaacaacatcttggctgatgacgttccttgtcacctagtttcggcactgatcgctgggttttgtgccaccgccatgtcttctcctgtcgacgttgtcaaaacaaggtttataaactcaccaccaggtcagtataaaagtgtccctaactgtgccatgaaagtcttcaccaacgaagggcccacagctttcttcaaggggcttgtcccttcgtttctacgactcggatcttggaacgtgatcatgtttgtctgtttcgagcaattgaagcgagagctgtctaagtctaggcagacaatggactgcgcaacctga

SEQ ID NO: 85 - RNA, 924 nt, synthetic construct
  source 1..924: mol_type = other RNA
atgggggggctgacagcttctgacgtacatcctactctgggggtccaactcttcagtgccggaatcgccgcttgcctcgctgacgttatcaccttccctttagacacagctaaagtccgactgcaagttcaaggcgagtgccctacttcttccgtgatcagatacaagggcgtgctgggtacaattactgcggtggtgaaaactgaaggccgcatgaagctctatagcgggctgcctgcaggcttgcagaggcagataagctcagcctccctgcggattggtctgtacgatactgttcaggagttccttaccgctggaaaggagacagctcccagtttagggagcaaaatcctggccggactcactacaggcggcgtcgctgtcttcattggacagcccacagaggttgtaaaggtaaggctgcaagcccaaagccacctccacggcatcaaacctcgctatactgggacttacaacgcttaccgaatcatagctacaaccgaaggcttaactggactgtggaagggcacaacaccaaacttaatgcgatcagttattatcaactgcaccgaactggttacttatgatctcatgaaagaagcttttgtgaaaaataacatcctcgctgatgacgtcccttgtcatttggtgtctgcattaatcgccggattttgtgcgaccgctatgtcgagtcctgtcgacgttgtgaaaacacgattcataaattcacctcccgggcagtataaaagcgttcccaattgtgccatgaaggtattcaccaacgaaggtcctacggcattcttcaaggggctggttccttcctttcttcggctcggttcctggaatgttatcatgtttgtctgttttgaacagctgaaaagggagctatcaaagtccaggcagaccatggactgcgctacttga

SEQ ID NO: 86 - RNA, 924 nt, synthetic construct
  source 1..924: mol_type = other RNA
atgggcggactcacagcctcggacgtgcaccccacactgggagttcagcttttcagcgcaggcatagccgcctgcctcgcagatgtgattacctttcctctagacactgctaaggtccgcttgcaagtccagggcgagtgcccaacttctagtgtgatccgatataagggcgtgttggggaccattacggcggtcgttaaaacagagggccgcatgaagctatatagcgggctgccagccggcctgcagaggcagatcagctctgcctccctacgtatcggcctgtatgacaccgttcaggagttccttactgcaggaaaagaaactgctcctagcttgggcagcaaaattctcgccgggctcactacaggcggcgtggctgtgttcattggacagcctactgaagtggtcaaggtccgactgcaggctcaaagccacctccatggcatcaaacctcgctacactggcacatataacgcttacagaatcatagccacaactgaagggctcacaggactttggaaggggacaacacctaacttaatgcgatctgttatcataaactgcaccgaactcgtaacgtatgatctcatgaaggaggccttcgtgaaaaacaacatcctggccgacgacgttccatgtcatttggtttccgctctcatcgccgggttttgtgctactgctatgtcaagccctgtcgatgttgttaagacacgatttataaactccccacctggccagtataagagcgtccccaactgcgccatgaaggtgttcacaaacgaagggcccacagcgttctttaaggggctggtcccttcttttctacggctcggatcctggaacgttatcatgttcgtgtgtttcgaacaattaaaaagggagctctccaagtcacggcaaacaatggactgcgctacatag

SEQ ID NO: 87 - RNA, 924 nt, synthetic construct
  source 1..924: mol_type = other RNA
atgggcggtcttactgctagtgacgtgcacccaacgctgggggtgcaactcttcagtgctggcatcgccgcttgccttgccgacgttattacttttcccttagataccgctaaggtgcgattgcaggtccaaggcgaatgccctacatcatcagttattcgatataaaggtgttctaggtaccattaccgccgtggtgaaaaccgagggccgcatgaagctttacagcgggcttcctgccggcctacagcgccagataagctcagcatctctacggatcggactgtacgacaccgtgcaggagttccttacagccggaaaggaaacagctcctagtctgggcagcaaaatcctcgccgggctcaccacaggcggtgtcgctgtattcattggtcaacccactgaggtcgtaaaggtccggctgcaggctcaaagtcacctgcacggtataaaaccgcggtacactggtacttacaacgcctataggatcatcgctacaacggagggcttaactggattgtggaaggggacgactcctaacttaatgcgctctgtcatcataaattgcaccgaactggttacttacgacctcatgaaggaagccttcgtgaaaaacaacatcctggcagatgacgttccatgccacctcgtgtctgcactcatcgcaggattctgtgctaccgctatgtctagtccagtcgatgttgtcaagacacggtttatcaattcaccaccaggacaatataaaagtgtcccaaactgtgccatgaaagtcttcacaaacgaggggcctacagcattcttcaaggggctcgtcccctcttttctgcggctcgggtcttggaacgttatcatgtttgtgtgtttcgaacagctaaaaagggaactgtccaagtcaaggcaaacaatggactgcgccacctaa

SEQ ID NO: 88 - RNA, 924 nt, synthetic construct
  source 1..924: mol_type = other RNA
atggggggtctgaccgcgagcgacgtccatcctaccctgggtgttcagctcttcagtgcgggcatcgccgcgtgcctggccgacgttattacctttcccttagacacagcaaaggtacgactacaggtacaaggcgaatgtcctacttcttccgttattcgctataagggcgtcttgggaaccattactgctgtggtaaaaacggaaggccgcatgaagctgtatagcgggctgcccgcgggccttcagaggcagatcagctcggcctccctgcgtattggattgtacgacacagtccaagaattccttaccgccgggaaagaaacagcacctagcctgggcagcaaaatactcgccggactcactacaggcggcgtggccgtgtttattggacagcctactgaggtggtcaaggtccgactgcaggcccagagtcacttgcacggcatcaaaccacggtatactggcacgtacaacgcttacaggatcatcgcaaccaccgaaggcttaacaggactctggaagggtacaacgcctaatttaatgcgctccgtcatcataaattgcaccgagctcgtgacttatgatctcatgaaggaagcctttgtgaagaataacatcctggcagacgacgttccctgtcacctagtatctgcactcatcgcaggattctgcgccaccgctatgtcttctcccgtagatgtcgttaagacgcggtttattaattcgccacccggtcaatataaaagtgtccctaattgtgccatgaaggtctttacaaacgaaggccctaccgccttttttaaggggctggttccttcttttttacggctcgggtcgtggaacgtcatcatgttcgtgtgttttgaacagctgaaacgggaactttccaaatccaggcagacaatggactgcgctacttga

SEQ ID NO: 89 - RNA, 924 nt, synthetic construct
  source 1..924: mol_type = other RNA
atgggtggtctgaccgcaagtgacgtgcatcctacactgggagtgcagctcttcagtgcgggcattgccgcttgccttgccgatgtaattacctttccattagacacagccaaggtgcgcctgcaggttcagggagaatgtccaacgtcttccgtgattcggtataagggcgttctgggcaccattacggctgtggtgaaaacagaaggccgtatgaaactttacagcggactccctgcagggctacagcgacagatcagctctgcttctctgcgtatcggtttgtatgacacagtacaggagttcctcactgccggaaaggagactgcacccagtttaggcagcaaaatccttgcagggctcacaacaggtggcgttgccgtttttatcggacagcccacagaagtggtgaaggtacgactgcaagctcagagtcacctgcacgggatcaagcctcgctatactggaacttataacgcttatagaatcattgctactacagaaggtctaactggactctggaaagggacaacacctaatttaatgcgatctgttatcatcaactgcacagaactggtgacttatgacctcatgaaggaagcttttgtgaaaaacaacatcctcgcagacgacgttccttgtcacctggtgtctgcactcattgcagggttttgtgcaaccgccatgtcttctcctgttgacgttgtcaaaacacgttttataaactcacctcctggacagtacaagagtgtccccaattgtgccatgaaggtattcacaaacgaagggcccaccgcattctttaaggggctggttccctcttttctgcgcctcggttcttggaacgtgataatgtttgtgtgctttgaacagttaaaaagagaactatccaaatcaaggcagacaatggactgtgccacatga

SEQ ID NO: 90 - RNA, 924 nt, synthetic construct
  source 1..924: mol_type = other RNA
atgggaggactgacagccagcgacgttcaccctacactgggggttcagctcttcagcgcgggaatcgctgcttgcctcgctgatgtcattacttttcctcttgatacagctaaggtccgactgcaagttcagggcgaatgccctacttcttccgtcatccgctacaagggcgtgctgggcaccatcacagctgtcgtaaaaacagagggccgcatgaaattatatagcggcttgcccgcagggctgcaaaggcagatcagctctgcgtccctccggatcggactgtacgacacagtgcaggaatttcttacggccggaaaggagactgctcctagcttgggtagcaaaattctcgccgggctgaccacaggcggtgtggctgtgttcatcgggcagcctaccgaggttgttaaggtgcgactgcaggctcaaagtcacctgcatggaatcaagcctcgctacactggtacatataacgcttatcggattattgcaaccactgaaggcctcacaggcctctggaagggcaccactcctaatttaatgcgatcagtcatcataaattgcaccgaactcgtgacttatgatctcatgaaggaagcttttgtaaaaaacaacattctggctgacgacgttccctgccatctggtgtcggctctcatcgcaggattttgtgcaaccgccatgtcttctcctgtcgacgttgtgaaaacaaggtttataaactcaccacctggccaatataagtctgttcccaattgtgccatgaaggtgttcacaaatgaaggtcccacagcattcttcaaagggcttgttccctcctttcttcggctcgggtcctggaacgtcatcatgttcgtgtgctttgaacagttaaaaagggaactgagtaagtccaggcagacaatggactgtgctacatga

SEQ ID NO: 91 - RNA, 924 nt, synthetic construct
  source 1..924: mol_type = other RNA
atgggtggcctaactgccagtgacgttcatcccactttgggagtgcagcttttcagcgcaggaattgctgcttgccttgctgacgttattacctttcccttagataccgcgaaggtccgcttgcaagtccaaggcgagtgcccaacttcatccgtcattcgctacaaaggcgttctgggtaccatcacagccgtcgtgaagaccgaaggccgcatgaaactctatagcgggctgcccgcgggcctccagaggcaaatcagctcagcttccttgcgcatcggattatacgacactgttcaagagtttcttacagccggaaaggagacagcgcctagtctggggagtaaaatcttggccgggctcacaacaggcggcgtcgccgtattcatcggacagcccaccgaagtagtcaaggtccgactgcaggctcaatctcatctacatggtatcaagccccgctacactggcacatataacgcttatagaattattgctacaacagaaggcttaactggactatggaagggcactacacctaatttgatgcggtccgtcatcatcaattgcaccgagctcgtaacttacgatctaatgaaggaggccttcgtgaaaaacaacattctggccgatgatgtcccttgtcacttggtctctgcactgatcgccggattctgtgccaccgccatgtctagccctgtcgacgtcgtgaaaacgcgattcatcaattctccccccggtcagtataaatctgttcccaattgtgctatgaaggtgttcacaaacgaaggcccaactgcattctttaaagggctagtaccctcttttctgcggcttggctcctggaatgtcatcatgttcgtgtgtttcgaacaactgaaaagggagctgtccaaatccaggcagactatggactgcgccacctga

SEQ ID NO: 92 - RNA, 924 nt, synthetic construct
  source 1..924: mol_type = other RNA
atgggtggactcacagcttcagacgtccatccaaccctgggagtgcagctcttcagtgccggaatcgccgcatgcctggcagacgtcattacctttcccctggatacagccaaggtgcgactacaggttcagggggaatgccctacatcctctgtgatccggtataagggcgtgctggggaccattacagccgtggtaaagaccgagggccgcatgaagctttatagcgggctccccgccggcttgcagaggcagatcagctccgcatcactgcgaatcgggttgtacgacacagtgcaggaatttcttactgctggaaaagagactgcccctagtctgggcagcaaaatcctggccgggctgactacaggtggcgttgctgtctttattggacagcccactgaggttgtaaaggtgcgactgcaagctcaatcccacctccacggaattaagccacgctatactggaacgtataacgcctataggatcattgctacaacagaaggtttgactgggctttggaagggtacaacacccaatttaatgcgatccgtcattatcaactgcaccgaactagtgacatacgatctcatgaaggaagcatttgtgaaaaataacatcctggctgacgacgttccttgtcacttagtatctgcactgatcgctgggttttgtgcaaccgcaatgtcttcccccgtcgatgtcgtcaaaacaagatttataaattctccacctggacaatataaaagtgttcccaactgcgctatgaaggtcttcacaaacgaaggcccaaccgcattctttaaggggctagttccttcttttcttcggcttggttcttggaacgtcatcatgttcgtgtgtttcgaacagttgaagcgggagctgtcaaaatcgaggcagaccatggactgcgctacgtaa

SEQ ID NO: 93 - RNA, 924 nt, synthetic construct
  source 1..924: mol_type = other RNA
atggggggactgactgcctcggacgtccatccgacactgggtgtgcagctcttcagtgcagggatcgctgcttgtctcgccgacgttataacctttcccctagacacggccaaggtacgtctacaggttcagggcgaatgccctacttcttctgtcattcgttacaaaggtgtgttagggaccataacagcggtagtgaagaccgaaggccgcatgaaactctacagcgggcttcctgccggactgcaacggcagattagctctgcatccctgcgcattgggctgtacgacactgtgcaggaattcctcacagcaggaaaggagacagcgcctagtctggggagtaaaatcctcgcaggactaacgacaggtggcgtcgctgtgttcattggacagcctaccgaggttgtgaaggtaaggctgcaagcccaatcacacctgcacggaattaagcctcgctacactggaacttacaacgcctacaggattattgccacaaccgagggccttactggactatggaaggggacaacacctaatcttatgcgatcagttattatcaactgcactgagttggtaacctacgatctcatgaaggaagcgttcgtgaaaaacaatatcctggccgacgacgtgccctgccatttggtgtctgcactgatcgcaggattttgtgctaccgcaatgtcttctcctgtcgatgttgtgaagacacggtttatcaattccccaccgggccagtataaaagtgttcctaactgtgccatgaaagtattcacaaacgagggccccacagccttcttcaaggggctagttccctcattccttcggctcgggtcctggaatgtcatcatgtttgtgtgttttgaacagttaaaacgagagctctcgaagtccaggcagacaatggactgtgccacgtga

SEQ ID NO: 94 - RNA, 924 nt, synthetic construct
  source 1..924: mol_type = other RNA
atgggcggtctcacggcctcagacgttcatccaacgctgggcgtgcagctcttcagtgcaggaatcgccgcttgcctggctgatgtcattacctttcctcttgacactgccaaggtgcggttgcaggtccagggtgaatgcccaacttcttctgttattcgctacaaaggcgttttgggaaccatcaccgctgtggtgaaaacagagggtcgcatgaagctgtacagcgggctgccagcgggactacagcgacagataagctcagcatcacttcggattggtctgtatgacacagttcaggagttcctcactgctggaaaggagacagcacctagtctgggtagcaaaatcctggctgggctaacaacaggcggcgtagctgtatttattggacaacccactgaggtggtcaaggtccgactgcaggcccaatctcacctgcatggcatcaagcctcgctacactggtacttacaacgcttacaggatcatagctacaaccgaaggcctaactgggctgtggaagggcacgacacccaatttaatgcgatccgtcatcataaactgcaccgaactcgttacatatgatcttatgaaggaagcatttgtgaaaaataacatcctggcagatgacgttccttgtcatctggtttctgcactcatcgcgggattttgtgctaccgctatgtcaagtcccgtcgacgtcgtgaagacacggttcataaattccccacccggccagtacaaaagtgtccccaactgtgcaatgaaagtcttcacaaacgaaggccccacagcattcttcaagggactagttccctcctttctacgcctcggctcttggaacgtaatcatgttcgtgtgttttgaacaattaaaaagggagctgtctaaatcacgtcaaactatggactgcgccacatga

SEQ ID NO: 95 - RNA, 924 nt, synthetic construct
  source 1..924: mol_type = other RNA
atgggaggcctaacagcttctgatgtccaccccacccttggagtgcagctcttcagtgcaggtatcgctgcttgcctggccgatgttataacctttcctctggatacagccaaggtgcgattgcaggtccagggcgagtgcccgacctcaagcgtgatccgatataaaggcgtgttgggaactatcacagccgtcgtgaagaccgaaggccgaatgaagctttacagcggacttcctgccggcctgcagaggcagataagctctgcatctctccggatcggactgtacgacacagtccaagagttccttactgcaggaaaggagactgcacctagtttagggagcaagatcttggccggactcacaacaggtggcgttgctgtttttataggacagcctactgaagtggtgaaggtgagactgcaggctcaaagccatctgcacggcatcaaacctcgctatactggaacatataatgcttataggatcattgcgaccacagagggcctcacagggctgtggaaagggacaacccctaatttgatgcggtcagtcatcatcaactgcactgagctagttacttatgatctcatgaaagaagcatttgtcaaaaataacattctggcagatgatgtgccttgtcacctggtgtctgcattaatcgcaggattctgcgccaccgctatgtcatctcctgtcgatgttgtgaagacacgattcataaactcccctcctggtcaatataagagcgttcccaactgcgctatgaaagtcttcaccaacgagggcccaacagctttcttcaaggggctcgtgccctcctttctccgactgggatcctggaatgtgatcatgtttgtctgctttgagcagttgaaacgggaattgtctaaatcgaggcaaactatggactgcgccacatga

SEQ ID NO: 96 - RNA, 924 nt, synthetic construct
  source 1..924: mol_type = other RNA
atgggcggcctgacagcctcagacgttcatcccacattgggtgttcagctcttcagtgcaggtattgccgcctgtctcgccgatgtaataacctttccactcgacaccgctaaggtccgccttcaggttcagggcgagtgccccaccagttccgttatcagatacaaaggtgtcctagggaccattacagcggtggtgaagaccgagggccgtatgaagctctatagcgggctgccagcaggcctccagcgccagataagctctgcctccctgcggatcggcctctatgacacagtccaagaattcctcacagctggaaaggagactgcgcctagtctgggtagcaagatccttgccggactcaccacaggcggcgttgctgtattcattggacagcctactgaagtagtcaaggttcgactgcaggcccaaagtcacctccacggcattaagcctcgctacactggtacgtataacgcttatagaataatagccacaacagaaggcttaacgggactctggaaggggactacacctaatttaatgcgatccgtcatcatcaactgcactgagcttgtaacttacgacctcatgaaggaagccttcgtgaaaaataacattttggctgatgacgttccgtgtcacctggtgtctgcactcattgcagggttctgtgccaccgccatgtcttctccggtcgacgttgtgaagacgcgattcatcaattcccctcccggtcaatataaatctgtccccaattgtgccatgaaagtattcactaacgagggcccaacagcctttttcaaggggcttgttccttcctttttgcggctcggatcttggaacgttatcatgttcgtgtgttttgaacaattgaaacgggagttgtctaagtcccgccaaacgatggactgcgctacctga

SEQ ID NO: 97 - RNA, 924 nt, synthetic construct
  source 1..924: mol_type = other RNA
atgggtgggctgaccgcaagtgacgtgcaccccacgttaggggtgcaactcttcagtgccggaattgccgcctgcctcgctgatgttattacttttcctctggacaccgcgaaggtacggttgcaagtccaaggcgaatgtccaacctcttccgtcattcggtataaaggcgtccttgggaccatcactgctgtcgtgaaaacggagggccgtatgaaactctacagcgggcttcctgcaggcctgcaaaggcagatcagttcagcttccctgcggatcgggctgtatgacaccgtgcaggaattccttactgcaggtaaggagaccgcacccagtttggggtccaaaatcttggcaggactcacaacaggaggcgttgctgtattcatcggacagcccaccgaagtggtaaaggtaagactgcaggcccagtctcacctgcacggcatcaagcctcgctacactggaacctacaacgcctacaggatcattgccaccactgaaggtctcactggattatggaaggggaccactcccaatttaatgcgatcagtcatcataaattgcaccgaattggttacttacgatctcatgaaagaagcgtttgtgaaaaataatatccttgcagacgacgttccttgtcacctggtatctgcgctcatcgccggattttgcgcgaccgccatgtcttctcctgtcgatgttgtcaaaacacggtttatcaactctcctcctggtcaatataaaagtgttcccaattgtgccatgaaggtcttcaccaacgaaggccctacagcattctttaaggggcttgttccctcctttctgcgccttggttcttggaatgtcatcatgtttgtctgtttcgaacaactaaaaagggagctgagcaagagtagacaaaccatggactgtgcaacatga

SEQ ID NO: 98 - RNA, 924 nt, synthetic construct
  source 1..924: mol_type = other RNA
atgggtggccttaccgcttcggacgttcatcccaccctgggcgttcagctgtttagcgccggaatagccgcatgtctcgccgatgtaatcacctttcctctggataccgccaaggtgcgcctacaagtccaaggcgaatgcccgacctcttctgttattcggtataaaggtgtcttgggaaccatcactgcagtcgtaaaaacagaaggccgtatgaaactctacagcggacttcctgctgggctccagaggcagataagctctgcctccttgaggattggtctgtacgacaccgttcaggagttccttacagcgggaaaggagacagctcccagcttaggcagcaaaatacttgccgggcttacaacaggtggcgtggcggtgttcattggacagcctaccgaggtggtaaaggtgcgactgcaggcccagtctcatttgcatgggatcaagccgcgctacactgggacttataacgcctatagaatcattgctacgacggagggcttaactggactttggaaggggacaacacctaatttgatgcggtcggtcataatcaattgcaccgaactggtgacttatgacctcatgaaagaggcttttgtgaagaacaacatcctggctgacgacgttccttgtcatcttgtatcagcacttatcgcaggattctgtgcaacagcaatgtccagccctgtcgatgttgtcaaaacacggttcataaattctccacctggccaatataaaagcgtccccaattgtgccatgaaagtattcacaaacgaagggccaaccgccttcttcaagggactcgtcccctcctttctccgtctcggatcctggaatgttatcatgttcgtgtgctttgagcaattaaagcgagagctctccaagtcaaggcaaacaatggactgcgctacatag

SEQ ID NO: 99 - RNA, 924 nt, synthetic construct
  source 1..924: mol_type = other RNA
atgggaggtctcaccgcatctgatgtgcatccaacactgggggttcagctcttcagtgcgggaatagccgcctgtctggctgatgttattacctttcccctggacaccgcaaaggtacgactccaagtccagggcgaatgtccgacctcatcggtgattagatacaaaggcgtgttggggaccatcaccgcagtcgtgaaaacagaaggcaggatgaaactttacagtgggctgcccgcaggcctgcagaggcagatcagctctgcatctctacgcatcggcttgtacgataccgtccaggaattcctgacagcaggcaaagaaaccgcccccagtctgggtagtaagattttggcagggctgactacaggtggtgtcgctgtcttcatcgggcaacccactgaagtggtgaaggtgcgactgcaagctcagagtcacctgcacggaataaaacctcgctatactggtacctacaacgcttatagaattatcgcgaccaccgaaggcctcactggactctggaaagggacgacacctaacttgatgcgatccgtaatcatcaattgcactgagctagttacatacgatctcatgaaggaggcattcgtgaaaaacaatatcctggccgatgacgtcccttgtcacttggtttccgcgctcatcgcaggattctgtgctaccgccatgtcatcccccgtcgatgtcgttaaaactcggttcattaactcaccccctggccagtacaaaagtgttcctaactgtgccatgaaggtgttcacaaatgagggccccacagcattcttcaagggactagtcccttcttttcttcggctcggcagctggaatgtcatcatgttcgtgtgcttcgaacagctaaaacgagagctgtccaagtcaaggcaaaccatggactgtgccacatag

SEQ ID NO: 100 - RNA, 924 nt, synthetic construct
  source 1..924: mol_type = other RNA
atgggggggctcaccgcctccgatgtgcaccccacccttggagttcagctcttcagtgcaggaatcgccgcttgcctcgccgatgttattacttttcccttagacacagcgaaggtacgcctgcaggttcaaggcgaatgtccaacttcaagcgtgattcgatacaaaggcgtgctcggaacaatcacagctgtggtgaagacagagggccgaatgaagctgtactctggtctcccagcaggcctccagaggcagatcagctccgcatccttgcgtatcggactgtacgacacagtccaagaatttcttacagctggaaaggaaacagctcctagtctgggaagcaaaatccttgcaggtcttaccacaggcggcgtcgccgtgttcattggacagcctactgaggttgtgaaggtccgactgcaggctcaatcccatctgcatggtattaagccacgctacactggaacatataacgcttacagaatcatcgcaaccacagagggcttgacaggactctggaaaggcacgacgcccaatttaatgcgatccgtcatcatcaactgcacagaactggtaacgtatgatctcatgaaagaagcttttgtgaagaataacatcttggcagacgacgttccatgtcacctggtttctgcattgatcgccggattctgtgctactgctatgtcatcgcctgttgacgtcgtaaagacacgctttataaactccccacctggtcaatataaaagtgtccccaactgtgcaatgaaagtcttcacaaacgaaggccctacagcattttttaagggactggttccctcctttcttcgtctcggatcctggaatgtcattatgtttgtatgctttgaacagctgaaaagagagctgtccaaatccagacaaacaatggactgtgcaacctga

SEQ ID NO: 101 - RNA, 924 nt, synthetic construct
  source 1..924: mol_type = other RNA
atgggcggtctgacagccagcgacgtgcatcccacactgggtgtgcagctcttcagtgcaggaattgccgcttgcctcgctgatgttattacatttcctctagatactgccaaggtacgattgcaggttcaaggcgaatgcccaacaagtagcgttatccgatataaaggcgtgttgggaaccatcaccgctgtagtgaaaaccgaaggcaggatgaagctctacagcgggctcccagccggcctacaaaggcagataagctccgcctccctgcgtatcggattgtacgacacggtccaagagttccttacagcgggaaaagagacagcgcctagcttggggtccaaaattctggccggactcacaacaggaggtgtggctgtgttcattggacagcctacagaagttgtcaaggtgcgactacaggctcaatctcacctgcatgggataaaacctcgctacactgggacttacaacgcttacaggattattgccacaacagaaggcctgacgggactttggaaaggtacaacacctaatttaatgcgttccgttatcatcaattgcactgagctggtcacttacgatctcatgaaggaagcattcgtgaaaaataacatcttggcagatgacgttccctgtcacttggtgtccgcactgatcgcaggattctgtgccactgcaatgtcaagccctgtcgatgtcgtgaaaacaaggtttatcaattccccacctggtcagtataaatctgttcctaattgtgccatgaaggtattcaccaacgaaggccccacagccttctttaagggactggttccctcttttctccggcttggatcctggaacgtcataatgttcgtatgttttgagcagttaaaaagagagctgtctaaatccaggcagacaatggactgtgctacatag

SEQ ID NO: 102 - RNA, 924 nt, synthetic construct
  source 1..924: mol_type = other RNA
atgggcggacttacagcatctgacgtgcaccctacattaggtgttcagctcttcagtgcaggcatcgctgcttgcctcgccgatgtcattacctttcctctggataccgctaaggtacgcctccaggttcagggcgaatgcccaacttcctccgtgattcgatataagggagttttgggaaccattacggctgtggtgaagactgagggccgaatgaaactttacagcgggctgcctgcgggactgcagcggcagatcagctcagcttcgttgcggatcgggctatatgacaccgttcaggaattccttacggccggaaaggaaaccgcacctagtctgggtagtaaaattcttgccgggcttacgacaggcggcgtcgctgtgttcattggccaacccactgaggtggtaaaggtccggcttcaggctcaaagccacctgcacggaatcaagcctcgctacactggaacatacaacgcttacaggatcatagccactacagaaggcctgacagggctttggaagggcacgacacccaatttaatgcggtctgtgatcatcaattgcactgaactggtgacttacgacctcatgaaggaagctttcgtgaaaaacaacatactggccgatgacgtcccatgtcatctggtgtccgcattgatcgccggattttgtgctacagctatgtcttcccctgtcgacgtcgttaagacacgatttataaactctccaccaggacaatacaagagtgtccccaattgcgccatgaaagtcttcacaaacgaaggccctacagccttctttaaggggctcgttccctcctttcttcggctcggctcttggaacgtcatcatgttcgtgtgcttcgagcaactgaaacgggagttatccaaaagtagacagaccatggactgcgctacttga

SEQ ID NO: 103 - RNA, 924 nt, synthetic construct
  source 1..924: mol_type = other RNA
atgggcggattaactgccagtgacgtccaccccactctgggggtacaactcttcagtgccggaatcgccgcttgtctcgccgatgttattacctttcccttagacaccgctaaggtgcgcctgcaagttcaaggcgaatgcccaacatcaagcgttatacgatataagggcgttctcgggaccatcacagctgttgtaaaaacagaaggccgtatgaaactctacagcggactgcctgccggcctgcagaggcagatcagttcggcgtccctgcgcatcggtttgtacgataccgtgcaagaattcctgaccgccggaaaagagacagctcccagtttgggtagtaaaattctggcggggctcacaacaggtggcgttgctgtgttcatcggacagcctacagaggtagtcaaggtccgactccaggctcaatcacacctgcacggaatcaaacctcgctacactgggacatacaacgcttacaggattatcgccaccacagaaggcttgactgggttgtggaagggcacaacacctaatttaatgcgatctgtcataatcaattgcacggaactagtaacgtacgatctcatgaaagaagcttttgtgaaaaataacattctggcagatgacgtcccctgtcacttggtgtctgcactcatagctggattttgtgcaactgctatgtcatcccccgtcgatgtcgtgaaaacacggtttatcaattcaccaccaggccaatacaaatctgtacctaattgtgccatgaaggtgttcacgaacgaagggccaacagccttcttcaaggggcttgttccatcctttctccgactcgggtcatggaatgtgatcatgttcgtgtgtttcgagcaactgaaaagagagttgtcaaagagtagacaaacgatggactgcgccacatga

SEQ ID NO: 104 - RNA, 924 nt, synthetic construct
  source 1..924: mol_type = other RNA
atgggtggattgactgcctccgatgtccatcctacattgggggttcagctcttcagtgcgggcatcgccgcttgccttgccgatgtcattacctttccactagacacagctaaggtacgtctacaagtccaaggagaatgccccacctcttctgtgattagatacaagggcgtactgggaaccatcacagctgtcgtgaagaccgagggacgcatgaagctttacagcgggctgccagcaggactgcagaggcagataagctcagcatccctgcgaatcggactatacgacacagtccaggaatttcttactgcgggaaaagagacagctcccagtttagggagtaaaatactcgccgggctgacaacaggcggcgttgccgttttcataggacagccgactgaagtggttaaggtgcgactccaagctcagtctcacctgcatggtatcaaacctcgctatactggtacatataacgcctacaggattatagcaacaacagaaggtttgactggactctggaaggggacaacacccaatttgatgcgatccgtcattattaactgcaccgagctcgtaacctatgatctcatgaaggaggcctttgtaaaaaacaacatcctggcagacgacgttccttgccacttggtttcagcactcatcgccgggttctgtgctaccgctatgtctagccctgtggatgttgtcaaaacacgattcatcaattcaccaccgggccaatataaatctgtccccaattgcgccatgaaggtcttcacaaatgaaggcccgactgccttcttcaaggggctcgttccttcttttctccggctcgggtcctggaatgtgataatgttcgtgtgtttcgaacagctgaaaagggagctgtcaaaatcaaggcaaaccatggactgcgccacctga

SEQ ID NO: 105 - RNA, 924 nt, synthetic construct
  source 1..924: mol_type = other RNA
atgggaggtctgacagcttcagacgtacaccccacattaggcgtgcaactcttcagtgccggcatagccgcttgcctcgccgatgttattacctttcctctggacaccgctaaggtgcgacttcaggttcagggcgagtgtccaacctcatccgttattcgatacaaaggcgtgctaggaaccataaccgccgtggtcaagacggaaggccgtatgaaactttacagcggactcccagcgggactccagagacaaataagctctgcatctcttcgaatcggactgtacgataccgtccaggagtttctaacagctggaaaggagacagcacctagtttgggcagcaaaattcttgccgggctcactacaggaggcgtcgccgtgtttattggacagcccacagaggtagtgaaggtgcgactgcaggcccaatctcatctgcacggaattaaaccgcgctacaccggtacatataacgcctacagaatcatcgctaccacagaaggtctgactgggttgtggaaggggacaacccccaatttaatgcgatctgtgatcatcaactgcactgagctggtcacttacgatctcatgaaagaagcattcgtgaaaaacaacattttggctgacgacgtcccatgtcatttagtttctgcgctcatcgctgggttttgtgctacagcaatgtcttctcccgttgatgtcgtcaaaacacgattcataaactctccacctggccaatacaaaagtgttcctaactgtgccatgaaagtcttcacgaacgaaggccccacagccttcttcaaggggctggttccctcctttcttcggctcggatcctggaatgtaatcatgtttgtgtgcttcgaacagttaaaaagagagctctcaaaatcaaggcagacaatggattgtgccacttga

SEQ ID NO: 106 - RNA, 924 nt, synthetic construct
  source 1..924: mol_type = other RNA
atgggtggactgacagctagcgatgtgcaccccacactgggcgtgcaactcttcagtgctggtattgccgcttgcctcgccgacgtcataacgtttcccttagacactgctaaagtccggcttcaggtccagggtgaatgcccaacctcttcagtcatccgttataaaggcgtgctgggaaccatcacagcggtggtaaaaacagagggccgcatgaagctgtatagcgggctccccgcaggcctccagaggcagataagctctgcttctctgcggatcggtttgtatgacactgttcaagaattccttaccgccggcaaagaaaccgcacctagtctgggtagtaaaatcctcgccgggctcactacaggcggtgtcgccgtattcatcggacaacctaccgaggtggtcaaggtaagactgcaggcccaaagccacctgcacggcatcaaacctcgctacaccggcacttacaacgcctacaggattatagctaccaccgaaggcttgacgggactgtggaaaggcacaaccccaaatttaatgcggtccgttattatcaactgcaccgagctggtaacttatgatctcatgaaggaagcatttgtgaaaaataacatcttggcagacgacgttccatgtcacttggtttcggcgctaatcgccggattttgtgctacagccatgtcatcccctgtcgatgtcgtgaagacaaggttcatcaattcaccaccaggtcagtacaaaagtgtccccaattgcgcaatgaaagtattcacaaacgaaggcccaaccgcgttttttaaggggcttgttccctcctttctgcgcctcggttcttggaatgtcatcatgtttgtatgcttcgagcagctgaaacgggagctgtccaagtccagacagactatggactgtgcgacatga

SEQ ID NO: 107 - RNA, 924 nt, synthetic construct
  source 1..924: mol_type = other RNA
atgggggggctgacagcttcagacgttcaccccaccctgggagttcaactctttagtgctggaatcgccgcctgcctcgcagatgtcatcacctttcctctggatacagcaaaggtgcggcttcaggttcagggcgaatgtccaacttcttcagtgatccgctataaaggcgtcttgggaaccatcaccgcagtcgtgaagaccgaaggccgaatgaagctttacagcgggctgccagccgggctgcaacgacagataagctcagcatccctgcgtataggcctatacgacaccgtccaagagtttctcactgccggaaaggagacagctcccagtctgggaagcaagatcttagccggactcaccacaggcggcgtggccgtgttcattggacaacctaccgaggttgttaaggtgcgactacaggctcagagccatctgcatgggattaaaccacgctatactggaacttacaacgcttataggattatcgccaccactgaaggattaactggactttggaagggcacaacacccaacttgatgcgatcagtgatcataaattgcaccgaactggtgacctacgatctcatgaaggaggctttcgtgaagaacaacatacttgccgacgacgttccttgtcacctcgtctctgcgttgatcgccgggttctgcgctactgctatgtcatctcccgtcgatgttgtgaaaacaaggtttatcaattcgcctccaggccagtataaaagtgtccctaactgtgccatgaaagtgttcacaaatgaaggccccacagcatttttcaagggactcgttccttcctttcttcgtctcggatcctggaatgtcatcatgtttgtgtgcttcgagcaattgaaacgagagctgtcgaagtcacgacaaaccatggactgcgcaacctga

SEQ ID NO: 108 - RNA, 924 nt, synthetic construct
  source 1..924: mol_type = other RNA
atgggtggtctcactgcaagcgacgtgcatccaacactgggggttcagctcttcagcgccggaatcgccgcttgccttgccgacgtcataacttttcctttagataccgctaaggtacgcctgcaagtccagggcgagtgccccacatcaagtgtgattcgctacaaaggcgttttggggaccatcacagcagtggtgaagacggaaggccgaatgaaactttatagcggactgccagcaggactgcagcgccaaataagctctgcttcactgcgtatcggtctatatgataccgtgcaggaatttcttactgcaggaaaggagacggcacccagtttggggagcaaaatcttggccggcctgactacaggcggtgtcgctgtattcattggacagccaactgaagtggtgaaggtgcgactccaggctcaatctcacctgcacggtatcaaaccccgctacactggcacatataacgcttacaggattatcgctacgacagaaggcttaacgggcctctggaaaggcaccacccctaatttaatgcgatctgtcatcatcaattgcactgaactcgtaacgtatgatctcatgaaagaagcgtttgtgaaaaataatatcctggcggacgacgtcccttgtcacctggtgtctgcacttatcgccggattttgtgccaccgccatgtccagtcctgttgacgttgtcaaaacaaggttcatcaattcacctcctggtcaatataaaagtgttcccaactgtgccatgaaagtcttcacaaatgaaggtcccacagctttttttaaaggactagttccttccttcctgcggcttggatcctggaatgtcatcatgtttgtgtgttttgaacagctgaaacgggaacttagtaaatccagacagaccatggactgtgccacctga

SEQ ID NO: 109 - RNA, 924 nt, synthetic construct
  source 1..924: mol_type = other RNA
atgggtggtctaaccgcttcagacgtgcatcctactctgggagtgcagctcttcagtgcaggtatcgccgcttgccttgccgacgtgatcacctttcccctggacaccgctaaggtgcgcctgcaggtccagggcgaatgtcctacatcaagcgtgattcggtataaaggcgtattgggaaccatcactgctgtggtaaagactgaaggaagaatgaagctatacagcgggcttccagctggacttcagaggcagatcagctccgcttctttgcgtatcggcctgtacgacaccgttcaggaatttctcactgcaggaaaggagacagcacccagcctgggcagcaaaattctagccgggctcaccacaggtggcgttgctgtatttatagggcaaccaacagaggtggtcaaggtgaggctgcaagctcaatcccacttacacggtattaagccacgctatactggaacttacaacgcctacaggattattgctacaacagaaggtcttactggactttggaaggggacaacacctaatttaatgcggtccgtgatcattaactgcaccgaattggttacctacgatctcatgaaagaagcctttgtgaagaataacatcttggccgatgacgttccttgtcacctcgtgtctgcactaatagccggattctgtgctacagctatgtcatctcctgtcgatgttgtcaaaacgaggtttatcaattctcctccgggtcagtataaaagtgtccctaattgtgccatgaaagtattcacgaacgaaggacccaccgccttcttcaagggacttgttccttcttttctccgtctcggttcctggaatgtcatcatgttcgtgtgttttgaacagctgaaaagggaacttagcaaatcaagacaaactatggactgtgctacgtga

SEQ ID NO: 110 - RNA, 924 nt, synthetic construct
  source 1..924: mol_type = other RNA
atggggggtctgactgcctcagacgttcatccgacactgggggtgcagctcttcagtgccggcattgccgcttgcctcgccgatgtcattacttttcctctggacaccgcgaaggtccgcctgcaagttcagggcgagtgtcctacatcttccgtcatccggtataaaggcgttctgggaaccatcacagccgtcgtgaagaccgagggccgaatgaagctttacagcgggctgcctgctgggctgcagcgacagataagctccgcttctcttcggatcgggctgtacgacacagtacaggagtttctcacagctggaaaggagactgcacccagtttgggtagtaaaattctcgcggggctcaccactggcggtgtcgctgtgtttattggtcagcctactgaggtcgtcaaggtcaggctgcaggctcagagccatctgcatgggatcaaacctcgctacactggcacatataatgcctacaggataattgcaacaacagaaggcctgactggactgtggaaagggacaacacccaatttaatgcgatccgtcatcattaattgcaccgagctcgtaacatacgatctcatgaaggaggcatttgtgaaaaataacattttagcagatgatgttccatgtcacctggtatcagcactcatcgcaggattctgtgctactgctatgtcttctccagtggacgtggtgaagactcggtttatcaattctccacctggccaatacaaaagtgtccctaactgtgccatgaaggtattcactaacgaaggcccgactgccttctttaagggactcgtcccttcttttctgcggcttggctcgtggaacgtgattatgtttgtgtgttttgaacagctgaaaagggagctaagtaagagcaggcaaacaatggactgtgccacttga

SEQ ID NO: 111 - RNA, 924 nt, synthetic construct
  source 1..924: mol_type = other RNA
atgggtgggctcaccgcatcagacgttcaccccacactgggagttcaactcttcagtgcgggcatcgccgcctgcctggctgacgttattacttttcctctggacacagctaaggtacgtttacaggttcaaggagaatgtcccacttcttccgtgattcggtataaaggcgttttggggactatcacagccgtagtgaagacagaaggccgcatgaagctctacagtggtctgccagcaggcctgcagcgacaaatcagctctgcttcactgcgtatcggtttgtacgacactgtgcaagaattcctcaccgcaggaaaggagaccgcacccagcttagggagcaaaatccttgccgggctcacaacaggcggcgtcgctgtgttcatcggacagcctacagaggtggttaaggtaagacttcaggcccagtcacaccttcatggtataaaacctcgctatactggaacgtacaatgcttacaggatcatcgccaccacagaaggcctcacagggctgtggaaaggtacaacccctaatttgatgcgatcagtcatcatcaattgcactgagctggtgacttacgatctcatgaaggaagcttttgtcaaaaacaacatactggccgatgacgtcccatgccacctggtttccgcactcatcgcaggattctgtgccaccgcaatgtcttctcctgtcgatgtcgtgaagacacgatttatcaattcacctcctggacagtacaaatctgtccccaattgtgccatgaaagtgttcacaaacgaagggccaacagccttttttaaggggctcgtgccctcctttcttcgactcggatcttggaacgtcattatgtttgtgtgttttgaacagttaaaaagagagctctccaaatctaggcagacaatggattgcgccacttag

SEQ ID NO: 112 - RNA, 924 nt, synthetic construct
  source 1..924: mol_type = other RNA
atgggcggtctcacagctagcgatgtgcatcccacgctgggggtgcaacttttcagcgcgggcatcgccgcttgcttagctgatgtcattacctttcccttagatacggctaaggtccgccttcaggtccagggcgaatgtcccacctcatccgtgattcgctataagggcgtattagggacaatcacagccgtcgtgaagaccgagggccgtatgaaactctatagcgggctgcccgccggattgcagcgacagatcagctctgcatccctgcgaattggcctgtatgacactgtgcaggaattcctcaccgccgggaaagagacggcgcccagtctgggcagcaagatactggccggactcactacaggtggcgtggcggtgttcattggacagcctacagaggtggttaaggttagactgcaggctcaatcccatctgcacgggatcaaacctcgctatactggcacttataatgcttacaggatcattgccacgacagagggcctcactggactttggaaagggacaacacccaatttaatgcgaagcgttatcatcaactgcaccgagctcgtgacctatgatctgatgaaggaagcctttgtaaaaaacaacatcctggcagatgacgtcccatgtcacttggtatctgcactcatagctggcttttgtgctaccgccatgtcttcccctgtggatgtggtaaagacacgattcatcaattctccacctggtcaatataaaagtgttcctaattgtgcaatgaaagtattcactaacgaaggcccaacagcgttcttcaaggggctcgtgccatcttttctgcggctcgggtcctggaatgtcatcatgtttgtgtgcttcgaacagttaaaaagggaactgtcaaaatcaaggcagacaatggattgcgctacttga

SEQ ID NO: 113 - RNA, 924 nt, synthetic construct
  source 1..924: mol_type = other RNA
atgggcggtctgaccgcatctgacgtacatcctacccttggggtgcagctctttagtgctggaatcgccgcttgccttgcggacgttattacttttcccttagatactgctaaggtgcgcctccaggtccagggcgagtgtcctacatcatccgttattcgctacaagggcgtattaggcactattacagccgtggtgaaaaccgaaggccgcatgaaattgtatagcgggctccctgctggtttgcaaaggcagatcagctccgcttctctgcggatcggtttatacgacaccgttcaggagtttcttaccgcaggaaaggagacggcacctagtttagggagcaaaattctcgccgggctcactacaggtggcgtcgccgtattcatcggacagcctacagaggtggtaaaggtacgcctgcaagctcagtctcatctgcacggcattaaacctcgctacactgggacatataacgcttataggattatcgccacaacagaaggcctaacgggactatggaaaggcacaacccccaacttaatgcggtctgtcattatcaactgcactgaactcgtgacttacgatcttatgaaagaagctttcgtgaagaacaacatcctggccgatgatgttccttgtcacttggtgtccgcacttatcgctggattctgtgctaccgctatgtcatcccctgtcgacgtcgttaaaacacgcttcataaattctccacctggtcaatataaaagtgttcccaattgtgccatgaaggtgtttacaaatgagggacccacagcctttttcaagggcctggttccatcgtttctacggctcggatcctggaacgtcatcatgttcgtctgttttgagcaactgaaacgggagctttccaaatccagacagacgatggactgtgctacttga

SEQ ID NO: 114 - RNA, 924 nt, synthetic construct
  source 1..924: mol_type = other RNA
atggggggtttaaccgcttcagatgtgcatccaacgctgggggtgcaacttttcagtgcagggattgccgcttgcctcgcagacgtcattacttttccattggacaccgcaaaggtgcgcctgcaggtccagggcgaatgcccaacttcatcggttatcagatataagggcgtcttgggaaccatcacagccgtggtgaagactgaaggcaggatgaaactctacagcgggctcccagccggcctacagcggcaaatcagctccgcatcgctgagaatcggactgtatgacaccgttcaagaattcctaaccgcaggaaaggagacagcacccagtctgggaagtaaaattctcgctgggctgactactggcggcgtggccgtattcatcggacaacctactgaagtggttaaggtacggctacaagctcaaagccacctgcacggcattaaaccacgctatactggcacttacaatgcttataggataattgcaacaacagaaggcctcactggactttggaagggcacaacacctaatctgatgcgatctgttattattaattgcactgagctcgtgacttacgatctcatgaaagaggcctttgtgaagaataatatcctggcagacgacgttccttgccaccttgtgtccgcactaatcgcgggattctgtgccaccgcaatgtctagccctgtggatgttgtcaaaacacggtttataaactcccctcccggccagtataaaagtgtccccaattgtgcaatgaaggtcttcactaatgaaggccccacagcatttttcaaagggctggtgccctcctttcttcggctcggatcgtggaacgtgatcatgttcgtgtgtttcgaacagctgaaaagggagttgtcaaagagcagacaaaccatggactgtgctacctga

SEQ ID NO: 115 - RNA, 924 nt, synthetic construct
  source 1..924: mol_type = other RNA
atgggaggtttaactgccagcgacgttcatccaactctgggggtgcagctcttcagtgcaggcatcgccgcatgcctcgctgacgtgattacgtttcctttagacaccgctaaggtccgtcttcaagtccaaggcgaatgtcctacttcttccgtcattcgttacaagggcgtgttggggaccatcacagccgtggtgaagactgaaggcaggatgaaactttatagcgggctccctgctggcttacagaggcaaatcagctctgcttcactccggattgggctctacgacactgtccaggaatttcttaccgccggaaaggagacagctcccagcttggggagtaaaatcctcgcgggactcactacaggcggcgtcgccgtcttcattgggcaacccactgaggtggtgaaggtccggttgcaggctcaatcccaccttcacggcattaagcctcgctacacaggcacatacaacgcctacagaatcattgccaccacagaagggttaaccggactttggaaggggacaacacccaacttaatgcgatctgtcatcatcaattgcactgaactcgtgacttacgacctcatgaaggaagcctttgtgaagaataacatcttggccgacgacgttccttgtcacctggtttctgcattaattgctggtttttgtgctactgccatgtcatctcctgttgatgtcgtcaaaacacggttcatcaactcccctcccgggcagtacaaatctgtccccaattgtgccatgaaagtcttcacaaacgagggtccaaccgccttcttcaaagggcttgttccctcttttcttcgtctcggctcctggaacgtgatcatgttcgtgtgcttcgaacagctgaagagggaactgtccaaatccaggcaaacgatggattgtgccacatga

SEQ ID NO: 116 - RNA, 924 nt, synthetic construct
  source 1..924: mol_type = other RNA
atgggcggtttaacggcaagcgacgtgcatcccacgctgggcgtgcagcttttcagtgccggaatcgctgcttgcttagcagacgttataacttttccccttgacacggccaaggtccgcctgcaagtccaaggcgaatgtccaacctcatcggttatccgctataaaggcgttttgggaactattacagccgtcgtgaagaccgagggcaggatgaaactttacagcgggctccctgctggtttgcagaggcagataagctcagcatcccttcgaattggtctgtacgacaccgttcaggagttccttactgcgggaaaggaaacagcccctagtttgggcagcaaaattctggccgggctgactacaggtggtgtcgccgtatttattggacaacccacagaagtagtaaaggttcgactgcaggcccagagccatttgcatggaatcaagccgcgctacactggtacttacaacgcctatagaatcatcgccaccacagagggattaactggactttggaaggggacaacccctaatttaatgcgatcagttatcatcaattgcaccgagctagtgacgtacgatctcatgaaagaagcatttgtcaaaaacaacattttggcagatgacgttccatgtcacttggtctctgcgttaatcgccggattttgtgcaaccgctatgtcttctcctgtcgacgttgtgaagacaaggttcataaactctccacctggccaatataaaagtgtccctaattgtgccatgaaagtctttactaatgaagggcctaccgcattttttaaggggctcgttccgtcctttctgcggctcggatcctggaacgtgatcatgttcgtgtgttttgagcagttgaaaagggagctgtctaagtcaaggcaaacgatggattgtgccacctga

SEQ ID NO: 117 - RNA, 924 nt, synthetic construct
  source 1..924: mol_type = other RNA
atggggggtcttactgcatcagacgttcatcccacactgggggtgcagctcttcagtgcgggcatcgccgcgtgcctcgcagatgtcattacgtttcccctggatacagctaaggtgcgcctgcaggtccagggcgaatgcccaacttcctccgtaatccgttacaaaggcgtgttgggcaccatcacagccgtcgtgaagaccgagggacgtatgaaactttacagcgggctccctgccgggctgcaacggcagatcagctccgcttccctccgtatcggactatacgacacagtgcaggaattccttacggctggaaaagagacggcacctagcttgggcagcaaaatcttggcagggctcactacaggtggcgttgctgtctttatcggacagcctactgaggtagtgaaggtccgtctccaagctcaatcccacctgcacggcatcaaacctcgatacaccggcacttataacgcctacaggattatcgctactacagaaggccttactgggctgtggaaggggacaacacctaatttaatgcggtcggttatcatcaattgcactgagctcgtgacctacgatctcatgaaggaagcgtttgtgaaaaacaacattctggcagatgacgtcccctgccacttggtgtctgctctcatcgccggattttgtgccaccgccatgtcttcccctgttgacgttgtgaaaacaagatttatcaattctcctccaggtcaatataaaagtgtccccaattgtgccatgaaggtgttcacaaatgagggcccaacggcattcttcaaggggctcgtcccttcctttcttcgactggggtcgtggaacgtcatcatgtttgtatgctttgaacaattgaaacgggagctgtccaagtcccggcagacaatggactgtgccacttag

SEQ ID NO: 118 - RNA, 924 nt, synthetic construct
  source 1..924: mol_type = other RNA
atgggcggcctgacagcttctgatgtgcatcctacactgggtgtgcaacttttcagtgcgggaatcgctgcttgcctcgccgacgtcattacctttcccttagacacagcaaaggtccgcctgcaggtccaaggcgagtgtcctacttcctccgtcattcgatacaagggtgtgctgggaaccatcaccgctgttgtaaaaacagaaggccgtatgaagctttacagcggactgccagccggtttacagaggcagatcagctctgcgtcgctgcgtatcgggctctatgacaccgtccaagagttcctaacagcagggaaggagacagcgcccagcctggggagtaaaatcctcgcaggactcacaacaggtggtgtcgccgtgttcattggacagccaactgaggtggtgaaggtccgactgcaggcccagtcccacctccacgggatcaaacctcgctacaccgggacgtacaacgcttatagaatcatcgccaccacagaagggctcacaggactctggaaggggacaacacctaatttgatgcgatctgttattattaattgtactgaactcgtgacttatgatctcatgaaggaggcctttgtgaaaaacaacatcctggccgacgacgtcccatgtcacctggtatctgcactgatcgccggattctgtgcaaccgccatgtcatctcctgttgacgttgtcaagacacgcttcataaactctcctccgggtcagtataaaagtgtccccaattgtgccatgaaagtatttaccaacgaaggcccaaccgcattctttaaggggcttgtgccatcttttctgcggctgggctcctggaatgtcatcatgttcgtgtgtttcgaacaattaaaaagagagctgtcgaagtcccggcagacgatggactgcgccacttag

SEQ ID NO: 119 - RNA, 924 nt, synthetic construct
  source 1..924: mol_type = other RNA
atggggggtctcacagctagtgacgttcatcctacactcggagtgcagctcttcagtgcaggaatcgccgcttgccttgcagacgttatcacctttcctcttgacaccgctaaggtccgactgcaggtccaaggcgagtgtcccacctcctccgtaatcagatacaagggtgtcctggggacaatcaccgcagtcgtaaagacagagggccgtatgaaactttacagcgggctcccggcaggattgcagcgccagatcagctccgcatctctgagaattggtctctacgacactgttcaagaattcctaacagccggaaaggagactgcacccagtctgggaagtaaaatactagccgggctcaccacaggcggcgttgctgtattcataggacagcctaccgaagtagtcaaggtcagacttcaggctcagtctcatctgcacggaatcaagccccgttataccggaacttataatgcttacaggatcatcgccaccacagaaggcttaactggactttggaaggggaccacacctaatttaatgcgttccgttataatcaactgcaccgagctagtgacttacgacctcatgaaggaggcatttgtgaaaaacaacatcctggcagatgacgttccttgtcacttagtgtctgcactcatcgccggattttgtgcaaccgccatgtccagtcccgtcgatgtcgttaagactcggttcattaactcccctccgggccaatacaaatctgtccctaactgtgccatgaaagtatttacaaacgagggtcctacagccttctttaaggggctcgtcccgtcttttctccggctaggctcgtggaacgttatcatgtttgtgtgtttcgaacaactaaaaagagagctttccaaatccaggcaaaccatggattgcgcaacttga

SEQ ID NO: 120 - RNA, 924 nt, synthetic construct
  source 1..924: mol_type = other RNA
atggggggcctgacagcttccgacgtccaccccacattgggagttcagctcttcagtgcaggaatcgccgcttgccttgccgatgtcattacttttcctttggataccgctaaggtgcgcctgcaagtccagggcgagtgtcctacttcttctgtgattcgatacaagggcgtcttaggaactattacagccgtcgtgaaaaccgaaggtcgtatgaagctctacagtgggctgccagccggactgcagcggcagataagctcagcttctctccgaatcggactgtacgacaccgttcaggagtttcttactgccgggaaggaaaccgcacccagtctggggagcaaaattcttgccgggctcaccacaggcggcgtcgccgtcttcatcggacagcctaccgaagtcgtgaaggtgcgactgcaagcccagtcacacctgcatggcatcaagccacgttatactggaacttacaacgcttacaggattatcgccaccactgaaggccttactggactctggaaggggacaactcctaatttaatgcgatcagttatcattaactgcacagagctcgttacttatgaccttatgaaggaagcattcgtgaaaaacaacatcttagcagacgacgtcccctgtcacctggtatctgcactaatcgccggattttgtgctaccgccatgtcatcccctgtggacgtcgtaaagacgcgattcataaattctcctcccggtcaatacaaatcggtccccaattgtgccatgaaagtgttcacaaacgaggggccaaccgcctttttcaaagggcttgtgccctcttttctgcgactcggctcctggaatgtcatcatgttcgtgtgcttcgaacagctaaaaagggagctgtctaaatccaggcaaacaatggactgtgctacttaa

SEQ ID NO: 121 - RNA, 924 nt, synthetic construct
  source 1..924: mol_type = other RNA
atgggcggtcttacagcaagcgacgttcatcccacactcggggtgcagctctttagtgcgggaatcgccgcttgcctcgccgacgtgataacctttccgttggacacagccaaagtacgccttcaagtccagggcgagtgcccaacttcttccgtgattcgatataaaggcgtgttgggaaccattactgccgtcgtgaagaccgaaggccgcatgaagttatacagcgggctgcccgctgggttgcagaggcagataagctctgcctccctgcgtatcggcttgtatgacacggttcaagaattcctcactgccgggaaggagaccgcccccagcttgggcagcaaaatcctggccgggctcactacaggcggcgtggctgtatttattggacaacctaccgaagttgttaaggtacgacttcaagctcaatctcacctgcacggaattaaaccgcgctacactggaacttacaacgcttatcgaattattgccactacagaaggcttaacaggactttggaaggggacgacccctaatttaatgcggtcggttatcatcaattgcaccgaattggtgacatacgatcttatgaaagaggctttcgtgaaaaacaacatcttggcagatgacgttccttgtcacttagtatccgcattaatcgccggattctgtgctactgccatgtcctcccctgttgatgttgtcaagacaaggttcataaattcgcctcctggccaatataaaagtgtccccaattgtgccatgaaagtcttcaccaacgaagggcctaccgccttctttaaggggctcgtcccgtcttttctgcgtctggggtcctggaacgtgatcatgtttgtgtgttttgagcaattgaaaagagagctgtcgaagtccagacagacgatggactgtgctacatga

SEQ ID NO: 122 - RNA, 924 nt, synthetic construct
  source 1..924: mol_type = other RNA
atgggtggactgaccgcttcggacgttcaccctaccctgggagtgcagctcttcagtgcaggaatcgccgcgtgcctcgccgatgtcattacttttcctttagatacagctaaagtgcggctgcaagtccaaggagaatgccccacatcctcagtcattcgatacaaaggcgttttggggaccatcactgccgtggtgaaaacagagggccgaatgaaactctacagcgggctgccagccggattgcagaggcagatcagctccgcatctctgcgaatcgggttgtacgacacagttcaggagtttctgactgccggaaaggagaccgcacctagtttgggcagtaaaatcctggccggactcactacaggtggcgtcgccgtatttatcgggcagcccaccgaagttgtgaaggtccggctgcaggctcagtctcacctacacgggattaagccacgctacactgggacatacaacgcttataggatcatcgccaccactgaaggtcttacgggactttggaaaggcacaacacccaacctaatgcgatctgttatcatcaattgtactgagctggtcacttacgatctgatgaaagaagcgtttgtgaaaaacaacattctcgcagatgacgttccatgtcatctagtgtccgcactcatcgccgggttctgtgcaaccgccatgtccagccctgttgacgtcgttaagacaagatttattaattctccaccaggccaatacaaaagcgtccctaactgtgccatgaaagtgttcacaaacgagggccctactgccttctttaaggggctggttccctcctttctgcgactcggatcctggaatgtcatcatgttcgtatgctttgagcaattgaaaagagagttgtctaagtcaagacaaaccatggactgcgccacatga

SEQ ID NO: 123 - RNA, 924 nt, synthetic construct
  source 1..924: mol_type = other RNA
atgggcggcctaaccgcgagtgacgttcaccctactttaggggtgcagcttttcagcgcaggcatcgcagcctgcctcgctgacgtcattacatttcccctcgacaccgccaaggtccgcctgcaagttcagggtgaatgtccgacttcctccgtcattcgttataaaggcgtgctcgggaccattaccgctgtggtgaagaccgaaggccgcatgaaactgtatagcgggctgcctgcgggtctccagagacaaatcagctcagcctctctccgtatcggcctatacgatacagttcaagaattcttaacggccgggaaagaaaccgcaccttctctgggaagcaaaattctcgccgggctcactacaggcggcgtcgccgtcttcatcggccaaccaactgaagttgttaaggtacgactgcaagctcaaagtcaccttcacggcatcaagcctcgctacactggcacttacaatgcttacaggattatcgctacaaccgaaggcctaactggactctggaaggggacaacacccaatttgatgcggtctgttattatcaactgcacagaactcgtgacatacgatctcatgaaggaagctttcgtgaagaataacatcctggctgatgatgtcccttgccacctcgtatctgctctgatcgcaggattttgtgctaccgctatgtcaagccctgtcgacgtcgtaaaaacacggtttataaactccccccctggccagtacaagtctgtccctaattgtgccatgaaagtcttcaccaatgaaggccccactgcctttttcaaggggctcgtgccctcctttctgcggctcgggtcctggaatgtcatcatgtttgtgtgttttgagcaactgaaaagggagttaagtaaatccaggcagaccatggactgcgccacatga

SEQ ID NO: 124 - RNA, 924 nt, synthetic construct
  source 1..924: mol_type = other RNA
atgggcggtctgacagcatccgacgtgcatcctactctgggggtgcagctcttcagcgccgggattgccgcgtgcctcgctgatgttataacttttcccctggacaccgctaaggtccgtctgcaagttcagggcgagtgccccacctcttctgtaattcgctataaaggcgtccttggaaccataactgctgttgtgaagactgaaggccgtatgaagctctatagcgggctgcctgcgggcctgcagaggcagatcagctccgcctccctccgtatcggcctgtacgacactgtccaagaattcctcactgctggaaaagaaactgcacccagtctggggagtaagatcctcgccgggcttactacaggtggcgtcgctgtctttattggacagccaacagaggtcgtgaaggtgcgactgcaggctcaaagccacctgcatggaattaaacctcgctacactggaacctataacgcctataggattatcgcaaccaccgaaggtttaactgggctctggaagggaacaactcctaatctaatgcgatcagttattatcaattgcactgaactcgttacttatgatctcatgaaggaagcctttgtgaaaaataatatattggcggacgacgtcccttgtcacctggtatccgcacttatcgccggattttgtgctaccgctatgtccagccctgtcgatgttgttaaaacaagatttataaactctccccctggtcaatataaaagtgttcctaactgtgccatgaaggtcttcactaacgaaggccccacagctttttttaaggggcttgttccctcctttttgcggctgggttcttggaatgtgatcatgttcgtctgttttgaacagctgaaaagggagctatccaaatccagacagacgatggactgtgccacctga

SEQ ID NO: 125 - RNA, 924 nt, synthetic construct
  source 1..924: mol_type = other RNA
atgggtggtctgactgctagtgatgtgcatccaacactgggggtgcaacttttcagtgctgggatcgccgcctgcctcgccgacgttataacttttccgctggataccgccaaggtgcgcctacaggttcagggcgagtgtcctacctcttcagtgattcgatataaaggcgtcttgggtaccatcactgctgtggtgaagactgaaggccgcatgaagctctacagcggcctgccagcgggcttacagaggcagatcagctccgcttccctgcgcatcggcctgtacgacacagtccaagaatttcttactgccgggaaggaaaccgctccaagtttggggtccaaaattctcgccgggctcaccacaggtggtgtcgctgtctttattggccagcctaccgaagtggtgaaggtccgactccaagctcagtcccatctccacggaattaagcctcggtacaccgggacttacaatgcttatagaatcatcgctaccacagaaggattaactggactttggaagggcacgacacccaatttgatgcggtcagtcattatcaactgcacagagttggttacctatgatctcatgaaggaagcctttgtgaaaaataacattctggcagacgacgttccatgtcacctggtttctgcattaatcgctgggttttgtgctaccgccatgtcatctcctgtcgacgttgtgaaaacccggtttataaattcaccacctggccagtataaaagcgttcccaattgtgccatgaaggtgttcacaaatgagggccctaccgcattcttcaaagggctcgtcccatcctttctccgtctaggatcctggaatgtcataatgttcgtctgttttgaacagttaaaaagggaactgagtaaatccaggcaaacaatggactgcgctacctga

SEQ ID NO: 126 - RNA, 924 nt, synthetic construct
  source 1..924: mol_type = other RNA
atgggaggtctgactgcaagcgacgtacacccaactctaggagtgcagctctttagtgcaggaattgccgcttgtctcgctgatgtcattacttttcccttggataccgctaaggtccgtctgcaggtccagggcgagtgtcctacttcatctgtcattcgttataaaggcgtattgggaaccattacagcagtcgtgaaaactgagggccgcatgaagctctacagtgggcttccagctggcctgcagcgacagatcagctcggcgtccctgcgtattggcctatacgacacagtccaagagttcctcacagctgggaaggagaccgcgccaagtttgggcagcaaaatactcgccggtctcacaacaggcggcgtggccgtattcattggacagcccactgaggtcgttaaggtacgactacaggctcagtctcatctccatgggataaaacctcgctatactggcacatacaatgcttatcgaatcatcgcaacaaccgaaggcctcacagggctgtggaaagggacgacacctaacttaatgcggtcagtgatcatcaactgcactgaactggtgacctacgatctgatgaaggaagctttcgtaaagaataacattctggcagacgacgtcccatgtcatctagtatccgcgttaatcgccggattttgtgccactgctatgtcttcccctgtcgatgttgtgaagacacgattcataaactctcctcccggccaatacaaatctgtccctaattgtgccatgaaggtcttcacaaacgagggtccaacagccttttttaaggggctcgttccttcgtttcttcggcttgggtcctggaatgtgatcatgtttgtgtgttttgaacaattgaagagagagctctctaagtccaggcagacaatggattgtgccacctag

SEQ ID NO: 127 - RNA, 924 nt, synthetic construct
  source 1..924: mol_type = other RNA
atgggtggactgacagccagcgatgtgcaccctacgctcggagttcagctctttagcgccggcatcgccgcctgccttgccgatgtcattacatttcctctagacactgctaaggtacgcctgcaggtccaaggagaatgtccaacatcatccgtaattcggtacaaaggtgtattggggaccatcaccgctgtcgtgaagactgagggccgcatgaaactttacagcgggctgccagcgggcctgcagaggcaaataagctctgcatccctgcgcatcggcctgtacgacacagttcaggaatttcttactgccggaaaggagactgcacccagtctggggagcaaaattctcgccggcctcactacaggcggcgtagccgtattcataggacaacctaccgaagtcgtcaaggtaagactgcaggcccaatcacatctgcatgggatcaagccacgctacaccgggacatacaatgcctataggattatcgccacaaccgaaggcttaactgggctctggaaggggacaacccctaatttaatgcgatcagttatcattaattgtacggaactggtgacatacgacctcatgaaggaagcttttgtaaaaaataacatcctggcagacgacgtcccatgtcatttggtctctgcattaatcgccggattttgtgccaccgctatgtcaagccctgtcgatgttgtcaagacgcggttcataaattcccctcccggccagtacaaaagtgttcctaactgtgccatgaaggtgttcaccaacgaaggccccactgcattcttcaaggggttggttccttcctttctgcggctcggatcctggaatgtcatcatgtttgtgtgtttcgagcagctgaaaagagagctctctaagtctagacaaacaatggactgcgcaacctga

SEQ ID NO: 128 - RNA, 924 nt, synthetic construct
  source 1..924: mol_type = other RNA
atggggggtctcacagcttctgacgttcatccaacactgggagtgcagctctttagcgcaggaattgccgcttgtctcgccgacgtcataacttttcccttagacaccgctaaggtccgcctgcaggtccagggcgaatgtcctacatcttcagtgatccgctataaaggcgtcttgggaaccatcactgcagtcgtgaagaccgaaggccgtatgaagctgtacagcggactgccggcagggctccagaggcaaataagttccgcatcactgcgaattggtctgtatgacaccgttcaggaattcctgacagccggaaaagaaacagcacctagcttgggtagcaaaattctggccggtctcaccacaggcggcgttgctgttttcataggacagcccaccgaggtagtaaaggtgcgacttcaggcacagagtcatctgcacggcatcaaaccgcgctacacgggcacttacaacgcttataggatcatcgccactacagaaggcctaactggtctgtggaagggcacaacacctaatttaatgcgatctgttattatcaattgtactgaactcgtaacatatgacctcatgaaggaagcctttgtaaaaaacaacatcctcgcagatgacgtcccttgccatttggtttcagctctaatagcaggattttgtgctactgccatgtcttctcccgtcgacgttgtaaagacacgattcattaattctccacctggtcagtataaatctgtccctaattgtgcaatgaaagtcttcacaaacgaaggcccaaccgcattcttcaaagggcttgtgccttcctttctgcgactcggctcctggaacgttatcatgttcgtgtgcttcgaacaactgaagcgggagctgtcaaaatccaggcagacaatggactgtgccacatga

SEQ ID NO: 129 - RNA, 924 nt, synthetic construct
  source 1..924: mol_type = other RNA
atgggcgggctcacagcaagcgacgtccatcctacactgggtgtgcagctcttcagtgcaggaatagccgcctgtctcgccgatgttattacttttcccttagatacggcaaaggtccgccttcaagtccagggcgagtgcccaacatcatctgtcatacgttataagggcgtattgggcaccatcacagccgtggtgaaaaccgaaggacggatgaagctctatagcggactgccagcagggctacaaaggcagatcagctcagcctctctacggatcggattgtacgacactgtccaagaattcctcactgcggggaaggaaacagcacccagtttggggagtaaaatcctggccggtctcaccacaggcggcgtcgccgtcttcattggacagcctacagaggtggtgaaggttcgactgcaagcccaaagtcacctgcacggaatcaagccacgctacactggaacttataacgcttacaggattatcgccaccaccgaagggctcactgggctgtggaaggggacaactcctaacttgatgcggtctgtcattatcaactgcaccgaattggtgacttacgatctcatgaaagaagcttttgtgaaaaacaacatccttgcagacgacgttccttgtcacctggtgtccgcactcatcgccggattctgtgccaccgctatgtctagccctgtcgacgttgtcaaaacacgttttataaactctccgcccggccagtacaaaagcgtccccaactgtgccatgaaggtgttcaccaacgagggtcccactgccttcttcaagggtctggttccttcttttctgcggctcggatcttggaatgtgatcatgttcgtctgctttgaacaattaaaacgggagctgtccaagagtagacaaacgatggactgcgccacgtga

SEQ ID NO: 130 - RNA, 1134 nt, synthetic construct
  source 1..1134: mol_type = other RNA
gagacccaagctggctagcggaattcgtcgactggatccggtaccgaggagatctgccgccgcgatcgccatgggcgggctgacagccagcgacgtgcacccaaccctgggagtgcagctgttttccgccggcattgctgcctgtctggctgatgtgatcactttcccactggacacagctaaagtgcggctgcaggtgcagggcgagtgcccaacaagctctgtgatcaggtacaagggcgtgctggggaccatcaccgccgtggtgaagaccgagggcagaatgaaactgtatagtggtcttcccgccggcctgcagagacagatttctagcgcctcactgcgcattggcctgtatgatacagtgcaggagtttctgactgccggcaaggaaactgctccatccctgggcagcaaaatcctggccggactcacaactggcggcgtggccgtcttcatcgggcagcccactgaggtggtgaaggtgcgcctgcaggcccagagccacttgcatgggatcaaacccagatacacaggaacctacaacgcttacagaatcatcgccaccaccgagggcctgactgggctgtggaagggcactaccccaaacctgatgcggagtgtgatcattaattgtactgagctggtgacctatgatctgatgaaggaggcctttgtgaagaacaacatcctggccgacgacgtgccttgtcacctggtgagcgccctgatcgccggcttctgcgccacagccatgagctcccccgtggacgtggtgaagacaagattcatcaatagcccacctggccagtataaatccgtgcctaattgcgccatgaaggtgttcaccaatgaggggcccaccgcctttttcaaggggctggtgccctccttcctgaggctgggcagttggaacgtgatcatgttcgtgtgtttcgaacagctgaaaagagagctgtccaagtctagacagaccatggactgcgccacatgatgacccgggagggcccgcggccgctcgagtctagagggcccgtttaaacgtctgagtgggcggcgaattcaaaaaaaaaaaaaaaaaaaaaaaaaaaaaaaaaaaaaaaaaaaaaaaaaaaaaaaaaaaaaaaaaaaaaa